SEQUENCE LISTING (4 sequences)

SEQ ID NO: 1 - protein, 138 aa, Artificial Sequence
  Chemically synthesized
MGSSHHHHHHSSGLVPRGSHMNTKKNGRLTNQLQYLQKVVLKDLWKHSFSWPFQRPVDAVKLQLPDYYTIIKNPMDLNTIKKRLENKYYAKASECIEDFNTMFSNCYLYNKPGDDIVLMAQALEKLFMQKLSQMPQEE

SEQ ID NO: 2 - protein, 135 aa, Artificial Sequence
  Chemically synthesized
MGSSHHHHHHSSGLVPRGSHMASVTEQLRHCSEILKEMLAKKHFSYAWPFYNPVDVNALGLHNYYDVVKNPMDLGTIKEKMDNQEYKDAYKFAADVRLMFMNCYKYNPPDHEVVTMARMLQDVFETHFSKIPIEP

SEQ ID NO: 3 - protein, 146 aa, Artificial Sequence
  Chemically synthesized
MGSSHHHHHHSSGLVPRGSHMNPPPPETSNPNKPKRQTNQLQYLLRVVLKTLWKHQFAWPFQQPVDAVKLNLPDYYKIIKTPMDMGTIKKRLENNYYWNAQECIQDFNTMFTNCYIYNKPGDDIVLMAEALEKLFLQKINELPTEE

SEQ ID NO: 4 - protein, 152 aa, Artificial Sequence
  Chemically synthesized
MHHHHHHSSGVDLGTENLYFQSNAKDVPDSQQHPAPEKSSKVSEQLKCCSGILKEMFAKKHAAYAWPFYKPVDVEALGLHDYCDIIKHPMDMSTIKSKLEAREYRDAQEFGADVRLMFSNCYKYNPPDHEVVAMARKLQDVFEMRFAKMPDE

(19) United States
(12) Patent Application Publication
(10) Pub. No.: US 2024/0158355 A1
(43) Pub. Date: May 16, 2024

(54) BET SUBFAMILY INHIBITORS AND METHODS USING SAME

(71) Applicant: Baylor College of Medicine, Houston, TX (US)

(72) Inventors: Martin Matzuk, Houston, TX (US); Zhifeng Yu, Houston, TX (US); Angela Ku, Houston, TX (US); Justin Anglin, Houston, TX (US); Feng Li, Houston, TX (US); Damian Young, Houston, TX (US); Ying-Chu Chen, Houston, TX (US); Melek Ucisik, Houston, TX (US); John Faver, Houston, TX (US); Stephen Palmer, Houston, TX (US); Rajesh Sharma, Houston, TX (US); Choel Kim, Houston, TX (US)

(21) Appl. No.: 18/262,037

(22) PCT Filed: Jan. 19, 2022
(86) PCT No.: PCT/US2022/012926

§ 371 Date: Jul. 19, 2023

Related U.S. Application Data
(60) Provisional application No. 63/139,555, filed on Jan. 20, 2021.

(51) Int. Cl. C07D 231/56; C07D 401/04; C07D 403/04

(52) U.S. Cl. CPC C07D 231/56; C07D 401/04; C07D 403/04

(57) ABSTRACT

In one aspect, the present disclosure provides compounds which inhibit bromodomain testis (BRDT). In some embodiments, the compounds inhibit bromodomain-2 of BRDT. In another aspect, the present disclosure provides a method of inhibiting BRDT in a male subject, the method comprising administering to the male subject a therapeutically effective amount of a compound of the disclosure. In some embodiments, the method provides a contraceptive effect in the male subject.

### CROSS-REFERENCE TO RELATED APPLICATIONS

The present application claims priority under 35 U.S.C. § 119(e) to U.S. Provisional Application No. 63/139,555, filed Jan. 20, 2021, which application is hereby incorporated herein by reference in its entirety.

### STATEMENT REGARDING FEDERALLY SPONSORED RESEARCH OR DEVELOPMENT

This invention was made with government support under HD087157 awarded by National Institutes of Health. The government has certain rights in the invention.

### SEQUENCE LISTING

The ASCII text file named “046641-7048WO1 Sequence Listing,” created on Jan. 14, 2022, comprising 5.7 Kbytes, is hereby incorporated by reference in its entirety.

### BACKGROUND OF THE DISCLOSURE

Although women have had small molecule contraceptive pill options for over 50 years, there is no effective, non-hormonal oral contraceptive pill for men. Thus, there is a need to identify and validate contraceptives that target novel proteins and structures that are not based on endocrine interventions.

BRDT is a tissue-restricted bromodomain and extraterminal (BET) subfamily member expressed in pachytene spermatocytes, diplotene spermatocytes, and round spermatids. There is a need in the art for novel compounds that inhibit BRDT and methods of using these compounds. There is also a need in the art for novel compounds that inhibit one or more members of the BET subfamily (BRD2, BRD3, BRD4, and BRDT) and methods of using these compounds. There is also a need in the art for novel compounds that inhibit specifically bind and inhibit one of the bromodomains (BD2 versus BD1) of the BET subfamily (e.g., BRD2-BD2, BRD3-BD2, BRD4-BD2, and BRDT-BD2) and methods of using these compounds. The present disclosure addresses these needs.

### BRIEF SUMMARY OF THE DISCLOSURE

In one aspect, the present disclosure provides a compound of formula (I):

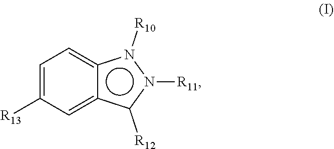

wherein:
R₁₀ is present and R₁₁ is absent, or R₁₀ is absent and R₁₁ is present,
   and when present R₁₀ and R₁₁ are each independently selected from hydrogen, deuterium, and C₁-C₁₂ alkyl;
R₁₃ is selected from optionally substituted C₆-C₁₂ aryl and optionally substituted C₄-C₁₀ heteroaryl;
R₁₃ is selected from

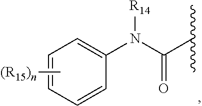

—C(═O)NHR₁₆, and optionally substituted C₄-C₁₀ heteroaryl;
R₁₄ is selected from hydrogen, deuterium, and C₁-C₁₂ alkyl;
each R₁₅ is independently selected from hydrogen, deuterium, halogen, C₁-C₁₂ alkyl, C₁-C₁₂ alkoxy, C₆-C₁₂ aryl, —CN, —CO₂H, —NHS(═O)₂CH₃, —S(═O)₂NHCH₃, —S(═O)₂NH₂, —O—(CH₂)ₘ—C₃-C₈ cycloalkyl, —NHC(═O)—R₁₇, —C(═O)NH—R₁₈, and —C(R₁₉)₃;
R₁₆ is selected from C₁-C₁₂ alkyl and optionally substituted C₄-C₁₀ heterocyclyl;
R₁₇ and R₁₈ are each independently selected from hydrogen, deuterium, C₁-C₁₂ alkyl, optionally substituted C₆-C₁₂ aryl, optionally substituted C₃-C₈ cycloalkyl, and optionally substituted C₄-C₁₀ heterocyclyl,
each R₁₉ is independently selected from fluorine, chlorine, bromine, and iodine;
m is 1, 2, 3, 4, or 5; and
n is 0, 1, 2, 3, 4, or 5;
   or a salt, solvate, stereoisomer, tautomer, or geometric isomer thereof.

The present disclosure further provides a compound of formula (I), which is selected from a compound of formula (II), formula (III), and formula (IV):

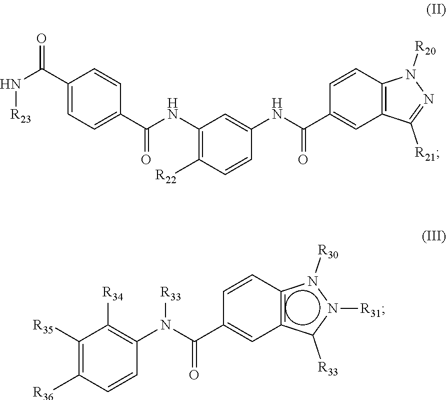

wherein the substituents in each of the compound of formula (II), formula (III), and formula (IV) are defined within the scope of the present disclosure.

The present disclosure further provides a pharmaceutical composition comprising a compound of the present disclosure and a pharmaceutically acceptable carrier.

The present disclosure further provides a method of inhibiting bromodomain testis (BRDT) in a male subject, the method comprising administering to the male subject a therapeutically effective amount of at least one compound of the present disclosure, or at least one pharmaceutical composition of the present disclosure.

### BRIEF DESCRIPTION OF THE DRAWINGS

The following detailed description of exemplary embodiments of the disclosure will be better understood when read in conjunction with the appended drawings. For the purpose of illustrating the disclosure, non-limiting embodiments are shown in the drawings. It should be understood, however, that the disclosure is not limited to the precise arrangements and instrumentalities of the embodiments shown in the drawings.

FIG. 1 depicts that the recombinant bromodomain proteins purified for the DEC-Tec selection were pure. Five micrograms His-BRDT-BD1 (lane 1), His-BRDT-BD2 (lane 2), His-BRD4-BD1 (lane 3), and His-BRD4-BD2 (lane 4) were loaded onto NuPAGE 4 to 12%, Bis-Tris gradient gel (Invitrogen) and stained with Instant blue protein stain.

FIGS. 2A-2B depicts comparisons of normalized enrichment from parallel DEC-Tec screens against the isolated bromodomains of BRDT. Selected library members are highlighted which showed significant and selective enrichment for BRDT-BD2 compared to BRDT-BD1 (FIG. 2A), and strong dependence on the presence of (+)-JQ1 (FIG. 2B). For each highlighted library member, the building blocks for cycles 1, 2, and 3 are as follows: 7 (N¹-(5-amino-2-methoxyphenyl)-N⁴-methylterephthalamide, 3-iodo-1-methyl-1H-indazole-5-carboxylic acid, and 4-(4,4,5,5-tetramethyl-1,3,2-dioxaborolan-2-yl)aniline), 9 (N¹-(5-amino-2-methylphenyl)-N⁴-methylterephthalamide, 3-iodo-1-methyl-1H-indazole-5-carboxylic acid, and 4-(4,4,5,5-tetramethyl-1,3,2-dioxaborolan-2-yl)-3-methyl-aniline), 8 (N¹-(5-amino-2-methylphenyl)-N⁴-methylterephthalamide, 3-iodo-1-methyl-1H-indazole-5-carboxylic acid, and 1-methyl-2-(4,4,5,5-tetramethyl-1,3,2-dioxaborolan-2-yl)-1H-indole), and 3 (N¹-(5-amino-2-methoxyphenyl)-N⁴-methylterephthalamide, 3-iodo-1-methyl-1H-indazole-5-carboxylic acid, and 1-methyl-2-(4,4,5,5-tetramethyl-1,3,2-dioxaborolan-2-yl)-1H-indole).

FIG. 3 depicts that compound 9 is a potent and selective inhibitor of the 2^{nd} bromodomain of BRDT and BRD4. Competitive inhibition of human BET bromodomain (BRDT-BD1, BRDT-BD2, BRD4-BD1, and BRD4-BD2) binding to biotinylated (+)-JQ1 by 9, and (+)-JQ1 using proximity detection assays. GraphPad Prism software was used to generate inhibition fitting curves and to determine IC₅₀ values.

FIG. 4 depicts that compound 9 significantly stabilized the second but not the first bromodomain of BRDT and BRD4 in the protein thermal shift stability assay. (+)-JQ1 at the same concentration was run in parallel as a positive control. Data analysis and protein melting temperature (Tm) calculation were run on a Roche Lightcycler 480 real-time PCR instrument (n=2).

FIG. 5 depicts that compound 9 selectively inhibited the second bromodomain of BRDT and BRD4 in NanoBRET target engagement assay. NanoBRET tracer competition by 9 was performed in transiently transfected HEK293 cells expressing NanoLuc-BRDT-BD2, NanoLuc-BRDT-BD1, NanoLuc-BRD4-BD2, or NanoLuc-BRD4-BD1 fusion protein. (+)-JQ1 was measured in parallel as a positive control. GraphPad Prism software was used to generate inhibition fitting curves and to determine IC₅₀ values. Compound 9 (▴) and JQ1 (●).

FIG. 6 provides a summary and comparison of BRDT and BRD4 activities for (+)-JQ1 and potent compounds from DEC-Tec hits and analogs (i.e., compounds 8-10, 15, 17, and 20-22). Legend: ^{a}11% inhibition at 10 μM; ^{b}16% inhibition at 10 μM for BRDT-BD1 and 20% inhibition at 10 μM for BRD4-BD1; ^{c}No effect at 20 μM in AlphaScreen assay; ^{d}t_{1/2} measured using liver microsomal stability assay—assay data>60 min is an extrapolated estimate and included for relative determination of half-life.

FIGS. 7A-7B depict BROMOscan bromodomain profiling of (+)-JQ1, 9 and 20 on various bromodomains. FIG. 7A: Phylogenetic tree of bromodomains demonstrating preferential compound binding of 9 and 20 for the BET subfamily BD2 domains using the BROMOscan bromodomain competition binding assay performed by the Eurofins DiscoverX Corporation, wherein: I comprises CECR2, FALZ, GCN5L2, and PCAF; II comprises BRDT(1), BRD4(1), BRD3(1), BRD2(1), BRD2(2), BRD3(2), BRD4(2), and BRDT(2); III comprises CREBBP, EP300, BRWD3(2), PHIP(2), WDR9(2), BAZ1B, BRD8(1), and BRD8(2); IV comprises BRD1, BRPF3, BRPF1, BRD7, BRD9, BAZ1A, ATAD2A, and ATAD2B; V comprises BAZ2A, BAZ2B, SP140, LOC93349, SP100, SP110, TRM33, TRIM24, and TRIM66; VI comprises MLL, and TRM28; VII comprises ASH1L, ZMYND11, TAF1L(2), FAF1(2), PRKCBP1, TAF1L(1), PHIP(1), WDR9(1), and BRWD3(1); and VIII comprises PB1(2), PB1(3), PB1(1), PB1(4), PB1(6), PB1(5), SMARCA4, and SMARCA2. FIG. 7B: BromoKdELECT dose-response curves and the calculated Kd confirmed that 9 and 20 are strong binders to the BET subfamily BD2 domains, with the highest affinity for BRDT-BD2. (+)-JQ1 binding to both BET bromodomains served as a control in these assays.

FIGS. 8A-8C depict detailed BRDT-BD2-inhibitor interactions. FIG. 8A: BRDT-BD2/9 complex. Electrostatic potential surface is shown with the secondary structure in the left and a zoom-in view of detailed interactions in the right. Compound 9 is shown in sticks. The ordered waters are shown in spheres. The hydrogen bonds are shown in dotted lines. FIG. 8B: BRDT-BD2/20 complex with one 20 bound. Electrostatic potential surface is shown with the secondary structure in the left and a zoom-in view of detailed interactions in the right. FIG. 8C: BRDT-BD2/20 complex with two 20 bound. Electrostatic potential surface is shown with the secondary structure in cartoon at the center. Zoom-in views of detailed interactions for 20 bound at the KAc pocket are shown in the left and the second site in the right.

FIGS. 9A-9B depict the two molecules captured in the BRDT-BD2/9 complex crystal. FIG. 9A: Two BD2 molecule are captured in asymmetric unit with one 9 molecule bound to each molecule. Compound 9 shown in sticks. N and C terminus are labelled. FIG. 9B: Superimposition of two BD2/9 complexes. Two molecules are nearly identical (showing an RMSD of 0.25 Å between 97 CA atoms).

FIGS. 10A-10B depict the four molecules captured in the BRDT-BD2/20 complex crystal. FIG. 10A: Four BRDT-BD2/20 complexes captured in asymmetric unit. The molecules A and B show one 20 bound at the KAc binding pocket whereas molecules C and D show additional 20 bound at the unseen site near the BC loop. Compound 20 and Jeffamine ED-2000 are shown in sticks. A zoom in view in right shows a unique crystal contact between molecule B and C that causes a different conformation of the ZA loop segment in molecules A and B. FIG. 10B: Alignment of four BRDT-BD2/20 complexes captured in the asymmetric unit. Four molecules show nearly identical structures except for their ZA loops. Molecules C and D show different conformations at the ZA loop. A zoom-in view of the ZA loop with key residues showing in sticks.

FIGS. 11A-11B depict the H-bonding interactions between the KAc binding site residues of BRDT-BD2 and the small molecule inhibitors. To better show these, the co-crystal structures of 9 and 20 were subjected to a restrained geometry optimization after addition of hydrogens, estimation of protonation states, and optimization of hydrogen bond networks via the Schrodinger Suite of Programs. The residues interacting with the ligand are shown in stick representation and labeled. The water molecules are shown as dots. The black dashed lines represent H-bonds. The point of view angle was adjusted to display the H-bonding interactions as clearly as possible. FIG. 11A: 9. FIG. 11B: 20.

FIGS. 12A-12B depict structure comparisons. FIG. 12A: Superposition of the BD2/9 and BD2/20 complexes. The BD2/9 complex is superimposed with the BD2/20 complex. Only the Electrostatic potential surface belonging to the BD2/20 complex is shown. FIG. 12B: Superposition with the BRDT-BD1/JQ1 complex. The BRDT-BD2/9 complex is superimposed with the BRDT-BD1/JQ1 complex (PDB ID: 4FLP). Key interacting residues are shown in sticks. BD2 specific contact residues and corresponding BD1 residues are labeled.

FIG. 13 depicts the modeled (M) and co-crystal (C) poses of 9. The modeled pose was obtained through an extensive small molecule docking study using a homology model of human BRDT-BD2 containing the ZA channel waters of bromodomains (shown as dots), as these water molecules frequently play a role in binding inside the KAc pocket. The aniline—NH₂ group of both 9 and 20 successfully replaced a ZA channel water, which allowed the ligands to penetrate into the KAc pocket very deeply. This water molecule usually bridges the conserved Y308 (BRDT-BD2 numbering) and the KAc of the acetylated histone or small molecule BET inhibitors. The computational modeling was performed using the Schrodinger Suite of Programs.

### DETAILED DESCRIPTION OF THE DISCLOSURE

Based on proof-of-principle knockout and in vivo chemical biology studies, bromodomain testis (BRDT) is a germ-cell target for small-molecule male contraceptives. The present disclosure provides, in one aspect, compounds that inhibit one or more members of the bromodomain and extraterminal (BET) subfamily. In some embodiments, the compounds selectively inhibit bromodomain 2 (BD2) of the BET subfamily over bromodomain 1 (BD1). In some embodiments, the compounds inhibit BRDT, BRD4, or a combination thereof. In some embodiments, the compounds inhibit BRDT, BRD4, BRD2, BRD3, or a combination thereof. In some embodiments, the compounds inhibit BRDT-BD2, BRD4-BD2, or a combination thereof. In some embodiments, the compounds inhibit BRDT-BD2, BRD4-BD2, BRD3-BD2, BRD2-BD2, or a combination thereof. In some embodiments, the compounds do not inhibit BRDT-BD1 and/or BRD4-BD1. In some embodiments, the compounds do not inhibit BRDT-BD1, BRD4-BD1, BRD3-BD1, BRD2-BD1, or a combination thereof.

The present disclosure further provides a method of inhibiting BRDT in a male subject, the method comprising administering to the male subject a therapeutically effective amount of a compound of the disclosure. In some embodiments, the compound of the disclosure is administered to the male subject at a dosage and dosage timing necessary to provide a contraceptive effect in the male subject. In some embodiments, the compound is orally administered to the male subject.

Deletions of either BD1 or both BD1 and BD2 in the mouse BRDT gene result in male sterility, indicating that BRDT is a testis-specific target for male contraception. (+)-JQ1, a selective small-molecule inhibitor of the BET subfamily, has been shown to induce a complete and reversible contraceptive effect in mice. Specifically, (+)-JQ1 easily crosses the blood-testis boundary (100% bioavailable) to specifically inhibit BRDT function in spermatocytes and spermatids, and reduce spermatozoa number and motility. Daily intraperitoneal or subcutaneous injections of (+)-JQ1 resulted in grossly smaller testes compared to control males. These proof-of-principle small-molecule studies have validated BRDT as a spermatogenic-specific contraceptive target and have shown that (+)-JQ1 is the first lead compound targeting the male germ cell for reversible contraception.

Despite the above findings, there are two features of (+)-JQ1 that limit its contraceptive development. First, the half-life of (+)-JQ1 is too short and it is metabolized rapidly. Second, (+)-JQ1 binds to the somatic bromodomain family member BRD4 with higher affinity than for BRDT, which influences its side effect profile. Thus, to identify highly potent and selective inhibitors of BRDT for male contraception and to find molecules that bind selectively to the individual bromodomains of BRDT, a DNA-encoded chemistry technology (DEC-Tec) platform was developed and applied for a drug screening campaign.

While high-throughput screening (HTS) with million-compound drug libraries is extensively utilized by pharmaceutical companies in early drug development, DNA-encoded chemical libraries are an alternative technology for ligand discovery that addresses the limitations and economic shortcomings of HTS. DEC-Tec has been utilized to develop potent and specific small-molecule inhibitors to various target proteins by several pharmaceutical companies and academic institutions, including EPHX2, RIP1 kinase, Mycobacterium tuberculosis InhA, autotaxin, BTK, β₂-adrenergic receptor, thrombin, and OXA-48 carbapenemase, and so forth.

Recent studies have shown the potential to identify selective inhibitors of BET bromodomains for cancer and immunoinflammation. BET proteins are believed to be tethered to chromatin predominantly via the first bromodomain, based on results with RVX-208 (higher affinity BD2 inhibitor than BD1 inhibitor), that could not cause significant displacement of BRD4 from chromatin.

The recent identification of a better BD2-specific inhibitor, GSK046 with >300 fold selectivity over BD1, revealed an important role of the second bromodomain in the recruitment of BET proteins for the induction of gene expression, particularly in the context of immunoinflammation. An improved analog of GSK046 (GSK620) that exhibited similar selective BRD4-BD2 inhibition provided better resolution of immunoinflammatory diseases in their preclinical models, suggesting a more effective therapeutic option.

Alternatively, several published BD2 selective inhibitors provided contradictory results in various cancer models, based on their variable BD2 selectivity and the cellular context of the cancer models used. However, ABBV-744, a small molecule inhibitor with 95-290-fold selectivity for BRD4-BD2, showed similar anti-cancer efficacy with improved tolerability in prostate cancer xenografts compared to dual-bromodomain BET inhibitors. These recent results suggest the potential for BD2-specific inhibitors to avoid the toxicity associated with recent BET inhibitors that have advanced to clinical trials.

In the present studies, the discovery and development of an inhibitor series both potent and highly selective for the BD2 domains of BET subfamily members versus BD1 domains is demonstrated. Initial hits from the DEC-Tec screening underwent pharmacophore identification, and high resolution co-crystal structures between human BRDT-BD2 and two high affinity analogs were solved. Optimization of microsomal stability properties of the exemplar compounds in vitro and in vivo is also described.

Reference will now be made in detail to certain embodiments of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In the methods described herein, the acts can be carried out in any order, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

### Definitions

The term “about” as used herein can allow for a degree of variability in a value or range, for example, within 10%, within 5%, or within 1% of a stated value or of a stated limit of a range, and includes the exact stated value or range.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. All publications, patents, and patent documents referred to in this document are incorporated by reference herein in their entirety, as though individually incorporated by reference.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to—C≡CH, —C≡C(CH₃), —C≡C(CH₂CH₃), —CH₂C≡CH, —CH₂C≡C(CH₃), and —CH₂C≡C(CH₂CH₃) among others.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “aminoalkyl” as used herein refers to amine connected to an alkyl group, as defined herein. The amine group can appear at any suitable position in the alkyl chain, such as at the terminus of the alkyl chain or anywhere within the alkyl chain.

The term “aralkyl” as used herein refers to alkyl groups as defined herein in which a hydrogen or carbon bond of an alkyl group is replaced with a bond to an aryl group as defined herein. Representative aralkyl groups include benzyl and phenylethyl groups and fused (cycloalkylaryl)alkyl groups such as 4-ethyl-indanyl. Aralkenyl groups are alkenyl groups as defined herein in which a hydrogen or carbon bond of an alkyl group is replaced with a bond to an aryl group as defined herein.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

As used herein, the term “C₆₋₁₀— C₆₋₁₀ biaryl” means a C₆₋₁₀ aryl moiety covalently bonded through a single bond to another C₆₋₁₀ aryl moiety. The C₆₋₁₀ aryl moiety can be any of the suitable aryl groups described herein. Non-limiting example of a C₆₋₁₀— C₆₋₁₀ biaryl include biphenyl and binaphthyl.

As used herein, the term “composition” or “pharmaceutical composition” refers to a mixture of at least one compound described herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary and topical administration.

The term “cycloalkyl” as used herein refers to cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7. Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted, or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-1-dibenz[b,f]azepine (5H-1-dibenz[b,f]azepin-1-yl, 5H-1-dibenz[b,f]azepine-2-yl, 5H-1-dibenz[b,f]azepine-3-yl, 5H-1-dibenz[b,f]azepine-4-yl, 5H-1-dibenz[b,f]azepine-5-yl), 10,11-dihydro-5H-dibenz[b,f]azepine (10,11-dihydro-5H-1-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heteroarylalkyl” as used herein refers to alkyl groups as defined herein in which a hydrogen or carbon bond of an alkyl group is replaced with a bond to a heteroaryl group as defined herein.

As used herein, the term “C₆₋₁₀-5-6 membered heterobiaryl” means a C₆₋₁₀ aryl moiety covalently bonded through a single bond to a 5- or 6-membered heteroaryl moiety. The C₆₋₁₀ aryl moiety and the 5-6-membered heteroaryl moiety can be any of the suitable aryl and heteroaryl groups described herein. Non-limiting examples of a C₆₋₁₀-5-6 membered heterobiaryl include:

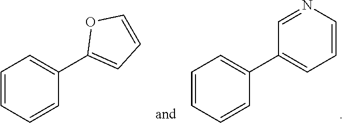 [SMILES: c1ccc(-c2ccco2)cc1 ; c1ccc(-c2cccnc2)cc1]

When the C₆₋₁₀-5-6 membered heterobiaryl is listed as a substituent (e.g., as an “R” group), the C₆₋₁₀-5-6 membered heterobiaryl is bonded to the rest of the molecule through the C₆₋₁₀ moiety.

As used herein, the term “5-6 membered- C₆₋₁₀ heterobiaryl” is the same as a C₆₋₁₀-5-6 membered heterobiaryl, except that when the 5-6 membered C₆₋₁₀ heterobiaryl is listed as a substituent (e.g., as an “R” group), the 5-6 membered- C₆₋₁₀ heterobiaryl is bonded to the rest of the molecule through the 5-6-membered heteroaryl moiety.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise, a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “heterocyclylalkyl” as used herein refers to alkyl groups as defined herein in which a hydrogen or carbon bond of an alkyl group as defined herein is replaced with a bond to a heterocyclyl group as defined herein. Representative heterocyclyl alkyl groups include, but are not limited to, furan-2-yl methyl, furan-3-yl methyl, pyridine-3-yl methyl, tetrahydrofuran-2-yl ethyl, and indol-2-yl propyl.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, wherein X¹, X², and X³ are all different, wherein X¹ and X² are the same but X³ is different, and other analogous permutations.

The term “(+)-JQ1” or “JQ1” as used herein refers to

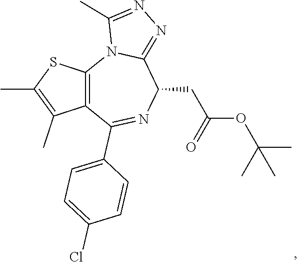 [SMILES: Cc1sc2c(c1C)C(c1ccc(Cl)cc1)=N[C@@H](CC(=O)OC(C)(C)C)c1nnc(C)n1-2]

tert-butyl (S)-2-(4-(4-chlorophenyl)-2,3,9-trimethyl-6H-thieno[3,2-f][1,2,4]triazolo[4,3-a][1,4]diazepin-6-yl)acetate.

The term “monovalent” as used herein refers to a substituent connecting via a single bond to a substituted molecule. When a substituent is monovalent, such as, for example, F or Cl, it is bonded to the atom it is substituting by a single bond.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, μ-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The term “solvent” as used herein refers to a liquid that can dissolve a solid, liquid, or gas. Non-limiting examples of solvents are silicones, organic compounds, water, alcohols, ionic liquids, and supercritical fluids.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The term “thioalkyl” as used herein refers to a sulfur atom connected to an alkyl group, as defined herein. The alkyl group in the thioalkyl can be straight chained or branched. Examples of linear thioalkyl groups include but are not limited to thiomethyl, thioethyl, thiopropyl, thiobutyl, thiopentyl, thiohexyl, and the like. Examples of branched alkoxy include but are not limited to iso-thiopropyl, sec-thiobutyl, tert-thiobutyl, iso-thiopentyl, iso-thiohexyl, and the like. The sulfur atom can appear at any suitable position in the alkyl chain, such as at the terminus of the alkyl chain or anywhere within the alkyl chain.

The terms “treat,” “treating” and “treatment,” as used herein, means reducing the frequency or severity with which symptoms of a disease or condition are experienced by a subject by virtue of administering an agent or compound to the subject.

Throughout this disclosure, various aspects of the disclosure can be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### Compounds

In one aspect, the present disclosure relates to an inhibitor of bromodomain testis (BRDT). In some embodiments, the BRDT inhibitor is selective for BRDT over other members of the bromodomain and extraterminal (BET) subfamily. In some embodiments, the BRDT inhibitor is a selective BRDT-BD1 inhibitor or a selective BRDT-BD2 inhibitor. In some embodiments, the BRDT inhibitor inhibits both BRDT-BD1 and BRDT-BD2. In some embodiments, the BRDT inhibitor binds to BRDT-BD1 and/or BRDT-BD2 with a higher affinity than the BRDT inhibitor binds to BRD2, BRD3, and/or BRD4. In some embodiments, the BRDT inhibitor binds to BRDT-BD2 and BRD4-BD2. In some embodiments, the BRDT inhibitor binds to BRDT-BD2.

In some embodiments, the BRDT inhibitor is a compound of formula (I):

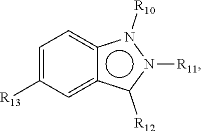

wherein:
R₁₀ is present and R₁₁ is absent, or R₁₀ is absent and R₁₁ is present,
   and when present R₁₀ and R₁₁ are each independently selected from hydrogen, deuterium, and C₁-C₁₂ alkyl;
R₁₂ is selected from optionally substituted C₆-C₁₂ aryl and optionally substituted C₄-C₁₀ heteroaryl;
R₁₃ is selected from

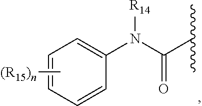

—C(═O)NHR₁₆, and optionally substituted C₄-C₁₀ heteroaryl;
R₁₄ is selected from hydrogen, deuterium, and C₁-C₁₂ alkyl;
each R₁₅ is independently selected from hydrogen, deuterium, C₁-C₁₂ alkyl, C₁-C₁₂ alkoxy, C₆-C₁₂ aryl, —CN, —CO₂H, —NHS(═O)₂CH₃, —S(═O)₂NHCH₃, —S(═O)₂NH₂, —O—(CH₂)ₘ—C₃-C₈ cycloalkyl, —NHC(═O)—R₁₇, —C(═O)NH—R₁₈, —C(R₁₉)₃, and halogen;
R₁₆ is selected from C₁-C₁₂ alkyl and optionally substituted C₄-C₁₀ heterocyclyl;
R₁₇ and R₁₈ are each independently selected from hydrogen, deuterium, C₁-C₁₂ alkyl, optionally substituted C₆-C₁₂ aryl, optionally substituted C₃-C₈ cycloalkyl, and optionally substituted C₄-C₁₀ heterocyclyl,
each R₁₉ is independently selected from fluorine, chlorine, bromine, and iodine;
m is 1, 2, 3, 4, or 5; and
n is 0, 1, 2, 3, 4, or 5;
   or a salt, solvate, stereoisomer, tautomer, or geometric isomer thereof.

In some embodiments, the compound of formula (I) is

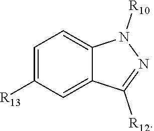

In some embodiments, the compound of formula (I) is

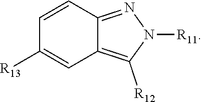

In some embodiments, the optional substituent is independently selected from C₁-C₁₂ alkyl, C₁-C₁₂ alkoxy, C₁-C₁₂ acyl, C₁-C₁₂ acyloxy, amide (e.g. —C(═O)NH₂, —C(═O)NHCH₃, - and NHC(═O)CH₃), —NH₂, —CO₂H, —CF₃, —CBr₃, —CCl₃, and —CI₃.

In some embodiments, R₁₀ is present and is C₁-C₁₂ alkyl. In some embodiments, R₁₀ is present and selected from methyl, ethyl, and isopropyl. In other embodiments, R₁₀ is absent and R₁₁ is present. In some embodiments, R₁₀ is absent and R₁₁ is methyl.

In some embodiments, R₁₂ is selected from phenyl, indolyl, pyridyl, and pyrimidyl, which are each optionally substituted with one or more substituents selected from morpholine, piperazine, halogen, C₁-C₁₂ alkyl, amide, C₁-C₁₂ alkoxy, C₆-C₁₂ benzyloxy, C₆-C₁₂ aryloxy, and/or —NH₂. In some embodiments, the optional substituent is selected from morpholine, piperazine, methyl, methoxy, benzyloxy, chlorine, —NHC(═O)CH₃, —NH₂, and combinations thereof.

In some embodiments, R₁₂ is

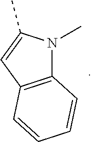

In some embodiments, R₁₂ is

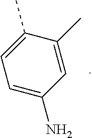

In some embodiments, R₁₂ is

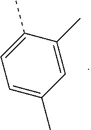

In some embodiments, R₁₂ is

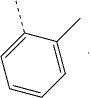

In some embodiments, R₁₂ is

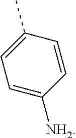

In some embodiments, R₁₂ is

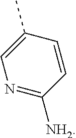

In some embodiments, R₁₂ is

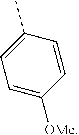

In some embodiments, R₁₂ is

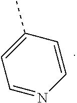

In some embodiments, R₁₂ is

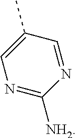

In some embodiments, R₁₂ is

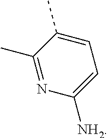

In some embodiments, R₁₂ is

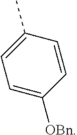

In some embodiments, R₁₂ is

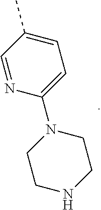

In some embodiments, R₁₂ is

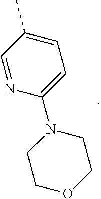

In some embodiments, R₁₂ is

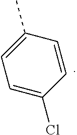

In some embodiments, R₁₂ is

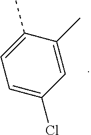

In some embodiments, R₁₂ is

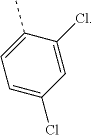

In some embodiments, R₁₂ is

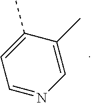

In some embodiments, R₁₂ is

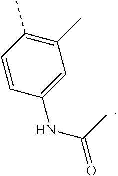

In some embodiments, R₁₂ is

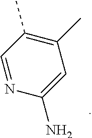

In some embodiments, R₁₃ is

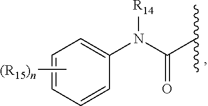

wherein one R₁₅ group is hydrogen or deuterium and four R₁₅ groups are not hydrogen or deuterium. In some embodiments, two R₁₅ groups are independently hydrogen or deuterium and three R₁₅ groups are not hydrogen or deuterium. In other embodiments, three R₁₅ groups are independently hydrogen or deuterium and two R₁₅ groups are not hydrogen or deuterium. In yet other embodiments, four R₁₅ groups are independently hydrogen or deuterium and one R₁₅ group is not hydrogen or deuterium. In yet other embodiments, five R₁₅ groups are independently hydrogen or deuterium.

In some embodiments, each R₁₅ group that is not hydrogen or deuterium is selected from methyl, methoxy, ethoxy,

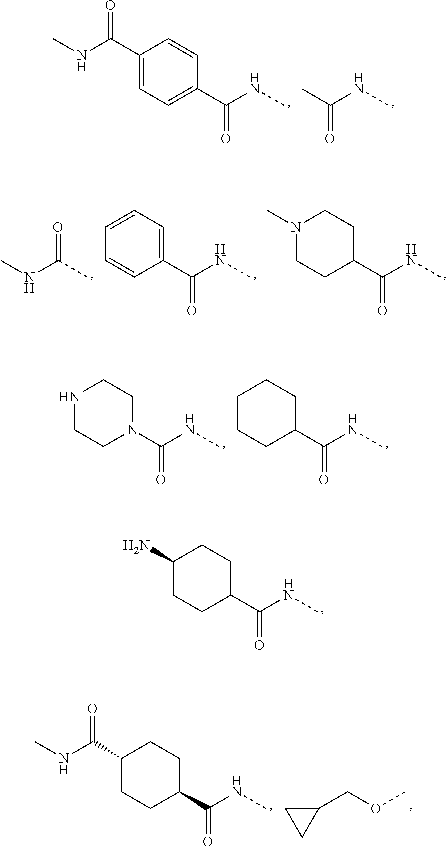

phenyl, —CO₂H, —CN, —C(═O)NH₂, —NHS(═O)₂CH₃, —S(═O)₂NHCH₃, —S(═O)₂NH₂, —CF₃, —F, —Cl, —Br, and —I.

In some embodiments, R₁₅ is methyl. In some embodiments, R₁₅ is methoxy. In some embodiments, R₁₅ is ethoxy. In some embodiments, R₁₅ is

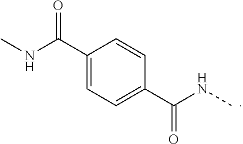

In some embodiments, R₁₅ is

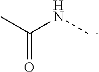

In some embodiments, R₁₅ is

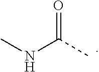

In some embodiments, R₁₅ is

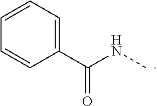

In some embodiments, R₁₅ is

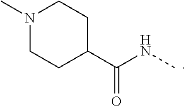

In some embodiments, R₁₅ is

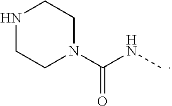

In some embodiments, R₁₅ is

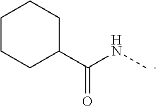

In some embodiments, R₁₅ is

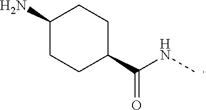

In some embodiments, R₁₅ is

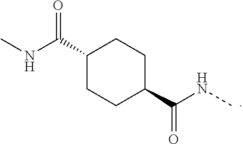

In some embodiments, R₁₅ is

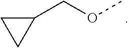

In some embodiments, R₁₅ is phenyl. In some embodiments, R₁₅ is —CO₂H. In some embodiments, R₁₅ is —CN. In some embodiments, R₁₅ is —C(═O)NH₂. In some embodiments, R₁₅ is —NHS(═O)₂CH₃. In some embodiments, R₁₅ is —S(═O)₂NHCH₃. In some embodiments, R₁₅ is —S(═O)₂NH₂. In some embodiments, R₁₅ is —CF₃. In some embodiments, R₁₅ is —F. In some embodiments, R₁₅ is —Cl. In some embodiments, R₁₅ is —Br. In some embodiments, R₁₅ is —I.

In some embodiments, R₁₃ is

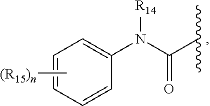

wherein R₁₄ is hydrogen. In other embodiments, R₁₃ is

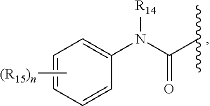

wherein R₁₄ is C₁-C₁₂ alkyl. In some embodiments, R₁₃ is

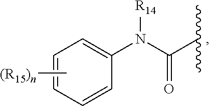

wherein R₁₄ is methyl.

In other embodiments, R₁₃ is —C(═O)NH—R₁₆, wherein R₁₆ is an optionally substituted saturated C₄-C₁₀ heterocyclyl. In certain embodiments, R₁₆ is an optionally substituted piperidine. In some embodiments, R₁₆ is

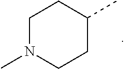

In some embodiments, R₁₆ is

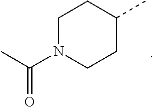

In some embodiments, R₁₆ is

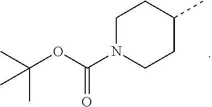

In some embodiments, R₁₆ is

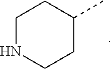

In yet another embodiment, R₁₃ is an optionally substituted C₄-C₁₀ heteroaryl. In some embodiments, R₁₃ is optionally substituted pyrimidyl. In some embodiments, R₁₃ is optionally substituted pyridyl. In certain embodiments, R₁₃ is

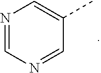

In certain embodiments, R₁₃ is

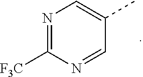

In certain embodiments, R₁₃ is

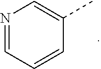

In certain embodiments, R₁₃ is

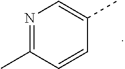

In certain embodiments, R₁₃ is

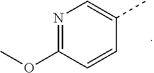

In some embodiments, the compound of formula (I) is a compound of formula (II):

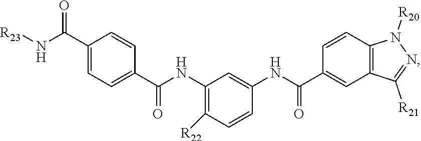

wherein
R₂₀ is C₁-C₁₂ alkyl;
R₂₁ is selected from C₆-C₁₂ aryl and C₄-C₁₀ heteroaryl, each of which is optionally substituted with one or more substituents selected from C₁-C₁₂ alkyl, C₁-C₁₂ alkoxy, C₆-C₁₂ benzyloxy, C₄-C₁₀ heterocyclyl, halogen, and —NH₂;
R₂₂ is selected from C₁-C₁₂ alkyl and C₁-C₁₂ alkoxy; and
R₂₃ is C₁-C₁₂ alkyl.

In some embodiments, R₂₀ is methyl. In other embodiments, R₂₀ is ethyl. In yet other embodiments, R₂₀ is isopropyl.

In some embodiments, R₂₁ is selected from phenyl, indole, pyridine, and pyrimidine, each of which is independently optionally substituted with one or more substituents selected from C₁-C₁₂ alkyl, C₁-C₁₂ alkoxy, C₆-C₁₂ benzyloxy, C₆-C₁₂ aryloxy, C₄-C₁₀ heterocyclyl, halogen, and —NH₂. In some embodiments, the optional substituent is methyl, methoxy, benzyloxy, morpholine, piperazine, chlorine, and/or —NH₂. In some embodiments, R₂₁ is

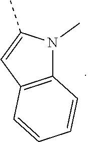

In some embodiments, R₂₁ is

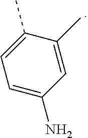

In some embodiments, R₂₁ is

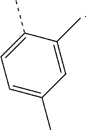

In some embodiments, R₂₁ is

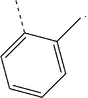

In some embodiments, R₂₁ is

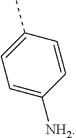

In some embodiments, R₂₁ is

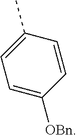

In some embodiments, R₂₁ is

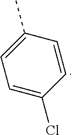

In some embodiments, R₂₁ is

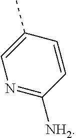

In some embodiments, R₂₁ is

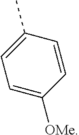

In some embodiments, R₂₁ is

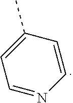

In some embodiments, R₂₁ is

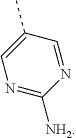

In some embodiments, R₂₁ is

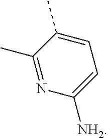

In some embodiments, R₂₁ is

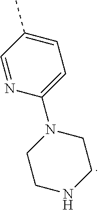

In some embodiments, R₂₁ is

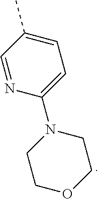

In some embodiments, R₂₁ is

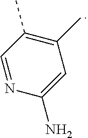

In some embodiments, R₂₂ is C₁-C₁₂ alkyl. In some embodiments, R₂₂ is methyl. In other embodiments, R₂₂ is C₁-C₁₂ alkoxy. In some embodiments, R₂₂ is methoxy.

In some embodiments, R₂₃ is methyl.

In some embodiments, the compound of formula (II) is

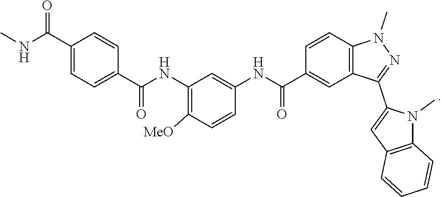

In some embodiments, the compound of formula (II) is

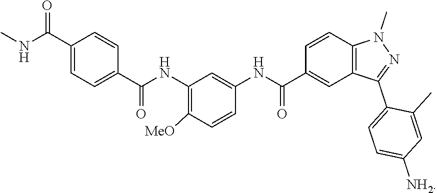

In some embodiments, the compound of formula (II) is

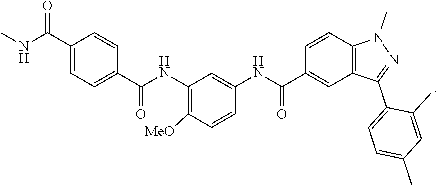

In some embodiments, the compound of formula (II) is

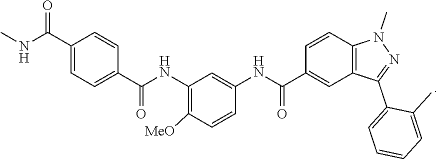

In some embodiments, the compound of formula (II) is

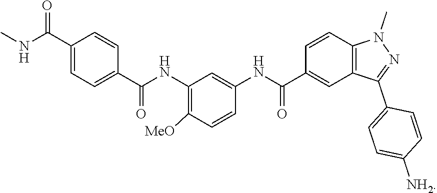 [SMILES: CNC(=O)c1ccc(C(=O)Nc2cc(NC(=O)c3ccc4c(c3)c(-c3ccc(N)cc3)nn4C)ccc2OC)cc1]

In some embodiments, the compound of formula (II) is

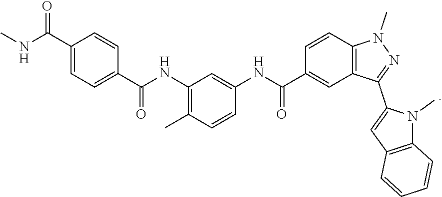

In some embodiments, the compound of formula (II) is

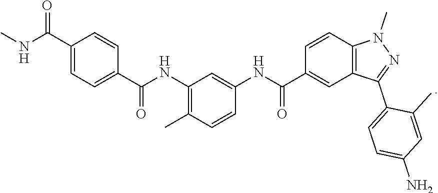

In some embodiments, the compound of formula (II) is

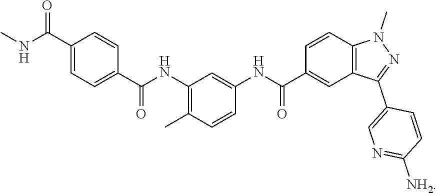 [SMILES: CNC(=O)c1ccc(C(=O)Nc2cc(NC(=O)c3ccc4c(c3)c(-c3ccc(N)nc3)nn4C)ccc2C)cc1]

In some embodiments, the compound of formula (II) is

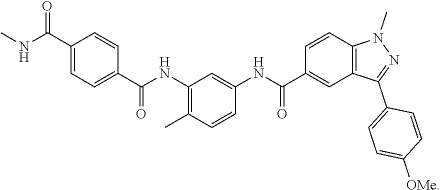 [SMILES: CNC(=O)c1ccc(C(=O)Nc2cc(NC(=O)c3ccc4c(c3)c(-c3ccc(OC)cc3)nn4C)ccc2C)cc1]

In some embodiments, the compound of formula (II) is

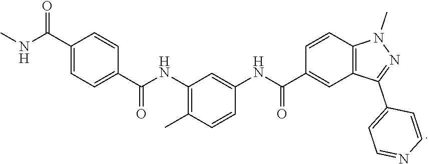 [SMILES: CNC(=O)c1ccc(C(=O)Nc2cc(NC(=O)c3ccc4c(c3)c(-c3ccncc3)nn4C)ccc2C)cc1]

In some embodiments, the compound of formula (II) is

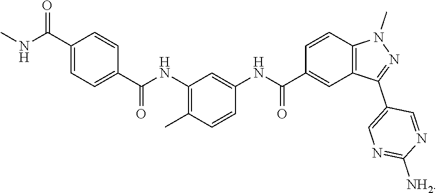 [SMILES: CNC(=O)c1ccc(C(=O)Nc2cc(NC(=O)c3ccc4c(c3)c(-c3cnc(N)nc3)nn4C)ccc2C)cc1]

In some embodiments, the compound of formula (II) is

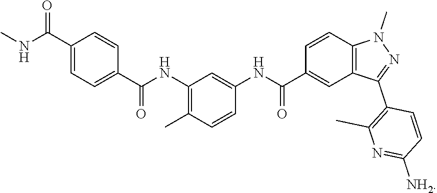 [SMILES: CNC(=O)c1ccc(C(=O)Nc2cc(NC(=O)c3ccc4c(c3)c(-c3ccc(N)nc3C)nn4C)ccc2C)cc1]

In some embodiments, the compound of formula (II) is

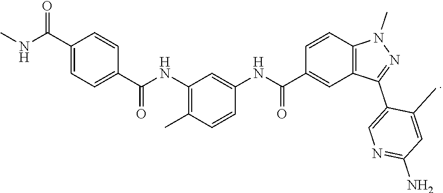

In some embodiments, the compound of formula (II) is

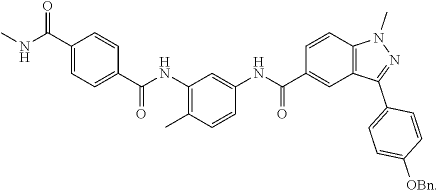 [SMILES: CNC(=O)c1ccc(C(=O)Nc2cc(NC(=O)c3ccc4c(c3)c(-c3ccc(OCc5ccccc5)cc3)nn4C)ccc2C)cc1]

In some embodiments, the compound of formula (II) is

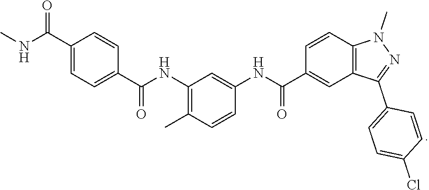 [SMILES: CNC(=O)c1ccc(C(=O)Nc2cc(NC(=O)c3ccc4c(c3)c(-c3ccc(Cl)cc3)nn4C)ccc2C)cc1]

In some embodiments, the compound of formula (II) is

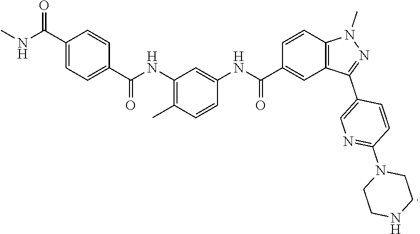 [SMILES: CNC(=O)c1ccc(C(=O)Nc2cc(NC(=O)c3ccc4c(c3)c(-c3ccc(N5CCNCC5)nc3)nn4C)ccc2C)cc1]

In some embodiments, the compound of formula (II) is

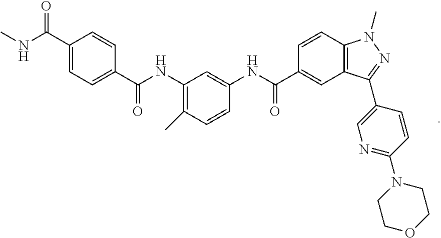 [SMILES: CNC(=O)c1ccc(C(=O)Nc2cc(NC(=O)c3ccc4c(c3)c(-c3ccc(N5CCOCC5)nc3)nn4C)ccc2C)cc1]

In other embodiments, the compound of formula (I) is a compound of formula (III):

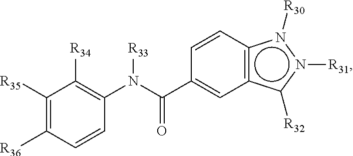

wherein:
R₃₀ is present and R₃₁ is absent, or R₃₀ is absent and R₃₁ is present,
   and when present R₃₀ and R₃₁ are each independently C₁-C₁₂ alkyl;
R₃₂ is selected from C₆-C₁₂ aryl and C₄-C₁₀ heteroaryl, each of which is independently optionally substituted with one or more substituents selected from C₁-C₁₂ alkyl, C₁-C₁₂ alkoxy, halogen, amide, and —NH₂;
R₃₃ is selected from hydrogen, deuterium, and C₁-C₁₂ alkyl;
R₃₄, R₃₅, and R₃₆ are each independently selected from hydrogen, deuterium, C₁-C₁₂ alkyl, C₁-C₁₂ alkoxy, C₆-C₁₂ aryl, —CN, —CO₂H, —NHS(═O)²CH₃, —S(═O)²NHCH₃, —S(═O)₂NH₂, —O—(CH₂)ₘ—C₃-C₈ cycloalkyl, —C(═O)—NH—R₃₇, —NHC(═O)—R₃₈, —C(R₃₉)₃, and halogen;

R₃₇ and R₃₈ are each independently selected from hydrogen, deuterium, optionally substituted C₁-C₁₂ alkyl, optionally substituted C₆-C₁₂ aryl, optionally substituted C₃-C₈ cycloalkyl, optionally substituted C₄-C₁₀ heterocyclyl,

each R₃₉ is independently selected from fluorine, chlorine, bromine, and iodine; and

m is 1, 2, 3, 4, or 5.

In some embodiments, the compound of formula (III) is

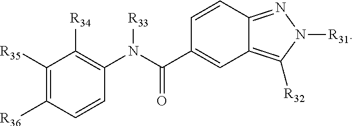

In some embodiments, the compound of formula (III) is

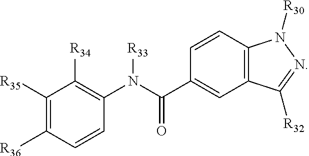

In some embodiments, the C₆-C₁₂ aryl in R₃₇ or R₃₈ is selected from the group consisting of C₁-C₁₂ alkyl, C₁-C₁₂ alkoxy, halogen, and —NH₂.

In some embodiments, R₃₀ is present and is C₁-C₁₂ alkyl. In some embodiments, R₃₀ is present and selected from methyl, ethyl, and isopropyl. In other embodiments, R₃₀ is absent and R₃₁ is present. In some embodiments, R₃₀ is absent and R₃₁ is methyl.

In some embodiments, R₃₂ is selected from phenyl, indolyl, and pyridyl, each of which is independently optionally substituted with one or more substituents selected from C₁-C₁₂ alkyl, C₁-C₁₂ alkoxy, halogen, amide, and —NH₂. In some embodiments, the optional substituent is methyl, methoxy, chlorine, —NHC(═O)CH₃, and/or —NH₂. In some embodiments, R₃₂ is

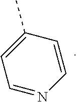

In some embodiments, R₃₂ is

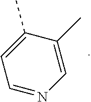

In some embodiments, R₃₂ is

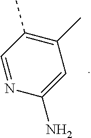

In some embodiments, R₃₂ is

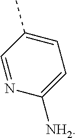

In some embodiments, R₃₂ is

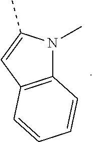

In some embodiments, R₃₂ is

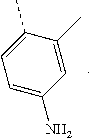

In some embodiments, R₃₂ is

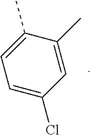

In some embodiments, R₃₂ is

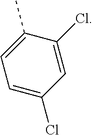

In some embodiments, R₃₂ is

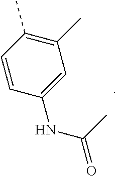

In some embodiments, R₃₃ is hydrogen. In other embodiments, R₃₃ is methyl.

In some embodiments, R₃₄ is hydrogen. In other embodiments, R₃₄ is selected from methyl, methoxy, —C(═O)NH₂, fluorine, and chlorine.

In some embodiments, R₃₅ is hydrogen. In other embodiments, R₃₅ is methyl. In other embodiments, R₃₅ is methoxy. In other embodiments, R₃₅ is phenyl. In other embodiments, R₃₅ is fluorine. In other embodiments, R₃₅ is chlorine. In other embodiments, R₃₅ is bromine. In other embodiments, R₃₅ is iodine, —CO₂H. In other embodiments, R₃₅ is —C(═O)NH₂. In other embodiments, R₃₅ is —NHS(═O)₂CH₃. In other embodiments, R₃₅ is —S(═O)₂NHCH₃. In other embodiments, R₃₅ is —S(═O)₂NH₂. In other embodiments, R₃₅ is —CF₃. In other embodiments, R₃₅ is —CN. In other embodiments, R₃₅ is

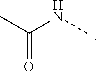

In other embodiments, R₃₅ is

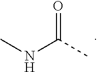

In other embodiments, R₃₅ is

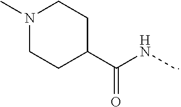

In other embodiments, R₃₅ is

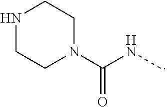

In other embodiments, R₃₅ is

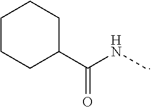

In other embodiments, R₃₅ is

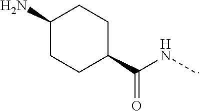

In other embodiments, R₃₅ is

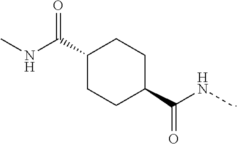

In other embodiments, R₃₅ is

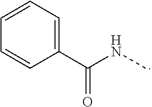

In some embodiments, R₃₆ is hydrogen. In other embodiments, R₃₆ is methyl. In other embodiments, R₃₆ is methoxy. In other embodiments, R₃₆ is ethoxy. In other embodiments, R₃₆ is —CF₃. In other embodiments, R₃₆ is fluorine. In other embodiments, R₃₆ is -chlorine. In other embodiments, R₃₆ is

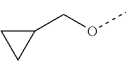

In some embodiments, the compound of formula (III) is

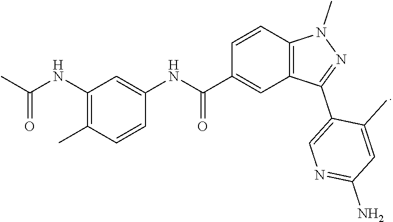 [SMILES: CC(=O)Nc1cc(NC(=O)c2ccc3c(c2)c(-c2cnc(N)cc2C)nn3C)ccc1C]

In some embodiments, the compound of formula (III) is

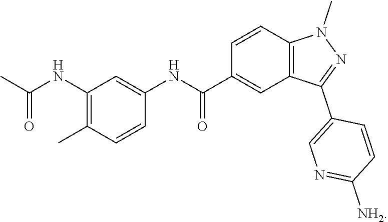 [SMILES: CC(=O)Nc1cc(NC(=O)c2ccc3c(c2)c(-c2ccc(N)nc2)nn3C)ccc1C]

In some embodiments, the compound of formula (III) is

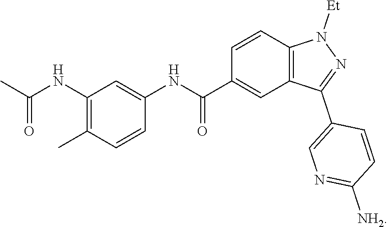 [SMILES: CCn1nc(-c2ccc(N)nc2)c2cc(C(=O)Nc3ccc(C)c(NC(C)=O)c3)ccc21]

In some embodiments, the compound of formula (III) is

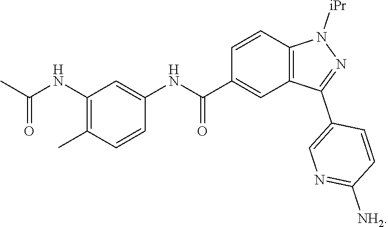 [SMILES: CC(=O)Nc1cc(NC(=O)c2ccc3c(c2)c(-c2ccc(N)nc2)nn3C(C)C)ccc1C]

In some embodiments, the compound of formula (III) is

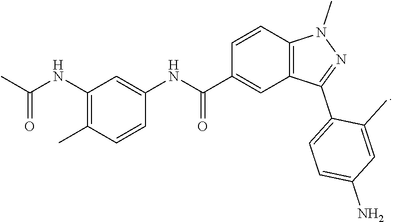 [SMILES: CC(=O)Nc1cc(NC(=O)c2ccc3c(c2)c(-c2ccc(N)cc2C)nn3C)ccc1C]

In some embodiments, the compound of formula (III) is

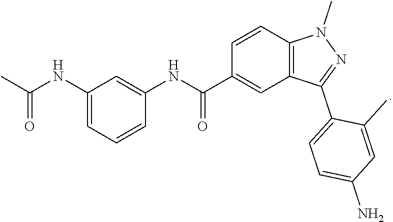 [SMILES: CC(=O)Nc1cccc(NC(=O)c2ccc3c(c2)c(-c2ccc(N)cc2C)nn3C)c1]

In some embodiments, the compound of formula (III) is

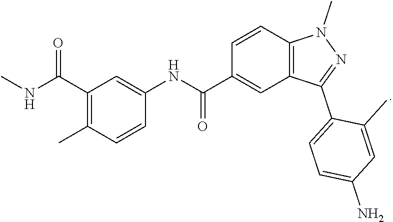

In some embodiments, the compound of formula (III) is

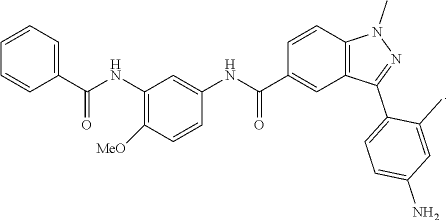 [SMILES: COc1ccc(NC(=O)c2ccc3c(c2)c(-c2ccc(N)cc2C)nn3C)cc1NC(=O)c1ccccc1]

In some embodiments, the compound of formula (III) is

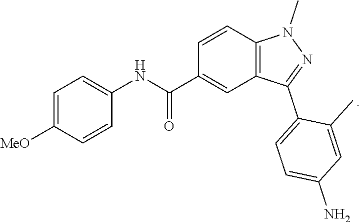 [SMILES: COc1ccc(NC(=O)c2ccc3c(c2)c(-c2ccc(N)cc2C)nn3C)cc1]

In some embodiments, the compound of formula (III) is

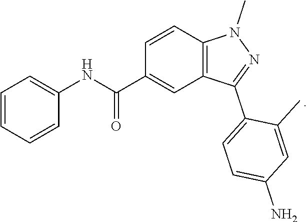 [SMILES: Cc1cc(N)ccc1-c1nn(C)c2ccc(C(=O)Nc3ccccc3)cc12]

In some embodiments, the compound of formula (III) is

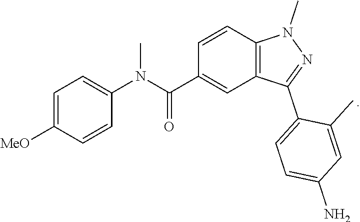 [SMILES: COc1ccc(N(C)C(=O)c2ccc3c(c2)c(-c2ccc(N)cc2C)nn3C)cc1]

In some embodiments, the compound of formula (III) is

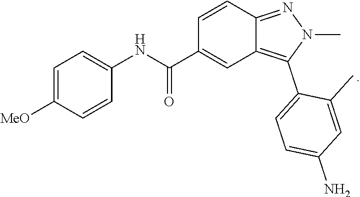 [SMILES: COc1ccc(NC(=O)c2ccc3nn(C)c(-c4ccc(N)cc4C)c3c2)cc1]

In some embodiments, the compound of formula (III) is

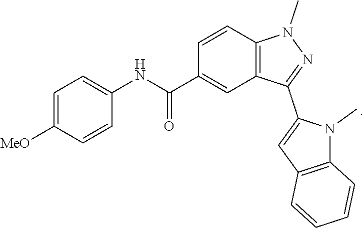 [SMILES: COc1ccc(NC(=O)c2ccc3c(c2)c(-c2cc4ccccc4n2C)nn3C)cc1]

In some embodiments, the compound of formula (III) is

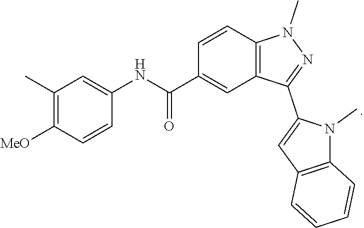 [SMILES: COc1ccc(NC(=O)c2ccc3c(c2)c(-c2cc4ccccc4n2C)nn3C)cc1C]

In some embodiments, the compound of formula (III) is

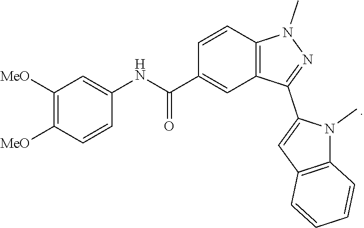 [SMILES: COc1ccc(NC(=O)c2ccc3c(c2)c(-c2cc4ccccc4n2C)nn3C)cc1OC]

In some embodiments, the compound of formula (III) is

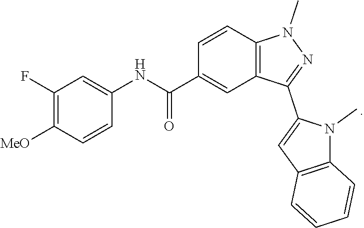 [SMILES: COc1ccc(NC(=O)c2ccc3c(c2)c(-c2cc4ccccc4n2C)nn3C)cc1F]

In some embodiments, the compound of formula (III) is

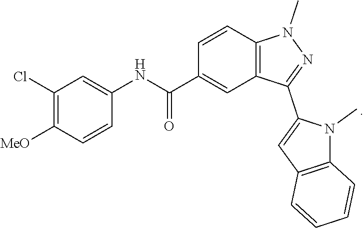 [SMILES: COc1ccc(NC(=O)c2ccc3c(c2)c(-c2cc4ccccc4n2C)nn3C)cc1Cl]

In some embodiments, the compound of formula (III) is

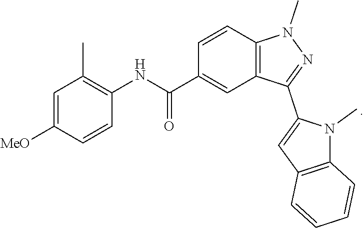 [SMILES: COc1ccc(NC(=O)c2ccc3c(c2)c(-c2cc4ccccc4n2C)nn3C)c(C)c1]

In some embodiments, the compound of formula (III) is

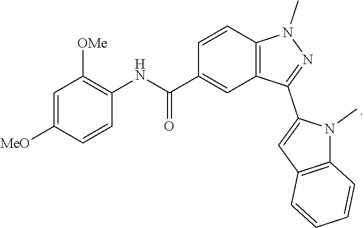 [SMILES: COc1ccc(NC(=O)c2ccc3c(c2)c(-c2cc4ccccc4n2C)nn3C)c(OC)c1]

In some embodiments, the compound of formula (III) is

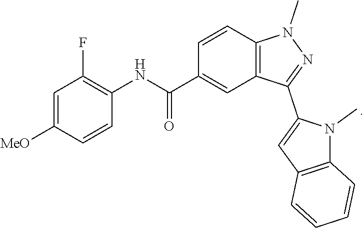 [SMILES: COc1ccc(NC(=O)c2ccc3c(c2)c(-c2cc4ccccc4n2C)nn3C)c(F)c1]

In some embodiments, the compound of formula (III) is

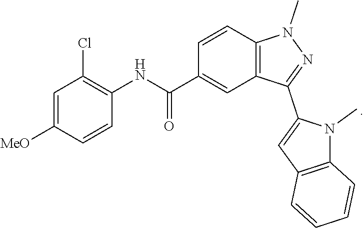 [SMILES: COc1ccc(NC(=O)c2ccc3c(c2)c(-c2cc4ccccc4n2C)nn3C)c(Cl)c1]

In some embodiments, the compound of formula (III) is

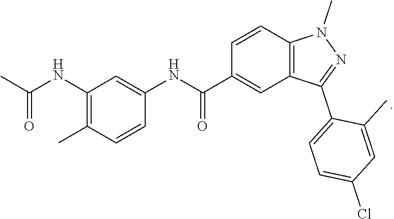 [SMILES: CC(=O)Nc1cc(NC(=O)c2ccc3c(c2)c(-c2ccc(Cl)cc2C)nn3C)ccc1C]

In some embodiments, the compound of formula (III) is

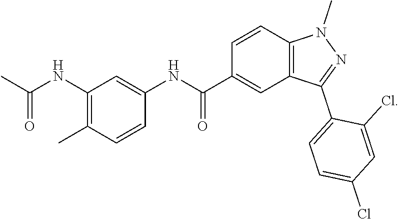 [SMILES: CC(=O)Nc1cc(NC(=O)c2ccc3c(c2)c(-c2ccc(Cl)cc2Cl)nn3C)ccc1C]

In some embodiments, the compound of formula (III) is

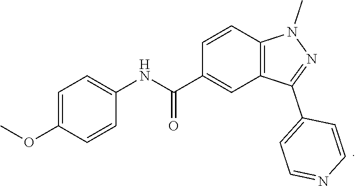 [SMILES: COc1ccc(NC(=O)c2ccc3c(c2)c(-c2ccncc2)nn3C)cc1]

In some embodiments, the compound of formula (III) is

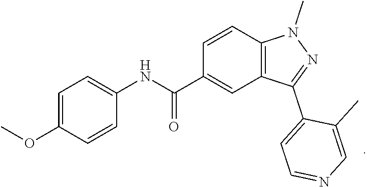

In some embodiments, the compound of formula (III) is

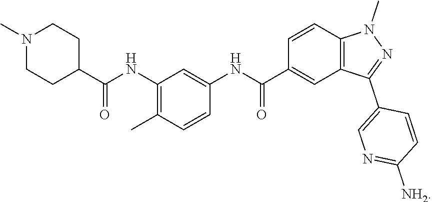 [SMILES: Cc1ccc(NC(=O)c2ccc3c(c2)c(-c2ccc(N)nc2)nn3C)cc1NC(=O)C1CCN(C)CC1]

In some embodiments, the compound of formula (III) is

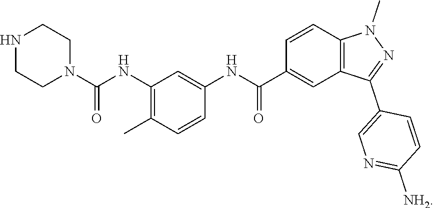 [SMILES: Cc1ccc(NC(=O)c2ccc3c(c2)c(-c2ccc(N)nc2)nn3C)cc1NC(=O)N1CCNCC1]

In some embodiments, the compound of formula (III) is

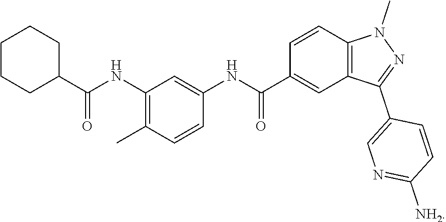 [SMILES: Cc1ccc(NC(=O)c2ccc3c(c2)c(-c2ccc(N)nc2)nn3C)cc1NC(=O)C1CCCCC1]

In some embodiments, the compound of formula (III) is

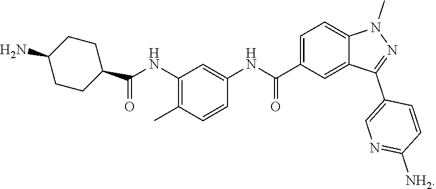 [SMILES: Cc1ccc(NC(=O)c2ccc3c(c2)c(-c2ccc(N)nc2)nn3C)cc1NC(=O)[C@H]1CC[C@@H](N)CC1]

In some embodiments, the compound of formula (III) is

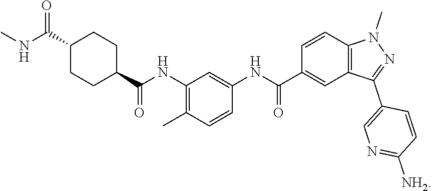 [SMILES: CNC(=O)[C@H]1CC[C@H](C(=O)Nc2cc(NC(=O)c3ccc4c(c3)c(-c3ccc(N)nc3)nn4C)ccc2C)CC1]

In some embodiments, the compound of formula (III) is

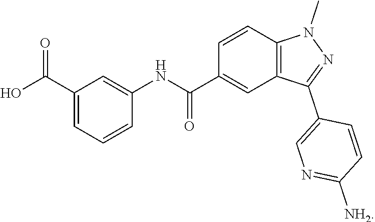 [SMILES: Cn1nc(-c2ccc(N)nc2)c2cc(C(=O)Nc3cccc(C(=O)O)c3)ccc21]

In some embodiments, the compound of formula (III) is

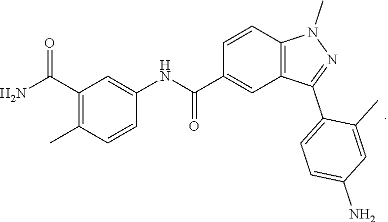 [SMILES: Cc1ccc(NC(=O)c2ccc3c(c2)c(-c2ccc(N)cc2C)nn3C)cc1C(N)=O]

In some embodiments, the compound of formula (III) is

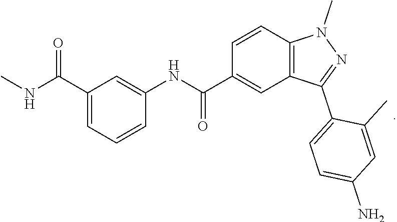

In some embodiments, the compound of formula (III) is

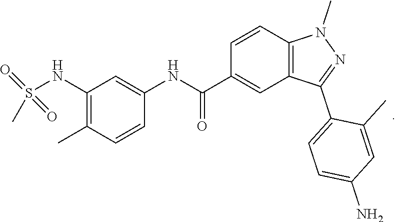 [SMILES: Cc1ccc(NC(=O)c2ccc3c(c2)c(-c2ccc(N)cc2C)nn3C)cc1NS(C)(=O)=O]

In some embodiments, the compound of formula (III) is

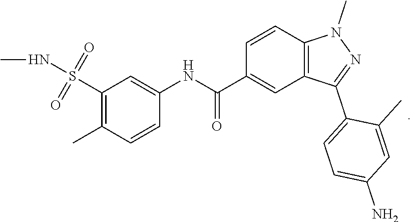

In some embodiments, the compound of formula (III) is

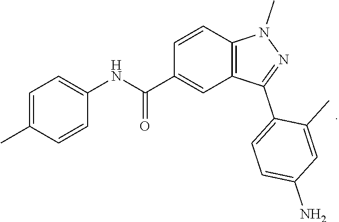

In some embodiments, the compound of formula (III) is

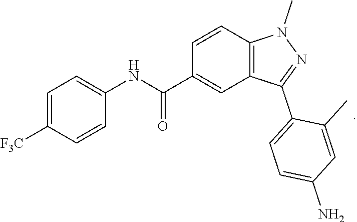 [SMILES: Cc1cc(N)ccc1-c1nn(C)c2ccc(C(=O)Nc3ccc(C(F)(F)F)cc3)cc12]

In some embodiments, the compound of formula (III) is

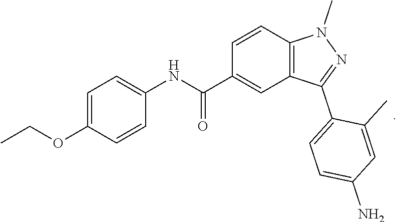 [SMILES: CCOc1ccc(NC(=O)c2ccc3c(c2)c(-c2ccc(N)cc2C)nn3C)cc1]

In some embodiments, the compound of formula (III) is

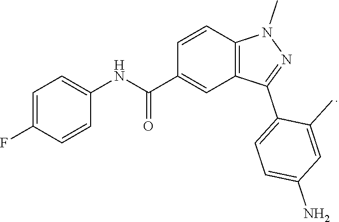 [SMILES: Cc1cc(N)ccc1-c1nn(C)c2ccc(C(=O)Nc3ccc(F)cc3)cc12]

In some embodiments, the compound of formula (III) is

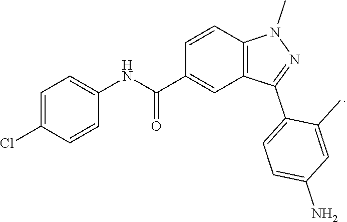 [SMILES: Cc1cc(N)ccc1-c1nn(C)c2ccc(C(=O)Nc3ccc(Cl)cc3)cc12]

In some embodiments, the compound of formula (III) is

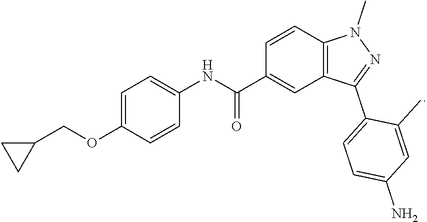 [SMILES: Cc1cc(N)ccc1-c1nn(C)c2ccc(C(=O)Nc3ccc(OCC4CC4)cc3)cc12]

In some embodiments, the compound of formula (III) is

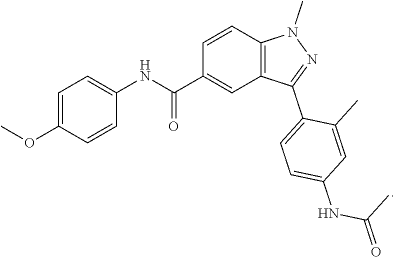 [SMILES: COc1ccc(NC(=O)c2ccc3c(c2)c(-c2ccc(NC(C)=O)cc2C)nn3C)cc1]

In some embodiments, the compound of formula (III) is

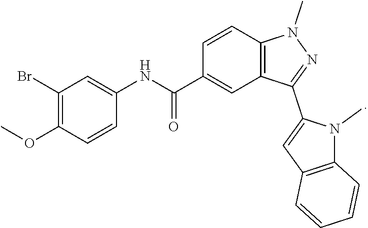

In some embodiments, the compound of formula (III) is

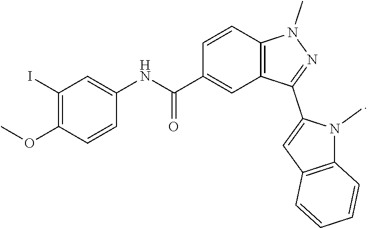

In some embodiments, the compound of formula (III) is

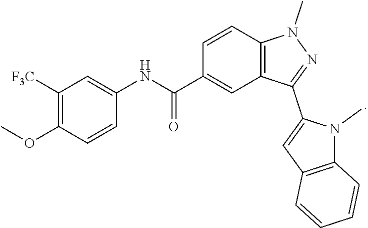

In some embodiments, the compound of formula (III) is

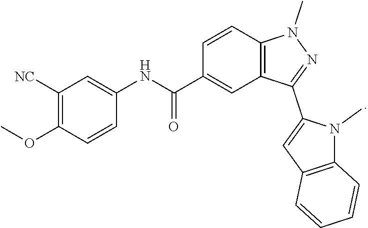

In some embodiments, the compound of formula (III) is

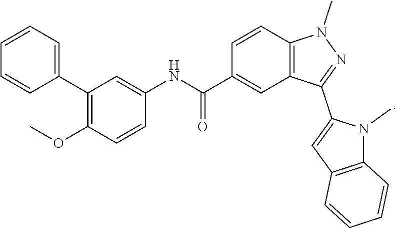 [SMILES: COc1ccc(NC(=O)c2ccc3c(c2)c(-c2cc4ccccc4n2C)nn3C)cc1-c1ccccc1]

In some embodiments, the compound of formula (III) is

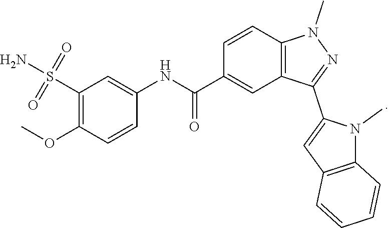

In some embodiments, the compound of formula (III) is

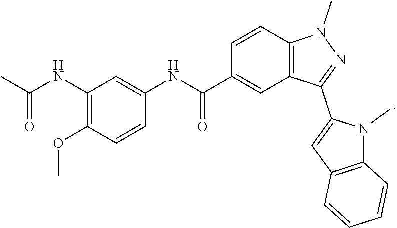 [SMILES: COc1ccc(NC(=O)c2ccc3c(c2)c(-c2cc4ccccc4n2C)nn3C)cc1NC(C)=O]

In some embodiments, the compound of formula (III) is

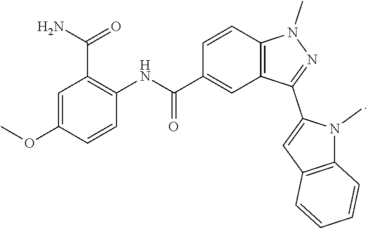

In other embodiments, the compound of formula (I) is a compound of formula (IV):

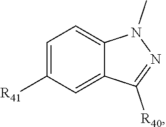

wherein:
R₄₀ is selected from C₆-C₁₂ aryl and C₄-C₁₀ heteroaryl, each of which is independently optionally substituted with one or more substituents selected from C₁-C₁₂ alkyl and —NH₂;
R₄₁ is selected from —C(═O)NHR₄₂ and C₄-C₁₀ heteroaryl optionally substituted with one or more substituents selected from C₁-C₁₂ alkyl, C₁-C₁₂ alkoxy, and —C(R₄₃)₃;
R₄₂ is selected from C₁-C₁₂ alkyl and C₄-C₁₀ heterocyclyl optionally substituted with one or more substituents selected from C₁-C₁₂ alkyl and —C(═O)R₄₄;
each R₄₃ is independently selected from fluorine, chlorine, bromine, and iodine; and R₄₄ is selected from C₁-C₁₂ alkyl and C₁-C₁₂ alkoxy.

In some embodiments, R₄₀ is selected from phenyl, indolyl, and pyridyl, each of which is independently optionally substituted with one or more substituents selected from C₁-C₁₂ alkyl and —NH₂. In some embodiments, the optional substituent is methyl and/or —NH₂.

In some embodiments, R₄₀

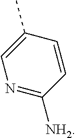

In some embodiments, R₄₀ is

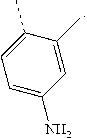

In some embodiments, R₄₀ is

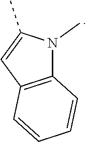

In some embodiments, R₄₁ is —C(═O)NHR₄₂. In certain embodiments, R₄₂ is methyl. In certain embodiments, R₄₂ is a saturated C₄-C₁₀ heterocyclyl optionally substituted with one or more substituents selected from C₁-C₁₂ alkyl and —C(═O)R₄₄. In certain embodiments, R₄₂ is piperidine which is optionally substituted with one or more substituents selected from C₁-C₁₂ alkyl and —C(═O)R₄₄. In certain embodiments, the optional substituent is methyl, —C(═O)CH₃, and/or —C(═O)OC(CH₃)₃. In certain embodiments, R₄₂ is

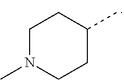

In certain embodiments, R₄₂ is

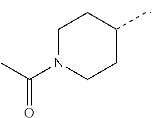

In certain embodiments, R₄₂ is

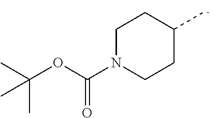

In certain embodiments, R₄₂ is

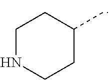

In other embodiments, R₄₁ is C₄-C₁₀ heteroaryl optionally substituted with one or more substituents selected from C₁-C₁₂ alkyl, C₁-C₁₂ alkoxy, and —C(R₄₃)₃. In certain embodiments, R₄₁ is selected from pyridyl and pyrimidyl, each of which is independently optionally substituted with one or more substituents selected from C₁-C₁₂ alkyl, C₁-C₁₂ alkoxy, and —C(R₄₃)₃. In some embodiments, the optional substituent is methyl, methoxy, and/or —CF₃. In certain embodiments, R₄₁ is selected from

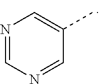

In certain embodiments, R₄₁

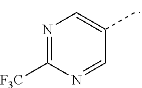

In certain embodiments, R₄₁

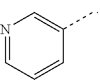

In certain embodiments, R₄₁

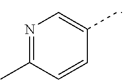

In certain embodiments, R₄₁

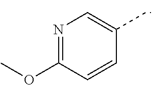

In some embodiments, the compound of formula (IV) is

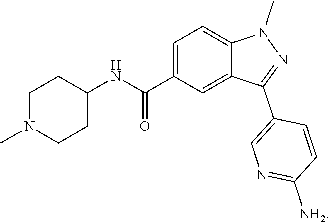 [SMILES: CN1CCC(NC(=O)c2ccc3c(c2)c(-c2ccc(N)nc2)nn3C)CC1]

In some embodiments, the compound of formula (IV) is

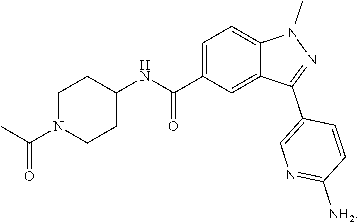 [SMILES: CC(=O)N1CCC(NC(=O)c2ccc3c(c2)c(-c2ccc(N)nc2)nn3C)CC1]

In some embodiments, the compound of formula (IV) is

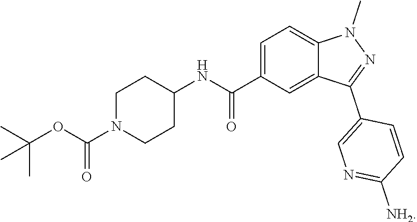 [SMILES: Cn1nc(-c2ccc(N)nc2)c2cc(C(=O)NC3CCN(C(=O)OC(C)(C)C)CC3)ccc21]

In some embodiments, the compound of formula (IV) is

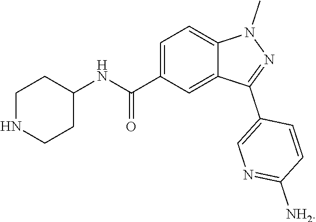 [SMILES: Cn1nc(-c2ccc(N)nc2)c2cc(C(=O)NC3CCNCC3)ccc21]

In some embodiments, the compound of formula (IV) is

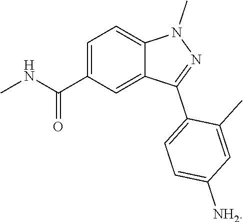

In some embodiments, the compound of formula (IV) is

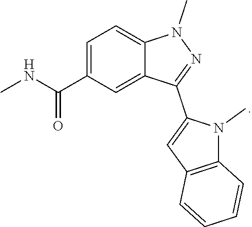

In some embodiments, the compound of formula (IV) is

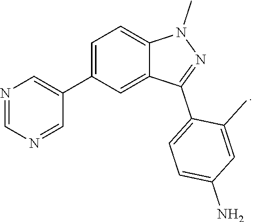 [SMILES: Cc1cc(N)ccc1-c1nn(C)c2ccc(-c3cncnc3)cc12]

In some embodiments, the compound of formula (IV) is

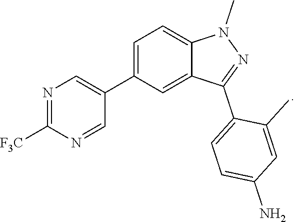 [SMILES: Cc1cc(N)ccc1-c1nn(C)c2ccc(-c3cnc(C(F)(F)F)nc3)cc12]

In some embodiments, the compound of formula (IV) is

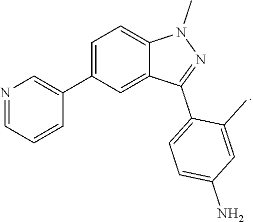 [SMILES: Cc1cc(N)ccc1-c1nn(C)c2ccc(-c3cccnc3)cc12]

In some embodiments, the compound of formula (IV) is

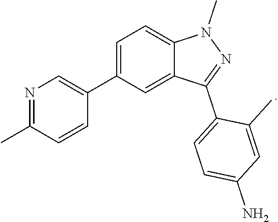

In some embodiments, the compound of formula (IV) is

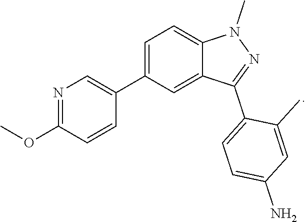

The compounds described herein can possess one or more stereocenters, and each stereocenter can exist independently in either the (R) or (S) configuration. In certain embodiments, compounds described herein are present in optically active or racemic forms. It is to be understood that the compounds described herein encompass racemic, optically-active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically-active starting materials, chiral synthesis, or chromatographic separation using a chiral stationary phase. In certain embodiments, a mixture of one or more isomer is utilized as the therapeutic compound described herein. In other embodiments, compounds described herein contain one or more chiral centers. These compounds are prepared by any means, including stereoselective synthesis, enantioselective synthesis and/or separation of a mixture of enantiomers and/or diastereomers. Resolution of compounds and isomers thereof is achieved by any means including, by way of non-limiting example, chemical processes, enzymatic processes, fractional crystallization, distillation, and chromatography.

The methods and formulations described herein include the use of N-oxides (if appropriate), crystalline forms (also known as polymorphs), solvates, amorphous phases, and/or pharmaceutically acceptable salts of compounds having the structure of any compound(s) described herein, as well as metabolites and active metabolites of these compounds having the same type of activity. Solvates include water, ether (e.g., tetrahydrofuran, methyl tert-butyl ether) or alcohol (e.g., ethanol) solvates, acetates and the like. In certain embodiments, the compounds described herein exist in solvated forms with pharmaceutically acceptable solvents such as water, and ethanol. In other embodiments, the compounds described herein exist in unsolvated form.

In certain embodiments, the compound(s) described herein can exist as tautomers. All tautomers are included within the scope of the compounds presented herein.

In certain embodiments, compounds described herein are prepared as prodrugs. A “prodrug” refers to an agent that is converted into the parent drug in vivo. In certain embodiments, upon in vivo administration, a prodrug is chemically converted to the biologically, pharmaceutically or therapeutically active form of the compound. In other embodiments, a prodrug is enzymatically metabolized by one or more steps or processes to the biologically, pharmaceutically or therapeutically active form of the compound.

In certain embodiments, sites on, for example, the aromatic ring portion of compound(s) described herein are susceptible to various metabolic reactions. Incorporation of appropriate substituents on the aromatic ring structures may reduce, minimize or eliminate this metabolic pathway. In certain embodiments, the appropriate substituent to decrease or eliminate the susceptibility of the aromatic ring to metabolic reactions is, by way of example only, a deuterium, a halogen, or an alkyl group.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In certain embodiments, isotopically-labeled compounds are useful in drug and/or substrate tissue distribution studies. In other embodiments, substitution with heavier isotopes such as deuterium affords greater metabolic stability (for example, increased in vivo half-life or reduced dosage requirements). In yet other embodiments, substitution with positron emitting isotopes, such as ¹¹C, ¹⁸F, ¹⁵O, and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

In certain embodiments, the compounds described herein are labeled by other means, including, but not limited to, the use of chromophores or fluorescent moieties, bioluminescent labels, or chemiluminescent labels.

The compounds described herein, and other related compounds having different substituents are synthesized using techniques and materials described herein and as described, for example, in Fieser & Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989), March, Advanced Organic Chemistry 4^{th} Ed., (Wiley 1992); Carey & Sundberg, Advanced Organic Chemistry 4th Ed., Vols. A and B (Plenum 2000, 2001), and Green & Wuts, Protective Groups in Organic Synthesis 3rd Ed., (Wiley 1999) (all of which are incorporated by reference for such disclosure). General methods for the preparation of compound as described herein are modified by the use of appropriate reagents and conditions, for the introduction of the various moieties found in the formula as provided herein.

Compounds described herein are synthesized using any suitable procedures starting from compounds that are available from commercial sources, or are prepared using procedures described herein.

In certain embodiments, reactive functional groups, such as hydroxyl, amino, imino, thio or carboxy groups, are protected in order to avoid their unwanted participation in reactions. Protecting groups are used to block some or all of the reactive moieties and prevent such groups from participating in chemical reactions until the protective group is removed. In other embodiments, each protective group is removable by a different means. Protective groups that are cleaved under totally disparate reaction conditions fulfill the requirement of differential removal.

In certain embodiments, protective groups are removed by acid, base, reducing conditions (such as, for example, hydrogenolysis), and/or oxidative conditions. Groups such as trityl, dimethoxytrityl, acetal and t-butyldimethylsilyl are acid labile and are used to protect carboxy and hydroxy reactive moieties in the presence of amino groups protected with Cbz groups, which are removable by hydrogenolysis, and Fmoc groups, which are base labile. Carboxylic acid and hydroxy reactive moieties are blocked with base labile groups such as, but not limited to, methyl, ethyl, and acetyl, in the presence of amines that are blocked with acid labile groups, such as t-butyl carbamate, or with carbamates that are both acid and base stable but hydrolytically removable.

In certain embodiments, carboxylic acid and hydroxy reactive moieties are blocked with hydrolytically removable protective groups such as the benzyl group, while amine groups capable of hydrogen bonding with acids are blocked with base labile groups such as Fmoc. Carboxylic acid reactive moieties are protected by conversion to simple ester compounds as exemplified herein, which include conversion to alkyl esters, or are blocked with oxidatively-removable protective groups such as 2,4-dimethoxybenzyl, while co-existing amino groups are blocked with fluoride labile silyl carbamates.

Allyl blocking groups are useful in the presence of acid- and base-protecting groups since the former are stable and are subsequently removed by metal or pi-acid catalysts. For example, an allyl-blocked carboxylic acid is deprotected with a palladium-catalyzed reaction in the presence of acid labile t-butyl carbamate or base-labile acetate amine protecting groups. Yet another form of protecting group is a resin to which a compound or intermediate is attached. As long as the residue is attached to the resin, that functional group is blocked and does not react. Once released from the resin, the functional group is available to react.

Typically blocking/protecting groups may be selected from:

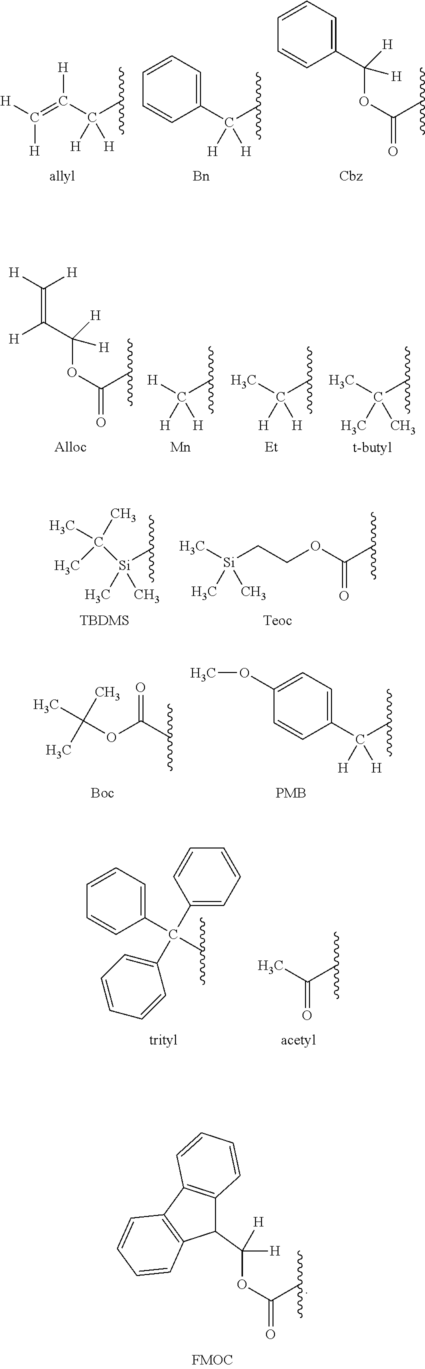

Other protecting groups, plus a detailed description of techniques applicable to the creation of protecting groups and their removal are described in Greene & Wuts, Protective Groups in Organic Synthesis, 3rd Ed., John Wiley & Sons, New York, NY, 1999, and Kocienski, Protective Groups, Thieme Verlag, New York, NY, 1994, which are incorporated herein by reference for such disclosure.

### Compositions

In one aspect, the present disclosure provides a pharmaceutical composition comprising at least one compound of the present disclosure and at least one pharmaceutically acceptable carrier.

The compositions containing the compound(s) described herein include a pharmaceutical composition comprising at least one compound as described herein and at least one pharmaceutically acceptable carrier. In some embodiments, the pharmaceutical composition comprises Kolliphor EL, and aqueous buffer, or a combination thereof. In certain embodiments, the aqueous buffer comprises phosphate buffered saline (PBS). In some embodiments, the aqueous buffer comprises 1×PBS. In certain embodiments, the pharmaceutical composition comprises about 5%, about 10%, about 15%, about 20%, about 25%, about 30%, about 35%, or about 40% Kolliphor EL. In some embodiments, the pharmaceutical composition comprises about 20% Kolliphor EL in 1×PBS.

In certain embodiments, the composition is formulated for an administration route such as oral or parenteral, for example, transdermal, transmucosal (e.g., sublingual, lingual, (trans)buccal, (trans)urethral, vaginal (e.g., trans- and perivaginally), (intra)nasal and (trans)rectal, intravesical, intrapulmonary, intraduodenal, intragastrical, intrathecal, subcutaneous, intramuscular, intradermal, intra-arterial, intravenous, intrabronchial, inhalation, and topical administration. In some embodiments, the composition is formulated as a pill, tablet, gelcap, or capsule for oral administration.

Methods of Treatment, Amelioration, and/or Prevention

In one aspect, the present disclosure relates to a method of promoting male contraception in a male subject. In another aspect, the present disclosure relates to a method of promoting male sterility in a male subject. In yet another aspect, the present disclosure relates to a method of minimizing and/or reducing spermatozoa number and/or motility in a male subject. In yet another aspect, the present disclosure relates to a method of reducing testes size in a male subject. In yet another aspect, the present disclosure relates to a method of inhibiting bromodomain testis (BRDT) in a male subject.

In certain embodiments, the method comprises administering to the male subject a therapeutically effective amount of a compound of the disclosure.

In certain embodiments, the method comprises administering to the male subject a therapeutically effective amount of a compound of formula (I).

In some embodiments, the compound of formula (I) is a compound of formula (II). Exemplary compounds of formula (II) are described elsewhere herein. In other embodiments, the compound of formula (I) is a compound of formula (III). Exemplary compounds of formula (III) are described elsewhere herein. In yet other embodiments, the compound of formula (I) is a compound of formula (IV). Exemplary compounds of formula (IV) are described elsewhere herein.

In some embodiments, the step of administering a compound of formula (I) selectively inhibits BRDT bromodomain 2 (BRDT-BD2) over BRDT bromodomain 1 (BRDT-BD1). In some embodiments, the compound of formula (I) binds BRDT-BD2 and does not bind, or binds minimally to, BRDT-BD1. In some embodiments, the step of administering a compound of formula (I) further inhibits BRD4 of the BET subfamily. In some embodiments, the compound of formula (I) selectively inhibits BRD4 bromodomain 2 (BRD4-BD2) over BRD4 bromodomain 1 (BRD4-BD1). In some embodiments, the compound of formula (I) binds BRD4-BD2 and does not bind, or binds minimally to, BRD4-BD1. In some embodiments, the compound of formula (I) binds selectively to both BRDT-BD2 and BRD4-BD2. Therefore, in some embodiments, the method further relates to a method of inhibiting both BRDT and BRD4 in the subject, the method comprising administering to the subject a therapeutically effective amount of a compound of formula (I). In some embodiments, the compound of formula (I) binds selectively to BRDT-BD2, BRD4-BD2, BRD3-BD2, BRD2-BD2, or a combination thereof. Therefore, in some embodiments, the method further relates to a method of inhibiting BRDT, BRD4, BRD3, BRD2, or a combination thereof in the subject, the method comprising administering to the subject a therapeutically effective amount of a compound of formula (I).

The methods described herein include administering to the subject a therapeutically effective amount of at least one compound described herein, which is optionally formulated in a pharmaceutical composition. In various embodiments, a therapeutically effective amount of at least one compound described herein present in a pharmaceutical composition is the only therapeutically active compound in a pharmaceutical composition. In certain embodiments, the method further comprises administering to the subject an additional agent that inhibits BRDT, BRD4, BRD3, BRD2, or a combination thereof.

The compound of formula (I) can be administered to the subject using any administration route known to a person of skill in the art. Exemplary routes of administration are described elsewhere herein. In some embodiments, a composition comprising a compound of formula (I) is orally administered to the subject. In some embodiments, a pill, tablet, gelcap, or capsule comprising a compound of formula (I) is orally administered to the subject.

The compound of formula (I) can be administered to the subject in any dosage with any timing of dosage administration necessary to inhibit BRDT, BRD4, BRD3, BRD2, or a combination thereof and to provide a desired therapeutic effect. In some embodiments, the compound of formula (I) is administered to the male subject in order to provide a contraceptive effect in the subject. Although not wishing to be limited by theory, it is believed that inhibition of BRDT function in spermatocytes and spermatids leads to a reduction in spermatozoa number and motility. Therefore, in some embodiments, the dosage of the compound of formula (I) and the timing of dosage administration provide a contraceptive effect in the male subject. In some embodiments, the compound of formula (I) is administered to the subject daily. In certain embodiments, the compound of formula (I) is administered to the subject at a dosage of between about 10 mg/kg and 100 mg/kg, about 20 mg/kg and 90 mg/kg, about 30 mg/kg and 80 mg/kg, about 40 mg/kg and 70 mg/kg, or about 40 mg/kg and 60 mg/kg. In some embodiments, the compound of formula (I) is administered daily to the subject at a dosage of between about 40 mg/kg and 60 mg/kg to provide a contraceptive effect.

In certain embodiments, the subject is a mammal. In other embodiments, the mammal is a human.

### Administration/Dosage/Formulations

The regimen of administration may affect what constitutes an effective amount. The therapeutic formulations may be administered to the subject either prior to or after the onset of the disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions described herein to a patient, preferably a mammal, more preferably a human, may be carried out using known procedures, at dosages and for periods of time effective to treat the disease or disorder in the patient. An effective amount of the therapeutic compound necessary to achieve a therapeutic effect may vary according to factors such as the state of the disease or disorder in the patient; the age, sex, and weight of the patient; and the ability of the therapeutic compound to treat the disease or disorder in the patient. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound described herein is from about 1 and 5,000 mg/kg of body weight/per day. One of ordinary skill in the art would be able to study the relevant factors and make the determination regarding the effective amount of the therapeutic compound without undue experimentation.

Actual dosage levels of the active ingredients in the pharmaceutical compositions described herein may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds described herein employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the compound(s) described herein are dictated by and directly dependent on (a) the unique characteristics of the therapeutic compound and the particular therapeutic effect to be achieved, and (b) the limitations inherent in the art of compounding/formulating such a therapeutic compound.

In certain embodiments, the compositions described herein are formulated using one or more pharmaceutically acceptable excipients or carriers. In certain embodiments, the pharmaceutical compositions described herein comprise a therapeutically effective amount of a compound described herein and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the compositions described herein are administered to the patient in dosages that range from one to five times per day or more. In other embodiments, the compositions described herein are administered to the patient in range of dosages that include, but are not limited to, once every day, every two, days, every three days to once a week, and once every two weeks. It is readily apparent to one skilled in the art that the frequency of administration of the various combination compositions described herein varies from individual to individual depending on many factors including, but not limited to, age, disease or disorder to be treated, gender, overall health, and other factors. Thus, administration of the compounds and compositions described herein should not be construed to be limited to any particular dosage regime and the precise dosage and composition to be administered to any patient is determined by the attending physician taking all other factors about the patient into account.

The compound(s) described herein for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 350 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound described herein is from about 1 mg and about 2,500 mg. In some embodiments, a dose of a compound described herein used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, a composition as described herein is a packaged pharmaceutical composition comprising a container holding a therapeutically effective amount of a compound described herein, alone or in combination with a second pharmaceutical agent; and instructions for using the compound to treat, or reduce one or more symptoms of a disease or disorder in a patient.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., other analgesic agents.

Routes of administration of any of the compositions described herein include oral, nasal, rectal, intravaginal, parenteral, buccal, sublingual or topical. The compounds for use in the compositions described herein can be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions described herein are not limited to the particular formulations and compositions that are described herein.

Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compound(s) described herein can be in the form of tablets or capsules prepared by conventional means with pharmaceutically acceptable excipients such as binding agents (e.g., polyvinylpyrrolidone, hydroxypropylcellulose or hydroxypropyl methylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Parenteral Administration

For parenteral administration, the compounds as described herein may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1, 3-butanediol.

Among the acceptable vehicles and solvents that may be employed are water, Ringer's solution and isotonic sodium chloride solution. Sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically acceptable oils, such as olive oil or castor oil, especially in their polyoxyethylated versions. These oil solutions or suspensions may also contain a long-chain alcohol diluent or dispersant, such as Ph. Helv or similar alcohol.

Additional Administration Forms

Additional dosage forms suitable for use with the compound(s) and compositions described herein include dosage forms as described in U.S. Pat. Nos. 6,340,475; 6,488,962; 6,451,808; 5,972,389; 5,582,837; and 5,007,790. Additional dosage forms suitable for use with the compound(s) and compositions described herein also include dosage forms as described in U.S. Patent Applications Nos. 20030147952; 20030104062; 20030104053; 20030044466; 20030039688; and 20020051820. Additional dosage forms suitable for use with the compound(s) and compositions described herein also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In certain embodiments, the formulations described herein can be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material which provides sustained release properties to the compounds. As such, the compounds for use with the method(s) described herein may be administered in the form of microparticles, for example, by injection or in the form of wafers or discs by implantation.

In some cases, the dosage forms to be used can be provided as slow or controlled-release of one or more active ingredients therein using, for example, hydropropylmethyl cellulose, other polymer matrices, gels, permeable membranes, osmotic systems, multilayer coatings, microparticles, liposomes, or microspheres or a combination thereof to provide the desired release profile in varying proportions. Suitable controlled-release formulations known to those of ordinary skill in the art, including those described herein, can be readily selected for use with the pharmaceutical compositions described herein. Thus, single unit dosage forms suitable for oral administration, such as tablets, capsules, gelcaps, and caplets that are adapted for controlled-release are encompassed by the compositions and dosage forms described herein.

Most controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency, and increased patient compliance. In addition, controlled-release formulations can be used to affect the time of onset of action or other characteristics, such as blood level of the drug, and thus can affect the occurrence of side effects.

Most controlled-release formulations are designed to initially release an amount of drug that promptly produces the desired therapeutic effect, and gradually and continually release of other amounts of drug to maintain this level of therapeutic effect over an extended period of time. In order to maintain this constant level of drug in the body, the drug must be released from the dosage form at a rate that will replace the amount of drug being metabolized and excreted from the body.

Controlled-release of an active ingredient can be stimulated by various inducers, for example pH, temperature, enzymes, water, or other physiological conditions or compounds. The term “controlled-release component” is defined herein as a compound or compounds, including, but not limited to, polymers, polymer matrices, gels, permeable membranes, liposomes, or microspheres or a combination thereof that facilitates the controlled-release of the active ingredient. In some embodiments, the compound(s) described herein are administered to a patient, alone or in combination with another pharmaceutical agent, using a sustained release formulation. In some embodiments, the compound(s) described herein are administered to a patient, alone or in combination with another pharmaceutical agent, using a sustained release formulation.

The term delayed release is used herein in its conventional sense to refer to a drug formulation that provides for an initial release of the drug after some delay following drug administration and that mat, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Dosing

The therapeutically effective amount or dose of a compound described herein depends on the age, sex and weight of the patient, the current medical condition of the patient and the progression of the disease or disorder in the patient being treated. The skilled artisan is able to determine appropriate dosages depending on these and other factors.

A suitable dose of a compound described herein can be in the range of from about 0.01 mg to about 5,000 mg per day, such as from about 0.1 mg to about 1,000 mg, for example, from about 1 mg to about 500 mg, such as about 5 mg to about 250 mg per day. The dose may be administered in a single dosage or in multiple dosages, for example from 1 to 4 or more times per day. When multiple dosages are used, the amount of each dosage may be the same or different. For example, a dose of 1 mg per day may be administered as two 0.5 mg doses, with about a 12-hour interval between doses.

It is understood that the amount of compound dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on.

In the case wherein the patient's status does improve, upon the doctor's discretion the administration of the compound(s) described herein is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced to a level at which the improved disease is retained. In certain embodiments, patients require intermittent treatment on a long-term basis upon any recurrence of symptoms and/or infection.

The compounds described herein can be formulated in unit dosage form. The term “unit dosage form” refers to physically discrete units suitable as unitary dosage for patients undergoing treatment, with each unit containing a predetermined quantity of active material calculated to produce the desired therapeutic effect, optionally in association with a suitable pharmaceutical carrier. The unit dosage form may be for a single daily dose or one of multiple daily doses (e.g., about 1 to 4 or more times per day). When multiple daily doses are used, the unit dosage form may be the same or different for each dose.

Toxicity and therapeutic efficacy of such therapeutic regimens are optionally determined in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index, which is expressed as the ratio between LD₅₀ and ED₅₀. The data obtained from cell culture assays and animal studies are optionally used in formulating a range of dosage for use in human. The dosage of such compounds lies preferably within a range of circulating concentrations that include the ED₅₀ with minimal toxicity. The dosage optionally varies within this range depending upon the dosage form employed and the route of administration utilized.

Those skilled in the art will recognize, or be able to ascertain using no more than routine experimentation, numerous equivalents to the specific procedures, embodiments, claims, and examples described herein. Such equivalents are considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present disclosure. However, they are in no way a limitation of the teachings or disclosure of the present disclosure as set forth herein.

### EXPERIMENTAL EXAMPLES

The disclosure is further described in detail by reference to the following experimental examples. These examples are provided for purposes of illustration only, and are not intended to be limiting unless so specified. Thus, the disclosure should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present disclosure and practice the claimed methods. The following working examples therefore, specifically point out the preferred embodiments of the present disclosure, and are not to be construed as limiting in any way the remainder of the disclosure.

### Materials and Methods

### Production of Recombinant Bromodomain Proteins

cDNAs encoding human BRDT and BRD4 bromodomains were either synthesized by Genewiz with codon optimization for bacterial expression or reverse-transcribed from mRNA isolated from the human testes. Human testes tissues were obtained from the Human Tissue Acquisition and Pathology core service (Baylor College of Medicine, USA). The polymerase chain reaction (PCR) using Phusion high-fidelity DNA polymerase (ThermoFisher Scientific, USA) was applied to amplify the bromodomain regions of BRDT and BRD4 from these cDNA templates. Sequence boundaries for individual recombinant bromodomains to be produced were specified as follows: BRDT-BD1, NP_001717.3 amino acid 21-137; BRDT-BD2, NP_001717.3 amino acid 269-380; BRD4, NP_055114.1 amino acid 44-168; and BRD4-BD2, NP_055114.1 amino acid 333-460. PCR products were purified using Zymo DNA clean & concentrator kit (Zymo Research, USA) and digested with restriction enzymes to generate sticky ends for further subcloning into pET15b or pET28b bacterial expression vectors (Addgene, USA) with an N-terminal polyhistidine tag. The constructs were transformed into TOP10 competent cells to yield the final plasmid DNA and sequence alignment was validated by Sanger sequencing.

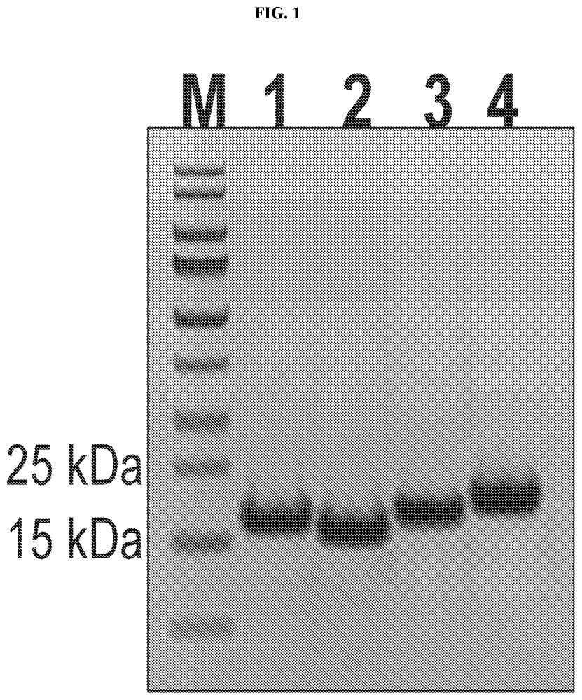

To produce the individual truncated binding domains of the recombinant bromodomain proteins, colonies from freshly transformed competent E. coli NiCo21 (DE3) cells were cultured overnight at 37° C. in Luria-Bertani medium (LB-broth, Merck) with 50 μg/mL kanamycin. These cells were diluted 1:50 in fresh medium and cultured at 37° C. until the optical density reached about 0.5 (OD600). The temperature of the culture was then decreased to 18° C. Once the system equilibrated at this temperature, OD600 was re-checked to assure not exceeding 0.8, and protein expression was induced overnight by the addition of 0.2 mM isopropyl-β-D-thiogalactopyranoside (IPTG). The bacterial cells were harvested by centrifugation at 4,000 rpm for 45 min at 4° C. The pellet of cells expressing His-tagged bromodomain proteins was resuspended in lysis buffer (20 mL buffer/cells from 250 ml culture) containing 50 mM Hepes pH 7.5, 500 mM NaCl, 1 mM TCEP (tris(2-carboxyethyl) phosphinehydrochloride)) and 10 mM imidazole, supplemented with Roche cOmplete™ Protease Inhibitor Cocktail. The well-resuspended cells were lysed using French Press in a pre-chilled tube. The lysate was cleared by centrifugation at 16,000 g for 1 h at 4° C. on a Beckman Coulter ultracentrifuge and incubated with activated Talon cobalt resins (Takara Bio, USA) overnight at 4° C. with end over end rotation. Talon cobalt resins with protein-lysate bound were packed onto a 10 mL glass column to enable a gravity-flow purification of His-tagged bromodomain proteins. The column was washed once with 30 mL of lysis buffer then twice with 10 mL of lysis buffer containing 30 mM imidazole. The protein was eluted using a step elution of imidazole in lysis buffer (50, 100, 150 and 250 mM imidazole in 50 mM Hepes, pH 7.5, 500 mM NaCl). All fractions were collected and monitored by SDS-polyacrylamide gel electrophoresis (Invitrogen NuPAGE™ 4 to 12%, Bis-Tris gradient gel). Fractions with enriched His-tagged bromodomain proteins were pooled and further purified with size exclusion chromatography on a superdex75 increase 10/300 GL gel filtration column (GE/Amersham Biosciences, USA) at a flow rate of 0.5 mL/min. Samples were monitored by SDS-polyacrylamide gel electrophoresis, pooled, concentrated in the gel filtration buffer (50 mM Hepes pH 7.5, 150 mM NaCl, 1 mM TCEP), aliquoted, flash frozen in liquid nitrogen and stored at −80° C. for DEC-Tec selection and assay validation (FIG. 1).

DEC-Tec Affinity Selection with Bromodomain Proteins

To identify BRDT-BD2 selective compounds, the DEC-Tec library pool was screened in five tubes: 1) absence of bromodomain proteins (bead binding non-target control, NTC), 2) presence of His-BRDT-BD2 at 0.3 μM, 3) presence of His-BRDT-BD1 at 0.3 μM (a counter-screen for bromodomain selective compounds), 4) presence of His-BRDT-BD2 plus (+)-JQ1 at 100 μM, and 5) presence of His-BRDT-BD1 plus (+)-JQ1 at 100 μM. (+)-JQ1 binds to BRDT bromodomains and the addition of (+)-JQ1 can block the binding of compounds in the library to bromodomain functional site so as to serve as controls. To initiate the screening experiment, the pool of the DEC-Tec libraries was quantitated using quantitative PCR (qPCR) so that each screening tube would start with the same amount of library molecules with each compound having one million copies. Three rounds of DEC-Tec selection have been performed to improve ligand enrichment. The DNA barcode from the last round of selection was PCR amplified and sequenced to identify the linked drug-like binders. In brief, DEC-Tec screening and sequencing was performed as follows: 1) His-BRDT-BD2 or His-BRDT-BD1 at designated concentration was incubated with DEC-Tec libraries in 50 mM HEPES buffer, pH 7.5, containing 150 mM NaCl, 10 mM imidazole, 1 mM TCEP, 1 mM CHAPS and 0.1 mg/ml sheared salmon sperm DNA for 45 min at room temperature with continuous shaking; 2) His-BRDT-BD2 or BD1 along with the binding molecules were then captured instantaneously with HisPur Ni-NTA magnetic beads; 3) The Ni-NTA magnetic beads were washed 1 time with the aforementioned selection buffer without sheared salmon sperm DNA by brief and vigorous vortex; 4) To dissociate the binding molecules from His-tagged protein, Ni-NTA magnetic beads in 100 μL washing buffer were heated at 80° C. for 10 min with continuous shaking; 5) The resulting eluent, in which protein binding molecules are enriched, were further incubated with fresh His-BRDT-BD2 or BD1 to initiate another round of selection following the same protocol described above; 6) After the last round of selection, the eluted encoding oligonucleotides were amplified using Platinum Taq DNA Polymerase High Fidelity using primers that incorporate complementary sequences to the library headpiece or tailpiece along with the Illumina READ 1 or READ 2 sequences required for clustering and Illumina sequencing; 7) The amplified DNA were cleaned by Agencourt AMPure XP beads and quantitated with Agilent high sensitivity DNA kit using a Bioanalyzer; 8) DNA was then denatured and sequenced along with a 3% PhiX spike-in in a single-read 105-cycle sequencing on an Illumina NextSeq 500 instrument; 9) The FASTQ format sequencing data was generated through Illumina BaseSpace and decoded and analyzed. Small molecules identified from the above screens were resynthesized off of DNA and evaluated in in vitro assays.

### Bromodomain Proximity Assay

The AlphaScreen™ assay was performed following a previous publication with minor modifications from the manufacturer's protocol (PerkinElmer, USA). A 20-μL reaction was set up in a PerkinElmer 384-well AlphaPlate where His-bromodomain at 10 nM was incubated with biotinylated (+)-JQ1 at 10 nM, nickel chelate acceptor beads at 12.5 μg/mL, and tested compound at various concentrations for 15 min at room temperature, followed by the addition of streptavidin donor beads at 12.5 μg/mL and another 60-min incubation at room temperature. The plate was read on a Tecan Infinite M1000 Pro plate reader.

### Thermal Shift Assay

The dye SYPRO Orange (ThermoFisher Scientific, USA) was used to perform the protein thermal shift assay. The assay was set up on a 384-well Roche plate where His-bromodomain at a concentration of 2 μM was incubated with the test compound at various concentrations, and SYPRO Orange dye at 5× in a 10-μL reaction. The melting curve experiment and data analysis was run on a Roche Lightcycler 480 real-time PCR instrument.

### NanoBRET Target Engagement Intracellular BET Bromodomain Assay

The NanoLuc-BRDT-BD1 and NanoLuc-BRDT-BD2 vectors were constructed by subcloning the same BRDT bromodomain sequences applied for protein expression into the NanoLuc-BRD4-BD1 and NanoLuc-BRD4-BD2 vectors (Promega, USA) to replace the corresponding BRD4 bromodomain sequences. The NanoBRET tracer competition assay was performed in transiently transfected HEK293 cells expressing each NanoLuc-bromodomain vector on a 384-well plate following the manufacturer's protocol (Promega, USA). Tracer titration was performed for each NanoLuc fusion vector to determine the optimized tracer concentration. The phusion protein was allowed to express for 36 h. The cells were then preincubated with tested compounds at different concentrations for 2 h followed by 2 h of incubation with tracer. Freshly prepared NanoBRET Nano-Glo substrate plus extracellular NanoLuc inhibitor were then added to initiate the subsequent bioluminescence resonance energy transfer (BRET) measurements using a CLARIOstar Plus BMG LABTECH plate reader. Data analysis was done by measuring the ratio of acceptor emission to donor emission (BRET ratio) and normalized by subtracting no-tracer-control-background.

### Metabolic Stability Assay in Liver Microsomes

Compounds 3-10, 15, 17, and 20-22 (2.0 μM) were incubated in the mouse or human liver microsomes (0.5 mg protein/mL) at 37° C. The samples are collected at specific time-points 0, 5, 10, 20, 40, and 60 min. The reactions were terminated by adding equivalent volume of ice-cold CH₃OH and vortexed. The reaction mixtures were centrifuged at rcf 15,000 for 15 min. Five μL of the supernatant was analyzed by UHPLC-Q Exactive Orbitrap MS (Thermo Fisher Scientific, USA) equipped with 50 mm×4.6 mm column (XDB C-18, Agilent Technologies, USA). The column temperature was maintained at 40° C. The flow rate was at 0.3 mL/min with a 30% mobile phase (acetonitrile containing 0.1% formic acid). Q Exactive MS was operated in positive or negative mode with electrospray ionization. Ultra-pure nitrogen was applied as the sheath (45 arbitrary unit), auxiliary (10 arbitrary unit), sweep (1.0 arbitrary unit), and the collision gas. The capillary gas temperature was set at 275° C. and the capillary voltage was set at 3.7 kV. MS data was acquired from 80 to 1,200 Da in profile mode.

### BROMOscan Bromodomain Profiling

BROMOscan bromodomain profiling was provided by Eurofins DiscoverX Corp. (San Diego, CA, USA, http://www.discoverx.com). Determination of the Kd between test compounds and DNA tagged bromodomains was achieved through binding competition against a proprietary reference immobilized ligand. Binding constants (Kds) were calculated with a standard dose-response curve using the Hill equation:

Response=Background+

Signal-Background

1+(KdHill Slope/DoseHill Slope)

The Hill Slope was set to −1.

Curves were fitted using a non-linear least square fit with the Levenberg-Marquardt algorithm.

### Crystallography

BRDT-BD2 was co-crystallized with 9 or 20 by hanging drop vapor diffusion method. For crystallization, the purified BRDT-BD2 at 10 mg/mL was mixed with 2 and 4 molar excess of 9 and 20, respectively. The protein-inhibitor mixture was concentrated using Amicon Ultra-15 centrifugal filters (Millipore Sigma) to 30 mg/mL. Mosquito (TTP labtech) was used to dispense equal volumes of protein and reservoir (250 nL each) against 70 μL reservoir buffer in 96 wells crystallization tray (96-Well clear polystyrene microplate from SPT labtech). For the 9 complex, crystals were observed after 10 days in drop containing 0.8 M succinic acid (pH 7.0), whereas for the 20 complex, crystals were observed after 15 days in drop containing 1.1 M sodium malonate 0.1 M, HEPES pH 7.0, 0.5% v/v Jeffamine ED-2001. The diffraction data were collected at Advanced Light Source (ALS), Beam Line 5.0.2 (UC Berkeley, USA) at wavelength (λ)=1.0000 Â, by using an ADSC Q210 CCD detector. The data was integrated and scaled by using iMosflm and SCALA, respectively. The crystal structures of the 9 and 20 complexes were determined by molecular replacement using crystal structure of the human C-terminal bromodomain BRD2 (PDB ID: 4UYG) and the determined structure of the 9 complex as search models. The final models have gone through several rounds of refinement using phenix.refine followed by manual model building using COOT. For all structural analysis and figure preparation, the visualization program PyMOL was used. The data collection and refinement statistics are summarized in Table 1.

TABLE 1
X-ray data collection and refinement statistics

| Complex | BRDT-BD2/9 | | BRDT-BD2/20 | |
|---|---|---|---|---|
| Data collection | | | | |
| Space group | P 3₂ 2 1 | | P 1 2₁ 1 | |
| Cell Dimensions | | | | |
| a, b, c (Å) | 56.47, 56.47, 191.90 | | 47.23, 61.97, 99.18 | |
| α, β, γ (°) | 90, 90, 120 | | 90, 97.28, 90 | |
| Resolution (Å) | 47.39-2.27 (2.35-2.27)^{a} | | 44.55-1.90 (1.96-1.90)^{a} | |
| R_{merge} (%) | 4.99 | (60.81) | 8.79 | (108.30) |
| I/σ (I) | 10.90 | (1.00) | 8.24 | (1.65) |
| CC _{1/2} | 0.99 | (0.67) | 0.98 | (0.20) |
| Completeness (%) | 99.73 | (99.47) | 98.17 | (94.68) |
| Multiplicity | 2.0 | (2.0) | 2.0 | (1.8) |
| Refinement | | | | |
| No. of unique | 17,207 | (1,684) | 44,413 | (4,301) |
| reflections | | | | |

| | | |
|---|---|---|
| R_{work}/R_{free}^{b} | 23.46/27.13 | 17.82/19.44 |
| | (32.01/35.15) | (28.46/26.83) |
| No. of atoms | | |
| Protein | 1,854 | 3,641 |
| Ligand | 82 | 233 |
| Water | 42 | 325 |
| R.m.s.^{c} deviations | | |
| Bond length (Å) | 0.009 | 0.007 |
| Bond angles (°) | 0.99 | 1.00 |
| Ramachandran | | |
| plot (%) | | |
| Favored region | 96.05 | 99.08 |
| Allowed region | 3.07 | 0.69 |
| Outliers | 0.88 | 0.23 |

ªHighest resolution shell is shown in parentheses;
^{b}10% of the observed intensities was excluded from refinement for cross validation purposes;
^{c}R.m.s. means root square.

### Synthesis of Compounds: Materials and Instrumentation

All reactions involving air-sensitive reagents were carried out in anhydrous solvents under an atmosphere of nitrogen. Reagents and solvents purchased from commercial supplies were used as received. Reactions were monitored by thin-layer chromatography (TLC) on Baker-flex® silica gel plates (IB2-F) using UV-light (254 and 365 nm) detection or high-performance liquid chromatography/mass spectrometry (HPLC-MS). Column chromatography was carried out using Teledyne ISCO CombiFlash system equipped with either a silica or C-18 column. NMR spectra were recorded at room temperature using a Bruker Avance III HD 600 MHz spectrometer (¹H NMR at 600 MHz). Chemical shifts (δ) are reported in parts per million (ppm) with reference to solvent signals [¹H-NMR: DMSO-d₆ (2.50 ppm) and CD₃OD (3.31 ppm)]. Signal patterns are reported as s (singlet), d (doublet), t (triplet), q (quartet), h (heptet), m (multiplet), and br (broad). Coupling constants (J) are given in Hz. HRMS measurements were performed using ThermoFisher Scientific Q Exactive instrument. Abbreviations presented in experimental procedures are referred to the following definitions: t-BuOK, potassium tert-butylate; CH₂Cl₂, dichloromethane; CH₃CN, acetonitrile; CH₃OH, methanol; DIEA, N,N-diisopropylethylamine; DME, 1,2-dimethoxyethane; DMF, N,N-dimethylformamide; EtOAc, ethyl acetate; HATU, O-(7-azabenzotriazol-1-yl)-N,N,N′,N′-tetramethyluronium hexafluorophosphate; LiOH·H₂O, lithium hydroxide monohydrate; NaOH_{(aq)}, aqueous sodium hydroxide solution; Na₂CO₃₍ₛₐₜ₎, saturated aqueous sodium carbonate solution; Na₂SO₄, sodium sulfate; NaHCO₃, sodium bicarbonate; NBS, N-bromosuccinimide; Pd(dppf)Cl₂·CH₂Cl₂, 1,1′-bis(diphenylphosphino)ferrocene]dichloropalladium(II) dichloromethane complex; TEA, triethylamine; THF, tetrahydrofuran; XPhos Pd G3, (2-dicyclohexylphosphino-2′,4′,6′-triisopropyl-1,1′-biphenyl)[2-(2′-amino-1,1′-biphenyl)]palladium(II) methanesulfonate.

### Example 1: Compounds Synthesis

General procedure for the Suzuki-Miyaura reaction: C—C bond formation between aryl halides and boronic acid derivatives

An oven-dried microwave vial, which was equipped with a magnetic stir bar, was charged with aryl halide (1.0 equiv.), boronic acid derivative (1.5-2.0 equiv.), and Pd(dppf)Cl₂·CH₂Cl₂ (10 mol %). The vial was sealed with a microwave cap, evacuated and backfilled with nitrogen. Anhydrous DME and nitrogen-flushed Na₂CO₃₍ₛₐₜ₎(v/v, 1:1) were then sequentially added via syringe. The mixture was vacuum-purged and refilled with nitrogen for three cycles and then heated by microwave reactor at 110° C. for 1 h (or at indicated temperature for indicated period of time). The resulting mixture was diluted with EtOAc and washed with water. The organic layer was dried over anhydrous Na₂SO₄, filtered, concentrated in vacuo, and purified by column chromatography using a silica-packed column with CH₃OH—CH₂Cl₂ gradient and/or C-18 column with water-CH₃CN gradient to afford desired cross-coupling product.

Synthesis of intermediate 2a

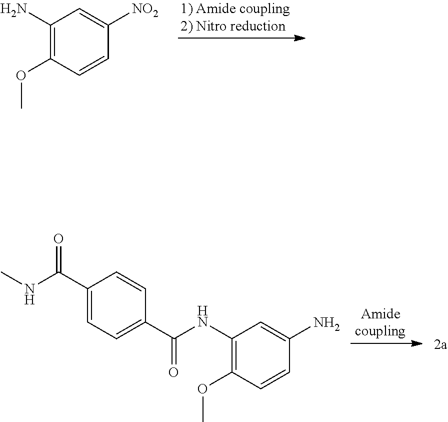 [SMILES: COc1ccc([N+](=O)[O-])cc1N ; CNC(=O)c1ccc(C(=O)Nc2cc(N)ccc2OC)cc1]

### Step 1: N¹-(2-Methoxy-5-nitrophenyl)-N⁴-methylterephthalamide

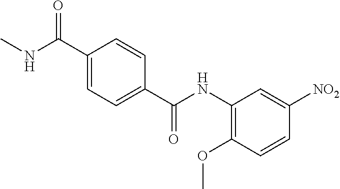 [SMILES: CNC(=O)c1ccc(C(=O)Nc2cc([N+](=O)[O-])ccc2OC)cc1]

To a solution of 4-(methylcarbamoyl)benzoic acid (198 mg, 1.1 mmol, 1.0 equiv.), 2-methoxy-5-nitroaniline (1a) (277 mg, 1.65 mmol, 1.5 equiv.) and HATU (502 mg, 1.32 mmol, 1.2 equiv.) in anhydrous DMF (2 mL) was added DIEA (290 μL, 1.65 mmol, 1.5 equiv.) under nitrogen. The reaction mixture was stirred at room temperature for 16 h and then quenched by the addition of water. The aqueous layer was extracted twice with EtOAc, and the combined organic extracts were washed with brine, dried over anhydrous Na₂SO₄, filtered, and concentrated. The residue was purified by column chromatography on silica gel (CH₃OH/CH₂Cl₂, 1:99 to 5:95) to afford N¹ 1-(2-methoxy-5-nitrophenyl)-N⁴-methylterephthalamide (364 mg, 99%) as a pale yellow solid; ¹H NMR (600 MHz, DMSO-d₆) δ 9.90 (s, 1H), 8.77 (d, J=2.6 Hz, 1H), 8.63 (d, J=4.4 Hz, 1H), 8.15 (dd, J=9.2, 2.6 Hz, 1H), 8.04 (d, J=8.2 Hz, 2H), 7.97 (d, J=8.2 Hz, 2H), 7.34 (d, J=9.2 Hz, 1H), 4.00 (s, 3H), 2.82 (d, J=4.4 Hz, 3H); HRMS (ESI) m/z calcd for C₁₆H₁₆N₃O₅[M+H]⁺330.1084, found 330.1079.

### Step 2: N¹ 1-(5-Amino-2-methoxyphenyl)-N⁴-methylterephthalamide

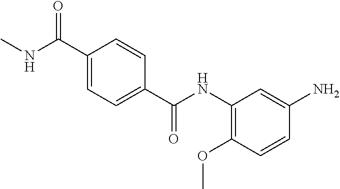 [SMILES: CNC(=O)c1ccc(C(=O)Nc2cc(N)ccc2OC)cc1]

To a solution of N¹-(2-methoxy-5-nitrophenyl)-N⁴-methylterephthalamide (344 mg, 1 mmol, 1.0 equiv.) and acetic acid (1.5 mL) in CH₃OH (5 mL) was added zinc powder (650 mg, 10 mmol, 10.0 equiv.) at 0° C. with vigorous stirring. The resulting mixture was allowed to warm up to room temperature and stirred for 1 h. The excess zinc powder was then filtered off through a pad of Celite, and the resulting solution was neutralized to pH 7 with 4M NaOH_{(aq)}. The residue was concentrated in vacuo and purified by column chromatography on silica gel (CH₃OH/CH₂Cl₂, 1:99 to 10:90) to afford N¹-(5-amino-2-methoxyphenyl)-N⁴-methylterephthalamide (90 mg, 29%) as a yellow solid; ¹H NMR (600 MHz, DMSO-d₆) δ 9.32 (s, 1H), 8.60-8.58 (m, 1H), 7.99 (d, J=8.4 Hz, 2H), 7.94 (d, J=8.4 Hz, 2H), 7.14 (d, J=2.6 Hz, 1H), 6.80 (d, J=8.7 Hz, 1H), 6.38 (dd, J=8.7, 2.6 Hz, 1H), 4.75 (br s, 2H), 3.70 (s, 3H), 2.81 (d, J=4.6 Hz, 3H); HRMS (ESI) m/z calcd for C₁₆H₁₈IN₃O₃[M+H]⁺300.1342, found 300.1338.

### Step 3: N¹-(5-(3-Iodo-1-methyl-1H-indazole-5-carboxamido)-2-methoxyphenyl)-N⁴-methylterephthalamide (2a)

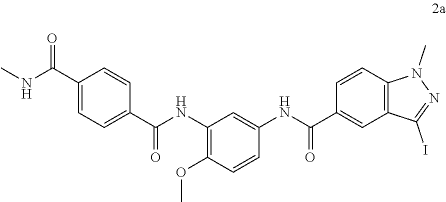 [SMILES: CNC(=O)c1ccc(C(=O)Nc2cc(NC(=O)c3ccc4c(c3)c(I)nn4C)ccc2OC)cc1]

To a solution of N¹-(5-amino-2-methoxyphenyl)-N⁴-methylterephthalamide (60 mg, 0.2 mmol, 1.0 equiv.), 3-iodo-1-methyl-1H-indazole-5-carboxylic acid (67 mg, 0.22 mmol, 1.1 equiv.), and HATU (91 mg, 0.24 mmol, 1.2 equiv.) in anhydrous DMF (1.5 mL) was added DIEA (53 μL, 0.3 mmol, 1.5 equiv.) under nitrogen. The reaction mixture was stirred at room temperature for 16 h and then quenched by the addition of water. The aqueous layer was extracted twice with EtOAc, and the combined organic extracts were washed with brine, dried over anhydrous Na₂SO₄, filtered, and concentrated. The residue was purified by column chromatography on silica gel (CH₃OH/CH₂Cl₂, 1:99 to 5:95) to afford 2a (111 mg, 95%) as a pale yellow solid; ¹H NMR (600 MHz, DMSO-d₆) δ 10.37 (s, 1H), 9.64 (s, 1H), 8.61 (d, J=4.4 Hz, 1H), 8.20 (d, J=2.0 Hz, 1H), 8.17 (s, 1H), 8.09 (d, J=8.8, 1H), 8.05 (d, J=8.2 Hz, 2H), 7.96 (d, J=8.2 Hz, 2H), 7.79 (d, J=8.8 Hz, 1H), 7.69 (dd, J=9.0, 2.0 Hz, 1H), 7.11 (d, J=9.0 Hz, 1H), 4.12 (s, 3H), 3.85 (s, 3H), 2.82 (d, J=4.4 Hz, 3H); HRMS (ESI) m/z calcd for C₂₅H₂₃IN₅O₄[M+H]⁺584.0789, found 584.0782.

### Synthesis of Intermediate 2b

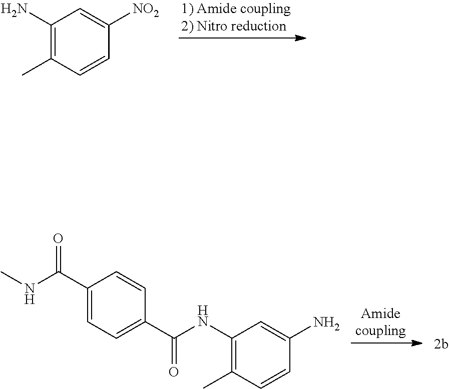 [SMILES: Cc1ccc([N+](=O)[O-])cc1N ; CNC(=O)c1ccc(C(=O)Nc2cc(N)ccc2C)cc1]

### Step 1: N¹-Methyl-N⁴-(2-methyl-5-nitrophenyl)terephthalamide

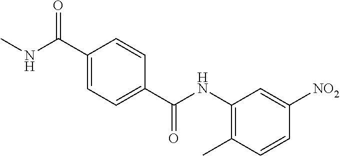 [SMILES: CNC(=O)c1ccc(C(=O)Nc2cc([N+](=O)[O-])ccc2C)cc1]

To a solution of 4-(methylcarbamoyl)benzoic acid (900 mg, 5 mmol, 1.0 equiv.), 2-methyl-5-nitroaniline (1b) (913 mg, 6 mmol, 1.2 equiv.), and HATU (2.28 g, 6 mmol, 1.2 equiv.) in anhydrous DMF (10 mL) was added DIEA (1.3 mL, 7.5 mmol, 1.5 equiv.) under nitrogen. The reaction mixture was stirred at room temperature for 16 h and then quenched by the addition of water. The resulting precipitate was collected, washed with ether, and dried in vacuo to afford N¹-methyl-N⁴-(2-methyl-5-nitrophenyl)terephthalamide (776 mg, 50%) as a pale yellow solid; ¹H NMR (600 MHz, DMSO-d₆) δ 10.26 (s, 1H), 8.63 (d, J=4.3 Hz, 1H), 8.36 (d, J=1.1 Hz, 1H), 8.07-8.04 (m, 3H), 7.99 (d, J=8.1 Hz, 2H), 7.58 (d, J=8.5 Hz, 1H), 2.82 (d, J=4.3 Hz, 3H), 2.40 (s, 3H); HRMS (ESI) m/z calcd for C₁₆H₁₆N₃O₄[M+H]⁺314.1135, found 314.1126.

### Step 2: N¹-(5-Amino-2-methylphenyl)-N⁴-methylterephthalamide

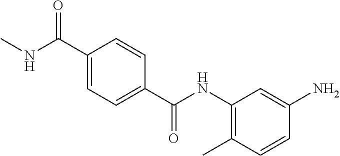 [SMILES: CNC(=O)c1ccc(C(=O)Nc2cc(N)ccc2C)cc1]

To a solution of N¹-methyl-N⁴-(2-methyl-5-nitrophenyl)terephthalamide (690 mg, 2.2 mmol, 1.0 equiv.) and acetic acid (3.3 mL) in THF/CH₃OH (15 mL, v/v=2:1) was added zinc powder (1.44 g, 22 mmol, 10.0 equiv.) at 0° C. with vigorous stirring. The resulting mixture was allowed to warm up to room temperature and stirred for 1 h. The excess zinc powder was then filtered off through a pad of Celite, and the filtrate was neutralized to pH 7 with 4M NaOH_{(aq)} and concentrated. The resulting residue was washed with water and dried in vacuo to afford N¹-(5-amino-2-methylphenyl)-N⁴-methylterephthalamide (543 mg, 87%) as a pale yellow solid, which was directly used in the next step without further purification.

### Step 3: N¹-(5-(3-Iodo-1-methyl-1H-indazole-5-carboxamido)-2-methylphenyl)-N⁴-methylterephthalamide (2b)

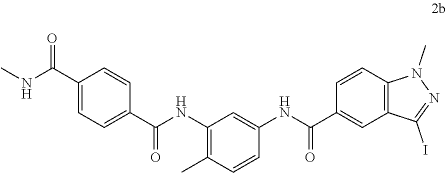 [SMILES: CNC(=O)c1ccc(C(=O)Nc2cc(NC(=O)c3ccc4c(c3)c(I)nn4C)ccc2C)cc1]

To a solution of N¹-(5-amino-2-methylphenyl)-N⁴-methylterephthalamide (255 mg, 0.9 mmol, 1.0 equiv.), 3-iodo-1-methyl-1H-indazole-5-carboxylic acid (270 mg, 0.9 mmol, 1.0 equiv.), and HATU (411 mg, 1.08 mmol, 1.2 equiv.) in anhydrous DMF (3 mL) was added DIEA (188 μL, 1.08 mmol, 1.2 equiv.) under nitrogen. The reaction mixture was stirred at room temperature for 16 h and then quenched by the addition of water. The precipitate was collected and washed with water. The aqueous solution was extracted twice with EtOAc, and the combined organic extracts were washed with brine, dried over anhydrous Na₂SO₄, filtered, and concentrated. The residue and collected precipitate were combined and purified by column chromatography on silica gel (CH₃OH/CH₂Cl₂, 1:99 to 5:95) to afford 2b (420 mg, 82%) as a pale yellow solid; ¹H NMR (600 MHz, DMSO-d₆) δ 10.41 (s, 1H), 10.06 (s, 1H), 8.62 (d, J=4.0 Hz, 1H), 8.17 (s, 1H), 8.09-8.06 (m, 3H), 7.98 (d, J=8.0 Hz, 2H), 7.88 (s, 1H), 7.79 (d, J=8.8 Hz, 1H), 7.64 (d, J=8.3 Hz, 1H), 7.27 (d, J=8.3 Hz, 1H), 4.12 (s, 3H), 2.82 (d, J=4.0 Hz, 3H), 2.23 (s, 3H); HRMS (ESI) m/z calcd for C₂₅H₂₃IN₅O₃[M+H]⁺568.084, found 568.0826.

Syntheses of compounds 3-15

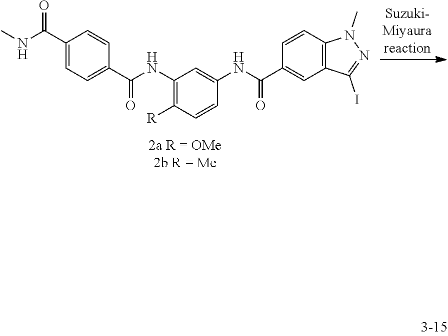

### N¹-(2-Methoxy-5-(1-methyl-3-(1-methyl-1H-indol-2-yl)-1H-indazole-5-carboxamido)phenyl)-N⁴-methylterephthalamide (3)

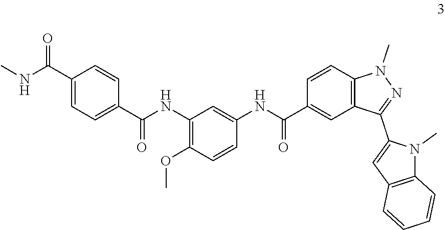

The general procedure employed for the Suzuki-Miyaura reaction was followed using aryl iodide 2a (29 mg, 0.05 mmol, 1.0 equiv.), (1-methyl-1H-indol-2-yl)boronic acid (17 mg, 0.1 mmol, 2.0 equiv.), and Pd(dppf)Cl₁·CH₂Cl₂ (4 mg, 0.005 mmol, 0.1 equiv.) in DME/Na₂CO₃₍ₛₐₜ₎(0.4 mL, v/v=1:1) at 90° C. for 5 h. Isolation and purification afforded title compound 3 (10 mg, 34%) as a pale yellow solid; ¹H NMR (600 MHz, DMSO-d₆) δ 10.37 (s, 1H), 9.64 (s, 1H), 8.67 (s, 1H), 8.62 (s, 1H), 8.19 (s, 1H), 8.11 (d, J=8.6 Hz, 1H), 8.04 (d, J=7.4 Hz, 2H), 7.96 (d, J=7.4 Hz, 2H), 7.86 (d, J=8.6 Hz, 1H), 7.69 (d, J=7.7 Hz, 2H), 7.57 (d, J=7.9 Hz, 1H), 7.26-7.22 (m, 2H), 7.11 (d, J=7.7 Hz, 2H), 4.22 (s, 3H), 4.08 (s, 3H), 3.85 (s, 3H), 2.82 (s, 3H); HRMS (ESI) m/z calcd for C₃₄H₃₁IN₆O₄[M+H]⁺587.2401, found 587.2401.

### N¹-(5-(3-(4-Amino-2-methylphenyl)-1-methyl-1H-indazole-5-carboxamido)-2-methoxyphenyl)-N⁴-methylterephthalamide (4)

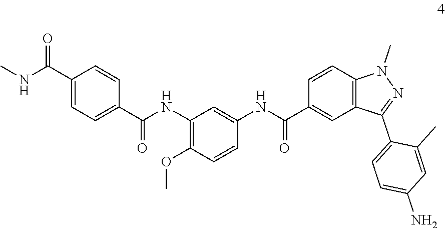

The general procedure employed for the Suzuki-Miyaura reaction was followed using aryl iodide 2a (13 mg, 0.022 mmol, 1.0 equiv.), 3-methyl-4-(4,4,5,5-tetramethyl-1,3,2-dioxaborolan-2-yl)aniline (10 mg, 0.044 mmol, 2.0 equiv.), and Pd(dppf)Cl₂·CH₂Cl₂ (1.6 mg, 0.002 mmol, 0.1 equiv.) in DME/Na₂CO₃₍ₛₐₜ₎(0.4 mL, v/v=1:1) at 85° C. for 90 min. Isolation and purification afforded title compound 4 (8.5 mg, 68%) as a pale yellow solid; ¹H NMR (600 MHz, DMSO-d₆) δ 10.25 (s, 1H), 9.62 (s, 1H), 8.61 (d, J=4.4 Hz, 1H), 8.31 (s, 1H), 8.15 (d, J=2.0 Hz, 1H), 8.04-8.02 (m, 3H), 7.95 (d, J=8.2 Hz, 2H), 7.73 (d, J=8.8 Hz, 1H), 7.66 (dd, J=9.0, 2.0 Hz, 1H), 7.23 (d, J=8.0 Hz, 1H), 7.08 (d, J=9.0 Hz, 1H), 6.58-6.55 (m, 2H), 5.24 (s, 2H), 4.10 (s, 3H), 3.83 (s, 3H), 2.81 (d, J=4.4 Hz, 3H), 2.25 (s, 3H); HRMS (ESI) m/z calcd for C₃₂H₃₁N₆O₄[M+H]⁺563.2401, found 563.2387.

### N¹-(5-(3-(2,4-Dimethylphenyl)-1-methyl-1H-indazole-5-carboxamido)-2-methoxyphenyl)-N⁴-methylterephthalamide (5)

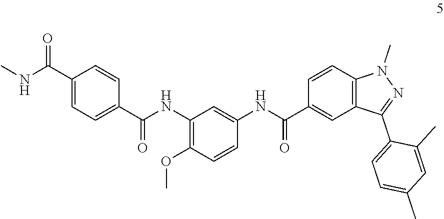

The general procedure employed for the Suzuki-Miyaura reaction was followed using aryl iodide 2a (13 mg, 0.022 mmol, 1.0 equiv.), (2,4-dimethylphenyl)boronic acid (7 mg, 0.044 mmol, 2.0 equiv.), and Pd(dppf)Cl₂·CH₂Cl₂ (1.6 mg, 0.002 mmol, 0.1 equiv.) in DME/Na₂CO₃₍ₛₐₜ₎(0.4 mL, v/v=1:1). Isolation and purification afforded title compound 5 (3.5 mg, 28%) as a yellow solid; ¹H NMR (600 MHz, DMSO-d₆) δ 10.25 (s, 1H), 9.62 (s, 1H), 8.62 (d, J=4.4 Hz, 1H), 8.32 (s, 1H), 8.14 (d, J=1.9 Hz, 1H), 8.06 (d, J=8.9, 1H), 8.03 (d, J=8.2 Hz, 2H), 7.95 (d, J=8.2 Hz, 2H), 7.79 (d, J=8.9 Hz, 1H), 7.66 (dd, J=8.9, 1.9 Hz, 1H), 7.46 (d, J=7.7 Hz, 1H), 7.23 (s, 1H), 7.19 (d, J=7.7 Hz, 1H), 7.08 (d, J=8.9 Hz, 1H), 4.15 (s, 3H), 3.83 (s, 3H), 2.81 (d, J=4.4 Hz, 3H), 2.37 (s, 3H), 2.35 (s, 3H); HRMS (ESI) m/z calcd for C₃₃H₃₂N₅O₄[M+H]⁺562.2448, found 562.2442.

### N¹-(2-Methoxy-5-(1-methyl-3-(o-tolyl)-1H-indazole-5-carboxamido)phenyl)-N⁴-methylterephthalamide (6)

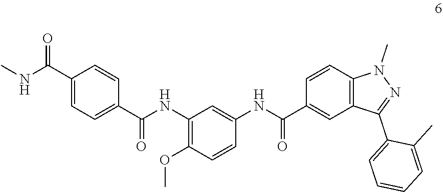

The general procedure employed for the Suzuki-Miyaura reaction was followed using aryl iodide 2a (13 mg, 0.022 mmol, 1.0 equiv.), o-tolylboronic acid (6 mg, 0.044 mmol, 2.0 equiv.), and Pd(dppf)Cl₂·CH₂Cl₂ (1.6 mg, 0.002 mmol, 0.1 equiv.) in DME/Na₂CO₃₍ₛₐₜ₎(0.4 mL, v/v=1:1). Isolation and purification afforded title compound 6 (12 mg, 99%) as a pale yellow solid; ¹H NMR (600 MHz, DMSO-d₆) δ 10.26 (s, 1H), 9.62 (s, 1H), 8.61 (d, J=4.4 Hz, 1H), 8.33 (s, 1H), 8.14 (d, J=2.2 Hz, 1H), 8.07 (d, J=8.9 Hz, 1H), 8.03 (d, J=8.2 Hz, 2H), 7.95 (d, J=8.2 Hz, 2H), 7.81 (d, J=8.9 Hz, 1H), 7.65 (dd, J=9.0, 2.2 Hz, 1H), 7.58-7.57 (m, 1H), 7.43-7.38 (m, 3H), 7.09 (d, J=9.0 Hz, 1H), 4.16 (s, 3H), 3.83 (s, 3H), 2.81 (d, J=4.4 Hz, 3H), 2.39 (s, 3H); HRMS (ESI) m/z calcd for C₃₂H₃₀N₅O₄[M+H]⁺548.2292, found 548.2286.

### N¹-(5-(3-(4-Aminophenyl)-1-methyl-1H-indazole-5-carboxamido)-2-methoxyphenyl)-N⁴-methylterephthalamide (7)

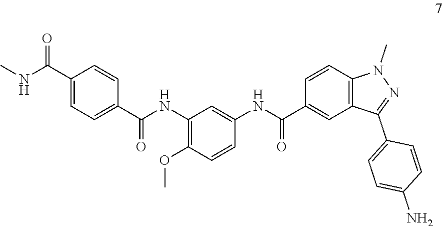 [SMILES: CNC(=O)c1ccc(C(=O)Nc2cc(NC(=O)c3ccc4c(c3)c(-c3ccc(N)cc3)nn4C)ccc2OC)cc1]

The general procedure employed for the Suzuki-Miyaura reaction was followed using aryl iodide 2a (13 mg, 0.022 mmol, 1.0 equiv.), 4-(4,4,5,5-tetramethyl-1,3,2-dioxaborolan-2-yl)aniline (10 mg, 0.044 mmol, 2.0 equiv.), and Pd(dppf)Cl₂·CH₂Cl₂ (1.6 mg, 0.002 mmol, 0.1 equiv.) in DME/Na₂CO₃₍ₛₐₜ₎(0.4 mL, v/v=1:1). Isolation and purification afforded title compound 7 (7 mg, 58%) as a pale yellow solid; ¹H NMR (600 MHz, DMSO-d₆) δ 10.31 (s, 1H), 9.64 (s, 1H), 8.63 (s, 1H), 8.62 (d, J=4.4 Hz, 1H), 8.18 (d, J=2.2 Hz, 1H), 8.04 (d, J=8.3 Hz, 2H), 8.01 (d, J=8.9 Hz, 1H), 7.96 (d, J=8.3 Hz, 2H), 7.72-7.71 (m, 3H), 7.69 (dd, J=9.0, 2.2 Hz, 1H), 7.11 (d, J=9.0 Hz, 1H), 6.73 (d, J=8.5 Hz, 2H), 5.37 (s, 2H), 4.08 (s, 3H), 3.85 (s, 3H), 2.82 (d, J=4.4 Hz, 3H); HRMS (ESI) m/z calcd for C₃₁H₂₉N₆O₄[M+H]⁺549.2244, found 549.2236.

### N¹-Methyl-N⁴-(2-methyl-5-(1-methyl-3-(1-methyl-1H-indol-2-yl)-1H-indazole-5-carboxamido)phenyl)terephthalamide (8)

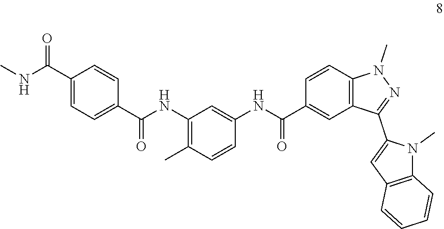

The general procedure employed for the Suzuki-Miyaura reaction was followed using aryl iodide 2b (11 mg, 0.02 mmol, 1.0 equiv.), (1-methyl-1H-indol-2-yl)boronic acid (10 mg, 0.04 mmol, 2.0 equiv.), and Pd(dppf)Cl₂·CH₂Cl₂ (1.6 mg, 0.002 mmol, 0.1 equiv.) in DME/Na₂CO₃₍ₛₐₜ₎(0.4 mL, v/v=1:1) at 110° C. for 2 h. Isolation and purification afforded title compound 8 (8 mg, 71%) as a pale yellow solid; ¹H NMR (600 MHz, DMSO-d₆) δ 10.41 (s, 1H), 10.06 (s, 1H), 8.66 (s, 1H), 8.62 (d, J=4.5 Hz, 1H), 8.09 (d, J=8.9 Hz, 1H), 8.06 (d, J=8.3 Hz, 2H), 7.97 (d, J=8.3 Hz, 2H), 7.87-7.86 (m, 2H), 7.69 (d, J=7.9 Hz, 1H), 7.65 (dd, J=8.2, 1.9 Hz, 1H), 7.57 (d, J=8.2 Hz, 1H), 7.28-7.24 (m, 2H), 7.21 (s, 1H), 7.12 (t, J=7.4 Hz, 1H), 4.22 (s, 3H), 4.07 (s, 3H), 2.82 (d, J=4.5 Hz, 3H), 2.23 (s, 3H); HRMS (ESI) m/z calcd for C₃₄H₃₁N₆O₃[M+H]⁺571.2452, found 571.2445.

### N¹-(5-(3-(4-Amino-2-methylphenyl)-1-methyl-1H-indazole-5-carboxamido)-2-methylphenyl)-N⁴-methylterephthalamide (9)

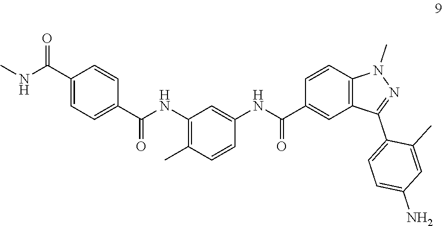

The general procedure employed for the Suzuki-Miyaura reaction was followed using aryl iodide 2b (28 mg, 0.05 mmol, 1.0 equiv.), 3-methyl-4-(4,4,5,5-tetramethyl-1,3,2-dioxaborolan-2-yl)aniline (18 mg, 0.075 mmol, 1.5 equiv.), and Pd(dppf)Cl₂·CH₂Cl₂ (4 mg, 0.005 mmol, 0.1 equiv.) in DME/Na₂CO₃₍ₛₐₜ₎(0.4 mL, v/v=1:1). Isolation and purification afforded title compound 9 (15 mg, 53%) as a pale yellow solid; ¹H NMR (600 MHz, DMSO-d₆) δ 10.28 (s, 1H), 10.04 (s, 1H), 8.61 (d, J=4.5 Hz, 1H), 8.31 (s, 1H), 8.05 (d, J=8.3 Hz, 2H), 8.02 (d, J=8.9 Hz, 1H), 7.97 (d, J=8.3 Hz, 2H), 7.84 (d, J=1.7 Hz, 1H), 7.74 (d, J=8.9 Hz, 1H), 7.61 (dd, J=8.3, 1.7 Hz, 1H), 7.25-7.22 (m, 2H), 6.58-6.55 (m, 2H), 5.25 (s, 2H), 4.10 (s, 3H), 2.82 (d, J=4.5 Hz, 3H), 2.25 (s, 3H), 2.21 (s, 3H); HRMS (ESI) m/z calcd for C₃₂H₃₁N₆O₃[M+H]⁺547.2452, found 547.2437.

### N¹-(5-(3-(6-Aminopyridin-3-yl)-1-methyl-1H-indazole-5-carboxamido)-2-methylphenyl)-N⁴-methylterephthalamide (10)

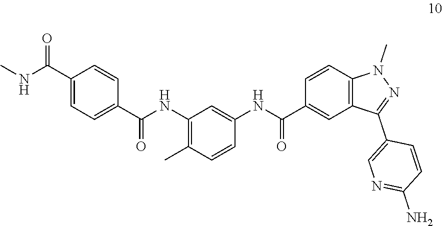 [SMILES: CNC(=O)c1ccc(C(=O)Nc2cc(NC(=O)c3ccc4c(c3)c(-c3ccc(N)nc3)nn4C)ccc2C)cc1]

The general procedure employed for the Suzuki-Miyaura reaction was followed using aryl iodide 2b (11 mg, 0.02 mmol, 1.0 equiv.), (6-aminopyridin-3-yl)boronic acid (6 mg, 0.04 mmol, 2.0 equiv.), and Pd(dppf)Cl₂·CH₂Cl₂ (1.6 mg, 0.002 mmol, 0.1 equiv.) in DME/Na₂CO₃₍ₛₐₜ₎(0.4 mL, v/v=1:1). Isolation and purification afforded title compound 10 (10 mg, 94%) as a pale yellow solid; ¹H NMR (600 MHz, DMSO-d₆) δ 10.37 (s, 1H), 10.05 (s, 1H), 8.64-8.60 (m, 3H), 8.06 (d, J=8.3 Hz, 2H), 8.02 (dd, J=8.9, 1.5 Hz, 1H), 7.99 (dd, J=8.6, 2.4 Hz, 1H), 7.97 (d, J=8.3 Hz, 2H), 7.86 (d, J=2.0 Hz, 1H), 7.76 (d, J=8.9 Hz, 1H), 7.63 (dd, J=8.4, 2.0 Hz, 1H), 7.27 (d, J=8.4 Hz, 1H), 6.62 (d, J=8.6 Hz, 1H), 6.23 (s, 2H), 4.11 (s, 3H), 2.82 (d, J=4.5 Hz, 3H), 2.23 (s, 3H); HRMS (ESI) m/z calcd for C₃₀H₂₈N₇O₃[M+H]⁺534.2248, found 534.224.

### N¹-(5-(3-(6-Aminopyridin-3-yl)-1-methyl-1H-indazole-5-carboxamido)-2-methylphenyl)-N⁴-methylterephthalamide (11)

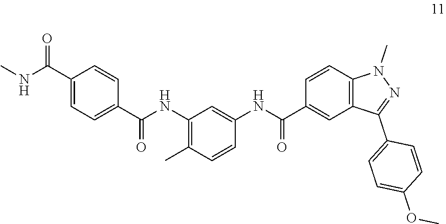

The general procedure employed for the Suzuki-Miyaura reaction was followed using aryl iodide 2b (9 mg, 0.015 mmol, 1.0 equiv.), (4-methoxyphenyl)boronic acid (5 mg, 0.03 mmol, 2.0 equiv.), and Pd(dppf)Cl₂·CH₂Cl₂ (1.3 mg, 0.0015 mmol, 0.1 equiv.) in DME/Na₂CO₃₍ₛₐₜ₎(0.4 mL, v/v=1:1). Isolation and purification afforded title compound 11 (7 mg, 80%) as a white solid; ¹H NMR (600 MHz, DMSO-d₆) δ 10.38 (s, 1H), 10.06 (s, 1H), 8.66 (s, 1H), 8.61 (d, J=4.5 Hz, 1H), 8.06 (d, J=8.3 Hz, 2H), 8.03 (d, J=8.9 Hz, 1H), 7.99-7.96 (m, 4H), 7.84 (d, J=1.5 Hz, 1H), 7.78 (d, J=8.9 Hz, 1H), 7.66 (dd, J=8.4, 1.5 Hz, 1H), 7.27 (d, J=8.4 Hz, 1H), 7.13 (d, J=8.7 Hz, 2H), 4.13 (s, 3H), 3.83 (s, 3H), 2.82 (d, J=4.5 Hz, 3H), 2.23 (s, 3H); HRMS (ESI) m/z calcd for C₃₂H₃₀N₅O₄[M+H]⁺548.2292, found 548.2285.

### N¹-Methyl-N⁴-(2-methyl-5-(1-methyl-3-(pyridin-4-yl)-1H-indazole-5-carboxamido)phenyl)terephthalamide (12)

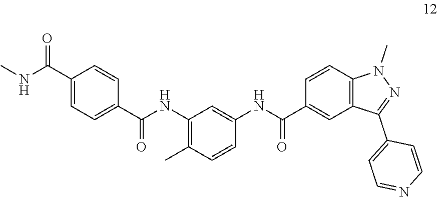 [SMILES: CNC(=O)c1ccc(C(=O)Nc2cc(NC(=O)c3ccc4c(c3)c(-c3ccncc3)nn4C)ccc2C)cc1]

The general procedure employed for the Suzuki-Miyaura reaction was followed using aryl iodide 2b (11 mg, 0.02 mmol, 1.0 equiv.), pyridin-4-ylboronic acid (4 mg, 0.03 mmol, 1.5 equiv.), and Pd(dppf)Cl₂·CH₂Cl₂ (1.6 mg, 0.002 mmol, 0.1 equiv.) in DME/Na₂CO₃₍ₛₐₜ₎(0.4 mL, v/v=1:1). Isolation and purification afforded title compound 12 (2.5 mg, 24%) as a pale yellow foam; ¹H NMR (600 MHz, DMSO-d₆) δ 10.42 (s, 1H), 10.06 (s, 1H), 8.79 (s, 1H), 8.75-8.74 (m, 2H), 8.61 (d, J=4.5 Hz, 1H), 8.08-8.06 (m, 5H), 7.97 (d, J=8.4 Hz, 2H), 7.89 (d, J=8.8 Hz, 1H), 7.85 (d, J=2.0 Hz, 1H), 7.67 (dd, J=8.4, 2.0 Hz, 1H), 7.28 (d, J=8.4 Hz, 1H), 4.21 (s, 3H), 2.82 (d, J=4.5 Hz, 3H), 2.24 (s, 3H); HRMS (ESI) m/z calcd for C₃₀H₂₇N₆O₃[M+H]⁺519.2139, found 519.2131.

### N¹-(5-(3-(2-Aminopyrimidin-5-yl)-1-methyl-1H-indazole-5-carboxamido)-2-methylphenyl)-N⁴-methylterephthalamide (13)

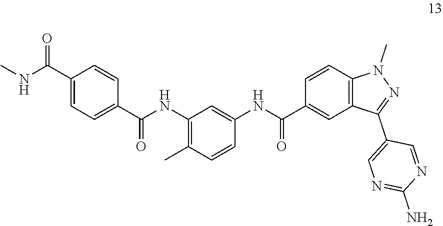 [SMILES: CNC(=O)c1ccc(C(=O)Nc2cc(NC(=O)c3ccc4c(c3)c(-c3cnc(N)nc3)nn4C)ccc2C)cc1]

The general procedure employed for the Suzuki-Miyaura reaction was followed using aryl iodide 2b (11 mg, 0.02 mmol, 1.0 equiv.), (2-aminopyrimidin-5-yl)boronic acid (4 mg, 0.03 mmol, 1.5 equiv.), and Pd(dppf)Cl₂·CH₂Cl₂ (1.6 mg, 0.002 mmol, 0.1 equiv.) in DME/Na₂CO₃₍ₛₐₜ₎(0.4 mL, v/v=1:1). Isolation and purification afforded title compound 13 (7.5 mg, 72%) as a pale yellow solid; ¹H NMR (600 MHz, DMSO-d₆) δ 10.36 (s, 1H), 10.06 (s, 1H), 8.89 (s, 2H), 8.66 (s, 1H), 8.62 (d, J=3.4 Hz, 1H), 8.07-8.03 (m, 3H), 7.97 (d, J=7.7 Hz, 2H), 7.86 (s, 1H), 7.79 (d, J=8.8 Hz, 1H), 7.62 (d, J=8.2 Hz, 1H), 7.27 (d, J=8.2 Hz, 1H), 6.96 (s, 2H), 4.13 (s, 3H), 2.82 (d, J=3.4 Hz, 3H), 2.23 (s, 3H); HRMS (ESI) m/z calcd for C₂₉H₂₇N₈O₃[M+H]+ 535.2200, found 535.2191.

### N¹-(5-(3-(6-Amino-2-methylpyridin-3-yl)-1-methyl-1H-indazole-5-carboxamido)-2-methylphenyl)-N⁴-methylterephthalamide (14)

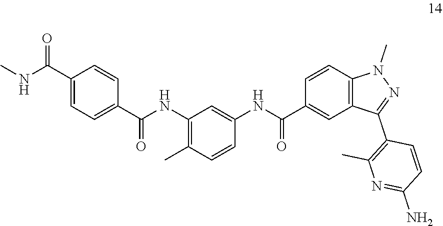 [SMILES: CNC(=O)c1ccc(C(=O)Nc2cc(NC(=O)c3ccc4c(c3)c(-c3ccc(N)nc3C)nn4C)ccc2C)cc1]

The general procedure employed for the Suzuki-Miyaura reaction was followed using aryl iodide 2b (11 mg, 0.02 mmol, 1.0 equiv.), 6-methyl-5-(4,4,5,5-tetramethyl-1,3,2-dioxaborolan-2-yl)pyridin-2-amine (7 mg, 0.03 mmol, 1.5 equiv.), and Pd(dppf)Cl₂·CH₂Cl₂ (1.6 mg, 0.002 mmol, 0.1 equiv.) in DME/Na₂CO₃₍ₛₐₜ₎(0.4 mL, v/v=1:1). Isolation and purification afforded title compound 14 (8 mg, 73%) as a pale yellow solid; ¹H NMR (600 MHz, DMSO-d₆) δ 10.27 (s, 1H), 10.04 (s, 1H), 8.61 (d, J=4.5 Hz, 1H), 8.31 (s, 1H), 8.06-8.03 (m, 3H), 7.96 (d, J=8.3 Hz, 2H), 7.83 (d, J=1.4 Hz, 1H), 7.76 (d, J=8.9 Hz, 1H), 7.60 (dd, J=8.5, 1.4 Hz, 1H), 7.55 (d, J=8.3 Hz, 1H), 7.24 (d, J=8.5 Hz, 1H), 6.45 (d, J=8.3 Hz, 1H), 6.13 (br s, 2H), 4.12 (s, 3H), 2.82 (d, J=4.5 Hz, 3H), 2.34 (s, 3H), 2.21 (s, 3H); HRMS (ESI) m/z calcd for C₃₁H₃₀N₇O₃[M+H]+ 548.2404, found 548.2393.

### N¹-(5-(3-(6-Amino-4-methylpyridin-3-yl)-1-methyl-1H-indazole-5-carboxamido)-2-methylphenyl)-N¹-methylterephthalamide (15)

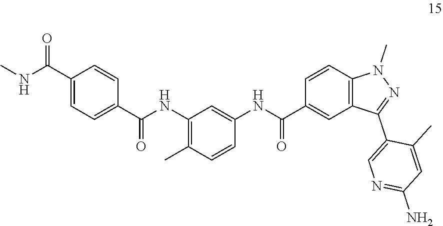

The general procedure employed for the Suzuki-Miyaura reaction was followed using aryl iodide 2b (11 mg, 0.02 mmol, 1.0 equiv.), 4-methyl-5-(4,4,5,5-tetramethyl-1,3,2-dioxaborolan-2-yl)-2-pyridinamine hydrochloride (8 mg, 0.03 mmol, 1.5 equiv.), and Pd(dppf)Cl₂·CH₂Cl₂ (1.6 mg, 0.002 mmol, 0.1 equiv.) in DME/Na₂CO₃₍ₛₐₜ₎(0.4 mL, v/v=1:1). Isolation and purification afforded title compound 15 (3.2 mg, 29%) as a pale yellow solid; ¹H NMR (600 MHz, DMSO-d₆) δ 10.31 (s, 1H), 10.04 (s, 1H), 8.62 (d, J=4.4 Hz, 1H), 8.36 (s, 1H), 8.11 (s, 1H), 8.06-8.03 (m, 3H), 7.97 (d, J=8.2 Hz, 2H), 7.83 (d, J=1.3 Hz, 1H), 7.77 (d, J=8.9 Hz, 1H), 7.61 (dd, J=8.4, 1.3 Hz, 1H), 7.24 (d, J=8.4 Hz, 1H), 6.46 (s, 1H), 6.08 (s, 2H), 4.12 (s, 3H), 2.82 (d, J=4.4 Hz, 3H), 2.25 (s, 3H), 2.22 (s, 3H); HRMS (ESI) m/z calcd for C₃₁H₃₀N₇O₃[M+H]⁺548.2404, found 548.2393.

Syntheses of compounds 16, 17, and 20

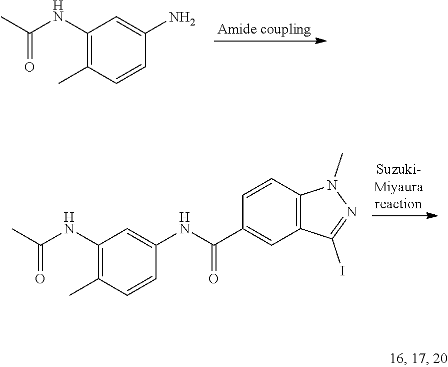 [SMILES: CC(=O)Nc1cc(N)ccc1C ; CC(=O)Nc1cc(NC(=O)c2ccc3c(c2)c(I)nn3C)ccc1C]

### Step 1: N-(3-Acetamido-4-methylphenyl)-3-iodo-1-methyl-1H-indazole-5-carboxamide

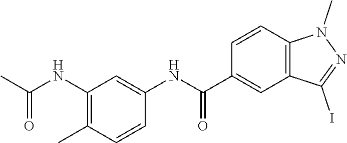 [SMILES: CC(=O)Nc1cc(NC(=O)c2ccc3c(c2)c(I)nn3C)ccc1C]

To a solution of 3-iodo-1-methyl-1H-indazole-5-carboxylic acid (604 mg, 2 mmol, 1.0 equiv.), N-(5-amino-2-methylphenyl)acetamide (493 mg, 3 mmol, 1.5 equiv.), and HATU (913 mg, 2.4 mmol, 1.2 equiv.) in anhydrous DMF (2 mL) was added DIEA (700 μL, 4 mmol, 2.0 equiv.) under nitrogen. The reaction mixture was stirred at room temperature for 16 h and then quenched by the addition of water. The precipitate was collected, sequentially washed with water and ether, and dried in vacuo to afford N-(3-acetamido-4-methylphenyl)-3-iodo-1-methyl-1H-indazole-5-carboxamide (810 mg, 90%) as a pale yellow solid, which was directly used in the next step without further purification; ¹H NMR (600 MHz, DMSO-d₆) δ 10.35 (s, 1H), 9.33 (s, 1H), 8.15 (s, 1H), 8.06 (d, J=8.8 Hz, 1H), 7.87 (s, 1H), 7.78 (d, J=8.8 Hz, 1H), 7.54 (d, J=8.2 Hz, 1H), 7.17 (d, J=8.2 Hz, 1H), 4.11 (s, 3H), 2.17 (s, 3H), 2.07 (s, 3H); HRMS (ESI) m/z calcd for C₁₈H₁₈N₄O₂[M+H]⁺449.0468, found 449.0461.

### Step 2a: N-(3-Acetamido-4-methylphenyl)-3-(6-amino-4-methylpyridin-3-yl)-1-methyl-1H-indazole-5-carboxamide (16)

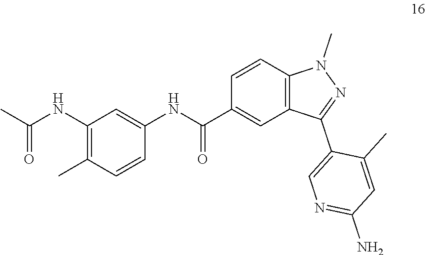 [SMILES: CC(=O)Nc1cc(NC(=O)c2ccc3c(c2)c(-c2cnc(N)cc2C)nn3C)ccc1C]

The general procedure employed for the Suzuki-Miyaura reaction was followed using N-(3-acetamido-4-methylphenyl)-3-iodo-1-methyl-1H-indazole-5-carboxamide (23 mg, 0.05 mmol, 1.0 equiv.), 4-methyl-5-(4,4,5,5-tetramethyl-1,3,2-dioxaborolan-2-yl)-2-pyridinamine hydrochloride (20 mg, 0.075 mmol, 1.5 equiv.), and Pd(dppf)Cl₂·CH₂Cl₂ (4 mg, 0.005 mmol, 0.1 equiv.) in DME/Na₂CO₃₍ₛₐₜ₎(0.4 mL, v/v=1:1). Isolation and purification afforded title compound 16 (9 mg, 42%) as a pale yellow solid; ¹H NMR (600 MHz, DMSO-d₆) δ 10.25 (s, 1H), 9.32 (s, 1H), 8.34 (s, 1H), 8.11 (s, 1H), 8.03 (d, J=8.8 Hz, 1H), 7.82 (s, 1H), 7.75 (d, J=8.8 Hz, 1H), 7.52 (d, J=8.3 Hz, 1H), 7.15 (d, J=8.3 Hz, 1H), 6.46 (s, 1H), 6.08 (s, 2H), 4.12 (s, 3H), 2.25 (s, 3H), 2.16 (s, 3H), 2.06 (s, 3H); HRMS (ESI) m/z calcd for C₂₄H₂₅N₆O₂[M+H]⁺429.2033, found 429.2024.

### Step 2b: N-(3-Acetamido-4-methylphenyl)-3-(6-aminopyridin-3-yl)-1-methyl-1H-indazole-5-carboxamide (17)

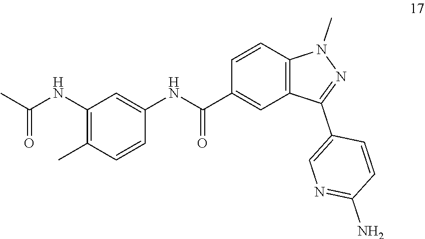 [SMILES: CC(=O)Nc1cc(NC(=O)c2ccc3c(c2)c(-c2ccc(N)nc2)nn3C)ccc1C]

The general procedure employed for the Suzuki-Miyaura reaction was followed using N-(3-acetamido-4-methylphenyl)-3-iodo-1-methyl-1H-indazole-5-carboxamide (225 mg, 0.5 mmol, 1.0 equiv.), (6-aminopyridin-3-yl)boronic acid (104 mg, 0.75 mmol, 1.5 equiv.), and Pd(dppf)Cl₂·CH₂Cl₂ (41 mg, 0.05 mmol, 0.1 equiv.) in DME/Na₂CO₃₍ₛₐₜ₎(4 mL, v/v=1:1). Isolation and purification afforded title compound 17 (145 mg, 70%) as a white solid; ¹H NMR (600 MHz, DMSO-d₆) δ 10.31 (s, 1H), 9.32 (s, 1H), 8.62 (s, 2H), 8.03-8.01 (m, 2H), 7.85 (s, 1H), 7.75 (d, J=8.8 Hz, 1H), 7.54 (d, J=8.3 Hz, 1H), 7.17 (d, J=8.3 Hz, 1H), 6.64 (d, J=8.6 Hz, 1H), 6.31 (s, 2H), 4.10 (s, 3H), 2.18 (s, 3H), 2.07 (s, 3H); HRMS (ESI) m/z calcd for C₂₃H₂₃N₆O₂[M+H]⁺415.1877, found 415.1865.

### Step 2c: N-(3-Acetamido-4-methylphenyl)-3-(4-amino-2-methylphenyl)-1-methyl-1H-indazole-5-carboxamide (20)

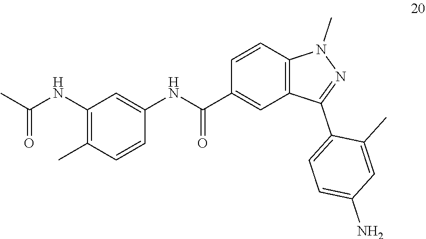 [SMILES: CC(=O)Nc1cc(NC(=O)c2ccc3c(c2)c(-c2ccc(N)cc2C)nn3C)ccc1C]

The general procedure employed for the Suzuki-Miyaura reaction was followed using N-(3-acetamido-4-methylphenyl)-3-iodo-1-methyl-1H-indazole-5-carboxamide (23 mg, 0.05 mmol, 1.0 equiv.), 3-methyl-4-(4,4,5,5-tetramethyl-1,3,2-dioxaborolan-2-yl)aniline (19 mg, 0.075 mmol, 1.5 equiv.), and Pd(dppf)Cl₂·CH₂Cl₂ (4 mg, 0.005 mmol, 0.1 equiv.) in DME/Na₂CO₃₍ₛₐₜ₎(0.4 mL, v/v=1:1). Isolation and purification afforded title compound 20 (18 mg, 84%) as a pale yellow solid; ¹H NMR (600 MHz, DMSO-d₆) δ 10.23 (s, 1H), 9.31 (s, 1H), 8.29 (s, 1H), 8.00 (d, J=8.8 Hz, 1H), 7.82 (s, 1H), 7.72 (d, J=8.8 Hz, 1H), 7.52 (d, J=8.3 Hz, 1H), 7.22 (d, J=8.1 Hz, 1H), 7.14 (d, J=8.3 Hz, 1H), 6.58 (s, 1H), 6.56 (d, J=8.1 Hz, 1H), 5.25 (s, 2H), 4.10 (s, 3H), 2.25 (s, 3H), 2.16 (s, 3H), 2.06 (s, 3H); HRMS (ESI) m/z calcd for C₂₅H₂₆N₅O₂[M+H]⁺428.2081, found 428.2070.

### Synthesis of Compound 18

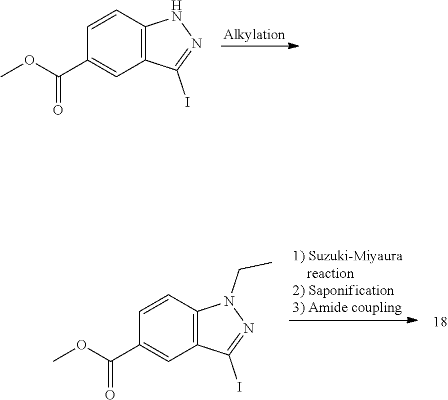 [SMILES: COC(=O)c1ccc2[nH]nc(I)c2c1 ; CCn1nc(I)c2cc(C(=O)OC)ccc21]

### Step 1: Methyl 1-ethyl-3-iodo-1H-indazole-5-carboxylate

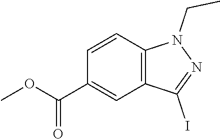 [SMILES: CCn1nc(I)c2cc(C(=O)OC)ccc21]

To a solution of methyl 3-iodo-1H-indazole-5-carboxylate (91 mg, 0.3 mmol, 1.0 equiv.) and t-BuOK (40 mg, 0.36 mmol, 1.2 equiv.) in anhydrous THF (3 mL) was added iodoethane (30 μL, 0.36 mmol, 1.2 equiv.) at 0° C. under nitrogen. The reaction mixture was allowed to warm up to room temperature and stirred for 16 h. After being quenched by the addition of water, the mixture was extracted twice with EtOAc, and the combined organic extracts were washed with brine, dried over anhydrous Na₂SO₄, filtered, and concentrated. The residue was purified by column chromatography on silica gel (EtOAc/hexane, 0:100 to 10:90) to afford methyl 1-ethyl-3-iodo-1H-indazole-5-carboxylate (48 mg, 48%) as a white solid; ¹H NMR (600 MHz, DMSO-d₆) δ 8.03 (s, 1H), 8.00 (d, J=8.9 Hz, 1H), 7.82 (d, J=8.9 Hz, 1H), 4.49 (q, J=7.2 Hz, 2H), 3.89 (s, 3H), 1.40 (t, J=7.2 Hz, 3H); HRMS (ESI) m/z calcd for C₁₁H₁₂IN₂O₂[M+H]⁺330.9937, found 330.9928.

### Step 2: N-(3-Acetamido-4-methylphenyl)-3-(6-aminopyridin-3-yl)-1-ethyl-1H-indazole-5-carboxamide (18)

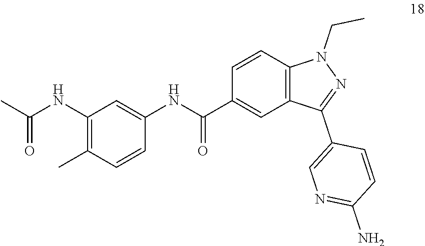 [SMILES: CCn1nc(-c2ccc(N)nc2)c2cc(C(=O)Nc3ccc(C)c(NC(C)=O)c3)ccc21]

The general procedure employed for the Suzuki-Miyaura reaction was followed using methyl 1-ethyl-3-iodo-1H-indazole-5-carboxylate (17 mg, 0.05 mmol, 1.0 equiv.), (6-aminopyridin-3-yl)boronic acid (10.5 mg, 0.075 mmol, 1.5 equiv.), and Pd(dppf)Cl₂·CH₂Cl₂ (4 mg, 0.005 mmol, 0.1 equiv.) in DME/Na₂CO₃₍ₛₐₜ₎(0.4 mL, v/v=1:1). To the solution of crude cross-coupling product in THF/water (2 mL, v/v=1:1) was added LiOH·H₂O (6 mg, 0.15 mmol, 3.0 equiv.), and the mixture was vigorously stirred at 45° C. for 16 h. The reaction mixture was neutralized to pH 7 with 4N HCl_{(aq)} and concentrated in vacuo. To the solution of crude hydrolyzed product, N-(5-amino-2-methylphenyl)acetamide (12 mg, 0.075 mmol, 1.5 equiv.), and HATU (23 mg, 0.06 mmol, 1.2 equiv.) in anhydrous DMF (0.2 mL) was added DIEA (26 μL, 0.15 mmol, 3.0 equiv.) under nitrogen. The reaction mixture was stirred at room temperature for 16 h and then quenched by the addition of water. The aqueous layer was extracted twice with EtOAc, and the combined organic extracts were washed with brine, dried over anhydrous Na₂SO₄, filtered, and concentrated. The residue was purified by column chromatography on silica gel (CH₃OH/CH₂Cl₂ 3:97 to 5:95) to afford title compound 18 (17 mg, 77%) as a yellow foam; ¹H NMR (600 MHz, DMSO-d₆) δ 10.30 (s, 1H), 9.32 (s, 1H), 8.62 (s, 2H), 8.01-7.99 (m, 2H), 7.85 (s, 1H), 7.78 (d, J=8.8 Hz, 1H), 7.54 (d, J=8.2 Hz, 1H), 7.17 (d, J=8.2 Hz, 1H), 6.62 (d, J=8.5 Hz, 1H), 6.22 (s, 2H), 4.50 (q, J=7.2 Hz, 2H), 2.18 (s, 3H), 2.07 (s, 3H), 1.44 (t, J=7.2 Hz, 3H); HRMS (ESI) m/z calcd for C₂₄H₂₅N₆O₂[M+H]⁺429.2033, found 429.2024.

### Synthesis of Compound 19

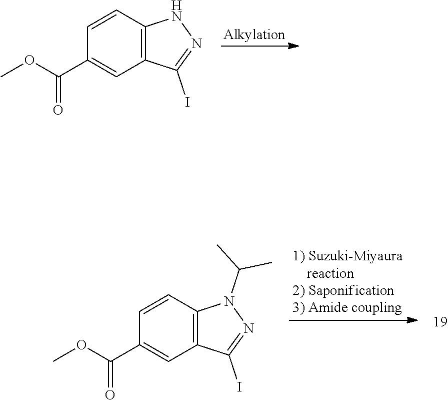 [SMILES: COC(=O)c1ccc2[nH]nc(I)c2c1 ; COC(=O)c1ccc2c(c1)c(I)nn2C(C)C]

### Step 1: Methyl 3-iodo-1-isopropyl-1H-indazole-5-carboxylate

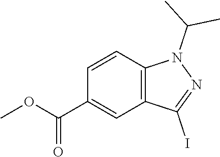 [SMILES: COC(=O)c1ccc2c(c1)c(I)nn2C(C)C]

To a solution of methyl 3-iodo-1H-indazole-5-carboxylate (91 mg, 0.3 mmol, 1.0 equiv.) and t-BuOK (40 mg, 0.36 mmol, 1.2 equiv.) in anhydrous THF (1 mL) was added 2-bromopropane (40 μL, 0.36 mmol, 1.2 equiv.) at 0° C. under nitrogen. The reaction mixture was allowed to warm up to room temperature and stirred for 16 h. After being quenched by the addition of water, the mixture was extracted twice with EtOAc, and the combined organic extracts were washed with brine, dried over anhydrous Na₂SO₄, filtered, and concentrated. The residue was purified by column chromatography on silica gel (EtOAc/hexane, 0:100 to 5:95) to afford methyl 3-iodo-1-isopropyl-1H-indazole-5-carboxylate (56 mg, 54%) as a white solid; ¹H NMR (600 MHz, DMSO-d₆) δ 8.04 (s, 1H), 8.00 (d, J=8.9 Hz, 1H), 7.84 (d, J=8.9 Hz, 1H), 5.06 (h, J=6.6 Hz, 1H), 3.89 (s, 3H), 1.48 (d, J=6.6 Hz, 6H); HRMS (ESI) m/z calcd for C₁₂H₁₄N₂O₂[M+H]⁺345.0094, found 345.009.

### Step 2: N-(3-Acetamido-4-methylphenyl)-3-(6-aminopyridin-3-yl)-1-isopropyl-1H-indazole-5-carboxamide (19)

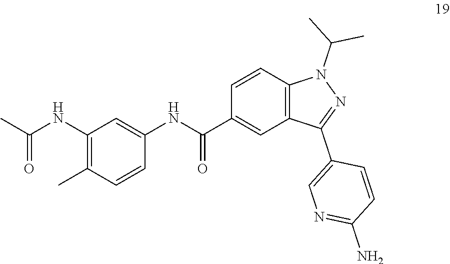 [SMILES: CC(=O)Nc1cc(NC(=O)c2ccc3c(c2)c(-c2ccc(N)nc2)nn3C(C)C)ccc1C]

The general procedure employed for the Suzuki-Miyaura reaction was followed using methyl 3-iodo-1-isopropyl-1H-indazole-5-carboxylate (18 mg, 0.05 mmol, 1.0 equiv.), (6-aminopyridin-3-yl)boronic acid (10.5 mg, 0.075 mmol, 1.5 equiv.), and Pd(dppf)Cl₂·CH₂Cl₂ (4 mg, 0.005 mmol, 0.1 equiv.) in DME/Na₂CO₃₍ₛₐₜ₎(0.4 mL, v/v=1:1). To the solution of crude cross-coupling product in THF/water (2 mL, v/v=1:1) was added LiOH·H₂O (6 mg, 0.15 mmol, 3.0 equiv.), and the mixture was vigorously stirred at 45° C. for 16 h. The resulting mixture was neutralized to pH 7 with 4N HCl_{(aq)} and concentrated in vacuo. To the solution of crude hydrolyzed product, N-(5-amino-2-methylphenyl)acetamide (12 mg, 0.075 mmol, 1.5 equiv.), and HATU (23 mg, 0.06 mmol, 1.2 equiv.) in anhydrous DMF (0.2 mL) was added DIEA (26 μL, 0.15 mmol, 3.0 equiv.) under nitrogen. The reaction mixture was stirred at room temperature for 16 h and then quenched by the addition of water. The aqueous layer was extracted twice with EtOAc, and the combined organic extracts were washed with brine, dried over anhydrous Na₂SO₄, filtered, and concentrated. The residue was purified by column chromatography on silica gel (CH₃OH/CH₂Cl₂, 3:97 to 5:95) to afford title compound 19 (17 mg, 81%) as a yellow foam; ¹H NMR (600 MHz, DMSO-d₆) δ 10.30 (s, 1H), 9.32 (s, 1H), 8.62 (s, 2H), 8.00-7.98 (m, 2H), 7.85 (s, 1H), 7.81 (d, J=8.9 Hz, 1H), 7.55 (d, J=8.2 Hz, 1H), 7.17 (d, J=8.2 Hz, 1H), 6.63 (d, J=8.5 Hz, 1H), 6.22 (s, 2H), 5.08-5.02 (m, 1H), 2.18 (s, 3H), 2.07 (s, 3H), 1.54 (s, 3H), 1.53 (s, 3H); HRMS (ESI) m/z calcd for C₂₅H₂₇N₆O₂[M+H]⁺443.2190, found 443.2179.

### Synthesis of Compounds 21-22

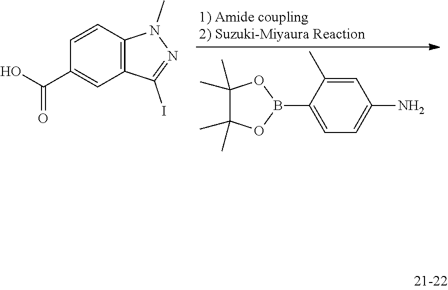

### Synthesis of Compound 21

### Step 1: N-(3-Acetamidophenyl)-3-iodo-1-methyl-1H-indazole-5-carboxamide

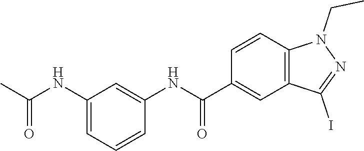 [SMILES: CCn1nc(I)c2cc(C(=O)Nc3cccc(NC(C)=O)c3)ccc21]

To a solution of 3-iodo-1-methyl-1H-indazole-5-carboxylic acid (31 mg, 0.1 mmol, 1.0 equiv.), N-(3-aminophenyl)acetamide (23 mg, 0.15 mmol, 1.5 equiv.), and HATU (46 mg, 0.12 mmol, 1.2 equiv.) in anhydrous DMF (0.2 mL) was added DIEA (35 μL, 0.2 mmol, 2.0 equiv.) under nitrogen. The resulting mixture was stirred at room temperature for 16 h and then quenched by the addition of water. The precipitate was collected, sequentially washed with water and ether, and dried in vacuo to afford N-(3-acetamidophenyl)-3-iodo-1-methyl-1H-indazole-5-carboxamide (37 mg, 85%) as a light brown solid, which was directly used in the next step without further purification; ¹H NMR (600 MHz, DMSO-d₆) δ 10.41 (s, 1H), 9.99 (s, 1H), 8.15 (s, 1H), 8.12 (s, 1H), 8.07 (d, J=8.7 Hz, 1H), 7.78 (d, J=8.7 Hz, 1H), 7.45 (d, J=7.4 Hz, 1H), 7.33 (d, J=7.4 Hz, 1H), 7.26 (t, J=7.4 Hz, 1H), 4.12 (s, 3H), 2.05 (s, 3H); HRMS (ESI) m/z calcd for C₁₇H₁₆N₄O₂[M+H]⁺435.0312, found 435.0305.

### Step 2: N-(3-Acetamidophenyl)-3-(4-amino-2-methylphenyl)-1-methyl-1H-indazole-5-carboxamide (21)

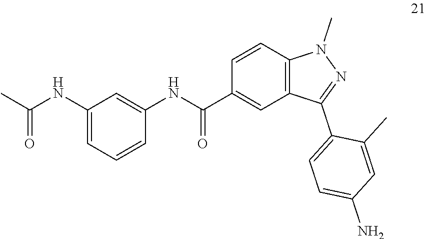 [SMILES: CC(=O)Nc1cccc(NC(=O)c2ccc3c(c2)c(-c2ccc(N)cc2C)nn3C)c1]

The general procedure employed for the Suzuki-Miyaura reaction was followed using N-(3-acetamidophenyl)-3-iodo-1-methyl-1H-indazole-5-carboxamide (22 mg, 0.05 mmol, 1.0 equiv.), 3-methyl-4-(4,4,5,5-tetramethyl-1,3,2-dioxaborolan-2-yl)aniline (19 mg, 0.075 mmol, 1.5 equiv.), and Pd(dppf)Cl₂·CH₂Cl₂ (4 mg, 0.005 mmol, 0.1 equiv.) in DME/Na₂CO₃₍ₛₐₜ₎(0.4 mL, v/v=1:1). Isolation and purification afforded title compound 21 (8.5 mg, 41%) as a pale yellow solid; ¹H NMR (600 MHz, DMSO-d₆) δ 10.28 (s, 1H), 9.96 (s, 1H), 8.29 (s, 1H), 8.06 (s, 1H), 8.00 (d, J=8.8 Hz, 1H), 7.73 (d, J=8.8 Hz, 1H), 7.42 (d, J=8.2 Hz, 1H), 7.32 (d, J=8.2 Hz, 1H), 7.24-7.21 (m, 2H), 6.57-6.54 (m, 2H), 5.25 (s, 2H), 4.10 (s, 3H), 2.25 (s, 3H), 2.04 (s, 3H); HRMS (ESI) m/z calcd for C₂₄H₂₄N₅O₂[M+H]⁺414.1924, found 414.1918.

### Synthesis of Compound 22

### Step 1: 3-Iodo-1-methyl-N-(4-methyl-3-(methylcarbamoyl)phenyl)-1H-indazole-5-carboxamide

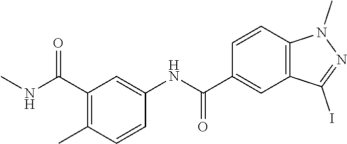 [SMILES: CNC(=O)c1cc(NC(=O)c2ccc3c(c2)c(I)nn3C)ccc1C]

To a solution of 3-iodo-1-methyl-1H-indazole-5-carboxylic acid (31 mg, 0.1 mmol, 1.0 equiv.), 5-amino-N,2-dimethylbenzamide (25 mg, 0.15 mmol, 1.5 equiv.), and HATU (46 mg, 0.12 mmol, 1.2 equiv.) in anhydrous DMF (0.2 mL) was added DIEA (35 μL, 0.2 mmol, 2.0 equiv.) under nitrogen. The resulting mixture was stirred at room temperature for 16 h and then quenched by the addition of water. The precipitate was collected, sequentially washed with water and ether, and dried in vacuo to afford 3-iodo-1-methyl-N-(4-methyl-3-(methylcarbamoyl)phenyl)-1H-indazole-5-carboxamide (34 mg, 75%) as a pale yellow solid, which was directly used in the next step without further purification; ¹H NMR (600 MHz, DMSO-d₆) δ 10.41 (s, 1H), 8.19 (d, J=4.5 Hz, 1H), 8.16 (s, 1H), 8.07 (d, J=8.9 Hz, 1H), 7.80-7.77 (m, 3H), 7.22 (d, J=8.2 Hz, 1H), 4.12 (s, 3H), 2.76 (d, J=4.5 Hz, 3H), 2.29 (s, 3H); HRMS (ESI) m/z calcd for C₁₈H₁₈IN₄O₂[M+H]⁺449.0468, found 449.0458.

### Step 2: 3-(4-Amino-2-methylphenyl)-1-methyl-N-(4-methyl-3-(methylcarbamoyl)phenyl)-1H-indazole-5-carboxamide (22)

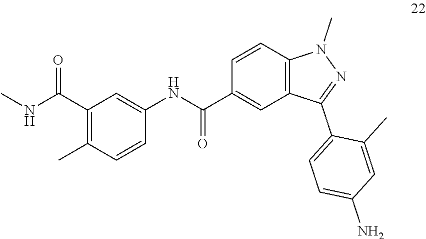

The general procedure employed for the Suzuki-Miyaura reaction was followed using 3-iodo-1-methyl-N-(4-methyl-3-(methylcarbamoyl)phenyl)-1H-indazole-5-carboxamide (22 mg, 0.05 mmol, 1.0 equiv.), 3-methyl-4-(4,4,5,5-tetramethyl-1,3,2-dioxaborolan-2-yl)aniline (19 mg, 0.075 mmol, 1.5 equiv.), and Pd(dppf)Cl₂·CH₂Cl₂ (4 mg, 0.005 mmol, 0.1 equiv.) in DME/Na₂CO₃₍ₛₐₜ₎(0.4 mL, v/v=1:1). Isolation and purification afforded title compound 22 (7 mg, 32%) as a pale yellow foam; ¹H NMR (600 MHz, DMSO-d₆) δ 10.28 (s, 1H), 8.29 (s, 1H), 8.16 (d, J=4.5 Hz, 1H), 8.01 (d, J=8.8 Hz, 1H), 7.75-7.73 (m, 3H), 7.22 (d, J=8.1 Hz, 1H), 7.19 (d, J=8.1 Hz, 1H), 6.58-6.55 (m, 2H), 5.25 (s, 2H), 4.10 (s, 3H), 2.74 (d, J=4.5 Hz, 3H), 2.28 (s, 3H), 2.24 (s, 3H); HRMS (ESI) m/z calcd for C₂₅H₂₆N₅O₂[M+H]⁺428.2081, found 428.2076.

### Synthesis of Compound 23

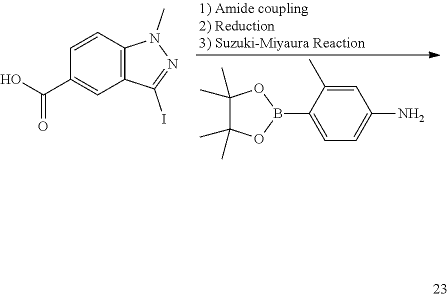

### Step 1: N-(2-Methoxy-5-nitrophenyl)benzamide

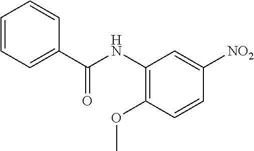 [SMILES: COc1ccc([N+](=O)[O-])cc1NC(=O)c1ccccc1]

To a solution of 2-methoxy-5-nitroaniline (1a) (37 mg, 0.22 mmol, 1.0 equiv.) and TEA (46 μL, 0.33 mmol, 1.5 equiv.) in anhydrous CH₂Cl₂ (2 mL) was added benzoyl chloride (30 μL, 0.24 mmol, 1.1 equiv.) under nitrogen. The reaction mixture was stirred at room temperature for 16 h and then quenched by the addition of water. The aqueous layer was extracted twice with EtOAc, and the combined organic extracts were washed with brine, dried over anhydrous Na₂SO₄, filtered, and concentrated. The residue was purified by column chromatography on silica gel (EtOAc/hexane, 20:80 to 40:60) to afford N-(2-methoxy-5-nitrophenyl)benzamide (60 mg, 99%) as a white solid; ¹H NMR (600 MHz, DMSO-d₆) δ 9.74 (s, 1H), 8.80 (d, J=2.8 Hz, 1H), 8.14 (dd, J=9.2, 2.8 Hz, 1H), 7.98 (d, J=7.4 Hz, 2H), 7.63 (t, J=7.4 Hz, 1H), 7.55 (t, J=7.4 Hz, 2H), 7.33 (d, J=9.2 Hz, 1H), 4.00 (s, 3H); HRMS (ESI) m/z calcd for C₁₄H₁₃N₂O₄[M+H]⁺273.0869, found 273.0865.

### Step 2: N-(3-Benzamido-4-methoxyphenyl)-3-iodo-1-methyl-1H-indazole-5-carboxamide

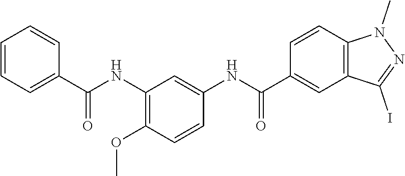 [SMILES: COc1ccc(NC(=O)c2ccc3c(c2)c(I)nn3C)cc1NC(=O)c1ccccc1]

To a solution of N-(2-methoxy-5-nitrophenyl)benzamide (60 mg, 0.22 mmol, 1.0 equiv.) and acetic acid (330 μL) in CH₃OH (3 mL) was added zinc powder (143 mg, 2.2 mmol, 10.0 equiv.) at 0° C. with vigorous stirring. The reaction mixture was allowed to warm up to room temperature and stirred for 1 h. The excess zinc powder was then filtered off through a pad of Celite, and the resulting solution was neutralized to pH 7 with 4M NaOH_{(aq)}. The residue was concentrated in vacuo and purified by reverse-phase column chromatography on C-18 (CH₃CN/water, 5:95 to 100:0) to afford amine as a light yellow solid. To the solution of the amine, 3-iodo-1-methyl-1H-indazole-5-carboxylic acid (72 mg, 0.24 mmol, 1.1 equiv.), and HATU (125 mg, 0.33 mmol, 1.5 equiv.) in anhydrous DMF (0.5 mL) was added DIEA (57 μL, 0.33 mmol, 1.5 equiv.) under nitrogen. The reaction mixture was stirred at room temperature for 16 h and then quenched by the addition of water. The aqueous layer was extracted twice with EtOAc, and the combined organic extracts were washed with brine, dried over anhydrous Na₂SO₄, filtered, and concentrated. The residue was purified by column chromatography on silica gel (CH₃OH/CH₂Cl₂, 1:99 to 2:98) to afford N-(3-benzamido-4-methoxyphenyl)-3-iodo-1-methyl-1H-indazole-5-carboxamide (84 mg, 72%) as a pale yellow foam; ¹H NMR (600 MHz, DMSO-d₆) δ 10.36 (s, 1H), 9.49 (s, 1H), 8.24 (d, J=2.3 Hz, 1H), 8.18 (s, 1H), 8.09 (d, J=8.8 Hz, 1H), 7.98 (d, J=7.4 Hz, 2H), 7.79 (d, J=8.8 Hz, 1H), 7.68 (dd, J=8.9, 2.3 Hz, 1H), 7.60 (t, J=7.4 Hz, 1H), 7.54 (t, J=7.4 Hz, 2H), 7.11 (d, J=8.9 Hz, 1H), 4.12 (s, 3H), 3.85 (s, 3H); HRMS (ESI) m/z calcd for C₂₃H₂₀IN₄O₃[M+H]⁺527.0574, found 527.0573.

### Step 3: 3-(4-Amino-2-methylphenyl)-N-(3-benzamido-4-methoxyphenyl)-1-methyl-1H-indazole-5-carboxamide (23)

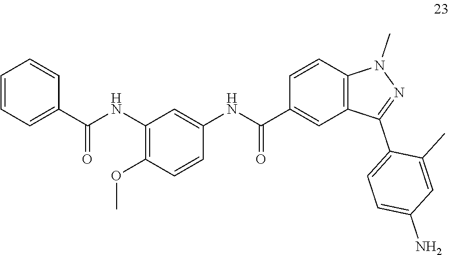 [SMILES: COc1ccc(NC(=O)c2ccc3c(c2)c(-c2ccc(N)cc2C)nn3C)cc1NC(=O)c1ccccc1]

The general procedure employed for the Suzuki-Miyaura reaction was followed using N-(3-benzamido-4-methoxyphenyl)-3-iodo-1-methyl-1H-indazole-5-carboxamide (42 mg, 0.08 mmol, 1.0 equiv.), 3-methyl-4-(4,4,5,5-tetramethyl-1,3,2-dioxaborolan-2-yl)aniline (28 mg, 0.12 mmol, 1.5 equiv.), and Pd(dppf)Cl₂·CH₂Cl₂ (6.5 mg, 0.008 mmol, 0.1 equiv.) in DME/Na₂CO₃₍ₛₐₜ₎(0.4 mL, v/v=1:1) at 110° C. for 2 h. Isolation and purification afforded title compound 23 (29 mg, 57%) as a pale yellow foam; ¹H NMR (600 MHz, DMSO-d₆) δ 10.24 (s, 1H), 9.47 (s, 1H), 8.32 (s, 1H), 8.19 (d, J=2.3 Hz, 1H), 8.03 (d, J=8.8 Hz, 1H), 7.97 (d, J=7.4 Hz, 2H), 7.73 (d, J=8.8 Hz, 1H), 7.65 (dd, J=9.0, 2.3 Hz, 1H), 7.60 (t, J=7.4 Hz, 1H), 7.53 (t, J=7.4 Hz, 2H), 7.23 (d, J=8.0 Hz, 1H), 7.08 (d, J=9.0 Hz, 1H), 6.58-6.55 (m, 2H), 5.25 (s, 2H), 4.10 (s, 3H), 3.83 (s, 3H), 2.25 (s, 3H); HRMS (ESI) m/z calcd for C₃₀H₂₈N₅O₃[M+H]⁺506.2186, found 506.2179.

### Synthesis of Compounds 24-26

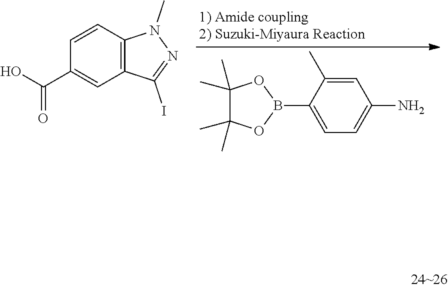

### Synthesis of Compound 24

Step 1: 3-Iodo-N-(4-methoxyphenyl)-1-methyl-1H-indazole-5-carboxamide

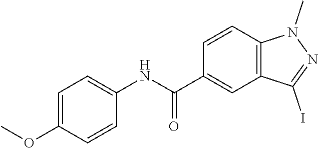 [SMILES: COc1ccc(NC(=O)c2ccc3c(c2)c(I)nn3C)cc1]

To a solution of 3-iodo-1-methyl-1H-indazole-5-carboxylic acid (60 mg, 0.2 mmol, 1.0 equiv.), 4-methoxyaniline (37 mg, 0.3 mmol, 1.5 equiv.), and HATU (91 mg, 0.24 mmol, 1.2 equiv.) in anhydrous DMF (0.2 mL) was added DIEA (42 μL, 0.24 mmol, 1.2 equiv.) under nitrogen. The reaction mixture was stirred at room temperature for 16 h and then quenched by the addition of water. The precipitate was collected, sequentially washed with water and ether, and dried in vacuo to afford 3-iodo-N-(4-methoxyphenyl)-1-methyl-1H-indazole-5-carboxamide (77 mg, 95%) as a pale yellow solid, which was directly used in the next step without further purification; ¹H NMR (600 MHz, DMSO-d₆) δ 10.28 (s, 1H), 8.14 (s, 1H), 8.07 (d, J=8.8 Hz, 1H), 7.78 (d, J=8.8 Hz, 1H), 7.69 (d, J=9.0 Hz, 2H), 6.94 (d, J=9.0 Hz, 2H), 4.11 (s, 3H), 3.75 (s, 3H); HRMS (ESI) m/z calcd for C₁₆H₁₅IN₃O₂[M+H]⁺408.0203, found 408.0191.

### Step 2: 3-(4-Amino-2-methylphenyl)-N-(4-methoxyphenyl)-1-methyl-1H-indazole-5-carboxamide (24)

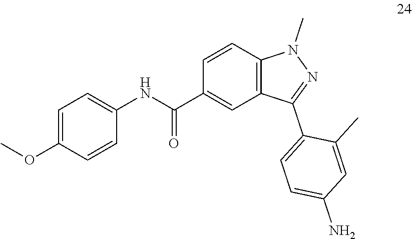

The general procedure employed for the Suzuki-Miyaura reaction was followed using 3-iodo-N-(4-methoxyphenyl)-1-methyl-1H-indazole-5-carboxamide (21 mg, 0.05 mmol, 1.0 equiv.), 3-methyl-4-(4,4,5,5-tetramethyl-1,3,2-dioxaborolan-2-yl)aniline (19 mg, 0.075 mmol, 1.5 equiv.), and Pd(dppf)Cl₂·CH₂Cl₂ (4 mg, 0.005 mmol, 0.1 equiv.) in DME/Na₂CO₃₍ₛₐₜ₎(0.4 mL, v/v=1:1) at 100° C. for 1 h. Isolation and purification afforded title compound 24 (6 mg, 31%) as a pale yellow foam; ¹H NMR (600 MHz, DMSO-d₆) δ 10.15 (s, 1H), 8.27 (s, 1H), 8.00 (d, J=8.8 Hz, 1H), 7.72 (d, J=8.8 Hz, 1H), 7.65 (d, J=9.0 Hz, 2H), 7.21 (d, J=8.1 Hz, 1H), 6.91 (d, J=9.0 Hz, 2H), 6.57-6.54 (m, 2H), 5.25 (s, 2H), 4.10 (s, 3H), 3.74 (s, 3H), 2.24 (s, 3H); HRMS (ESI) m/z calcd for C₂₃H₂₃N₄O₂[M+H]⁺387.1815, found 387.1808.

### Synthesis of Compound 25

### Step 1: 3-Iodo-1-methyl-N-phenyl-1H-indazole-5-carboxamide

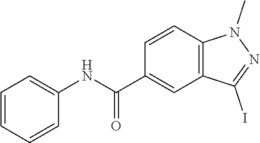 [SMILES: Cn1nc(I)c2cc(C(=O)Nc3ccccc3)ccc21]

To a solution of 3-iodo-1-methyl-1H-indazole-5-carboxylic acid (60 mg, 0.2 mmol, 1.0 equiv.), aniline (28 mg, 0.3 mmol, 1.5 equiv.), and HATU (91 mg, 0.24 mmol, 1.2 equiv.) in anhydrous DMF (0.5 mL) was added DIEA (42 μL, 0.24 mmol, 1.2 equiv.) under nitrogen. The reaction mixture was stirred at room temperature for 16 h and then quenched by the addition of water. The aqueous layer was extracted twice with EtOAc, and the combined organic extracts were washed with brine, dried over anhydrous Na₂SO₄, filtered, and concentrated. The residue was purified by column chromatography on silica gel (CH₃OH/CH₂Cl₂, 1:99 to 5:95) to afford 3-iodo-1-methyl-N-phenyl-1H-indazole-5-carboxamide (64 mg, 85%) as a pale yellow solid; ¹H NMR (600 MHz, DMSO-d₆) δ 10.39 (s, 1H), 8.15 (d, J=1.6 Hz, 1H), 8.07 (dd, J=8.8, 1.6 Hz, 1H), 7.81-7.79 (m, 3H), 7.37 (dd, J=8.4, 7.4 Hz, 2H), 7.11 (t, J=7.4 Hz, 1H), 4.12 (s, 3H); HRMS (ESI) m/z calcd for C₁₅H₁₃IN₃O [M+H]⁺378.0097, found 378.0090.

### Step 2: 3-(4-Amino-2-methylphenyl)-1-methyl-N-phenyl-1H-indazole-5-carboxamide (25)

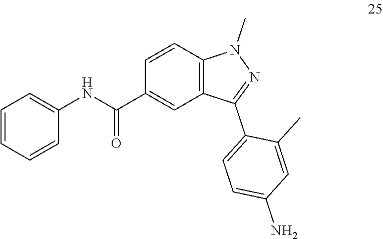 [SMILES: Cc1cc(N)ccc1-c1nn(C)c2ccc(C(=O)Nc3ccccc3)cc12]

The general procedure employed for the Suzuki-Miyaura reaction was followed using 3-iodo-1-methyl-N-phenyl-1H-indazole-5-carboxamide (38 mg, 0.1 mmol, 1.0 equiv.), 3-methyl-4-(4,4,5,5-tetramethyl-1,3,2-dioxaborolan-2-yl)aniline (35 mg, 0.15 mmol, 1.5 equiv.), and Pd(dppf)Cl₂·CH₂Cl₂ (7.5 mg, 0.01 mmol, 0.1 equiv.) in DME/Na₂CO₃₍ₛₐₜ₎(0.4 mL, v/v=1:1) at 90° C. for 3 h. Isolation and purification afforded title compound 25 (15 mg, 42%) as a pale yellow foam; ¹H NMR (600 MHz, DMSO-d₆) δ 10.27 (s, 1H), 8.29 (s, 1H), 8.01 (d, J=8.8 Hz, 1H), 7.76 (d, J=7.9 Hz, 2H), 7.74 (d, J=8.8 Hz, 1H), 7.34 (t, J=7.8 Hz, 2H), 7.22 (d, J=8.1 Hz, 1H), 7.08 (t, J=7.8 Hz, 1H), 6.58 (s, 1H), 6.56 (d, J=8.1 Hz, 1H), 5.25 (s, 2H), 4.10 (s, 3H), 2.25 (s, 3H); HRMS (ESI) m/z calcd for C₂₂H₂₁N₄O [M+H]⁺357.1709, found 357.1702.

### Synthesis of Compound 26

### Step 1: 3-Iodo-N-(4-methoxy phenyl)-N,1-dimethyl-1H-indazole-5-carboxamide

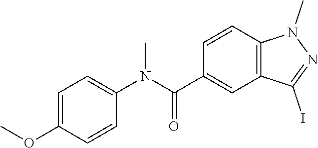 [SMILES: COc1ccc(N(C)C(=O)c2ccc3c(c2)c(I)nn3C)cc1]

To a solution of 3-iodo-1-methyl-1H-indazole-5-carboxylic acid (60 mg, 0.2 mmol, 1.0 equiv.), 4-methoxy-N-methylaniline (42 mg, 0.3 mmol, 1.5 equiv.), and HATU (91 mg, 0.24 mmol, 1.2 equiv.) in anhydrous DMF (0.5 mL) was added DIEA (42 μL, 0.24 mmol, 1.2 equiv.) under nitrogen. The reaction mixture was stirred at room temperature for 16 h and then quenched by the addition of water. The aqueous layer was extracted twice with EtOAc, and the combined organic extracts were washed with brine, dried over anhydrous Na₂SO₄, filtered, and concentrated. The residue was purified by column chromatography on silica gel (CH₃OH/CH₂Cl₂, 1:99 to 2:98) to afford 3-iodo-N-(4-methoxyphenyl)-N,1-dimethyl-1H-indazole-5-carboxamide (80 mg, 95%) as a light yellow oil; ¹H NMR (600 MHz, DMSO-d₆) δ 7.47 (d, J=8.7 Hz, 1H), 7.35-7.32 (m, 2H), 7.12 (d, J=8.8 Hz, 2H), 6.80 (d, J=8.8 Hz, 2H), 4.00 (s, 3H), 3.66 (s, 3H), 3.36 (s, 3H); HRMS (ESI) m/z calcd for C₁₇H₁₇IN₃O₂[M+H]⁺422.0359, found 422.0352.

### Step 2: 3-(4-Amino-2-methylphenyl)-N-(4-methoxyphenyl)-N,1-dimethyl-1H-indazole-5-carboxamide (26)

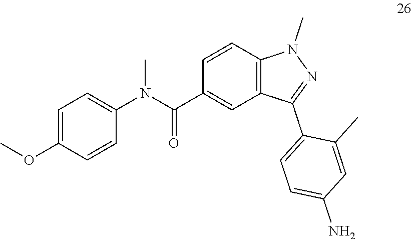

The general procedure employed for the Suzuki-Miyaura reaction was followed using 3-iodo-N-(4-methoxyphenyl)-N,1-dimethyl-1H-indazole-5-carboxamide (22 mg, 0.05 mmol, 1.0 equiv.), 3-methyl-4-(4,4,5,5-tetramethyl-1,3,2-dioxaborolan-2-yl)aniline (18 mg, 0.075 mmol, 1.5 equiv.), and Pd(dppf)Cl₂·CH₂Cl₂ (4 mg, 0.005 mmol, 0.1 equiv.) in DME/Na₂CO₃₍ₛₐₜ₎(0.4 mL, v/v=1:1) at 110° C. for 2 h. Isolation and purification afforded title compound 26 (17 mg, 85%) as a pale yellow oil; ¹H NMR (600 MHz, DMSO-d₆) δ 7.48 (d, J=8.7 Hz, 1H), 7.41 (d, J=8.7 Hz, 1H), 7.38 (s, 1H), 7.10 (d, J=8.5 Hz, 2H), 6.83 (d, J=8.5 Hz, 2H), 6.69 (d, J=7.9 Hz, 1H), 6.50 (s, 1H), 6.45 (d, J=7.9 Hz, 1H), 5.22 (s, 2H), 3.99 (s, 3H), 3.69 (s, 3H), 3.34 (s, 3H), 2.11 (s, 3H); HRMS (ESI) m/z calcd for C₂₄H₂₅N₄O₂[M+H]⁺401.1972, found 401.1965.

### Synthesis of Compound 27

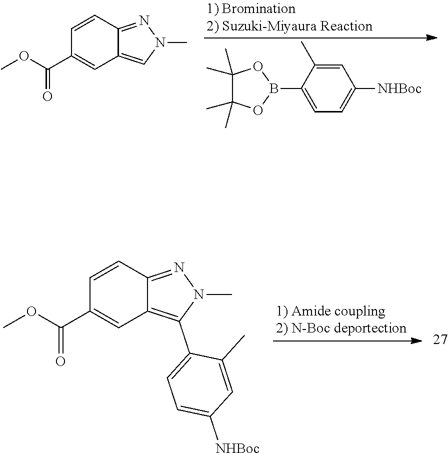

### Step 1: Methyl 3-bromo-2-methyl-2H-indazole-5-carboxylate

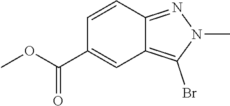

To a solution of methyl 2-methyl-2H-indazole-5-carboxylate (190 mg, 1 mmol, 1.0 equiv.) in anhydrous DMF (3 mL) was added NBS (267 mg, 1.5 mmol, 1.5 equiv.) at 0° C. under nitrogen. The reaction mixture was allowed to warm up to room temperature and stirred for 16 h. Saturated aqueous NaHCO₃ was added and the aqueous layer was extracted twice with EtOAc. The combined organic extracts were washed with brine, dried over anhydrous Na₂SO₄, filtered, and concentrated. The residue was purified by column chromatography on silica gel (EtOAc/hexane, 30:70) to afford methyl 3-bromo-2-methyl-2H-indazole-5-carboxylate (221 mg, 82%) as a white solid; ¹H NMR (600 MHz, DMSO-d₆) δ 8.18 (s, 1H), 7.79 (d, J=9.1 Hz, 1H), 7.70 (d, J=9.1 Hz, 1H), 4.18 (s, 3H), 3.87 (s, 3H); HRMS (ESI) m/z calcd for C₁₀H₁₀BrN₂O₂[M+H]⁺268.9920, found 268.9918.

### Step 2: 3-(4-((tert-Butoxycarbonyl)amino)-2-methylphenyl)-2-methyl-2H-indazole-5-carboxylic acid

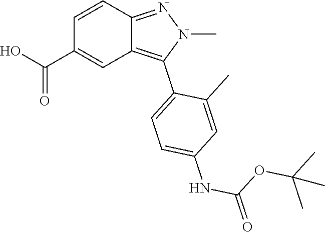 [SMILES: Cc1cc(NC(=O)OC(C)(C)C)ccc1-c1c2cc(C(=O)O)ccc2nn1C]

The general procedure employed for the Suzuki-Miyaura reaction was followed using methyl 3-bromo-2-methyl-2H-indazole-5-carboxylate (68 mg, 0.25 mmol, 1.0 equiv.), tert-butyl (3-methyl-4-(4,4,5,5-tetramethyl-1,3,2-dioxaborolan-2-yl)phenyl)carbamate (125 mg, 0.375 mmol, 1.5 equiv.), and Pd(dppf)Cl₂·CH₂Cl₂ (20 mg, 0.025 mmol, 0.1 equiv.) in DME/Na₂CO₃₍ₛₐₜ₎ (4 mL, v/v=1:1). To the solution of crude cross-coupling product in THF/water (1.5 mL, v/v=2:1) was added LiOH·H₂O (22 mg, 0.5 mmol, 2.0 equiv.), and the mixture was vigorously stirred at 45° C. for 16 h. The resulting mixture was neutralized to pH 7 with 4N HCl_{(aq)} and concentrated in vacuo. The residue was purified by reverse-phase column chromatography on C-18 (CH₃CN/water, 5:95 to 100:0) to afford 3-(4-((tert-butoxycarbonyl)amino)-2-methylphenyl)-2-methyl-2H-indazole-5-carboxylic acid (91 mg, 95%) as a white foam; ¹H NMR (600 MHz, DMSO-d₆) δ 9.61 (s, 1H), 7.87-7.86 (m, 2H), 7.61 (s, 1H), 7.44-7.42 (m, 2H), 7.23 (d, J=8.3 Hz, 1H), 3.88 (s, 3H), 2.01 (s, 3H), 1.50 (s, 9H); HRMS (ESI) m/z calcd for C₂₁H₂₄N₃O₄[M+H]⁺382.1761, found 382.1751.

### Step 3: 3-(4-Amino-2-methylphenyl)-N-(4-methoxyphenyl)-2-methyl-2H-indazole-5-carboxamide (27)

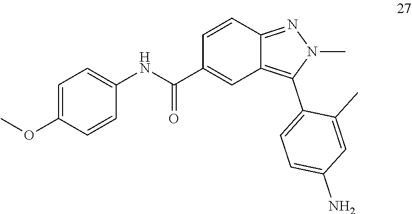

To a solution of 3-(4-((tert-butoxycarbonyl)amino)-2-methylphenyl)-2-methyl-2H-indazole-5-carboxylic acid (38 mg, 0.1 mmol, 1.0 equiv.), 4-methoxyaniline (13 mg, 0.1 mmol, 1.0 equiv.), and HATU (46 mg, 0.12 mmol, 1.2 equiv.) in anhydrous DMF (0.2 mL) was added DIEA (26 μL, 0.15 mmol, 1.5 equiv.) under nitrogen. The reaction mixture was stirred at room temperature for 16 h and then quenched by the addition of water. The aqueous layer was extracted twice with EtOAc, and the combined organic extracts were washed with brine, dried over anhydrous Na₂SO₄, filtered, and concentrated. The crude amide-coupling product was directly treated with a 4N HCl solution in 1,4-dioxane (1 mL), and the mixture was stirred at room temperature for 1 h. The resulting mixture was neutralized to pH 7 with 4M NaOH_{(aq)} and concentrated in vacuo. The residue was purified by reverse-phase column chromatography on C-18 (CH₃CN/water, 5:95 to 100:0) to afford title compound 27 (26 mg, 67%) as a white foam; ¹H NMR (600 MHz, DMSO-d₆) δ 10.06 (s, 1H), 8.06 (s, 1H), 7.82 (d, J=9.1 Hz, 1H), 7.65 (d, J=9.1 Hz, 1H), 7.63 (d, J=9.0 Hz, 2H), 7.03 (d, J=8.2 Hz, 1H), 6.89 (d, J=9.0 Hz, 2H), 6.63 (d, J=1.7 Hz, 1H), 6.58 (dd, J=8.2, 1.7 Hz, 1H), 5.45 (s, 2H), 3.92 (s, 3H), 3.73 (s, 3H), 1.94 (s, 3H); HRMS (ESI) m/z calcd for C₂₃H₂₃N₄O₂[M+H]⁺387.1815, found 387.1807.

### Syntheses of Compounds 28-36

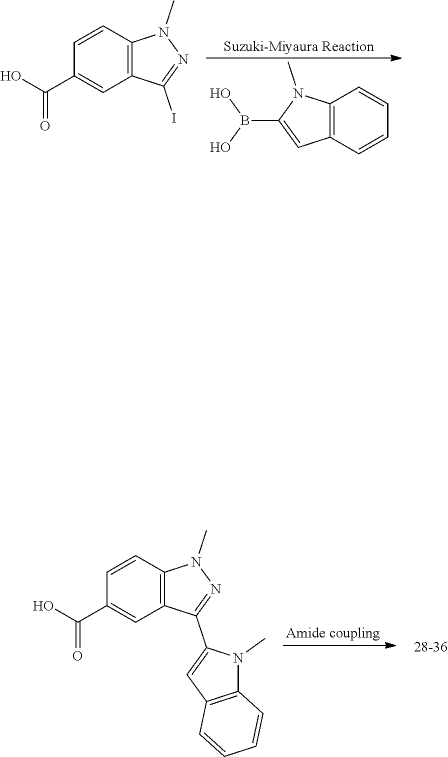 [SMILES: Cn1nc(I)c2cc(C(=O)O)ccc21 ; Cn1c(B(O)O)cc2ccccc21 ; Cn1nc(-c2cc3ccccc3n2C)c2cc(C(=O)O)ccc21]

### Step 1: 1-Methyl-3-(1-methyl-1H-indol-2-yl)-1H-indazole-5-carboxylic acid

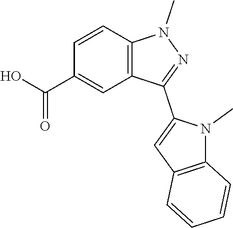 [SMILES: Cn1nc(-c2cc3ccccc3n2C)c2cc(C(=O)O)ccc21]

The general procedure employed for the Suzuki-Miyaura reaction was followed using 3-iodo-1-methyl-1H-indazole-5-carboxylic acid (362 mg, 1.2 mmol, 1.0 equiv.), 1-methyl-2-(4,4,5,5-tetramethyl-1,3,2-dioxaborolan-2-yl)-1H-indole (618 mg, 2.4 mmol, 2.0 equiv.), and Pd(dppf)Cl₂·CH₂Cl₂ (100 mg, 0.12 mmol, 0.1 equiv.) in DME/Na₂CO₃₍ₛₐₜ₎(4 mL, v/v=1:1). Isolation and purification afforded 1-methyl-3-(1-methyl-1H-indol-2-yl)-1H-indazole-5-carboxylic acid (300 mg, 80%) as a light yellow solid; ¹H NMR (600 MHz, DMSO-d₆) δ 12.98 (br s, 1H), 8.59 (s, 1H), 8.04 (d, J=8.8 Hz, 1H), 7.81 (d, J=8.8 Hz, 1H), 7.70 (d, J=7.8 Hz, 1H), 7.55 (d, J=7.8 Hz, 1H), 7.25 (t, J=7.8 Hz, 1H), 7.12 (t, J=7.8 Hz, 1H), 7.04 (s, 1H), 4.19 (s, 3H), 4.05 (s, 3H); HRMS (ESI) m/z calcd for C₁₈H₁₆N₃O₂[M+H]⁺306.1237, found 306.1228.

### Step 2a: N-(4-Methoxyphenyl)-1-methyl-3-(1-methyl-1H-indol-2-yl)-1H-indazole-5-carboxamide (28)

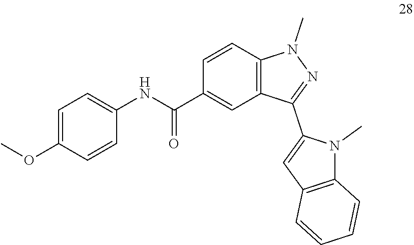

To the solution of 1-methyl-3-(1-methyl-1H-indol-2-yl)-1H-indazole-5-carboxylic acid (10 mg, 0.032 mmol, 1.0 equiv.), 4-methoxyaniline (5 mg, 0.038 mmol, 1.2 equiv.), and HATU (15 mg, 0.038 mmol, 1.2 equiv.) in anhydrous DMF (0.2 mL) was added DIEA (7 μL, 0.038 mmol, 1.2 equiv.) under nitrogen. The reaction mixture was stirred at room temperature for 16 h and then quenched by the addition of water. The aqueous layer was extracted twice with EtOAc, and the combined organic extracts were washed with brine, dried over anhydrous Na₂SO₄, filtered and concentrated. The residue was purified by column chromatography on silica gel (EtOAc/hexane, 30:70 to 40:60) to afford title compound 28 (8 mg, 60%) as a pale yellow solid; ¹H NMR (600 MHz, DMSO-d₆) δ 10.27 (s, 1H), 8.63 (s, 1H), 8.08 (d, J=8.8 Hz, 1H), 7.85 (d, J=8.8 Hz, 1H), 7.69-7.68 (m, 3H), 7.57 (d, J=7.9 Hz, 1H), 7.25 (t, J=7.9 Hz, 1H), 7.20 (s, 1H), 7.12 (t, J=7.9 Hz, 1H), 6.94 (d, J=9.0 Hz, 2H), 4.21 (s, 3H), 4.07 (s, 3H), 3.75 (s, 3H); HRMS (ESI) m/z calcd for C₂₅H₂₃N₄O₂[M+H]⁺411.1815, found 411.1810.

### Step 2b: N-(4-Methoxy-3-methylphenyl)-1-methyl-3-(1-methyl-1H-indol-2-yl)-1H-indazole-5-carboxamide (29)

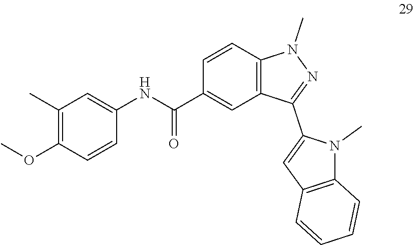

To the solution of 1-methyl-3-(1-methyl-1H-indol-2-yl)-1H-indazole-5-carboxylic acid (10 mg, 0.032 mmol, 1.0 equiv.), 4-methoxy-3-methylaniline (7 mg, 0.05 mmol, 1.5 equiv.), and HATU (19 mg, 0.05 mmol, 1.5 equiv.) in anhydrous DMF (0.2 mL) was added DIEA (9 μL, 0.05 mmol, 1.5 equiv.) under nitrogen. The reaction mixture was stirred at room temperature for 16 h and then quenched by the addition of water. The aqueous layer was extracted twice with EtOAc, and the combined organic extracts were washed with brine, dried over anhydrous Na₂SO₄, filtered and concentrated. The residue was purified by column chromatography on silica gel (EtOAc/hexane, 20:80 to 30:70) to afford title compound 29 (11 mg, 79%) as a pale yellow solid; ¹H NMR (600 MHz, DMSO-d₆) δ 10.20 (s, 1H), 8.63 (s, 1H), 8.08 (d, J=8.8 Hz, 1H), 7.85 (d, J=8.8 Hz, 1H), 7.69 (d, J=7.6 Hz, 1H), 7.57-7.56 (m, 2H), 7.54 (s, 1H), 7.25 (t, J=7.6 Hz, 1H), 7.20 (s, 1H), 7.11 (t, J=7.6 Hz, 1H), 6.93 (d, J=8.7 Hz, 1H), 4.21 (s, 3H), 4.07 (s, 3H), 3.78 (s, 3H), 2.17 (s, 3H); HRMS (ESI) m/z calcd for C₂₆H₂₅N₄O₂[M+H]⁺425.1972, found 425.1966.

### Step 2c: N-(3,4-Dimethoxyphenyl)-1-methyl-3-(1-methyl-1H-indol-2-yl)-1H-indazole-5-carboxamide (30)

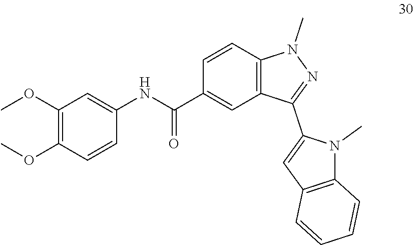

The same procedure for the synthesis of 29 was followed, except 4-methoxy-3-methylaniline was substituted for 3,4-dimethoxyaniline in the amide coupling reaction (step 2b). The residue was purified by column chromatography on silica gel (EtOAc/hexane, 30:70 to 35:65) to afford title compound 30 (10 mg, 69%) as a pale yellow solid; ¹H NMR (600 MHz, DMSO-d₆) δ 10.25 (s, 1H), 8.63 (s, 1H), 8.09 (d, J=8.7 Hz, 1H), 7.86 (d, J=8.7 Hz, 1H), 7.69 (d, J=7.6 Hz, 1H), 7.57 (d, J=7.6 Hz, 1H), 7.49 (s, 1H), 7.33 (d, J=8.6 Hz, 1H), 7.25 (t, J=7.6 Hz, 1H), 7.19 (s, 1H), 7.12 (t, J=7.6 Hz, 1H), 6.95 (d, J=8.6 Hz, 1H), 4.22 (s, 3H), 4.07 (s, 3H), 3.76 (s, 3H), 3.75 (s, 3H); HRMS (ESI) m/z calcd for C₂₆H₂₅N₄O₂[M+H]⁺441.1921, found 441.1912.

### Step 2d: N-(3-Fluoro-4-methoxyphenyl)-1-methyl-3-(1-methyl-1H-indol-2-yl)-1H-indazole-5-carboxamide (31)

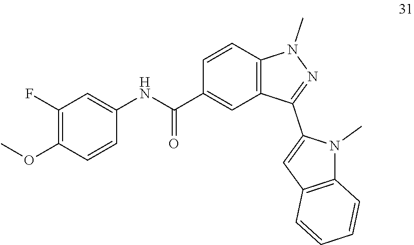

The same procedure for the synthesis of 29 was followed, except 4-methoxy-3-methylaniline was substituted for 3-fluoro-4-methoxyaniline in the amide coupling reaction (step 2b). The residue was purified by column chromatography on silica gel (EtOAc/hexane, 30:70 to 35:65) to afford title compound 31 (10 mg, 69%) as a pale yellow solid; ¹H NMR (600 MHz, DMSO-d₆) δ 10.40 (s, 1H), 8.63 (s, 1H), 8.07 (d, J=8.8 Hz, 1H), 7.86 (d, J=8.8 Hz, 1H), 7.77 (dd, J=13.7, 2.1 Hz, 1H), 7.69 (d, J=7.9 Hz, 1H), 7.57 (d, J=7.9 Hz, 1H), 7.51 (d, J=8.8 Hz, 1H), 7.25 (t, J=7.9 Hz, 1H), 7.19 (s, 1H), 7.17 (d, J=8.8 Hz, 1H), 7.12 (t, J=7.9 Hz, 1H), 4.21 (s, 3H), 4.07 (s, 3H), 3.83 (s, 3H); HRMS (ESI) m/z calcd for C₂₅H₂₂FN₄O₂[M+H]⁺429.1721, found 429.1717.

### Step 2e: N-(3-Chloro-4-methoxyphenyl)-1-methyl-3-(1-methyl-1H-indol-2-yl)-1H-indazole-5-carboxamide (32)

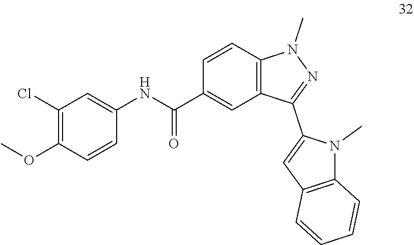

The same procedure for the synthesis of 29 was followed, except 4-methoxy-3-methylaniline was substituted for 3-chloro-4-methoxyaniline in the amide coupling reaction (step 2b). The residue was purified by column chromatography on silica gel (EtOAc/hexane, 30:70 to 35:65) to afford title compound 32 (12 mg, 84%) as a pale yellow solid; ¹H NMR (600 MHz, DMSO-d₆) δ 10.38 (s, 1H), 8.64 (s, 1H), 8.08 (d, J=8.8 Hz, 1H), 7.95 (d, J=2.3 Hz, 1H), 7.86 (d, J=8.8 Hz, 1H), 7.71-7.69 (m, 2H), 7.57 (d, J=7.5 Hz, 1H), 7.26 (t, J=7.5 Hz, 1H), 7.19 (s, 1H), 7.17 (d, J=8.9 Hz, 1H), 7.12 (t, J=7.5 Hz, 1H), 4.21 (s, 3H), 4.07 (s, 3H), 3.85 (s, 3H); HRMS (EST) m/z calcd for C₂₅H₂₂ClN₄O₂[M++H]⁺445.1425, found 445.1414.

### Step 2f: N-(4-Methoxy-2-methylphenyl)-1-methyl-3-(1-methyl-1H-indol-2-yl)-1H-indazole-5-carboxamide (33)

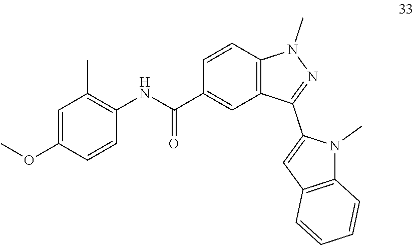

The same procedure for the synthesis of 29 was followed, except 4-methoxy-3-methylaniline was substituted for 4-methoxy-2-methylaniline in the amide coupling reaction (step 2b). The residue was purified by column chromatography on silica gel (EtOAc/hexane, 30:70 to 35:65) to afford title compound 33 (10 mg, 72%) as a pale yellow solid; ¹H NMR (600 MHz, DMSO-d₆) δ 9.98 (s, 1H), 8.69 (s, 1H), 8.09 (d, J=8.8 Hz, 1H), 7.85 (d, J=8.8 Hz, 1H), 7.67 (d, J=7.8 Hz, 1H), 7.57 (d, J=7.8 Hz, 1H), 7.26-7.20 (m, 3H), 7.11 (t, J=7.8 Hz, 1H), 6.87 (d, J=2.6 Hz, 1H), 6.80 (dd, J=8.6, 2.6 Hz, 1H), 4.21 (s, 3H), 4.08 (s, 3H), 3.76 (s, 3H), 2.21 (s, 3H); HRMS (ESI) m/z calcd for C₂₆H₂₅N₄O₂[M+H]⁺425.1972, found 425.1965.

### Step 2g: N-(2,4-Dimethoxyphenyl)-1-methyl-3-(1-methyl-1H-indol-2-yl)-1H-indazole-5-carboxamide (34)

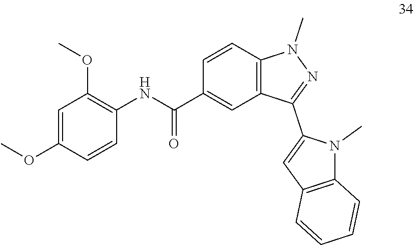

The same procedure for the synthesis of 29 was followed, except 4-methoxy-3-methylaniline was substituted for 2,4-dimethoxyaniline in the amide coupling reaction (step 2b). The residue was purified by column chromatography on silica gel (EtOAc/hexane, 30:70 to 35:65) to afford title compound 34 (11 mg, 76%) as a pale yellow solid; ¹H NMR (600 MHz, DMSO-d₆) δ 9.67 (s, 1H), 8.66 (s, 1H), 8.06 (d, J=8.8 Hz, 1H), 7.83 (d, J=8.8 Hz, 1H), 7.67 (d, J=7.8 Hz, 1H), 7.57 (d, J=7.8 Hz, 1H), 7.43 (d, J=8.6 Hz, 1H), 7.26-7.23 (m, 2H), 7.11 (t, J=7.8 Hz, 1H), 6.67 (d, J=2.5 Hz, 1H), 6.56 (dd, J=8.6, 2.5 Hz, 1H), 4.21 (s, 3H), 4.08 (s, 3H), 3.79 (s, 3H), 3.79 (s, 3H); HRMS (ESI) m/z calcd for C₂₆H₂₅N₄O₃[M+H]⁺441.1921, found 441.1909.

### Step 2h: N-(2-Fluoro-4-methoxyphenyl)-1-methyl-3-(1-methyl-1H-indol-2-yl)-1H-indazole-5-carboxamide (35)

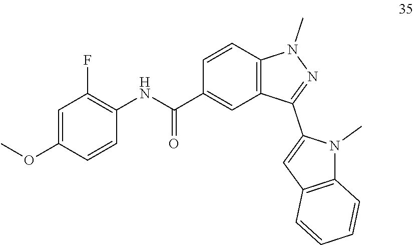

The same procedure for the synthesis of 29 was followed, except 4-methoxy-3-methylaniline was substituted for 2-fluoro-4-methoxyaniline in the amide coupling reaction (step 2b). The residue was purified by column chromatography on silica gel (EtOAc/hexane, 30:70 to 35:65) to afford title compound 35 (10.4 mg, 74%) as a pale yellow solid; ¹H NMR (600 MHz, DMSO-d₆) δ 10.15 (s, 1H), 8.69 (s, 1H), 8.09 (d, J=8.8 Hz, 1H), 7.85 (d, J=8.8 Hz, 1H), 7.68 (d, J=7.8 Hz, 1H), 7.57 (d, J=7.8 Hz, 1H), 7.43 (t, J=8.9 Hz, 1H), 7.25 (t, J=7.8 Hz, 1H), 7.21 (s, 1H), 7.12 (t, J=7.8 Hz, 1H), 6.95 (dd, J=12.2, 2.3 Hz, 1H), 6.82 (dd, J=8.9, 2.3 Hz, 1H), 4.21 (s, 3H), 4.08 (s, 3H), 3.79 (s, 3H); HRMS (ESI) m/z calcd for C₂₅H₂₂FN₄O₂[M+H]⁺429.1721, found 429.1711.

### Step 2i: N-(2-Chloro-4-methoxyphenyl)-1-methyl-3-(1-methyl-1H-indol-2-yl)-1H-indazole-5-carboxamide (36)

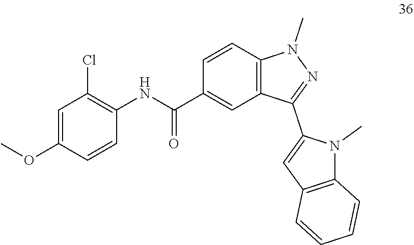

The same procedure for the synthesis of 29 was followed, except 4-methoxy-3-methylaniline was substituted for 2-chloro-4-methoxyaniline in the amide coupling reaction (step 2b). The residue was purified by reverse-phase column chromatography on C-18 (CH₃CN/water, 5:95 to 100:0) to afford title compound 36 (8 mg, 56%) as a yellow solid; ¹H NMR (600 MHz, DMSO-d₆) δ 10.17 (s, 1H), 8.71 (s, 1H), 8.09 (d, J=8.8 Hz, 1H), 7.86 (d, J=8.8 Hz, 1H), 7.67 (d, J=7.8 Hz, 1H), 7.57 (d, J=7.8 Hz, 1H), 7.44 (d, J=8.7 Hz, 1H), 7.25 (t, J=7.8 Hz, 1H), 7.21 (s, 1H), 7.16 (t, J=2.0 Hz, 1H), 7.12 (t, J=7.8 Hz, 1H), 6.99 (dd, J=8.7, 2.0 Hz, 1H), 4.21 (s, 3H), 4.08 (s, 3H), 3.81 (s, 3H); HRMS (EST) m/z calcd for C₂₅H₂₂ClN₄O₂[M+H]⁺445.1425, found 445.1427.

### Syntheses of Compounds 37-40

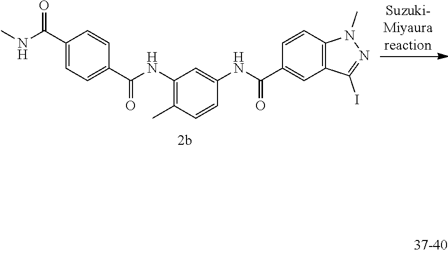 [SMILES: CNC(=O)c1ccc(C(=O)Nc2cc(NC(=O)c3ccc4c(c3)c(I)nn4C)ccc2C)cc1]

### N¹-(5-(3-(4-(Benzyloxy)phenyl)-1-methyl-1H-indazole-5-carboxamido)-2-methylphenyl)-N⁴-methylterephthalamide (37)

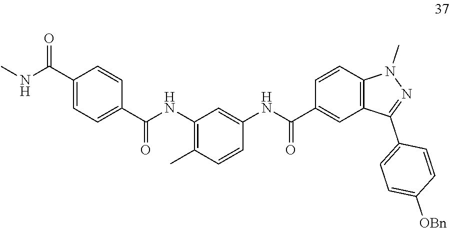 [SMILES: CNC(=O)c1ccc(C(=O)Nc2cc(NC(=O)c3ccc4c(c3)c(-c3ccc(OCc5ccccc5)cc3)nn4C)ccc2C)cc1]

The general procedure employed for the Suzuki-Miyaura reaction was followed using aryl iodide 2b (9 mg, 0.015 mmol, 1.0 equiv.), (4-(benzyloxy)phenyl)boronic acid (7 mg, 0.03 mmol, 2.0 equiv.), and Pd(dppf)Cl₂·CH₂Cl₂ (1.3 mg, 0.0015 mmol, 0.1 equiv.) in DME/Na₂CO₃₍ₛₐₜ₎(0.4 mL, v/v=1:1). Isolation and purification afforded title compound 37 (7 mg, 75%) as an off-white solid; ¹H NMR (600 MHz, DMSO-d₆) δ 10.37 (s, 1H), 10.05 (s, 1H), 8.66 (s, 1H), 8.62-8.60 (m, 1H), 8.06 (d, J=8.4 Hz, 2H), 8.03 (d, J=8.9 Hz, 1H), 7.99-7.97 (m, 4H), 7.84 (d, J=2.1 Hz, 1H), 7.78 (d, J=8.9 Hz, 1H), 7.66 (dd, J=8.4, 2.1 Hz, 1H), 7.49 (d, J=7.4 Hz, 2H), 7.41 (t, J=7.4 Hz, 2H), 7.34 (t, J=7.4 Hz, 1H), 7.27 (d, J=8.4 Hz, 1H), 7.21 (d, J=8.9 Hz, 2H), 5.20 (s, 2H), 4.13 (s, 3H), 2.82 (d, J=4.5 Hz, 3H), 2.23 (s, 3H).

### N¹-(5-(3-(6-Amino-2-methylpyridin-3-yl)-1-methyl-1H-indazole-5-carboxamido)-2-methylphenyl)-N⁴-methylterephthalamide (38)

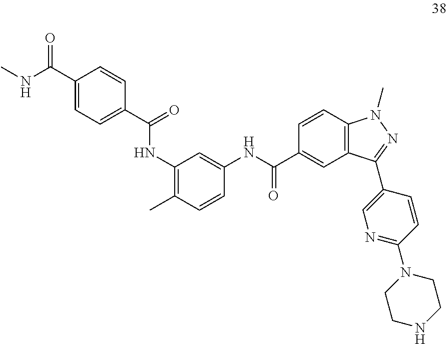 [SMILES: CNC(=O)c1ccc(C(=O)Nc2cc(NC(=O)c3ccc4c(c3)c(-c3ccc(N5CCNCC5)nc3)nn4C)ccc2C)cc1]

The general procedure employed for the Suzuki-Miyaura reaction was followed using aryl iodide 2b (11 mg, 0.02 mmol, 1.0 equiv.), 1-(5-(4,4,5,5-tetramethyl-1,3,2-dioxaborolan-2-yl)pyridin-2-yl)piperazine (9 mg, 0.03 mmol, 1.5 equiv.), and Pd(dppf)Cl₂·CH₂Cl₂ (1.6 mg, 0.002 mmol, 0.1 equiv.) in DME/Na₂CO₃₍ₛₐₜ₎(0.4 mL, v/v=1:1). Isolation and purification afforded title compound 38 (8 mg, 66%) as a pale yellow solid; ¹H NMR (600 MHz, DMSO-d₆) δ 10.41 (s, 1H), 10.07 (s, 1H), 8.86 (d, J=2.0 Hz, 1H), 8.69 (s, 1H), 8.66-8.63 (m, 1H), 8.17 (dd, J=8.9, 2.0 Hz, 1H), 8.07 (d, J=8.5 Hz, 2H), 8.04 (dd, J=8.7, 1.5 Hz, 1H), 7.98 (d, J=8.5 Hz, 2H), 7.85 (d, J=2.1 Hz, 1H), 7.78 (d, J=8.7 Hz, 1H), 7.66 (dd, J=8.5, 2.1 Hz, 1H), 7.27 (d, J=8.5 Hz, 1H), 7.03 (d, J=8.9 Hz, 1H), 4.13 (s, 3H), 3.63-3.61 (m, 4H), 2.95-2.93 (m, 4H), 2.82 (d, J=4.5 Hz, 3H), 2.23 (s, 3H).

### N¹-Methyl-N⁴-(2-methyl-5-(1-methyl-3-(6-morpholinopyridin-3-yl)-1H-indazole-5-carboxamido)phenyl)terephthalamide (39)

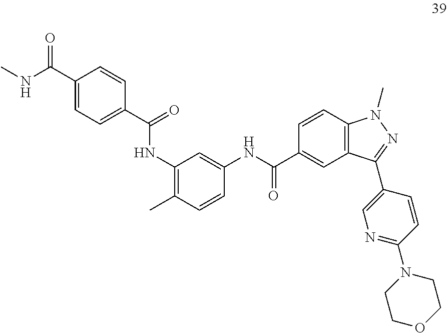 [SMILES: CNC(=O)c1ccc(C(=O)Nc2cc(NC(=O)c3ccc4c(c3)c(-c3ccc(N5CCOCC5)nc3)nn4C)ccc2C)cc1]

The general procedure employed for the Suzuki-Miyaura reaction was followed using aryl iodide 2b (11 mg, 0.02 mmol, 1.0 equiv.), (6-morpholinopyridin-3-yl)boronic acid (6.5 mg, 0.03 mmol, 1.5 equiv.), and Pd(dppf)Cl₂·CH₂Cl₂ (1.6 mg, 0.002 mmol, 0.1 equiv.) in DME/Na₂CO₃₍ₛₐₜ₎(0.4 mL, v/v=1:1). Isolation and purification afforded title compound 39 (9 mg, 76%) as a pale yellow solid; ¹H NMR (600 MHz, DMSO-d₆) δ 10.38 (s, 1H), 10.05 (s, 1H), 8.88 (d, J=2.5 Hz, 1H), 8.69 (s, 1H), 8.62-8.60 (m, 1H), 8.18 (dd, J=8.5, 2.5 Hz, 1H), 8.06 (d, J=8.5 Hz, 2H), 8.04 (dd, J=8.9, 1.5 Hz, 1H), 7.97 (d, J=8.5 Hz, 2H), 7.85 (d, J=2.1 Hz, 1H), 7.78 (d, J=8.9 Hz, 1H), 7.66 (dd, J=8.6, 2.1 Hz, 1H), 7.27 (d, J=8.6 Hz, 1H), 7.03 (d, J=8.5 Hz, 1H), 4.13 (s, 3H), 3.74-3.72 (m, 4H), 3.56-3.54 (m, 4H), 2.82 (d, J=4.5 Hz, 3H), 2.23 (s, 3H).

### N¹-(5-(3-(4-Chlorophenyl)-1-methyl-1H-indazole-5-carboxamido)-2-methylphenyl)-N⁴-methylterephthalamide (40)

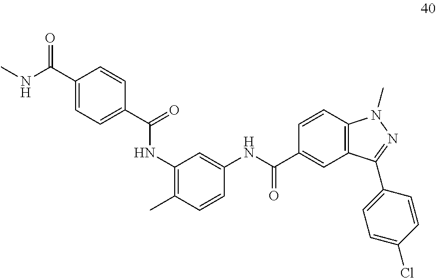 [SMILES: CNC(=O)c1ccc(C(=O)Nc2cc(NC(=O)c3ccc4c(c3)c(-c3ccc(Cl)cc3)nn4C)ccc2C)cc1]

The general procedure employed for the Suzuki-Miyaura reaction was followed using aryl iodide 2b (11 mg, 0.02 mmol, 1.0 equiv.), (4-chlorophenyl)boronic acid (4.7 mg, 0.03 mmol, 1.5 equiv.), and Pd(dppf)Cl₂·CH₂Cl₂ (1.6 mg, 0.002 mmol, 0.1 equiv.) in DME/Na₂CO₃₍ₛₐₜ₎(0.4 mL, v/v=1:1). Isolation and purification afforded title compound 40 (8.5 mg, 79%) as a pale yellow solid; ¹H NMR (600 MHz, DMSO-d₆) δ 10.35 (s, 1H), 10.05 (s, 1H), 8.69 (s, 1H), 8.62-8.60 (m, 1H), 8.09-8.04 (m, 5H), 7.97 (d, J=8.4 Hz, 2H), 7.85 (d, J=1.8 Hz, 1H), 7.83 (d, J=8.9 Hz, 1H), 7.66 (dd, J=8.4, 1.8 Hz, 1H), 7.63 (d, J=8.5 Hz, 2H), 7.27 (d, J=8.4 Hz, 1H), 4.16 (s, 3H), 2.82 (d, J=4.5 Hz, 3H), 2.23 (s, 3H).

### Syntheses of Compounds 41 and 42

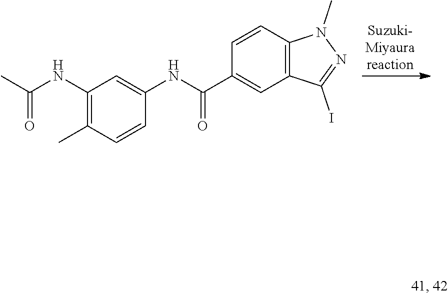 [SMILES: CC(=O)Nc1cc(NC(=O)c2ccc3c(c2)c(I)nn3C)ccc1C]

### N-(3-Acetamido-4-methylphenyl)-3-(4-chloro-2-methylphenyl)-1-methyl-1H-indazole-5-carboxamide (41)

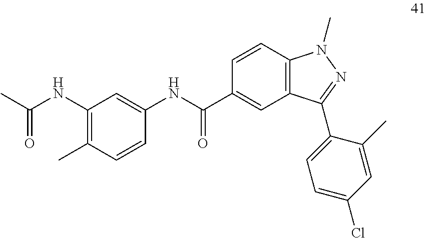 [SMILES: CC(=O)Nc1cc(NC(=O)c2ccc3c(c2)c(-c2ccc(Cl)cc2C)nn3C)ccc1C]

The same procedure for the synthesis of 16 was followed, except 4-methyl-5-(4,4,5,5-tetramethyl-1,3,2-dioxaborolan-2-yl)-2-pyridinamine hydrochloride was substituted for (4-chloro-2-methylphenyl)boronic acid in the Suzuki-Miyaura reaction (step 2). The residue was purified by column chromatography on silica gel (CH₃OH/CH₂Cl₂, 1:99 to 2:98) to afford title compound 41 (18 mg, 81%) as a white solid; ¹H NMR (600 MHz, DMSO-d₆) δ 10.21 (s, 1H), 9.30 (s, 1H), 8.31 (s, 1H), 8.05 (dd, J=8.9, 1.5 Hz, 1H), 8.02-8.01 (m, 2H), 7.60 (d, J=8.2 Hz, 1H), 7.53 (d, J=2.0 Hz, 1H), 7.51 (dd, J=8.3, 2.0 Hz, 1H), 7.44 (dd, J=8.2, 2.0 Hz, 1H), 7.15 (d, J=8.3 Hz, 1H), 4.16 (s, 3H), 2.39 (s, 3H), 2.16 (s, 3H), 2.06 (s, 3H).

### N-(3-Acetamido-4-methylphenyl)-3-(2,4-dichlorophenyl)-1-methyl-1H-indazole-5-carboxamide (42)

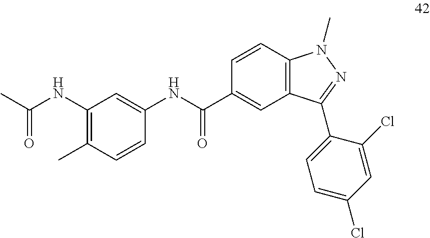 [SMILES: CC(=O)Nc1cc(NC(=O)c2ccc3c(c2)c(-c2ccc(Cl)cc2Cl)nn3C)ccc1C]

The same procedure for the synthesis of 16 was followed, except 4-methyl-5-(4,4,5,5-tetramethyl-1,3,2-dioxaborolan-2-yl)-2-pyridinamine hydrochloride was substituted for (2,4-dichlorophenyl)boronic acid in the Suzuki-Miyaura reaction (step 2). The residue was purified by column chromatography on silica gel (CH₃OH/CH₂Cl₂, 1:99 to 2:98) to afford title compound 42 (20 mg, 86%) as a pale yellow solid; ¹H NMR (600 MHz, DMSO-d₆) δ 10.20 (s, 1H), 9.30 (s, 1H), 8.32 (s, 1H), 8.06 (dd, J=8.9, 1.2 Hz, 1H), 7.88 (d, J=2.0 Hz, 1H), 7.84 (d, J=8.9 Hz, 1H), 7.81 (s, 1H), 7.69 (d, J=8.3 Hz, 1H), 7.62 (dd, J=8.3, 2.0 Hz, 1H), 7.51 (d, J=8.3 Hz, 1H), 7.15 (d, J=8.3 Hz, 1H), 4.18 (s, 3H), 2.16 (s, 3H), 2.06 (s, 3H).

### Syntheses of Compounds 43 and 44

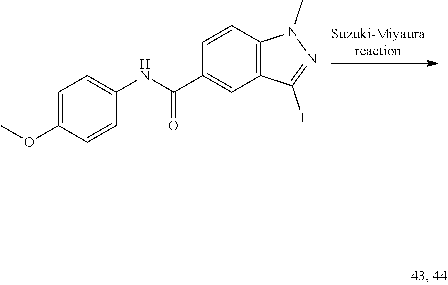 [SMILES: COc1ccc(NC(=O)c2ccc3c(c2)c(I)nn3C)cc1]

### N-(4-Methoxyphenyl)-1-methyl-3-(pyridin-4-yl)-1H-indazole-5-carboxamide (43)

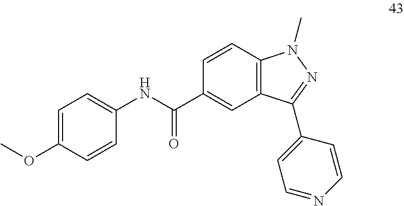 [SMILES: COc1ccc(NC(=O)c2ccc3c(c2)c(-c2ccncc2)nn3C)cc1]

The same procedure for the synthesis of 24 was followed, except 3-methyl-4-(4,4,5,5-tetramethyl-1,3,2-dioxaborolan-2-yl)aniline was substituted for pyridin-4-ylboronic acid in the Suzuki-Miyaura reaction (step 2). Isolation and purification afforded title compound 43 (72%) as a yellow oil; ¹H NMR (600 MHz, CDCl₃) δ 10.28 (s, 1H), 8.76 (s, 1H), 8.75-8.74 (m, 2H), 8.07-8.06 (m, 3H), 7.88 (d, J=8.8 Hz, 1H), 7.69 (d, J=9.0 Hz, 2H), 6.96 (d, J=9.0 Hz, 2H), 4.20 (s, 3H), 3.76 (s, 3H).

### N-(4-Methoxyphenyl)-1-methyl-3-(3-methylpyridin-4-yl)-1H-indazole-5-carboxamide (44)

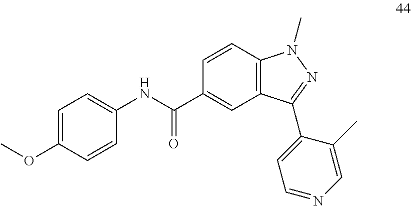

The same procedure for the synthesis of 24 was followed, except 3-methyl-4-(4,4,5,5-tetramethyl-1,3,2-dioxaborolan-2-yl)aniline was substituted for (3-methylpyridin-4-yl)boronic acid in the Suzuki-Miyaura reaction (step 2). Isolation and purification afforded title compound 44 (50%) as a light yellow oil; ¹H NMR (600 MHz, CDCl₃) δ 10.18 (s, 1H), 8.64 (s, 1H), 8.58 (d, J=4.9 Hz, 1H), 8.41 (s, 1H), 8.07 (dd, J=8.9, 1.4 Hz, 1H), 7.86 (d, J=8.9 Hz, 1H), 7.67 (d, J=4.9 Hz, 2H), 7.65 (d, J=9.0 Hz, 2H), 6.93 (d, J=9.0 Hz, 2H), 4.19 (s, 3H), 3.74 (s, 3H), 2.45 (s, 3H).

### Syntheses of Compounds 45-49

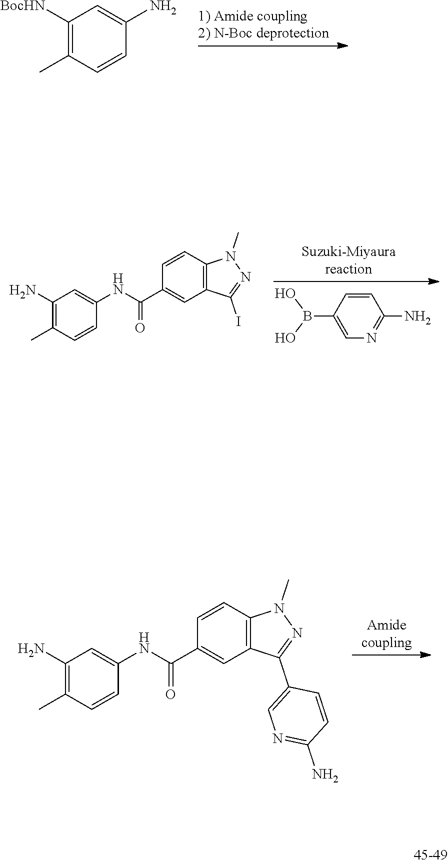 [SMILES: Cc1ccc(N)cc1NC(=O)OC(C)(C)C ; Cc1ccc(NC(=O)c2ccc3c(c2)c(I)nn3C)cc1N ; Nc1ccc(B(O)O)cn1 ; Cc1ccc(NC(=O)c2ccc3c(c2)c(-c2ccc(N)nc2)nn3C)cc1N]

### Step 1: N-(3-Amino-4-methylphenyl)-3-iodo-1-methyl-1H-indazole-5-carboxamide

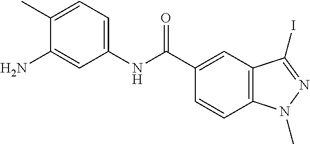 [SMILES: Cc1ccc(NC(=O)c2ccc3c(c2)c(I)nn3C)cc1N]

To a solution of 3-iodo-1-methyl-1H-indazole-5-carboxylic acid (1.2 g, 4 mmol, 1.0 equiv.), tert-butyl (5-amino-2-methylphenyl)carbamate (890 mg, 4 mmol, 1.0 equiv.), and HATU (1.8 g, 4.8 mmol, 1.2 equiv.) in anhydrous DMF (5 mL) was added DIEA (840 μL, 4.8 mmol, 1.2 equiv.) under nitrogen. The resulting mixture was stirred at room temperature for 16 h and then quenched by the addition of water. The aqueous layer was extracted twice with EtOAc, and the combined organic extracts were washed with brine, dried over anhydrous Na₂SO₄, filtered, and concentrated. To the solution of crude product in CH₂Cl₂ (5 mL) was added trifluoroacetic acid (1 mL), and the mixture was stirred at room temperature for 1 h. The residue was concentrated in vacuo and purified by reverse-phase column chromatography on C-18 (CH₃CN/water, 5:95 to 100:0) to afford N-(3-amino-4-methylphenyl)-3-iodo-1-methyl-1H-indazole-5-carboxamide (1.2 g, 74%).

### Step 2: N-(3-Amino-4-methylphenyl)-3-(6-aminopyridin-3-yl)-1-methyl-1H-indazole-5-carboxamide

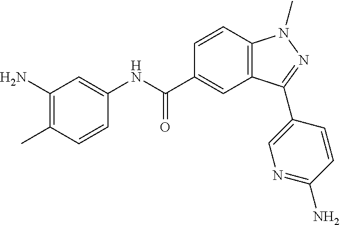 [SMILES: Cc1ccc(NC(=O)c2ccc3c(c2)c(-c2ccc(N)nc2)nn3C)cc1N]

The general procedure employed for the Suzuki-Miyaura reaction was followed using N-(3-amino-4-methylphenyl)-3-iodo-1-methyl-1H-indazole-5-carboxamide (203 mg, 0.5 mmol, 1.0 equiv.), (6-aminopyridin-3-yl)boronic acid (103 mg, 0.75 mmol, 1.5 equiv.), and Pd(dppf)Cl₂·CH₂Cl₂ (41 mg, 0.05 mmol, 0.1 equiv.) in DME/Na₂CO₃₍ₛₐₜ₎(2 mL, v/v=1:1). The residue was purified by column chromatography on silica gel (CH₃OH/CH₂Cl₂, 3:97 to 5:95) to afford N-(3-amino-4-methylphenyl)-3-(6-aminopyridin-3-yl)-1-methyl-1H-indazole-5-carboxamide (185 mg, 99%) as a light brown solid.

### Step 3: 3-(6-Aminopyridin-3-yl)-1-methyl-N-(4-methyl-3-(1-methylpiperidine-4-carboxamido)phenyl)-1H-indazole-5-carboxamide (45)

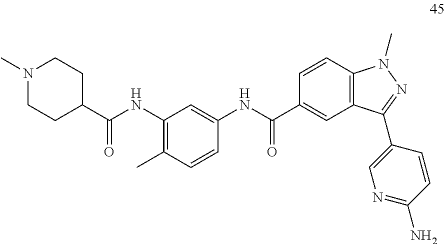 [SMILES: Cc1ccc(NC(=O)c2ccc3c(c2)c(-c2ccc(N)nc2)nn3C)cc1NC(=O)C1CCN(C)CC1]

To a solution of N-(3-amino-4-methylphenyl)-3-(6-aminopyridin-3-yl)-1-methyl-1H-indazole-5-carboxamide (19 mg, 0.05 mmol, 1.0 equiv.) and TEA (35 μL, 0.25 mmol, 5.0 equiv.) in anhydrous THF (0.5 mL) was added 1-methylpiperidine-4-carbonyl chloride hydrochloride (20 mg, 0.1 mmol, 2.0 equiv.) under nitrogen. The reaction mixture was stirred at room temperature for 16 h and then quenched by the addition of water. Isolation and purification afforded title compound 45 (5.5 mg, 22%) as a pale yellow foam; ¹H NMR (600 MHz, DMSO-d₆) δ 10.40 (s, 1H), 10.17 (br s, 1H), 9.54 (s, 1H), 8.70 (s, 2H), 8.35 (d, J=8.0 Hz, 1H), 8.04 (dd, J=8.9, 1.4 Hz, 1H), 7.85 (d, J=1.8 Hz, 1H), 7.80 (d, J=8.9 Hz, 1H), 7.61-7.54 (m, 2H), 7.19 (d, J=8.3 Hz, 1H), 6.99 (d, J=8.0 Hz, 1H), 4.13 (s, 3H), 3.48-3.46 (m, 2H), 2.98-2.97 (m, 2H), 2.75 (s, 3H), 2.71-2.67 (m, 1H), 2.17 (s, 3H), 2.06-2.03 (m, 2H), 1.98-1.91 (m, 2H).

### 3-(6-Aminopyridin-3-yl)-1-methyl-N-(4-methyl-3-(piperazine-1-carboxamido)phenyl)-1H-indazole-5-carboxamide (46)

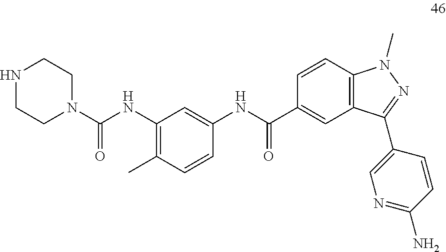 [SMILES: Cc1ccc(NC(=O)c2ccc3c(c2)c(-c2ccc(N)nc2)nn3C)cc1NC(=O)N1CCNCC1]

The same procedure for the synthesis of 45 was followed, except 1-methylpiperidine-4-carbonyl chloride hydrochloride was substituted for tert-butyl 4-(chlorocarbonyl)piperazine-1-carboxylate in the amide coupling reaction (step 3). To the solution of crude product in CH₂Cl₂ (0.5 mL) was added trifluoroacetic acid (0.1 mL), and the mixture was stirred at room temperature for 1 h. Isolation and purification afforded title compound 46 (15%) as a light yellow oil; ¹H NMR (600 MHz, CD₃OD) δ 8.56 (br s, 1H), 8.54 (br s, 1H), 8.09 (dd, J=8.6, 2.0 Hz, 1H), 8.02 (dd, J=8.9, 1.0 Hz, 1H), 7.65 (d, J=8.9 Hz, 1H), 7.60 (d, J=1.7 Hz, 1H), 7.46 (dd, J=8.2, 1.7 Hz, 1H), 7.21 (d, J=8.2 Hz, 1H), 6.75 (d, J=8.6 Hz, 1H), 4.13 (s, 3H), 3.53-3.51 (m, 4H), 2.87-2.85 (m, 4H), 2.23 (s, 3H).

### 3-(6-Aminopyridin-3-yl)-N-(3-(cyclohexanecarboxamido)-4-methylphenyl)-1-methyl-1H-indazole-5-carboxamide (47)

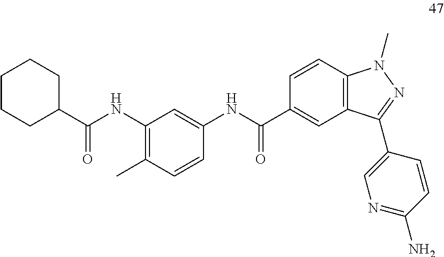 [SMILES: Cc1ccc(NC(=O)c2ccc3c(c2)c(-c2ccc(N)nc2)nn3C)cc1NC(=O)C1CCCCC1]

The same procedure for the synthesis of 45 was followed, except 1-methylpiperidine-4-carbonyl chloride hydrochloride was substituted for cyclohexanecarbonyl chloride in the amide coupling reaction (step 3). Isolation and purification afforded title compound 47 (41%) as a white solid; ¹H NMR (600 MHz, DMSO-d₆) δ 10.30 (s, 1H), 9.19 (s, 1H), 8.63 (s, 2H), 8.02-7.99 (m, 2H), 7.78 (s, 1H), 7.74 (d, J=8.8 Hz, 1H), 7.58 (d, J=8.1 Hz, 1H), 7.17 (d, J=8.1 Hz, 1H), 6.62 (d, J=8.4 Hz, 1H), 6.23 (s, 2H), 4.10 (s, 3H), 2.44-2.40 (m, 1H), 2.16 (s, 3H), 1.84-1.82 (m, 2H), 1.77-1.75 (m, 2H), 1.67-1.65 (m, 1H), 1.47-1.41 (m, 2H), 1.32-1.26 (m, 2H), 1.22-1.18 (m, 1H).

### N-(3-((1s,4s)-4-Aminocyclohexane-1-carboxamido)-4-methylphenyl)-3-(6-aminopyridin-3-yl)-1-methyl-1H-indazole-5-carboxamide (48)

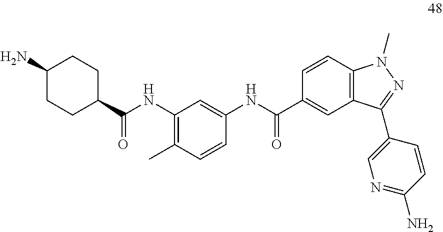 [SMILES: Cc1ccc(NC(=O)c2ccc3c(c2)c(-c2ccc(N)nc2)nn3C)cc1NC(=O)[C@H]1CC[C@@H](N)CC1]

To a solution 3-(6-aminopyridin-3-yl)-1-methyl-1H-indazole-5-carboxylic acid (19 mg, 0.05 mmol, 1.0 equiv.), (1s,4s)-4-((tert-butoxycarbonyl)amino)cyclohexane-1-carboxylic acid (13 mg, 0.05 mmol, 1.0 equiv.), and HATU (23 mg, 0.06 mmol, 1.2 equiv.) in anhydrous DMF (0.2 mL) was added DIEA (18 μL, 0.1 mmol, 2.0 equiv.) under nitrogen. The reaction mixture was stirred at room temperature for 16 h and then quenched by the addition of water. The aqueous layer was extracted twice with EtOAc, and the combined organic extracts were washed with brine, dried over anhydrous Na₂SO₄, filtered, and concentrated. To the solution of crude product in CH₂Cl₂ (1 mL) was added trifluoroacetic acid (0.5 mL), and the mixture was stirred at room temperature for 1 h. Isolation and purification afforded title compound 48 (23 mg, 92%) as a light yellow foam; ¹H NMR (600 MHz, DMSO-d₆) δ 10.31 (s, 1H), 9.30 (s, 1H), 8.62 (s, 2H), 8.02-7.99 (m, 2H), 7.83 (s, 1H), 7.79 (s, 2H), 7.75 (d, J=8.8 Hz, 1H), 7.49 (dd, J=8.3, 1.6 Hz, 1H), 7.19 (d, J=8.3 Hz, 1H), 6.63 (d, J=7.9 Hz, 1H), 6.26 (s, 2H), 4.11 (s, 3H), 3.24-3.20 (m, 1H), 2.62-2.61 (m, 1H), 2.17 (s, 3H), 1.98-1.96 (m, 2H), 1.79-1.72 (m, 4H), 1.69-1.67 (m, 2H).

### (1r,4r)-N¹-(5-(3-(6-Aminopyridin-3-yl)-1-methyl-1H-indazole-5-carboxamido)-2-methylphenyl)-N¹-methylcyclohexane-1,4-dicarboxamide (49)

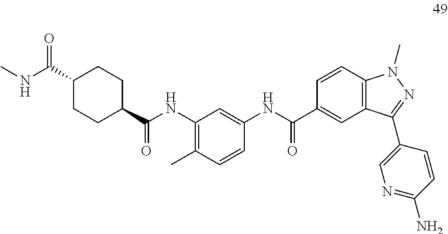 [SMILES: CNC(=O)[C@H]1CC[C@H](C(=O)Nc2cc(NC(=O)c3ccc4c(c3)c(-c3ccc(N)nc3)nn4C)ccc2C)CC1]

To a solution 3-(6-aminopyridin-3-yl)-1-methyl-1H-indazole-5-carboxylic acid (19 mg, 0.05 mmol, 1.0 equiv.), (1r,4r)-4-(methoxycarbonyl)cyclohexane-1-carboxylic acid (10 mg, 0.05 mmol, 1.0 equiv.), and HATU (23 mg, 0.06 mmol, 1.2 equiv.) in anhydrous DMF (0.2 mL) was added DIEA (18 μL, 0.1 mmol, 2.0 equiv.) under nitrogen. The reaction mixture was stirred at room temperature for 16 h and then quenched by the addition of water. The aqueous layer was extracted twice with EtOAc, and the combined organic extracts were washed with brine, dried over anhydrous Na₂SO₄, filtered, and concentrated. The residue was purified by column chromatography on silica gel (CH₃OH/CH₂Cl₂, 3:97 to 5:95) to give a partially pure product. To the solution of partially pure product in THF/water (2 mL, v/v=1:1) was added LiOH·H₂O (84 mg, 2 mmol, 3.0 equiv.), and the mixture was vigorously stirred at room temperature for 16 h. The reaction mixture was neutralized to pH 7 with 4N HCl_{(aq)} and concentrated in vacuo. To the solution of crude hydrolyzed product, methylamine hydrochloride (5 mg, 0.075 mmol, 1.5 equiv.), and HATU (23 mg, 0.06 mmol, 1.2 equiv.) in anhydrous DMF (0.2 mL) was added DIEA (26 μL, 0.15 mmol, 3.0 equiv.) under nitrogen. The reaction mixture was stirred at room temperature for 16 h and then quenched by the addition of water. Isolation and purification afforded title compound 49 (8.7 mg, 32%) as a pale yellow solid; ¹H NMR (600 MHz, DMSO-d₆) δ 10.30 (s, 1H), 9.26 (s, 1H), 8.62 (s, 2H), 8.02-7.98 (m, 2H), 7.78 (s, 1H), 7.75-7.71 (m, 2H), 7.56 (d, J=8.3 Hz, 1H), 7.17 (d, J=8.3 Hz, 1H), 6.63 (d, J=8.6 Hz, 1H), 6.23 (s, 2H), 4.10 (s, 3H), 2.56 (d, J=4.3 Hz, 3H), 2.41-2.38 (m, 1H), 2.15 (s, 3H), 2.11-2.08 (m, 1H), 1.91-1.89 (m, 2H), 1.80-1.79 (m, 2H), 1.49-1.38 (m, 4H).

### Syntheses of Compounds 50-54

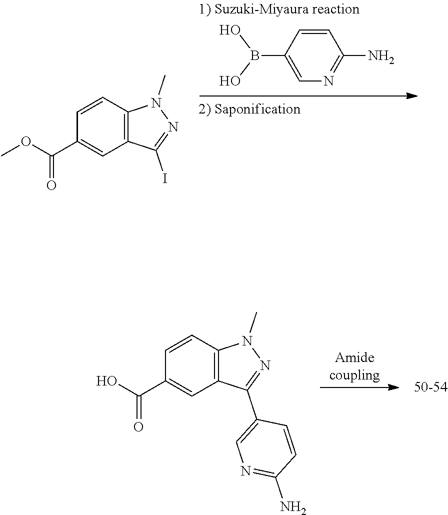 [SMILES: COC(=O)c1ccc2c(c1)c(I)nn2C ; Nc1ccc(B(O)O)cn1 ; Cn1nc(-c2ccc(N)nc2)c2cc(C(=O)O)ccc21]

### Step 1: 3-(6-Aminopyridin-3-yl)-1-methyl-1H-indazole-5-carboxylic acid

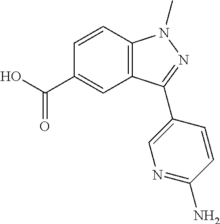 [SMILES: Cn1nc(-c2ccc(N)nc2)c2cc(C(=O)O)ccc21]

The general procedure employed for the Suzuki-Miyaura reaction was followed using methyl 3-iodo-1-methyl-1H-indazole-5-carboxylate (316 mg, 1 mmol, 1.0 equiv.), (6-aminopyridin-3-yl)boronic acid (207 mg, 1.5 mmol, 1.5 equiv.), and Pd(dppf)Cl₂·CH₂Cl₂ (82 mg, 0.1 mmol, 0.1 equiv.) in DME/Na₂CO₃₍ₛₐₜ₎(4 mL, v/v=1:1). To the solution of crude cross-coupling product in THF/water (6 mL, v/v=1:1) was added LiOH·H₂O (84 mg, 2 mmol, 2.0 equiv.), and the mixture was vigorously stirred at 45° C. for 16 h. The reaction mixture was neutralized to pH 7 with 4N HCl_{(aq)} and concentrated in vacuo. The residue was purified by reverse-phase column chromatography on C-18 (CH₃CN/water, 5:95 to 100:0) to afford 3-(6-aminopyridin-3-yl)-1-methyl-1H-indazole-5-carboxylic acid (168 mg, 62%).

### Step 2: 3-(6-Aminopyridin-3-yl)-1-methyl-N-(1-methylpiperidin-4-yl)-1H-indazole-5-carboxamide (50)

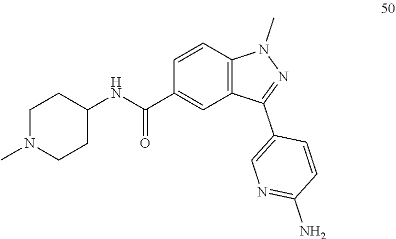 [SMILES: CN1CCC(NC(=O)c2ccc3c(c2)c(-c2ccc(N)nc2)nn3C)CC1]

To a solution 3-(6-aminopyridin-3-yl)-1-methyl-1H-indazole-5-carboxylic acid (14 mg, 0.05 mmol, 1.0 equiv.), 1-methylpiperidin-4-amine (9 mg, 0.075 mmol, 1.5 equiv.), and HATU (23 mg, 0.06 mmol, 1.2 equiv.) in anhydrous DMF (0.2 mL) was added DIEA (26 μL, 0.15 mmol, 3.0 equiv.) under nitrogen. The reaction mixture was stirred at room temperature for 16 h and then quenched by the addition of water. Isolation and purification afforded title compound 50 (10 mg, 55%) as a light yellow oil; ¹H NMR (600 MHz, DMSO-d₆) δ 8.58 (d, J=1.2 Hz, 1H), 8.47-8.46 (m, 2H), 7.95-7.92 (m, 2H), 7.69 (d, J=8.8 Hz, 1H), 6.61 (d, J=8.6 Hz, 1H), 6.22 (s, 2H), 4.08 (s, 3H), 3.91 (br s, 1H), 3.34 (br s, 2H), 3.09 (br s, 2H), 2.45 (br s, 3H), 1.92-1.91 (m, 2H), 1.72-1.70 (m, 2H).

### N-(1-Acetylpiperidin-4-yl)-3-(6-aminopyridin-3-yl)-1-methyl-1H-indazole-5-carboxamide (51)

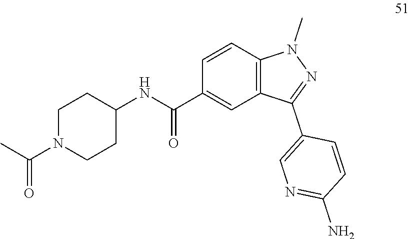 [SMILES: CC(=O)N1CCC(NC(=O)c2ccc3c(c2)c(-c2ccc(N)nc2)nn3C)CC1]

The same procedure for the synthesis of 50 was followed, except 1-methylpiperidin-4-amine was substituted for 1-(4-aminopiperidin-1-yl)ethan-1-one in the amide coupling reaction (step 2). Isolation and purification afforded title compound 51 (13 mg, 66%) as a light yellow oil; ¹H NMR (600 MHz, DMSO-d₆) δ 8.58 (d, J=2.1 Hz, 1H), 8.46 (s, 1H), 8.42 (d, J=7.7 Hz, 1H), 7.95-7.93 (m, 2H), 7.69 (d, J=8.9 Hz, 1H), 6.61 (d, J=8.6 Hz, 1H), 6.21 (s, 2H), 4.39-4.37 (m, 1H), 4.11-4.05 (m, 4H), 3.86-3.84 (m, 1H), 3.17-3.12 (m, 1H), 2.69-2.65 (m, 1H), 2.02 (s, 3H), 1.91-1.82 (m, 2H), 1.54-1.48 (m, 1H), 1.45-1.38 (m, 1H).

### tert-Butyl 4-(3-(6-aminopyridin-3-yl)-1-methyl-1H-indazole-5-carboxamido)piperidine-1-carboxylate (52)

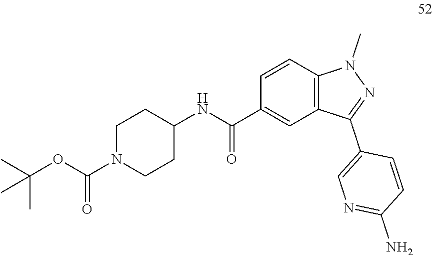 [SMILES: Cn1nc(-c2ccc(N)nc2)c2cc(C(=O)NC3CCN(C(=O)OC(C)(C)C)CC3)ccc21]

The same procedure for the synthesis of 50 was followed, except 1-methylpiperidin-4-amine was substituted for tert-butyl 4-aminopiperidine-1-carboxylate in the amide coupling reaction (step 2). Isolation and purification afforded title compound 52 (64%) as a light yellow foam; ¹H NMR (600 MHz, DMSO-d₆) δ 8.57 (d, J=2.0 Hz, 1H), 8.46 (s, 1H), 8.41 (d, J=7.8 Hz, 1H), 7.95-7.92 (m, 2H), 7.68 (d, J=8.8 Hz, 1H), 6.61 (d, J=8.6 Hz, 1H), 6.21 (s, 2H), 4.07 (s, 3H), 4.05-3.96 (m, 3H), 2.90-2.82 (m, 2H), 1.84-1.81 (m, 2H), 1.48-1.43 (m, 2H), 1.41 (s, 9H).

### 3-(6-Aminopyridin-3-yl)-1-methyl-N-(piperidin-4-yl)-1H-indazole-5-carboxamide (53)

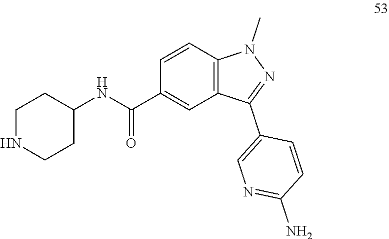 [SMILES: Cn1nc(-c2ccc(N)nc2)c2cc(C(=O)NC3CCNCC3)ccc21]

To a solution of 52 in CH₂Cl₂ (2 mL) was added trifluoroacetic acid (1 mL), and the mixture was stirred at room temperature for 1 h. The residue was concentrated in vacuo and purified by reverse-phase column chromatography on C-18 (CH₃CN/water, 5:95 to 100:0) to afford title compound 53 (50%) as a light yellow oil; ¹H NMR (600 MHz, DMSO-d₆) δ 8.74 (d, J=10.0 Hz, 1H), 8.57-8.55 (m, 3H), 8.49 (s, 1H), 8.41 (dd, J=9.1, 2.0 Hz, 1H), 8.11 (br s, 1H), 7.97 (dd, J=8.8, 1.4 Hz, 1H), 7.77 (d, J=8.8 Hz, 1H), 7.09 (d, J=9.1 Hz, 1H), 4.13-4.08 (m, 4H), 3.37-3.35 (m, 2H), 3.08-3.03 (m, 2H), 2.04-2.02 (m, 2H), 1.79-1.72 (m, 2H).

### 3-(3-(6-Aminopyridin-3-yl)-1-methyl-1H-indazole-5-carboxamido)benzoic acid (54)

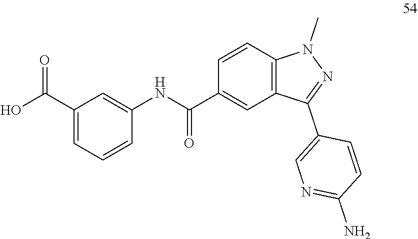 [SMILES: Cn1nc(-c2ccc(N)nc2)c2cc(C(=O)Nc3cccc(C(=O)O)c3)ccc21]

The same procedure for the synthesis of 50 was followed, except 1-methylpiperidin-4-amine was substituted for methyl 3-aminobenzoate in the amide coupling reaction (step 2). To the solution of crude product in THF/water (2 mL, v/v=1:1) was added LiOH·H₂O (4.0 equiv.), and the mixture was vigorously stirred at room temperature for 16 h. Isolation and purification afforded title compound 54 (50%) as a light yellow oil; ¹H NMR (600 MHz, DMSO-d₆) δ 10.31 (s, 1H), 8.67 (s, 1H), 8.64 (d, J=2.2 Hz, 1H), 8.13 (s, 1H), 8.05 (dd, J=8.8, 1.4 Hz, 1H), 8.02 (dd, J=8.6, 2.4 Hz, 1H), 7.86 (d, J=7.6 Hz, 1H), 7.74 (d, J=8.8 Hz, 1H), 7.60 (d, J=7.6 Hz, 1H), 7.23 (t, J=7.6 Hz, 1H), 6.63 (d, J=8.6 Hz, 1H), 6.22 (s, 2H), 4.10 (s, 3H).

### Syntheses of Compounds 55-65

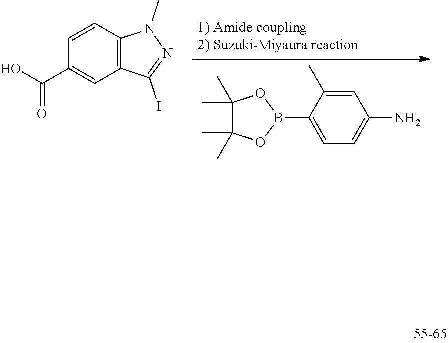 [SMILES: Cn1nc(I)c2cc(C(=O)O)ccc21 ; Cc1cc(N)ccc1B1OC(C)(C)C(C)(C)O1]

### 3-(4-Amino-2-methylphenyl)-N,1-dimethyl-1H-indazole-5-carboxamide (55)

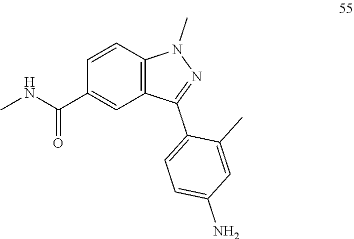

The same procedure for the synthesis of 21 was followed, except N-(3-aminophenyl)acetamide was substituted for methylamine hydrochloride in the amide coupling reaction (step 1). Isolation and purification afforded title compound 55 (50% over 2 steps) as a pale yellow foam; ¹H NMR (600 MHz, CDCl₃) δ 7.99 (br s, 1H), 7.88 (dd, J=8.8, 1.4 Hz, 1H), 7.42 (d, J=8.8 Hz, 1H), 7.27 (d, J=8.3 Hz, 1H), 6.68 (d, J=1.8 Hz, 1H), 6.63 (dd, J=8.3, 1.8 Hz, 1H), 6.15 (br s, 1H), 4.12 (s, 3H), 3.01 (d, J=4.8 Hz, 3H), 2.29 (s, 3H).

### 3-(4-Amino-2-methylphenyl)-N-(3-carbamoyl-4-methylphenyl)-1-methyl-1H-indazole-5-carboxamide (56)

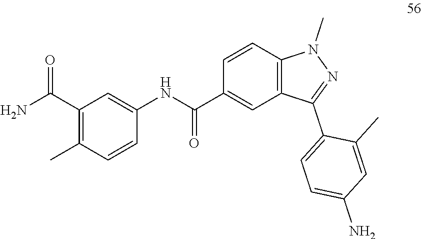 [SMILES: Cc1ccc(NC(=O)c2ccc3c(c2)c(-c2ccc(N)cc2C)nn3C)cc1C(N)=O]

The same procedure for the synthesis of 21 was followed, except N-(3-aminophenyl)acetamide was substituted for 3-amino-N-methylbenzamide in the amide coupling reaction (step 1). Isolation and purification afforded title compound 56 (15 mg, 67% over 2 steps) as a pale yellow solid; ¹H NMR (600 MHz, DMSO-d₆) δ 10.28 (s, 1H), 8.29 (s, 1H), 8.01 (d, J=8.8 Hz, 1H), 7.76 (s, 1H), 7.75-7.73 (m, 2H), 7.70 (s, 1H), 7.36 (s, 1H), 7.22 (d, J=8.1 Hz, 1H), 7.18 (d, J=8.3 Hz, 1H), 6.58 (s, 1H), 6.56 (d, J=8.1 Hz, 1H), 5.25 (s, 2H), 4.10 (s, 3H), 2.31 (s, 3H), 2.25 (s, 3H).

### 3-(4-Amino-2-methylphenyl)-1-methyl-N-(3-(methylcarbamoyl)phenyl)-1H-indazole-5-carboxamide (57)

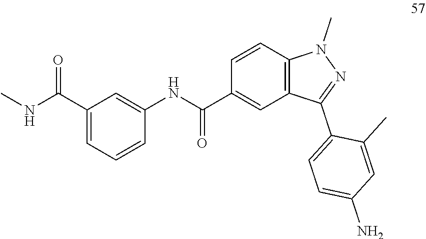

The same procedure for the synthesis of 21 was followed, except N-(3-aminophenyl)acetamide was substituted for 3-amino-N-methylbenzamide in the amide coupling reaction (step 1). Isolation and purification afforded title compound 57 (15 mg, 66% over 2 steps) as a pale yellow solid; ¹H NMR (600 MHz, DMSO-d₆) δ 10.40 (s, 1H), 8.42 (d, J=4.5 Hz, 1H), 8.33 (s, 1H), 8.20 (s, 1H), 8.03 (dd, J=8.9, 1.3 Hz, 1H), 7.95 (d, J=7.9 Hz, 1H), 7.74 (d, J=8.9 Hz, 1H), 7.52 (d, J=7.9 Hz, 1H), 7.41 (t, J=7.9 Hz, 1H), 7.23 (d, J=8.0 Hz, 1H), 6.58 (s, 1H), 6.56 (dd, J=8.0, 2.0 Hz, 1H), 5.25 (s, 2H), 4.10 (s, 3H), 2.78 (d, J=4.5 Hz, 3H), 2.25 (s, 3H).

### 3-(4-Amino-2-methylphenyl)-1-methyl-N-(4-methyl-3-(methylsulfonamido)phenyl)-1H-indazole-5-carboxamide (58)

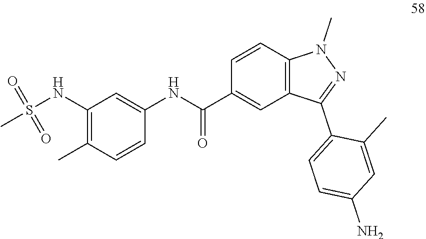

The same procedure for the synthesis of 21 was followed, except N-(3-aminophenyl)acetamide was substituted for N-(5-amino-2-methylphenyl)methanesulfonamide in the amide coupling reaction (step 1). Isolation and purification afforded title compound 58 (10 mg, 43% over 2 steps) as a light brown solid; ¹H NMR (600 MHz, DMSO-d₆) δ 10.28 (s, 1H), 9.06 (br s, 1H), 8.28 (s, 1H), 8.00 (dd, J=8.9, 1.1 Hz, 1H), 7.74-7.72 (m, 2H), 7.57 (dd, J=8.3, 1.4 Hz, 1H), 7.22 (d, J=8.0 Hz, 1H), 7.19 (d, J=8.3 Hz, 1H), 6.57 (s, 1H), 6.55 (dd, J=8.0, 2.0 Hz, 1H), 5.25 (s, 2H), 4.10 (s, 3H), 2.99 (s, 3H), 2.26 (s, 3H), 2.25 (s, 3H).

### 3-(4-Amino-2-methylphenyl)-1-methyl-N-(4-methyl-3-(methylsulfonamido)phenyl)-1H-indazole-5-carboxamide (59)

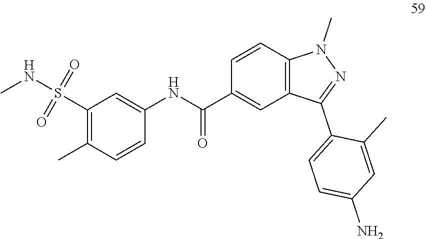

The same procedure for the synthesis of 21 was followed, except N-(3-aminophenyl)acetamide was substituted for 5-amino-N,2-dimethylbenzenesulfonamide in the amide coupling reaction (step 1). Isolation and purification afforded title compound 59 (7 mg, 30% over 2 steps) as a light brown solid; ¹H NMR (600 MHz, DMSO-d₆) δ 10.49 (s, 1H), 8.33 (s, 1H), 8.28 (d, J=2.1 Hz, 1H), 8.03 (dd, J=8.8, 1.2 Hz, 1H), 7.97 (dd, J=8.4, 2.1 Hz, 1H), 7.74 (d, J=8.8 Hz, 1H), 7.46 (br s, 1H), 7.37 (d, J=8.4 Hz, 1H), 7.22 (d, J=8.1 Hz, 1H), 6.58 (s, 1H), 6.56 (dd, J=8.1, 2.0 Hz, 1H), 5.25 (s, 2H), 4.10 (s, 3H), 2.51 (s, 3H), 2.44 (s, 3H), 2.25 (s, 3H).

### 3-(4-Amino-2-methylphenyl)-1-methyl-N-(p-tolyl)-1H-indazole-5-carboxamide (60)

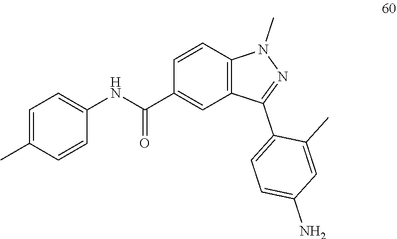

The same procedure for the synthesis of 21 was followed, except N-(3-aminophenyl)acetamide was substituted for p-toluidine in the amide coupling reaction (step 1). Isolation and purification afforded title compound 60 (39% over 2 steps) as a yellow oil; ¹H NMR (600 MHz, DMSO-d₆) δ 10.18 (s, 1H), 8.27 (br s, 1H), 8.00 (dd, J=8.8, 1.5 Hz, 1H), 7.73 (d, J=8.8 Hz, 2H), 7.64 (d, J=8.3 Hz, 2H), 7.21 (d, J=8.1 Hz, 1H), 7.14 (d, J=8.3 Hz, 2H), 6.57 (d, J=2.0 Hz, 1H), 6.55 (dd, J=8.1, 2.0 Hz, 1H), 5.25 (s, 2H), 4.10 (s, 3H), 2.27 (s, 3H), 2.24 (s, 3H).

### 3-(4-Amino-2-methylphenyl)-1-methyl-N-(4-(trifluoromethyl)phenyl)-1H-indazole-5-carboxamide (61)

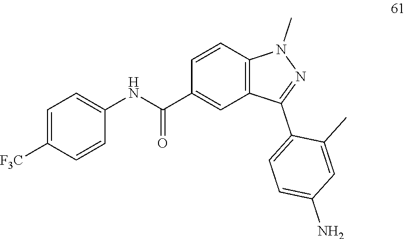 [SMILES: Cc1cc(N)ccc1-c1nn(C)c2ccc(C(=O)Nc3ccc(C(F)(F)F)cc3)cc12]

The same procedure for the synthesis of 21 was followed, except N-(3-aminophenyl)acetamide was substituted for 4-(trifluoromethyl)aniline in the amide coupling reaction (step 1). Isolation and purification afforded title compound 61 (31% over 2 steps) as a yellow oil; ¹H NMR (600 MHz, DMSO-d₆) δ 10.58 (s, 1H), 8.32 (br s, 1H), 8.03-8.00 (m, 3H), 7.76 (d, J=8.8 Hz, 1H), 7.71 (d, J=8.7 Hz, 2H), 7.22 (d, J=8.1 Hz, 1H), 6.58 (d, J=2.0 Hz, 1H), 6.55 (dd, J=8.1, 2.0 Hz, 1H), 5.26 (s, 2H), 4.11 (s, 3H), 2.25 (s, 3H).

### 3-(4-Amino-2-methylphenyl)-N-(4-ethoxyphenyl)-1-methyl-1H-indazole-5-carboxamide (62)

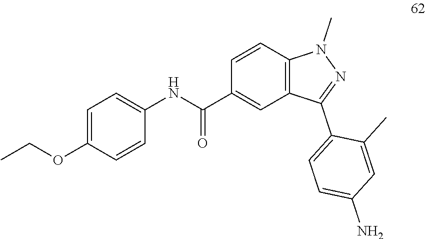 [SMILES: CCOc1ccc(NC(=O)c2ccc3c(c2)c(-c2ccc(N)cc2C)nn3C)cc1]

The same procedure for the synthesis of 21 was followed, except N-(3-aminophenyl)acetamide was substituted for 4-ethoxyaniline in the amide coupling reaction (step 1). Isolation and purification afforded title compound 62 (58% over 2 steps) as a yellow oil; ¹H NMR (600 MHz, DMSO-d₆) δ 10.28 (s, 1H), 8.29 (d, J=1.5 Hz, 1H), 8.01 (dd, J=8.8, 1.5 Hz, 1H), 7.75-7.73 (m, 3H), 7.28 (d, J=8.5 Hz, 2H), 7.22 (d, J=8.1 Hz, 1H), 6.58 (d, J=2.1 Hz, 1H), 6.55 (dd, J=8.1, 2.1 Hz, 1H), 5.25 (s, 2H), 4.37 (s, 2H), 4.10 (s, 3H), 3.27 (s, 3H), 2.25 (s, 3H).

### 3-(4-Amino-2-methylphenyl)-N-(4-fluorophenyl)-1-methyl-1H-indazole-5-carboxamide (63)

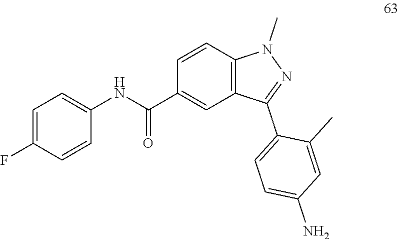 [SMILES: Cc1cc(N)ccc1-c1nn(C)c2ccc(C(=O)Nc3ccc(F)cc3)cc12]

The same procedure for the synthesis of 21 was followed, except N-(3-aminophenyl)acetamide was substituted for 4-fluoroaniline in the amide coupling reaction (step 1). Isolation and purification afforded title compound 63 (85% over 2 steps) as a yellow oil; ¹H NMR (600 MHz, DMSO-d₆) δ 10.32 (s, 1H), 8.28 (d, J=1.6 Hz, 1H), 8.00 (dd, J=8.8, 1.5 Hz, 1H), 7.79-7.76 (m, 2H), 7.74 (d, J=8.8 Hz, 1H), 7.21 (d, J=8.1 Hz, 1H), 7.18 (t, J=8.9 Hz, 2H), 6.57 (d, J=2.0 Hz, 1H), 6.55 (dd, J=8.1, 2.0 Hz, 1H), 5.25 (s, 2H), 4.10 (s, 3H), 2.24 (s, 3H).

### 3-(4-Amino-2-methylphenyl)-N-(4-chlorophenyl)-1-methyl-1H-indazole-5-carboxamide (64)

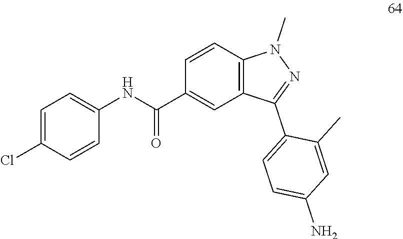 [SMILES: Cc1cc(N)ccc1-c1nn(C)c2ccc(C(=O)Nc3ccc(Cl)cc3)cc12]

The same procedure for the synthesis of 21 was followed, except N-(3-aminophenyl)acetamide was substituted for 4-chloroaniline in the amide coupling reaction (step 1). Isolation and purification afforded title compound 64 (50% over 2 steps) as a light yellow oil; ¹H NMR (600 MHz, DMSO-d₆) δ 10.38 (s, 1H), 8.28 (br s, 1H), 8.00 (dd, J=8.8, 1.5 Hz, 1H), 7.81 (d, J=8.9 Hz, 2H), 7.74 (d, J=8.8 Hz, 1H), 7.39 (d, J=8.9 Hz, 2H), 7.21 (d, J=8.1 Hz, 1H), 6.58 (d, J=2.0 Hz, 1H), 6.55 (dd, J=8.1, 2.0 Hz, 1H), 5.25 (s, 2H), 4.10 (s, 3H), 2.24 (s, 3H).

### 3-(4-Amino-2-methylphenyl)-N-(4-(cyclopropylmethoxy)phenyl)-1-methyl-1H-indazole-5-carboxamide (65)

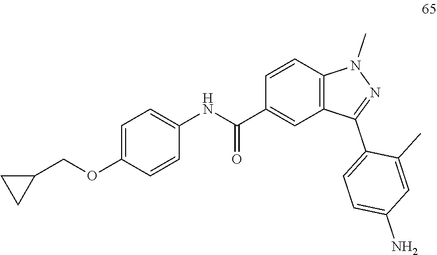 [SMILES: Cc1cc(N)ccc1-c1nn(C)c2ccc(C(=O)Nc3ccc(OCC4CC4)cc3)cc12]

The same procedure for the synthesis of 21 was followed, except N-(3-aminophenyl)acetamide was substituted for 4-(cyclopropylmethoxy)aniline in the amide coupling reaction (step 1). Isolation and purification afforded title compound 65 (40% over 2 steps) as a white foam; ¹H NMR (600 MHz, DMSO-d₆) δ 10.13 (s, 1H), 8.26 (s, 1H), 8.08 (d, J=8.8 Hz, 1H), 7.72 (d, J=8.8 Hz, 1H), 7.63 (d, J=8.6 Hz, 2H), 7.21 (d, J=8.0 Hz, 1H), 6.89 (d, J=8.6 Hz, 2H), 6.57 (s, 1H), 6.55 (d, J=8.0 Hz, 1H), 5.25 (s, 2H), 4.10 (s, 3H), 3.79 (d, J=6.9 Hz, 2H), 2.24 (s, 3H), 1.24-1.18 (m, 1H), 0.57-0.56 (m, 2H), 0.32-0.31 (m, 2H).

### Synthesis of Compound 66

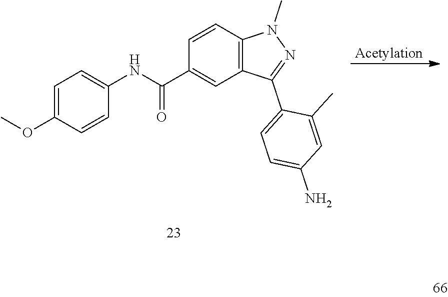

### 3-(4-Acetamido-2-methylphenyl)-N-(4-methoxyphenyl)-1-methyl-1H-indazole-5-carboxamide (66)

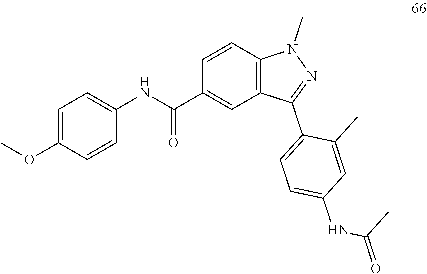 [SMILES: COc1ccc(NC(=O)c2ccc3c(c2)c(-c2ccc(NC(C)=O)cc2C)nn3C)cc1]

To a solution of 23 (13 mg, 0.03 mmol, 1.0 equiv.) and TEA (6 μL, 0.04 mmol, 1.2 equiv.) in anhydrous THF (1 mL) was added acetyl chloride (3 μL, 0.04 mmol, 1.2 equiv.) under nitrogen. The reaction mixture was stirred at room temperature for 16 h and then quenched by the addition of water. The aqueous layer was extracted twice with EtOAc, and the combined organic extracts were washed with brine, dried over anhydrous Na₂SO₄, filtered, and concentrated. The residue was purified by column chromatography on silica gel (CH₃OH/CH₂Cl₂, 1:99 to 2:98) to afford title compound 66 (6 mg, 42%) as a light yellow oil; ¹H NMR (600 MHz, CDCl₃) δ 8.24 (br s, 1H), 8.10 (s, 1H), 8.01 (d, J=8.6 Hz, 1H), 7.58 (d, J=8.9 Hz, 2H), 7.47-7.44 (m, 3H), 7.37-7.35 (m, 2H), 6.88 (d, J=8.9 Hz, 2H), 4.14 (s, 3H), 3.80 (s, 3H), 2.30 (s, 3H), 2.17 (s, 3H).

### Syntheses of Compounds 67-75

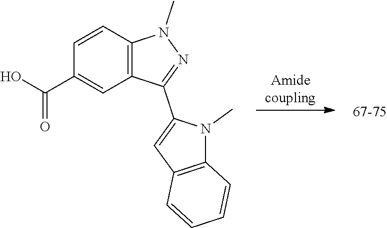 [SMILES: Cn1nc(-c2cc3ccccc3n2C)c2cc(C(=O)O)ccc21]

### N,1-Dimethyl-3-(1-methyl-1H-indol-2-yl)-1H-indazole-5-carboxamide (67)

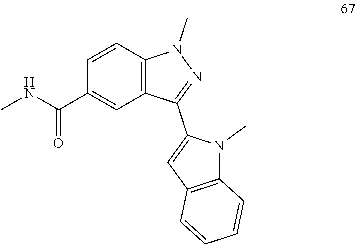

The same procedure for the synthesis of 29 was followed, except 4-methoxy-3-methylaniline was substituted for methylamine hydrochloride in the amide coupling reaction (step 2b). The residue was purified by column chromatography on silica gel (CH₃OH/CH₂Cl₂, 1:99 to 3:97) to afford title compound 67 (60%) as a pale yellow foam; ¹H NMR (600 MHz, DMSO-d₆) δ 8.63-8.61 (m, 1H), 8.51 (br s, 1H), 7.99 (dd, J=8.8, 1.6 Hz, 1H), 7.79 (d, J=8.8 Hz, 1H), 7.68 (d, J=7.8 Hz, 1H), 7.56 (dd, J=8.3, 0.6 Hz, 1H), 7.26-7.23 (m, 1H), 7.15 (d, J=0.6 Hz, 1H), 7.14-7.11 (m, 1H), 4.19 (s, 3H), 4.06 (s, 3H), 2.83 (d, J=4.5 Hz, 3H).

### N-(3-Bromo-4-methoxyphenyl)-1-methyl-3-(1-methyl-1H-indol-2-yl)-1H-indazole-5-carboxamide (68)

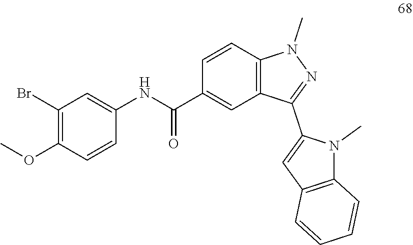

The same procedure for the synthesis of 29 was followed, except 4-methoxy-3-methylaniline was substituted for 3-bromo-4-methoxyaniline in the amide coupling reaction (step 2b). The residue was purified by column chromatography on silica gel (EtOAc/hexane, 30:70 to 35:65) to afford title compound 68 (13.5 mg, 84%) as a pale yellow solid; ¹H NMR (600 MHz, DMSO-d₆) δ 10.36 (s, 1H), 8.64 (s, 1H), 8.09-8.07 (m, 2H), 7.87 (d, J=8.8 Hz, 1H), 7.75 (dd, J=8.9, 2.5 Hz, 1H), 7.69 (d, J=8.1 Hz, 1H), 7.57 (d, J=8.1 Hz, 1H), 7.25 (t, J=8.1 Hz, 1H), 7.19 (s, 1H), 7.15-7.11 (m, 2H), 4.22 (s, 3H), 4.07 (s, 3H), 3.84 (s, 3H).

### N-(3-Iodo-4-methoxyphenyl)-1-methyl-3-(1-methyl-1H-indol-2-yl)-1H-indazole-5-carboxamide (69)

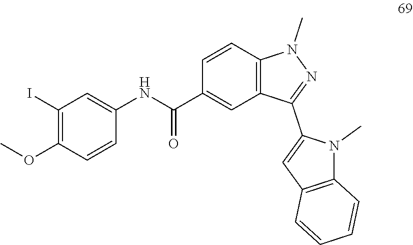

The same procedure for the synthesis of 29 was followed, except 4-methoxy-3-methylaniline was substituted for 3-iodo-4-methoxyaniline in the amide coupling reaction (step 2b). The residue was purified by column chromatography on silica gel (EtOAc/hexane, 30:70 to 35:65) to afford title compound 69 (13 mg, 91%) as a pale yellow solid; ¹H NMR (600 MHz, DMSO-d₆) δ 10.31 (s, 1H), 8.64 (s, 1H), 8.24 (d, J=2.5 Hz, 1H), 8.07 (dd, J=8.8, 1.4 Hz, 1H), 7.86 (d, J=8.8 Hz, 1H), 7.79 (dd, J=8.9, 2.5 Hz, 1H), 7.70 (d, J=8.1 Hz, 1H), 7.57 (d, J=8.1 Hz, 1H), 7.25 (t, J=8.1 Hz, 1H), 7.20 (s, 1H), 7.12 (t, J=8.1 Hz, 1H), 7.03 (d, J=8.9 Hz, 1H), 4.22 (s, 3H), 4.07 (s, 3H), 3.82 (s, 3H).

### N-(4-Methoxy-3-(trifluoromethyl)phenyl)-1-methyl-3-(1-methyl-1H-indol-2-yl)-1H-indazole-5-carboxamide (70)

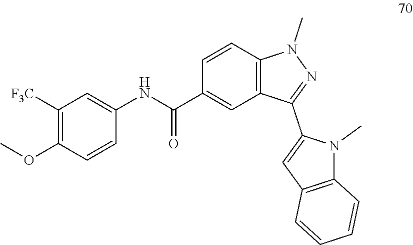

The same procedure for the synthesis of 29 was followed, except 4-methoxy-3-methylaniline was substituted for 4-methoxy-3-(trifluoromethyl)aniline in the amide coupling reaction (step 2b). The residue was purified by column chromatography on silica gel (EtOAc/hexane, 30:70 to 35:65) to afford title compound 70 (12 mg, 78%) as a pale yellow solid; ¹H NMR (600 MHz, DMSO-d₆) δ 10.48 (s, 1H), 8.66 (s, 1H), 8.12 (d, J=2.6 Hz, 1H), 8.10 (dd, J=8.8, 1.4 Hz, 1H), 8.03 (dd, J=9.0, 2.6 Hz, 1H), 7.88 (d, J=8.8 Hz, 1H), 7.69 (d, J=8.1 Hz, 1H), 7.57 (d, J=8.1 Hz, 1H), 7.30 (d, J=9.0 Hz, 1H), 7.26 (t, J=8.1 Hz, 1H), 7.19 (s, 1H), 7.12 (t, J=8.1 Hz, 1H), 4.22 (s, 3H), 4.07 (s, 3H), 3.89 (s, 3H).

### N-(3-Cyano-4-methoxyphenyl)-1-methyl-3-(1-methyl-1H-indol-2-yl)-1H-indazole-5-carboxamide (71)

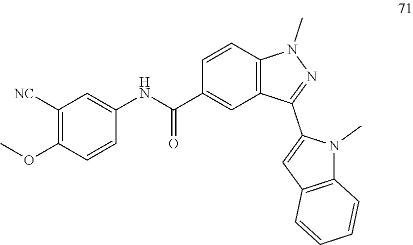

The same procedure for the synthesis of 29 was followed, except 4-methoxy-3-methylaniline was substituted for 5-amino-2-methoxybenzonitrile in the amide coupling reaction (step 2b). The precipitate was collected, sequentially washed with water and ether, and dried in vacuo to afford title compound 71 (9 mg, 63%) as a pale yellow solid; ¹H NMR (600 MHz, DMSO-d₆) δ 10.50 (s, 1H), 8.65 (s, 1H), 8.13 (d, J=2.6 Hz, 1H), 8.08 (dd, J=8.8, 1.4 Hz, 1H), 8.01 (dd, J=9.2, 2.6 Hz, 1H), 7.88 (d, J=8.8 Hz, 1H), 7.69 (d, J=8.1 Hz, 1H), 7.58 (d, J=8.1 Hz, 1H), 7.30 (d, J=9.2 Hz, 1H), 7.26 (t, J=8.1 Hz, 1H), 7.19 (s, 1H), 7.13 (t, J=8.1 Hz, 1H), 4.22 (s, 3H), 4.07 (s, 3H), 3.92 (s, 3H).

### N-(6-Methoxy-[1,1′-biphenyl]-3-yl)-1-methyl-3-(1-methyl-1H-indol-2-yl)-1H-indazole-5-carboxamide (72)

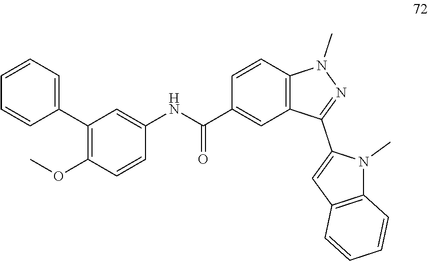 [SMILES: COc1ccc(NC(=O)c2ccc3c(c2)c(-c2cc4ccccc4n2C)nn3C)cc1-c1ccccc1]

The same procedure for the synthesis of 29 was followed, except 4-methoxy-3-methylaniline was substituted for 6-methoxy-[1,1′-biphenyl]-3-amine in the amide coupling reaction (step 2b). The residue was purified by column chromatography on silica gel (EtOAc/hexane, 30:70 to 35:65) to afford title compound 72 (13 mg, 82%) as a yellow solid; ¹H NMR (600 MHz, DMSO-d₆) δ 10.32 (s, 1H), 8.65 (s, 1H), 8.10 (dd, J=8.8, 1.3 Hz, 1H), 7.87 (d, J=8.8 Hz, 1H), 7.78 (dd, J=8.8, 2.6 Hz, 1H), 7.74 (d, J=2.6 Hz, 1H), 7.69 (d, J=8.0 Hz, 1H), 7.57 (d, J=8.0 Hz, 1H), 7.50 (d, J=7.4 Hz, 2H), 7.43 (t, J=7.4 Hz, 2H), 7.34 (t, J=7.4 Hz, 1H), 7.25 (t, J=8.0 Hz, 1H), 7.19 (s, 1H), 7.14-7.11 (m, 2H), 4.22 (s, 3H), 4.07 (s, 3H), 3.77 (s, 3H).

### N-(4-Methoxy-3-sulfamoylphenyl)-1-methyl-3-(1-methyl-1H-indol-2-yl)-1H-indazole-5-carboxamide (73)

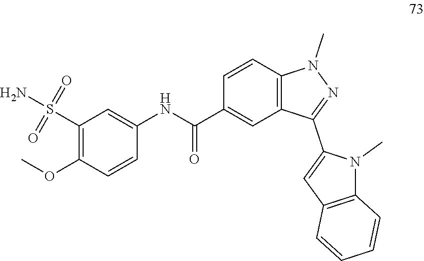

The same procedure for the synthesis of 29 was followed, except 4-methoxy-3-methylaniline was substituted for 5-amino-2-methoxybenzenesulfonamide in the amide coupling reaction (step 2b). The residue was purified by column chromatography on silica gel (EtOAc/hexane, 50:50 to 70:30) to afford title compound 73 (12 mg, 75%) as a pale yellow solid; ¹H NMR (600 MHz, DMSO-d₆) δ 10.47 (s, 1H), 8.68 (s, 1H), 8.22 (d, J=2.7 Hz, 1H), 8.11 (dd, J=8.9, 1.4 Hz, 1H), 8.03 (dd, J=9.0, 2.7 Hz, 1H), 7.87 (d, J=8.9 Hz, 1H), 7.70 (d, J=7.8 Hz, 1H), 7.57 (d, J=7.8 Hz, 1H), 7.27-7.22 (m, 2H), 7.20 (s, 1H), 7.13 (t, J=7.8 Hz, 1H), 7.10 (s, 2H), 4.22 (s, 3H), 4.08 (s, 3H), 3.90 (s, 3H).

### N-(3-Acetamido-4-methoxyphenyl)-1-methyl-3-(1-methyl-1H-indol-2-yl)-1H-indazole-5-carboxamide (74)

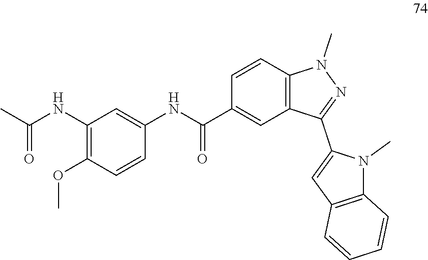 [SMILES: COc1ccc(NC(=O)c2ccc3c(c2)c(-c2cc4ccccc4n2C)nn3C)cc1NC(C)=O]

The same procedure for the synthesis of 29 was followed, except 4-methoxy-3-methylaniline was substituted for N-(5-amino-2-methoxyphenyl)acetamide in the amide coupling reaction (step 2b). The residue was purified by column chromatography on silica gel (EtOAc/hexane, 50:50 to 60:40) to afford title compound 74 (8 mg, 52%) as a yellow solid; ¹H NMR (600 MHz, DMSO-d₆) δ 10.30 (s, 1H), 9.15 (s, 1H), 8.65 (s, 1H), 8.28 (d, J=1.9 Hz, 1H), 8.09 (dd, J=8.8, 1.4 Hz, 1H), 7.85 (d, J=8.8 Hz, 1H), 7.69 (d, J=7.8 Hz, 1H), 7.59-7.56 (m, 2H), 7.25 (t, J=7.8 Hz, 1H), 7.21 (s, 1H), 7.12 (t, J=7.8 Hz, 1H), 7.02 (d, J=8.9 Hz, 1H), 4.21 (s, 3H), 4.08 (s, 3H), 3.83 (s, 3H), 2.10 (s, 3H).

### N-(2-Carbamoyl-4-methoxyphenyl)-1-methyl-3-(1-methyl-1H-indol-2-yl)-1H-indazole-5-carboxamide (75)

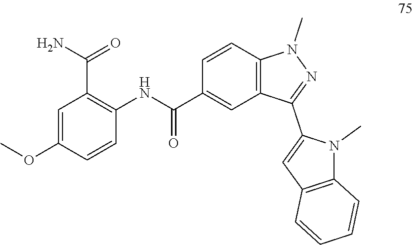

The same procedure for the synthesis of 29 was followed, except 4-methoxy-3-methylaniline was substituted for 2-amino-5-methoxybenzamide in the amide coupling reaction (step 2b). The precipitate was collected, sequentially washed with water and ether, and dried in vacuo to afford title compound 75 (11 mg, 77%) as a pale yellow solid; ¹H NMR (600 MHz, DMSO-d₆) δ 12.80 (s, 1H), 8.63-8.61 (m, 2H), 8.45 (s, 1H), 8.04 (dd, J=8.8, 1.4 Hz, 1H), 7.91 (d, J=8.8 Hz, 1H), 7.88 (s, 1H), 7.74 (d, J=7.8 Hz, 1H), 7.57 (d, J=7.8 Hz, 1H), 7.46 (d, J=2.9 Hz, 1H), 7.26 (t, J=7.8 Hz, 1H), 7.20 (dd, J=9.1, 2.9 Hz, 1H), 7.14-7.12 (m, 2H), 4.22 (s, 3H), 4.09 (s, 3H), 3.82 (s, 3H).

### Syntheses of Compounds 76-80

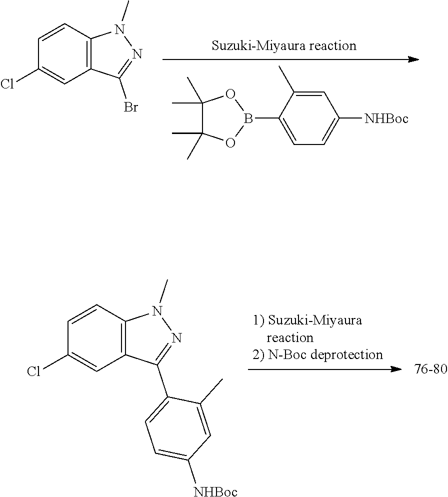 [SMILES: Cn1nc(Br)c2cc(Cl)ccc21 ; Cc1cc(NC(=O)OC(C)(C)C)ccc1B1OC(C)(C)C(C)(C)O1 ; Cc1cc(NC(=O)OC(C)(C)C)ccc1-c1nn(C)c2ccc(Cl)cc12]

### Step 1: tert-Butyl (4-(5-chloro-1-methyl-1H-indazol-3-yl)-3-methylphenyl)carbamate

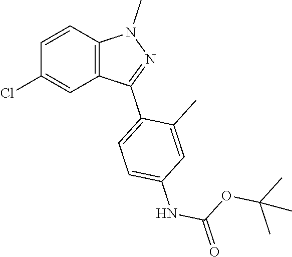 [SMILES: Cc1cc(NC(=O)OC(C)(C)C)ccc1-c1nn(C)c2ccc(Cl)cc12]

The general procedure employed for the Suzuki-Miyaura reaction was followed using 3-bromo-5-chloro-1-methyl-1H-indazole (246 mg, 1 mmol, 1.0 equiv.), tert-butyl (3-methyl-4-(4,4,5,5-tetramethyl-1,3,2-dioxaborolan-2-yl)phenyl)carbamate (500 mg, 1.5 mmol, 1.5 equiv.), and Pd(dppf)Cl₂·CH₂Cl₂ (82 mg, 0.1 mmol, 0.1 equiv.) in DME/Na₂CO₃₍ₛₐₜ₎(4 mL, v/v=1:1) at 100° C. for 1 h. The resulting mixture was extracted twice with EtOAc, and the combined organic extracts were washed with brine, dried over anhydrous Na₂SO₄, filtered, and concentrated. The residue was purified by column chromatography on silica gel (EtOAc/hexane, 20:80 to 30:70) to afford tert-butyl (4-(5-chloro-1-methyl-1H-indazol-3-yl)-3-methylphenyl)carbamate (120 mg, 32%) as a pale yellow foam.

### Step 2: ter t-Butyl (4-(5-chloro-1-methyl-1H-indazol-3-yl)-3-methylphenyl)carbamate (76)

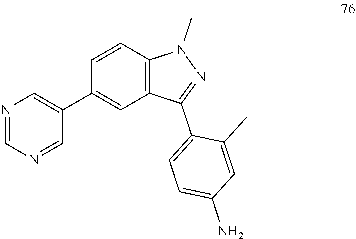 [SMILES: Cc1cc(N)ccc1-c1nn(C)c2ccc(-c3cncnc3)cc12]

The general procedure employed for the Suzuki-Miyaura reaction was followed using tert-butyl (4-(5-chloro-1-methyl-1H-indazol-3-yl)-3-methylphenyl)carbamate (19 mg, 0.05 mmol, 1.0 equiv.), pyrimidin-5-ylboronic acid (9 mg, 0.075 mmol, 1.5 equiv.), and XPhos Pd G3 (4 mg, 0.005 mmol, 0.1 equiv.) in DME/Na₂CO₃₍ₛₐₜ₎ (0.4 mL, v/v=1:1) at 110° C. for 1 h. The residue was purified by column chromatography on silica gel (EtOAc/hexane, 20:80 to 30:70) to give a partially pure product. The partially pure product was directly treated with a 4N HCl solution in 1,4-dioxane (0.5 mL), and the mixture was stirred at room temperature for 1 h. The resulting mixture was neutralized to pH 7 with 4M NaOH_{(aq)} and concentrated in vacuo. The residue was purified by reverse-phase column chromatography on C-18 (CH₃CN/water, 5:95 to 100:0) to afford title compound 76 (17 mg, 89%) as a pale yellow solid; ¹H NMR (600 MHz, DMSO-d₆) δ 9.17 (s, 2H), 9.14 (s, 1H), 7.94 (s, 1H), 7.83-7.80 (m, 2H), 7.24 (d, J=8.1 Hz, 1H), 6.56 (d, J=2.0 Hz, 1H), 6.53 (dd, J=8.1, 2.0 Hz, 1H), 5.22 (s, 2H), 4.10 (s, 3H), 2.26 (s, 3H).

### 3-Methyl-4-(1-methyl-5-(2-(trifluoromethyl)pyrimidin-5-yl)-1H-indazol-3-yl)aniline (77)

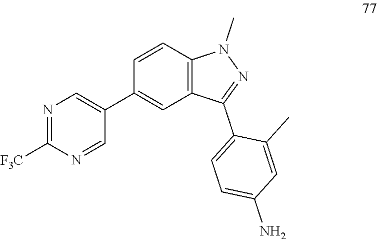 [SMILES: Cc1cc(N)ccc1-c1nn(C)c2ccc(-c3cnc(C(F)(F)F)nc3)cc12]

The same procedure for the synthesis of 76 was followed, except pyrimidin-5-ylboronic acid was substituted for (2-(trifluoromethyl)pyrimidin-5-yl)boronic acid in the Suzuki-Miyaura reaction (step 2). Isolation and purification afforded title compound 77 (9.5 mg, 60%) as a light yellow foam; ¹H NMR (600 MHz, DMSO-d₆) δ 9.43 (s, 2H), 8.09 (s, 1H), 7.92 (d, J=8.7 Hz, 1H), 7.85 (d, J=8.7 Hz, 1H), 7.25 (d, J=8.1 Hz, 1H), 6.56 (s, 1H), 6.53 (d, J=8.1 Hz, 1H), 5.24 (s, 2H), 4.11 (s, 3H), 2.26 (s, 3H).

### 3-Methyl-4-(1-methyl-5-(pyridin-3-yl)-1H-indazol-3-yl)aniline (78)

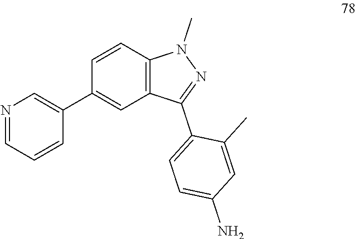 [SMILES: Cc1cc(N)ccc1-c1nn(C)c2ccc(-c3cccnc3)cc12]

The same procedure for the synthesis of 76 was followed, except pyrimidin-5-ylboronic acid was substituted for pyridin-3-ylboronic acid in the Suzuki-Miyaura reaction (step 2). Isolation and purification afforded title compound 78 (36%) as a white solid; ¹H NMR (600 MHz, DMSO-d₆) δ 8.90 (d, J=1.9 Hz, 1H), 8.54-8.53 (m, 1H), 8.10-8.09 (m, 1H), 7.81 (s, 1H), 7.77 (br s, 2H), 7.43-7.45 (m, 1H), 7.22 (d, J=8.1 Hz, 1H), 6.56 (d, J=1.9 Hz, 1H), 6.53 (dd, J=8.1, 1.9 Hz, 1H), 5.21 (s, 2H), 4.09 (s, 3H), 2.25 (s, 3H).

### 3-Methyl-4-(1-methyl-5-(6-methylpyridin-3-yl)-1H-indazol-3-yl)aniline (79)

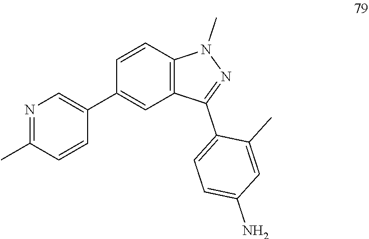

The same procedure for the synthesis of 76 was followed, except pyrimidin-5-ylboronic acid was substituted for (6-methylpyridin-3-yl)boronic acid in the Suzuki-Miyaura reaction (step 2). Isolation and purification afforded title compound 79 (79%) as a colorless oil; ¹H NMR (600 MHz, DMSO-d₆) δ 8.75 (d, J=2.3 Hz, 1H), 7.98 (d, J=8.0, 2.0 Hz, 1H), 7.77-7.72 (m, 3H), 7.31 (d, J=8.0 Hz, 1H), 7.21 (d, J=8.1 Hz, 1H), 6.56 (d, J=1.9 Hz, 1H), 6.52 (dd, J=8.1, 1.9 Hz, 1H), 5.21 (s, 2H), 4.09 (s, 3H), 2.50 (s, 3H), 2.24 (s, 3H).

### 4-(5-(6-Methoxypyridin-3-yl)-1-methyl-1H-indazol-3-yl)-3-methylaniline (80)

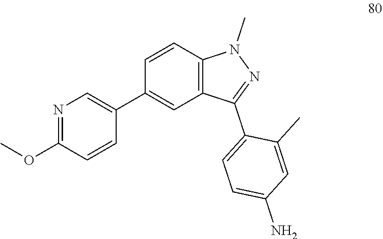

The same procedure for the synthesis of 76 was followed, except pyrimidin-5-ylboronic acid was substituted for (6-methoxypyridin-3-yl)boronic acid in the Suzuki-Miyaura reaction (step 2). Isolation and purification afforded title compound 80 (68%) as a colorless oil; ¹H NMR (600 MHz, DMSO-d₆) δ 8.47 (d, J=2.3 Hz, 1H), 8.03 (d, J=8.6, 2.3 Hz, 1H), 7.73-7.68 (m, 3H), 7.21 (d, J=8.0 Hz, 1H), 6.88 (d, J=8.6 Hz, 1H), 6.55 (d, J=1.9 Hz, 1H), 6.52 (dd, J=8.0, 1.9 Hz, 1H), 5.20 (s, 2H), 4.08 (s, 3H), 3.88 (s, 3H), 2.25 (s, 3H).

### Example 2: Production of Recombinant Bromodomain Proteins

To obtain quality binders from the DEC-Tec selection, it is critical that the target recombinant protein is well folded and highly pure. pET15b and pET28b bacterial expression vectors with an N-terminal polyhistidine tag (6-His; linear) were chosen to express and purify human BRDT and BRD4 recombinant bromodomain proteins as previously described in the literature. His-tagged recombinant proteins eluted from immobilized metal affinity chromatography were further purified with size exclusion gel filtration chromatography to assure the homogeneity of our preparation. To test whether the purified bromodomain proteins were properly folded, a fluorescence thermal shift stability assay was performed to measure the melting temperature. Further, to determine if the bromodomains were functional, an amplified luminescent proximity homogeneous assay (AlphaScreen), using biotinylated (+)-JQ1 was run to confirm the binding and stability of each bromodomain in DEC-Tec selection buffer.

### Example 3: DEC-Tec Affinity Selection with Bromodomain Proteins

To identify potent and selective BRDT-BD2 inhibitors for potential male contraception, a DEC-Tec platform was established for which >50 unique chemical libraries were generated cumulatively containing >4.5 billion drug-like compounds. The libraries were combinatorially constructed by sequential cycles of chemical building block attachment to a molecular scaffold and ligation of a corresponding DNA barcode that enabled precise identification in a pool of billions of compounds.

Using a split-and-pool strategy, each individual library of ˜100 million compounds was synthesized with a unique core scaffold. Quality assurance of libraries for selections was assured through optimization and validation of on-DNA chemistry reactions prior to library synthesis and the precision of the DNA barcode reads using Illumina sequencing after library synthesis. These individual DEC-Tec libraries were subsequently pooled and screened as multi-billion compound mixtures against His-tagged recombinant bromodomain proteins.

DNA-encoded molecules with affinity to the His-tagged bromodomain protein were separated from the mixture by using Nickel Nitrilotriacetic (Ni-NTA) magnetic beads that bind polyhistidine; these beads provide a rapid and easy capture of small scale His-tagged proteins and therefore can minimize the non-specific interaction between DNA-encoded chemical library materials and the beads, resulting with a clean background in a DNA environment.

Illumina next-generation sequencing and informatic analysis allowed the determination of the isolated DNA barcode sequences, and, thus, the structures of the enriched drug-like compounds could be identified. Enrichment of binding compounds were measured by a normalized Z-score metric. Enrichment was compared between different experimental conditions to identify potent and selective BRDT-BD2 inhibitors.

The selection for BRDT-BD2 binders comprised a pair of affinity selections with a protein concentration of 0.3 μM. One condition included (+)-JQ1 as a competitive inhibitor at a concentration of 100 μM, and the parallel condition contained no such inhibitor. To identify bromodomain-specific binders, an equivalent pair of affinity selections was conducted against BRDT-BD1. Finally, one additional affinity selection was conducted without protein to serve as a no-target control to identify any non-protein specific enrichment.

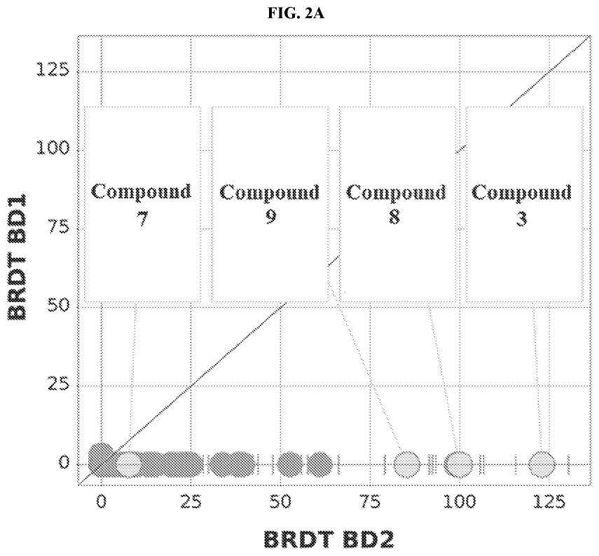

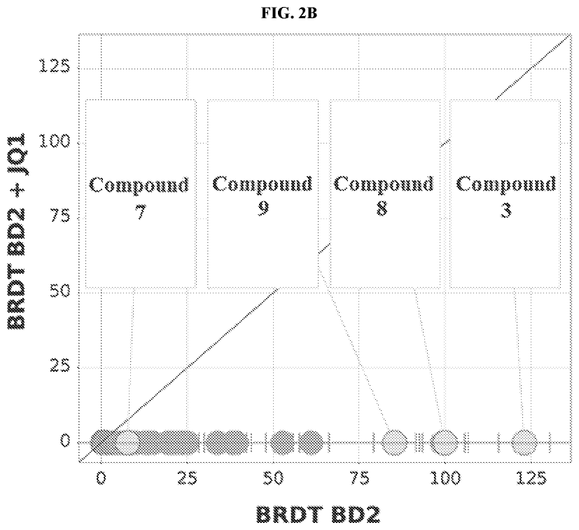

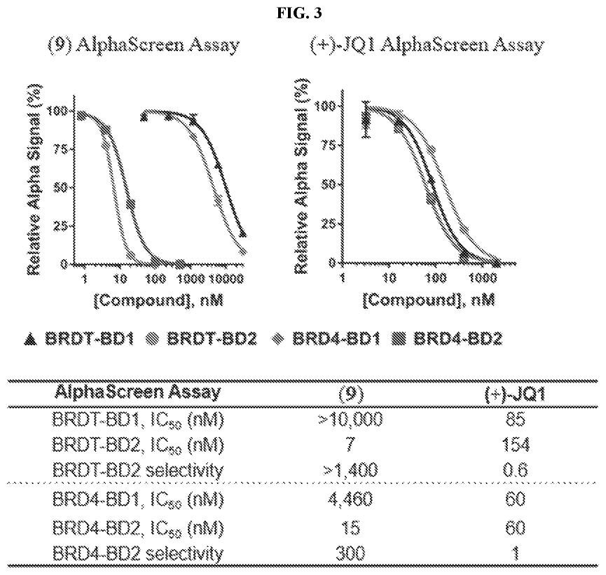

Comparison of the normalized enrichment of library members in the data sets highlighted the compound series shown in FIGS. 2A-2B, which demonstrated strong enrichment for BRDT-BD2 but not BRDT-BD1, and only in the absence of (+)-JQ1. Such an enrichment profile is consistent with selective binding to BRDT-BD2 in competition with (+)-JQ1. The observed compound series contained a conserved methyl indazole building block in cycle 2, a small number of cycle 1 linker building blocks, and several different aryl boronic acids in cycle 3.

Taken together, the strong enrichment and reasonable structure-enrichment relationship identified a promising chemical series.

The results in FIGS. 2A-2B utilize procedures to minimize non-specific binding of DEL members. Furthermore, the methods used to analyze, represent the selection results, and determine the structures of chemical binders use computer algorithms developed by the investigators that perform DEL selections. General summaries of this analysis method have been disclosed elsewhere.

### Example 4: Validation of BD2 DEC-Tec Selection Hits

The DEC-Tec selection results were first validated by resynthesizing hit molecules off-DNA, truncating the DNA barcode linkage to a methyl amide (Table 2). The synthetic pathway of DEC-Tec hits is illustrated in Scheme 1 (see Materials and Methods section for details). Using sequential HATU-mediated amide coupling, nitro reduction, and another HATU-mediated amide coupling, amines 1a and 1b were converted to intermediates 2a and 2b, respectively. Subsequent Suzuki-Miyaura cross-coupling reaction furnished the desired products 3-12.

These compounds were assayed for inhibition of BRDT-BD2 using an AlphaScreen competition assay with biotinylated (+)-JQ1 as the ligand. It was confirmed not only that the chemical series significantly inhibited BRDT-BD2 at low nanomolar concentrations but that all compounds showed poor inhibition of BRDT-BD1 with IC₅₀ values generally greater than 10 μM, an ˜1000-fold loss in activity from BRDT-BD2 (Table 2). Thus, the DEC-Tec screening accurately and rapidly guided discovery of potent and selective BRDT-BD2 binding compounds with confirmation through in vitro binding assays. While the correlation between observed sequence counts of hits identified in the DEC-Tec screening and inhibitory activity of compounds resynthesized off-DNA was not one-to-one, due to variance in synthetic yield among library members, compounds with low nanomolar affinity (e.g., compounds 3 and 8) could be separated from moderately lower affinity library members (e.g., compounds 11 and 12), and compounds with high selectivity were identified by using parallel screens.

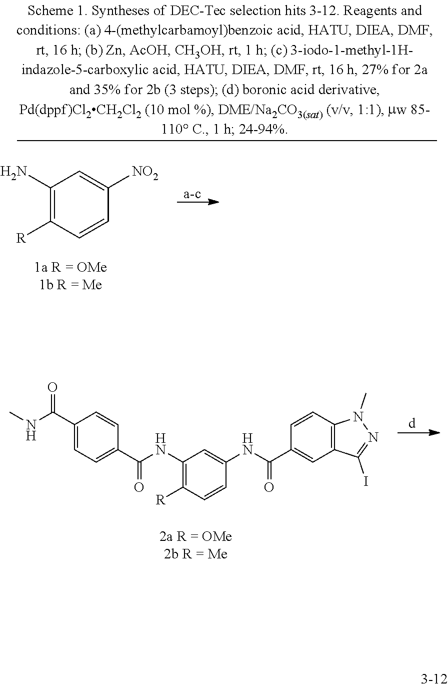

TABLE 2
Analysis of DEC-Tec hits and predicted library members.

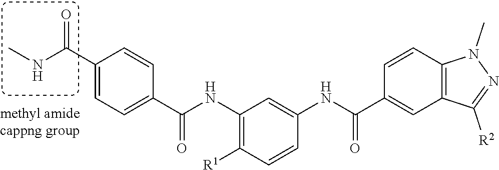

| | | | | BRDT-BD2 | BRDT-BD1 | MLM | HLM t_{1/2} |
|---|---|---|---|---|---|---|---|
| Compound | R¹ | R² | Count^{a} | IC₅₀ (nM)^{b} | IC₅₀ (nM)^{b} | t_{1/2} (min)^{c} | (min)^{c} |
| 3 | OMe | 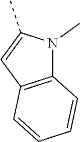 | 1039 | 22 | NE^{d} | 31 | 87 |
| 4 | OMe | 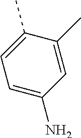 | 838 | 12 | >10,000 | 14 | 100 |
| 5 | OMe | 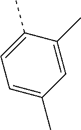 | 515 | 26 | NE^{d} | 20 | 18 |
| 6 | OMe | 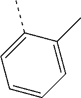 | 0 | 61 | 6,300 | 40 | 17 |
| 7 | OMe | 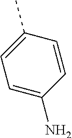 | 63 | 15 | >10,000 | 51 | 250 |
| 8 | Me | 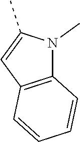 | 846 | 13 | >10,000 | 51 | 150 |
| 9 | Me | 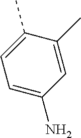 | 721 | 7 | >10,000 | 92 | 150 |
| 10 | Me | 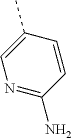 | 3 | 17 | >10,000 | 190 | 240 |
| 11 | Me | 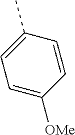 | 0 | 521 | >10,000 | | |
| 12 | Me | 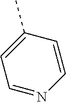 | 0 | 978 | NE^{d} | | |

^{a}Observed molecule count in the DEC-Tec screen against BRDT-BD2.
A “0” indicates that the compound was theoretically present in the library, but it was not detected
in the sequencing data.
^{b}IC₅₀ determined using AlphaScreen assay protocol.
^{c}t_{1/2} measured using liver microsomal stability assay; assay data >60 min is an extrapolated estimate
and included for relative determination of half-life.
^{d}NE: no effect at 10 μM in AlphaScreen assay.

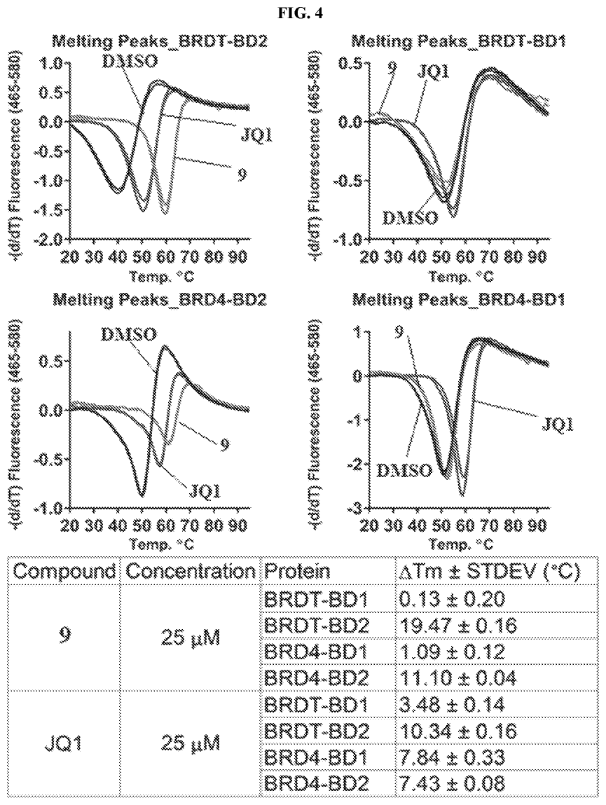

Among the validated compounds in the initial AlphaScreen assay, compound 9 emerged as an optimal BRDT-BD2 inhibitor with i) low nanomolar IC₅₀, ii) 22-fold higher potency than (+)-JQ1, iii) 1,400-fold selectivity over BRDT-BD1, iv) high metabolic stability in human liver microsomes (HLM), and v) greater stability in mouse liver microsomes (MLM) over other hits. To confirm the direct binding between compound 9 and BRDT-BD2, biophysical analysis was performed using a fluorescence thermal shift stability assay (FIG. 4). Upon protein binding, 9 showed a significant stabilization of the second bromodomain but not the first bromodomain of both BRDT and BRD4. Moreover, the increase in melting temperatures of the second bromodomains by 9 is more than that of (+)-JQ1, indicating a higher protein binding affinity of the compound and consistent with the AlphaScreen results.

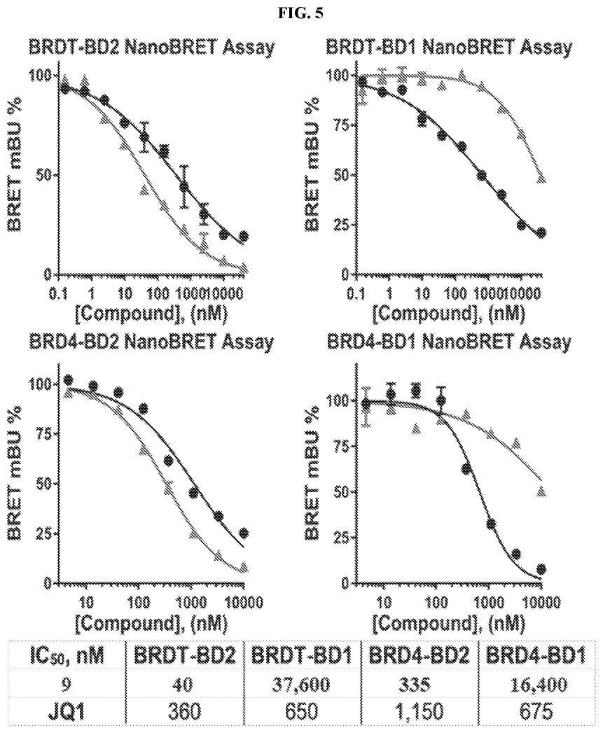

The binding of compound 9 to BET bromodomains was also studied using a NanoBRET target engagement assay which measures compound binding at select target proteins within intact cells. The inhibition of 9 on tracer binding was demonstrated to be potent and selective in NanoLuc-BET second bromodomain fusion proteins in transiently transfected HEK293 cells (FIG. 5).

### Example 5: Exploration of Structure-Activity Relationships (SARs)

Because BRDT is expressed in adult male germ cells in the testes, the blood-testis barrier (BTB), a lining of Sertoli cells impermeable to many small molecules, is a serious obstacle for drug development. Based on reports describing similarities between the BTB and blood-brain barrier (BBB) in terms of small molecule permeability and drug transporters, it was reasoned that using BBB permeability guidelines, such as molecular weight ≤400 g/mol, log P≤5, hydrogen bond donor≤3, and hydrogen bond acceptor≤7, would be a useful goal for hit optimization.

As compound 9 has a molecular weight of 537 g/mol, c Log P of 3.1, and 5 hydrogen bond donors, truncated analogs of the hit series with lower molecular weight and reduced number of hydrogen bond donors were explored, while maintaining c Log P.

All analogs were prepared by following a general synthetic pathway (route A or B) in Scheme 2 (see Materials and Methods section for details). In route A, commercially available or synthesized amines were converted to amides, and subsequent Suzuki coupling with a variety of boronic acid derivatives furnished analogs 13-17 and 20-26. In route B, Suzuki coupling reaction was carried out first. Subsequent saponification for the ester followed by amide coupling provided compounds 18, 19, and 28-36. The same procedure with additional acid treatment in the last step produced analog 27. These synthesized compounds are summarized into three groups (Tables 2, 3, and 4) based on the type of heterocycles attached to the indazole core.

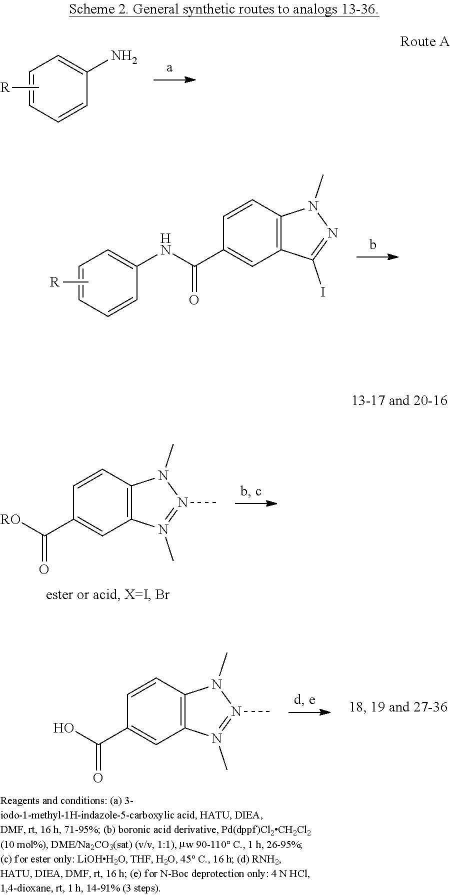

TABLE 3
Structures and activities of aminopyridine analogs 13-19

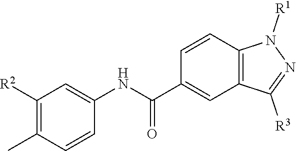

| | | | | BRDT-BD2 | BRDT-BD1 |
|---|---|---|---|---|---|
| Compound | R¹ | R² | R³ | IC₅₀ (nM) | IC₅₀ (nM) |
| 13 | Me | 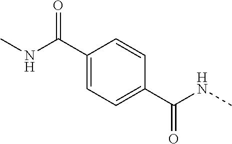 | 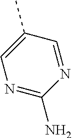 | 139 | >10,000 |
| 14 | Me | 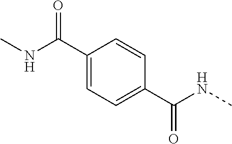 | 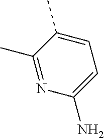 | 94 | NE^{a} |
| 15 | Me | 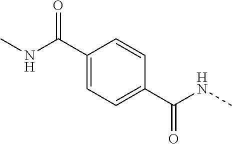 | 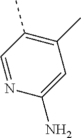 | 22 | NE^{b} |
| 16 | Me | 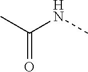 | 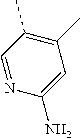 | 126 | NE^{a} |
| 17 | Me | 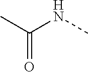 | 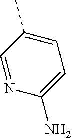 | 47 | >10,000 |
| 18 | Et | 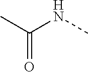 | 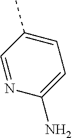 | 79 | >10,000 |
| 19 | i-Pr | 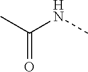 | 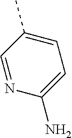 | 253 | >10,000 |

^{a}NE: no effect at 10 μM in AlphaScreen assay.
^{b}NE: no effect at 20 μM in AlphaScreen assay.

TABLE 4
Structures and activities of analogs 20-27

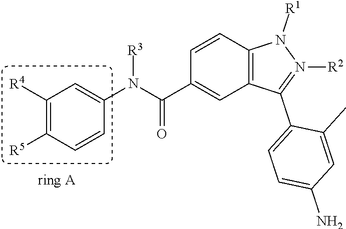

| Compound | R¹ | R² | R³ | R⁴ | R⁵ | BRDT-BD2 IC₅₀ (nM) | BRDT-BD1 IC₅₀ (nM) |
|---|---|---|---|---|---|---|---|
| 20 | Me | — | H | 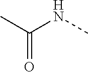 | Me | 8 | >10,000 |
| 21 | Me | — | H | 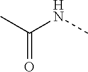 | H | 14 | >10,000 |
| 22 | Me | — | H | 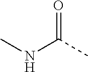 | Me | 18 | >10,000 |
| 23 | Me | — | H | 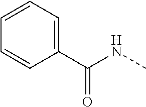 | OMe | 35 | NE^{a} |
| 24 | Me | — | H | H | OMe | 54 | NE^{a} |
| 25 | Me | — | H | H | H | 90 | >10,000 |
| 26 | Me | — | Me | H | OMe | >1,000 | >10,000 |
| 27 | — | Me | H | H | OMe | 17,200 | >10,000 |

^{a}NE: no effect at 10 μM in AlphaScreen assay.

TABLE 5
Exploration of functionalized ring A among indole derivatives 28-36

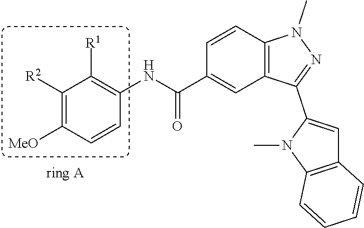

| | | | BRDT-BD2 |
|---|---|---|---|
| Compound | R¹ | R² | IC₅₀ (nM) |
| 28 | H | H | 121 |
| 29 | H | Me | 111 |
| 30 | H | OMe | 101 |
| 31 | H | F | 119 |
| 32 | H | Cl | 164 |
| 33 | Me | H | 183 |
| 34 | OMe | H | 926 |
| 35 | F | H | 203 |
| 36 | Cl | H | >1,250 |

Aminopyridine 10, despite slightly lower affinity than compound 9, showed high metabolic stability in mouse and human liver microsomes and mitigation of potential Ames positive behavior in 9 based on the exposed aniline. Aminopyrimidine analog (compound 13) and methyl substitution of the aminopyridine to mimic the 2-methyl-4-aminophenyl ring in 9 (compounds 14 and 15) displayed lower affinity than compound 10 (Table 3). On the indazole core, methyl homologation led to loss in potency (compounds 17-19), and switching the methyl from N1 to N2 caused a dramatic loss of activity (Table 4, compounds 24 and 27). Although not wishing to be limited by theory, the loss of potency in compound 26 suggested the amide adjacent to the indazole is critical.

A truncation series (compounds 4, 23-25) from the DNA attachment point towards ring A demonstrated progressive loss of activity while still retaining IC₅₀<100 nM (Table 4). Interestingly, truncation from 9 to compound 20 showed no loss in activity despite loss of >100 g/mol molecular weight. From 20, loss of the R³ methyl (compound 21) or reversing the amide (compound 22) imparted minimal effects on potency. Based on these findings, a series of simple substituents off ring A were investigated to assess whether other groups could substitute for the amide and retain activity while removing a hydrogen bond donor (Table 5). Relative to simplified comparator 28, methyl, methoxy, and fluoro substituents (compounds 29-31) at R¹ showed equivalent activity while Cl at R¹ and all examined R² substituents showed a loss in activity.

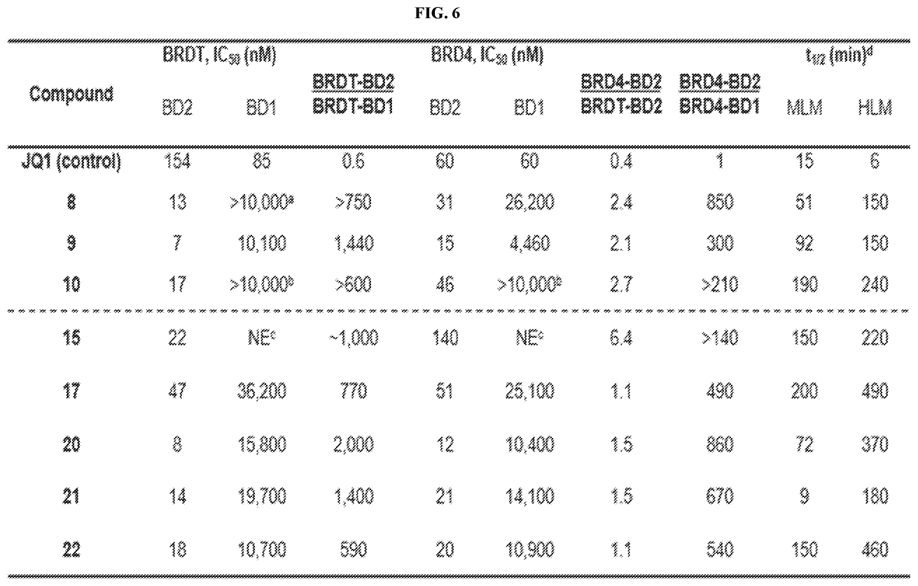

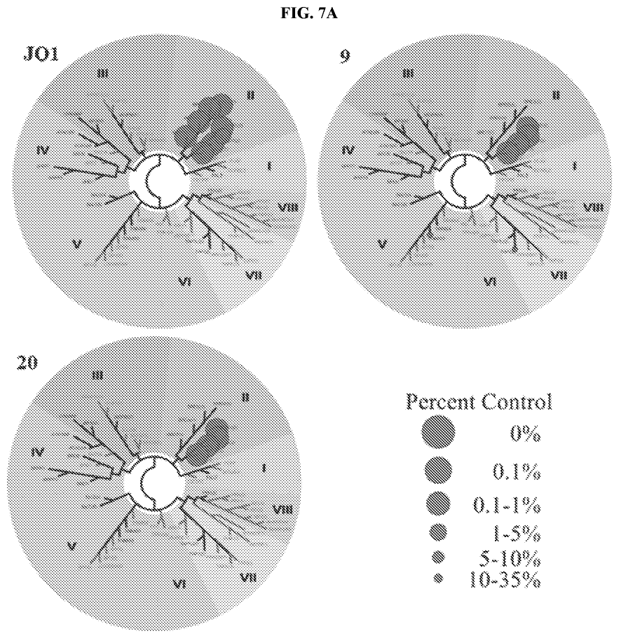

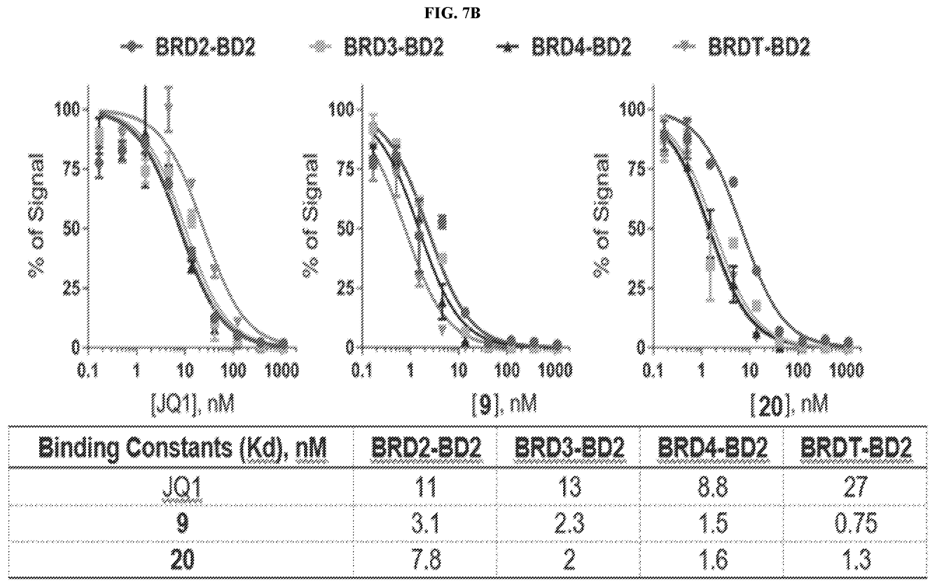

Selected potent compounds with IC₅₀<50 nM were further studied for BRD4 activity and microsomal stability (summarized in FIG. 6). All tested analogs demonstrated both high BRD4-BD2 versus BRD4-BD1 selectivity and BRDT-BD2 versus BRDT-BD1 selectivity, although intra-bromodomain selectivity was greater for BRDT than BRD4. Interestingly, aminopyridine analog 15 was observed to have slightly enhanced BRDT-BD2 versus BRD4-BD2 selectivity over other tested analogs. High human microsomal stability was observed for all tested derivatives while high mouse microsomal stability was measured for compounds 15, 17, 21, and 22. As 20 displayed a good balance of microsomal stability and activity, it was selected as an optimized hit for further analysis. BROMOscan bromodomain competition binding assays further confirmed the affinity and the selectivity of these compounds for the second bromodomain of BET subfamily members (FIG. 7A). While 9 demonstrated very weak binding to two bromodomains outside of the BET subfamily, 20 showed potent and selective binding to only the second bromodomains within the BET subfamily. Both of these compounds have the highest affinity for BRDT-BD2 among all of the bromodomains (FIG. 7A). BromoKdELECT dose-response curves show that 9 and 20 are more than 20-fold more potent than (+)-JQ1 at binding BRDT-BD2 in this assay (FIG. 7B), approximating the AlphaScreen findings presented in FIG. 6.

Tables 5 and 6 provide structure activity relationship studies of Type I compounds 37-75 (Table 6) and Type II compounds 76-80 (Table 7). Preparation of compounds 37-75 was achieved by following a general synthetic pathway in Scheme 2 (route A: 37-49 and 55-66; route B: 50-54 and 67-75; see Materials and Methods section for details). Compounds 76-80 were prepared by employing successive Suzuki-Miyaura reactions with different palladium catalysts (see Materials and Methods section for details).

TABLE 6
Structures and activities of compounds 37-75

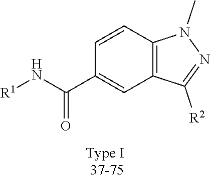

| | | | BRDT-BD2 | BRDT-BD1 |
|---|---|---|---|---|
| Cmpd | R¹ | R² | IC₅₀ (nM) | IC₅₀ (nM) |
| 37 | 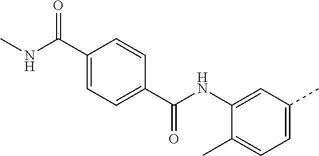 | 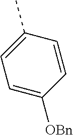 | >10,000 | |
| 38 | 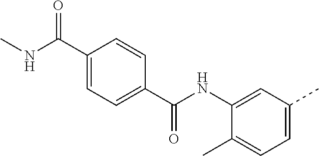 | 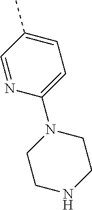 | >10,000 | |
| 39 | 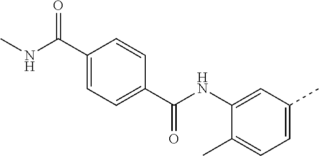 | 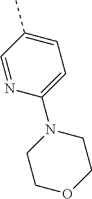 | >10,000 | |
| 40 | 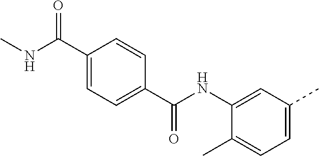 | 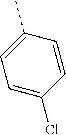 | 106 | |
| 41 | 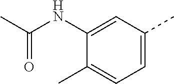 | 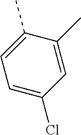 | 58 | |
| 42 | 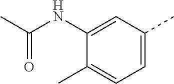 | 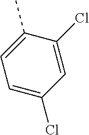 | 320 | |
| 43 | 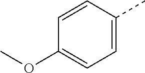 | 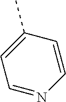 | 978 | |
| 44 | 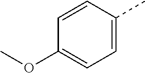 | 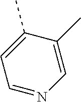 | 705 | |
| 45 | 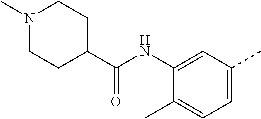 | 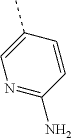 | 58 | |
| 46 | 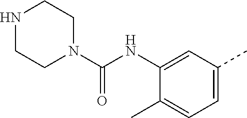 | 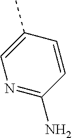 | 79 | |
| 47 | 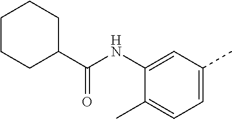 | 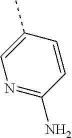 | 113 | |
| 48 | 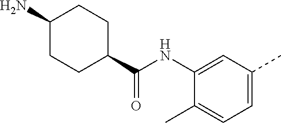 | 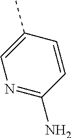 | 51 | |
| 49 | 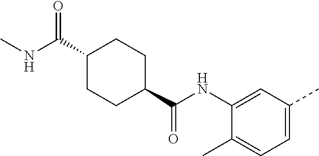 | 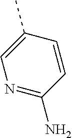 | 68 | |
| 50 | 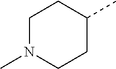 | 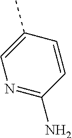 | >200 | |
| 51 | 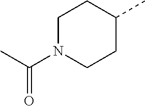 | 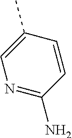 | >200 | |
| 52 | 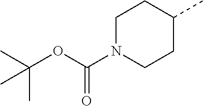 | 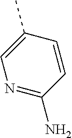 | >200 | |
| 53 | 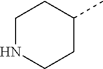 | 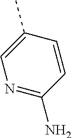 | >200 | |
| 54 | 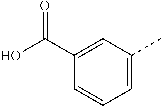 | 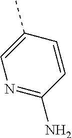 | 381 | |
| 55 | Me | 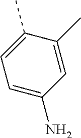 | 988 | >10,000 |
| 56 | 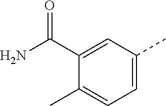 | 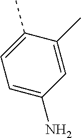 | 34 | |
| 57 | 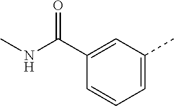 | 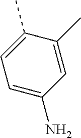 | 29 | |
| 58 | 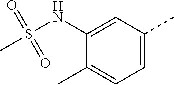 | 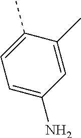 | 67 | |
| 59 | 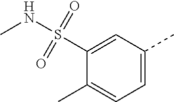 | 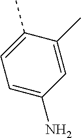 | 148 | |
| 60 | 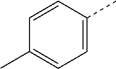 | 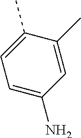 | 91 | |
| 61 | 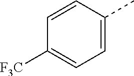 | 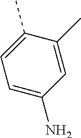 | 898 | |
| 62 | 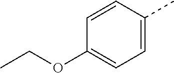 | 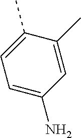 | 48 | |
| 63 | 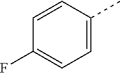 | 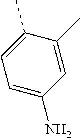 | 101 | |
| 64 | 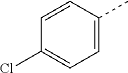 | 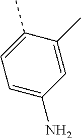 | 209 | |
| 65 | 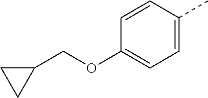 | 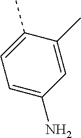 | 98 | >10,000 |
| 66 | 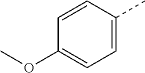 | 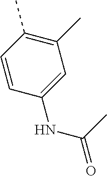 | >10,000 | |
| 67 | Me | 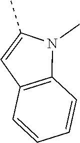 | 261 | >10,000 |
| 68 | 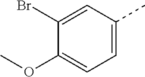 | 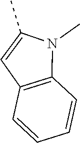 | 274 | |
| 69 | 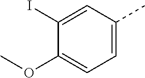 | 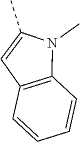 | 502 | |
| 70 | 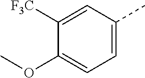 | 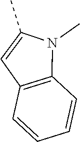 | 626 | |
| 71 | 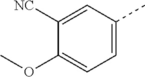 | 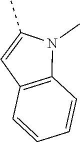 | 168 | |
| 72 | 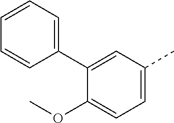 | 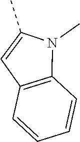 | 2,388 | |
| 73 | 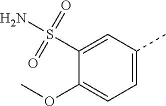 | 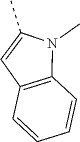 | 111 | |
| 74 | 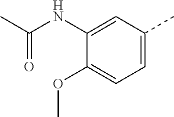 | 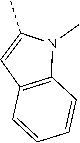 | 32 | |
| 75 | 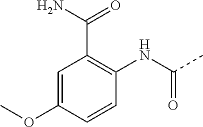 | 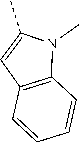 | 4,369 | |

TABLE 7
Structures and activities of compounds 76-80

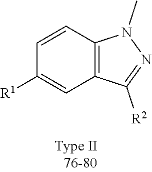

| | | | BRDT- | BRDT- |
|---|---|---|---|---|
| | | | BD2 | BD1 |
| Cmpd | R¹ | R² | IC₅₀ (nM) | IC₅₀ (nM) |
| 76 | 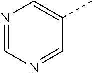 | 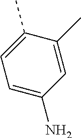 | 5,435 | 9,475 |
| 77 | 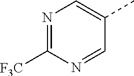 | 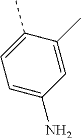 | 3,627 | >10,000 |
| 78 | 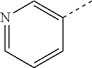 | 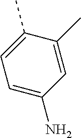 | >10,000 | 2,445 |
| 79 | 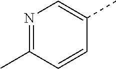 | 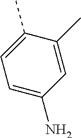 | >10,000 | 3,208 |
| 80 | 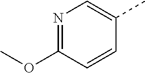 | 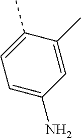 | >10,000 | 1,021 |

### Example 6: Co-Crystal Structures of 9 and 20 with BRDT-BD2

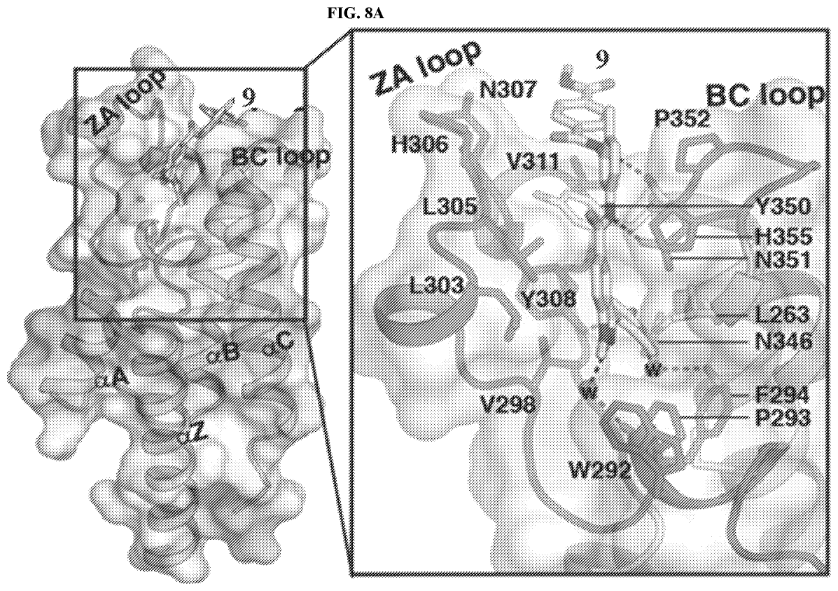

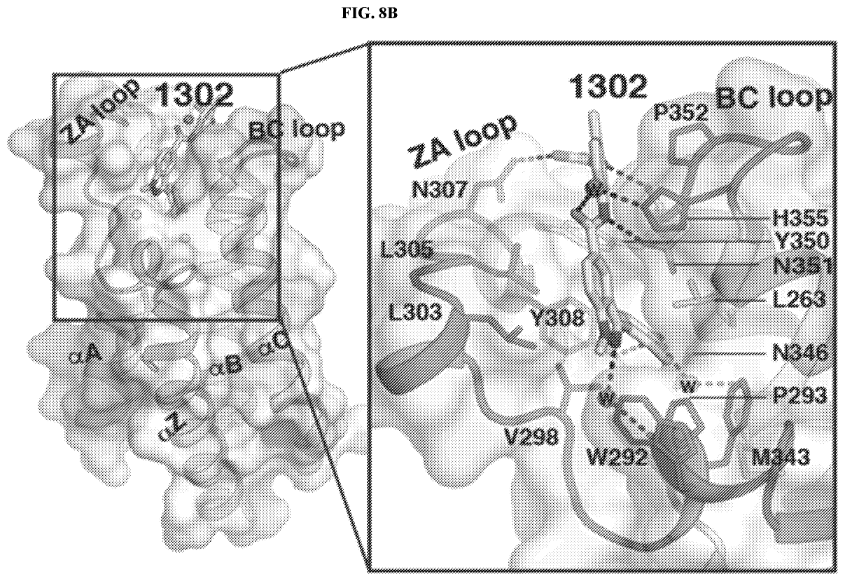

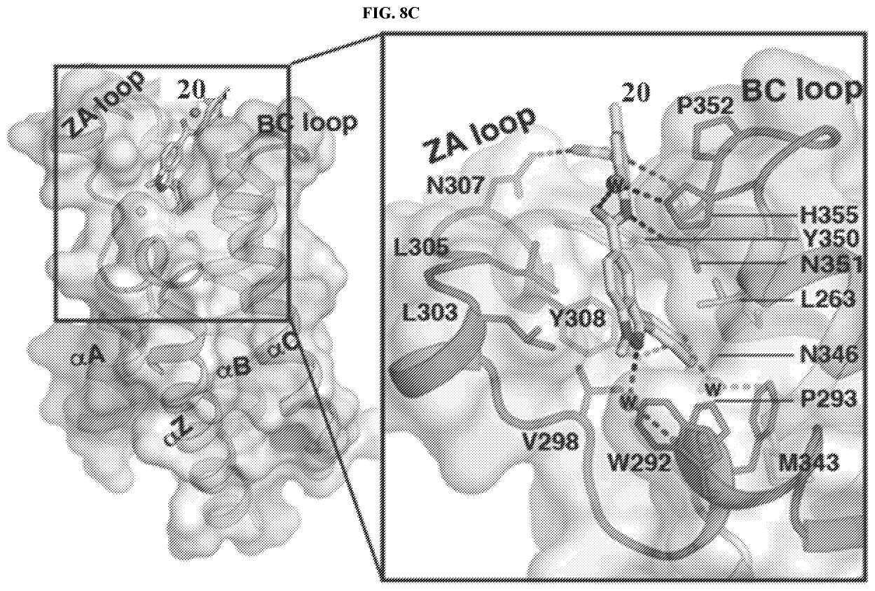

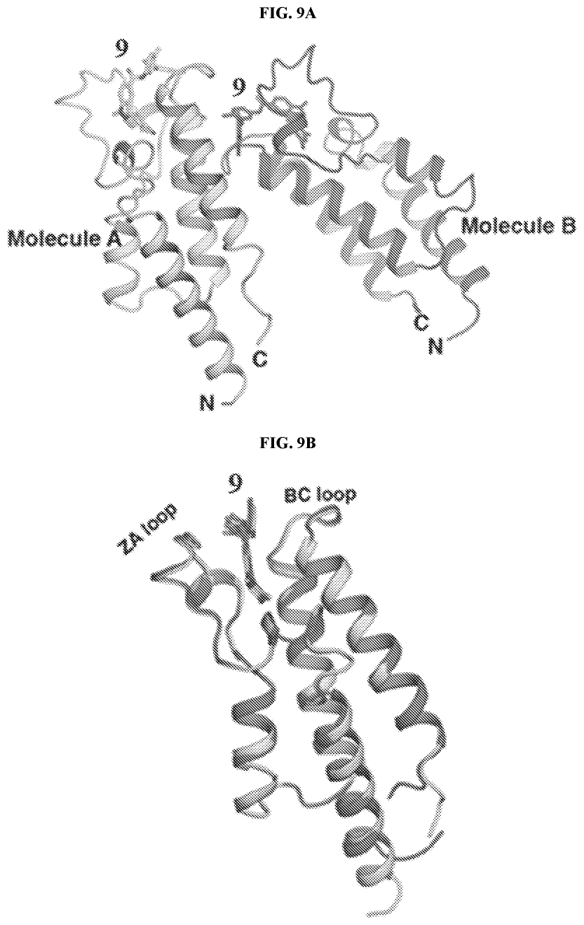

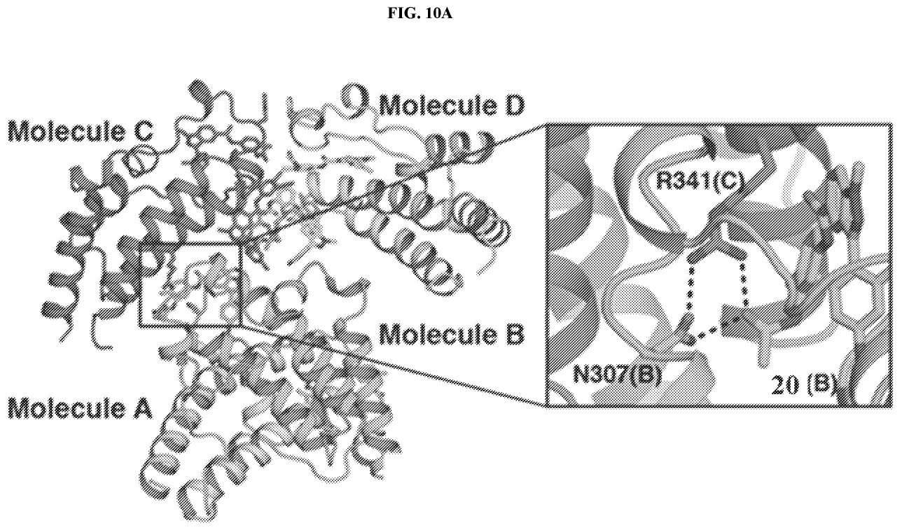

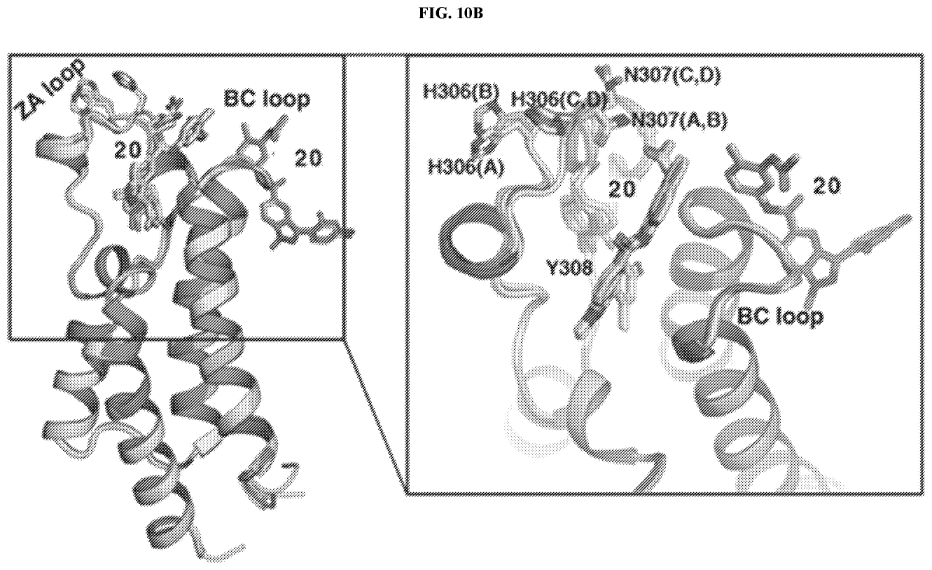

To understand the structural basis of high affinity and selectivity, crystal structures of BRDT-BD2 bound with 9 and 20 were solved at 2.27 and 1.90 Å resolutions, respectively (FIGS. 8A-8C). The BRDT-BD2/9 crystal contained two molecules per asymmetric unit, whereas the BRDT-BD2/20 crystal contained four (FIGS. 9A-9B and FIGS. 10A-10B). The overall structure of two molecules in the BRDT-BD2/9 crystal was similar with one 9 bound at each acetylated lysine (KAc) binding pocket (showing an RMSD of 0.25 Å between shared 97 CA atoms) (FIG. 9B). The overall structure of four molecules in the BRDT-BD2/20 crystal was nearly identical except for a short segment of the ZA loop (residues 305-308). In two of the BRDT-BD2/20 complexes in the crystal (molecule A and B), an arginine residue (R341) from the neighboring molecule in the crystal interacted with N307 and the bound 20 through hydrogen bonds (FIG. 10A). This caused a segment of the ZA loop to move away slightly from the core (FIG. 10B). Lastly, two molecules showed an additional 20 bound at a previously unobserved site near the BC loop (FIG. 10A). The additional 20 molecules bound at molecule C and D showed several contacts to other protein molecules in the asymmetric unit mediated through Jeffamine ED-2000 and ordered waters. These contacts suggested that the different ZA loop conformation and the second 20 binding were caused by crystal packing.

Compound 9 binds to the extended pocket consisting of the KAc binding pocket, ZA channel, WPF shelf and a channel formed between ZA and BC loops (FIG. 8A). Specifically, the aniline nitrogen interacts with the side chain of Y308 in the ZA-loop whereas the two internal amide nitrogens form hydrogen bonds with the backbone carbonyl of Y350 and the side chain of N351 at the BC-loop. The aniline ring binds deep into a hydrophobic pocket consisting of non-polar residues from the ZA loop (V298), B, and C helices (Y308 and C347). The indazole ring docks to the WPF shelf (W292, P293, F294), the ZA (V298, L303 and L305), the BC (H355) loops, and C-helix (V357). The following m-phenylenediamine ring is partially accessible to solvent and docks to the channel formed between the ZA (H306 and V311) and BC (Y350, P352, and H355) loops though hydrophobic contacts. The terminal amide does not provide any hydrogen bonding interactions and the terephthalamide ring docks to a shallow hydrophobic surface consisting of N307 and V311 side chains at the ZA loop (FIG. 8A). Two ordered water molecules mediate BRDT-BD2/9 interactions at the ZA channel and the KAc binding pocket. The backbone carbonyl oxygen atoms of P293 and M343 form hydrogen bonds with the aniline and indazole nitrogens through ordered waters.

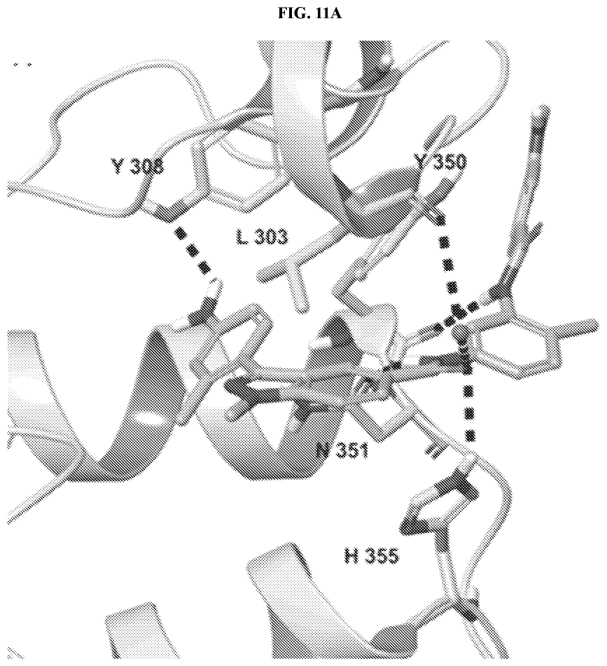

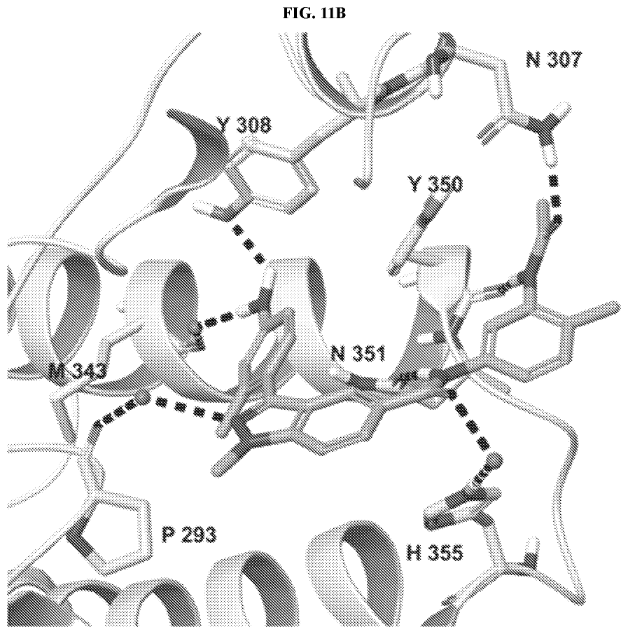

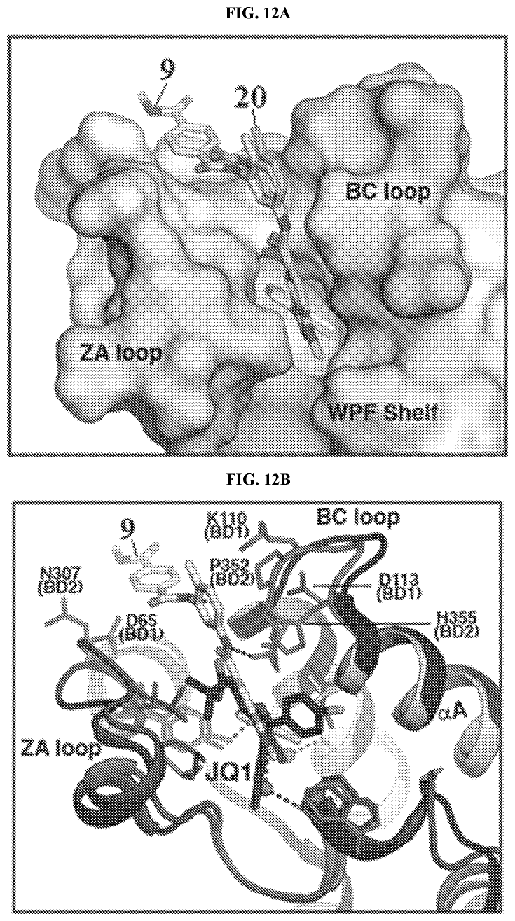

The BRDT-BD2/20 complex revealed that 20 binds similarly as 9 excluding the contacts with the missing methyl benzamide ring (FIG. 8B, FIGS. 11A-11B, and FIG. 12A). As mentioned, a segment of the ZA loop containing H306 and N307 moves away slightly (FIG. 10B) and this allows the side chain of N307 to move closer to 20 for a new hydrogen bond with the terminal amide oxygen (FIG. 8B). Also unique to the BRDT-BD2/20 complex is a water-mediated interaction between the side chain of H355 and the internal amide oxygen. To show the hydrogen bonds formed by the 9 and 20 to the binding pocket more clearly, the co-crystal structures of 9 and 20 were subjected to a restrained geometry optimization after the addition of hydrogens, estimation of protonation states, and optimization of hydrogen bond networks via the Schrodinger program suite (FIGS. 11A-11B).

Despite having two 20 molecules bound, molecules C and D show highly similar structures as the BRDT-BD2/9 complex including the ZA loop. As for the 20 bound at the KAc pocket, their interactions are nearly identical to those seen in the BRDT-BD2/9 complex excluding the contacts with the missing terephthalamide ring and one hydrogen bond (FIG. 8C and FIGS. 11A-11B). The side chain of Y308 moves away slightly and no longer forms a hydrogen bond with the aniline nitrogen. The secondary 20 molecule binds to the back side of the BC loop through hydrogen bonds and stacking interactions. Backbone carbonyl oxygens of Y348 and N351 form hydrogen bonds with the amide nitrogens. Additionally, two consecutive prolines, P352 and P353, are perfectly positioned to interact with the m-phenylenediamine and indazole rings through hydrophobic interactions (FIG. 8C). The aniline ring is fully exposed to solvent showing no interaction with BD2.

Comparison of the BRDT-BD2/9 complex with the BRDT-BD1/(+)-JQ1 complex explains the high BRDT-BD2 versus BRDT-BD1 selectivity observed for 9 and 20 (FIG. 12B). As shown in FIGS. 8A-8C, the indazole and m-phenylenediamine rings make extensive hydrophobic contacts with P352 and H355 at the BC loop as well as a water-mediated hydrogen bond with H355. These conserved BRDT/BRD4-BD2 domain residues are replaced with Lys and Asp (K110 and D113) in BD1, significantly different residues in terms of hydrophobicity and electrostatic charge; it is hypothesized that these changes are responsible for the observed selectivity for BRDT-BD2 versus BDRT-BD1 (FIG. 12B). Without wishing to be limited by any theory, the relatively compact (+)-JQ1 shows no interaction with any of the aforementioned residues which may explain its low BRDT-BD2 versus BRDT-BD1 selectivity. The BRDT-BD2/20 complex shows additional BD2-specific contacts that may contribute its selectivity. As mentioned, the internal amide nitrogen interacts with N351 through a direct hydrogen bond whereas the internal amide oxygen interacts with the BD2-specific H355 through a water (FIG. 8B and FIGS. 11A-11B).

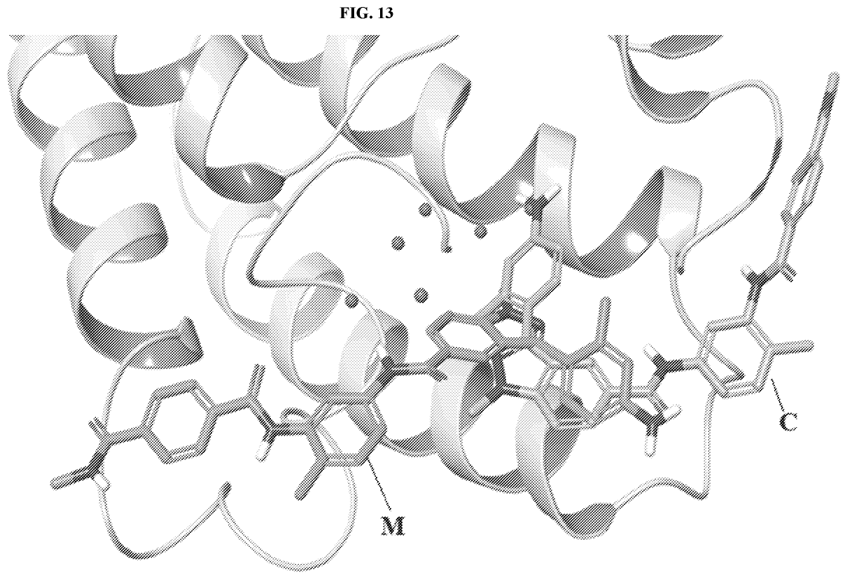

The structures revealed reorganization of solvent molecules upon inhibitor binding. Unseen in previously published structures, both 9 and 20 bind deep into the KAc pocket, replacing a conserved solvent molecule. This water molecule bridges a conserved Tyr (Y308 in BRDT-BD2) to the carbonyl oxygen of KAc on the histone or the bound small molecule inhibitor acting as a second handle in binding. The same carbonyl oxygen of KAc also interact with the conserved N351, further highlighting its crucial role. The computationally modeled binding poses prior to the production of co-crystals retained this water, whose replacement in the co-crystals was surprising (FIG. 13). To date, there has been only one other BET inhibitor documented to replace this conserved ZA channel water, albeit yet with only micromolar affinity and selective for BRD4-BD1 over BRD4-BD2 (Divakaran, A. et al., J. Med. Chem., 2018, 61:9316-9334). Lastly, the structures show a conserved water molecule in the ZA channel that bridges the indazole nitrogen and the backbone carbonyl group of P293 (FIGS. 11A-11B). P293 belongs to the conserved WPF shelf motif of BET proteins; hence, this water-mediated interaction of compound 27 is significant. P293's contribution to binding is also supported by the inactivity of compound 27, which possesses a 2-methyl-2H-indazole blocking water-mediated H-bonding to the WPF shelf.

In summary, the drug discovery campaign to identify BRDT selective inhibitors with DNA-encoded chemical libraries identified a chemical series with potent and selective binding to the second bromodomain of BET family proteins. After off-DNA hit confirmation, the potency and the selectivity of these compounds were validated in multiple in vitro biochemical and biophysical binding assays. Compound 9 was identified as a potent, BD2 versus BD1 selective compound with enhanced metabolic stability over other compounds. Optimization studies led to identification of 20 with similar potency, selectivity, and metabolic stability with significantly reduced molecular weight and less hydrogen bond donors. Co-crystal structures of BRDT-BD2 with 9 and 20 were solved and binding modes were determined to be similar to one another. The medicinal chemistry optimization strategy also allowed for the identification of 15 as a molecule with >6-fold higher affinity for BRDT-BD2 versus BRD4-BD2, indicating that this molecule can have a more potent effect in the testis versus somatic cells. Cumulatively, the results have identified structurally unique BET-BD2-specific compounds with both strong potency and improved selectivity compared to recent reports.

### Enumerated Embodiments

The following exemplary embodiments are provided, the numbering of which is not to be construed as designating levels of importance:

Embodiment 1 provides a compound of formula (I):

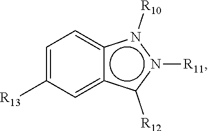

wherein
R₁₀ is present and R₁₁ is absent, or R₁₀ is absent and R₁₁ is present,
   and when present R₁₀ and Ru are each independently selected from hydrogen, deuterium, and C₁-C₁₂ alkyl;
R₁₃ is selected from optionally substituted C₆-C₁₂ aryl and optionally substituted C₄-C₁₀ heteroaryl;
R₁₃ is selected from

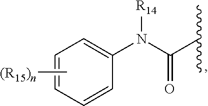

—C(═O)NHR₁₆, and optionally substituted C₄-C₁₀ heteroaryl;
R₁₄ is selected from hydrogen, deuterium, and C₁-C₁₂ alkyl;
each R₁₅ is independently selected from hydrogen, deuterium, halogen, C₁-C₁₂ alkyl, C₁-C₁₂ alkoxy, C₆-C₁₂ aryl, —CN, —CO₂H, —NHS(═O)₂CH₃, —S(═O)₂NHCH₃, —S(═O)₂NH₂, —O—(CH₂)ₘ—C₃-C₈ cycloalkyl, —NHC(═O)—R₁₇, —C(═O)NH—R₁₈, and —C(R₁₉)₃;
R₁₆ is selected from C₁-C₁₂ alkyl and optionally substituted C₄-C₁₀ heterocyclyl;
R₁₇ and R₁₈ are each independently selected from hydrogen, deuterium, C₁-C₁₂ alkyl, optionally substituted C₆-C₁₂ aryl, optionally substituted C₃-C₈ cycloalkyl, and optionally substituted C₄-C₁₀ heterocyclyl,
each R₁₉ is independently selected from fluorine, chlorine, bromine, and iodine;
m is 1, 2, 3, 4, or 5; and
n is 0, 1, 2, 3, 4, or 5;
   or a salt, solvate, stereoisomer, tautomer, or geometric isomer thereof.

Embodiment 2 provides the compound of Embodiment 1, wherein R₁₂ is selected from phenyl, indolyl, pyridyl, and pyrimidinyl, wherein each of which is independently optionally substituted with one or more substituents selected from morpholine, piperazine, halogen, C₁-C₁₂ alkyl, amide, C₁-C₁₂ alkoxy, C₆-C₁₂ benzyloxy, and —NH₂.

Embodiment 3 provides the compound of Embodiment 1 or 2, wherein R₁₂ is selected from

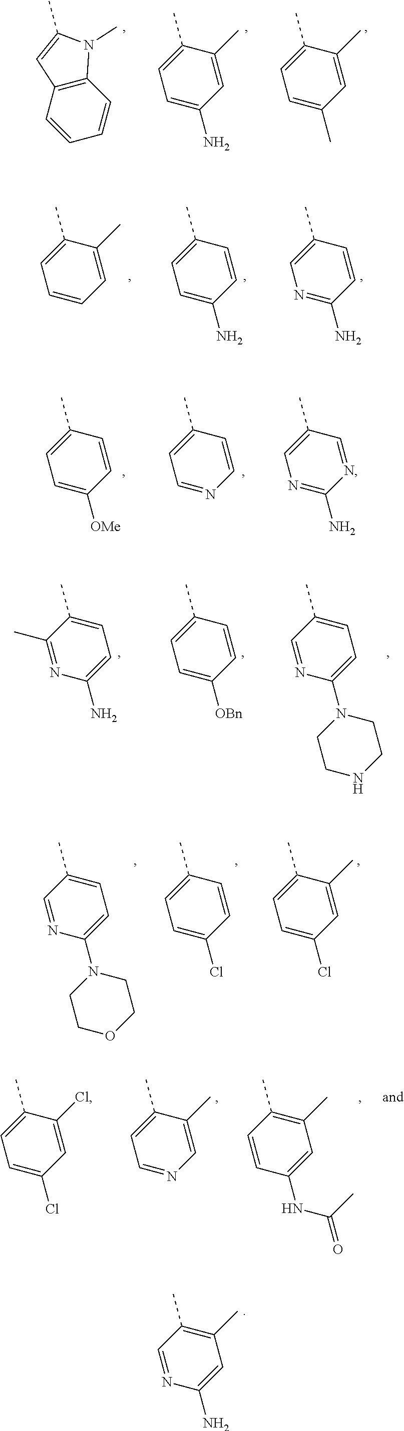

Embodiment 4 provides the compound of any one of Embodiments 1-3, wherein one of the following applies:

(i) R₁₃ is

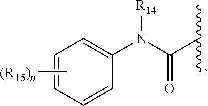

wherein at least one R₁₅ is selected from methyl, methoxy, ethoxy, phenyl, fluorine, chlorine, bromine, iodine, —CO₂H, —C(═O)NH₂, —NHS(═O)₂CH₃, —S(═O)₂NHCH₃, —S(═O)₂NH₂, —CF₃, —CN,

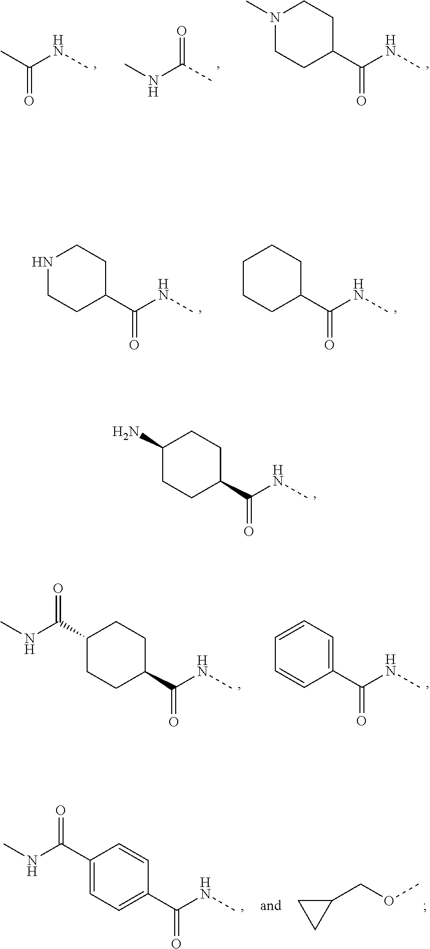

(ii) R₁₃ is —C(═O)NHR₁₆, wherein R₁₆ is selected from methyl,

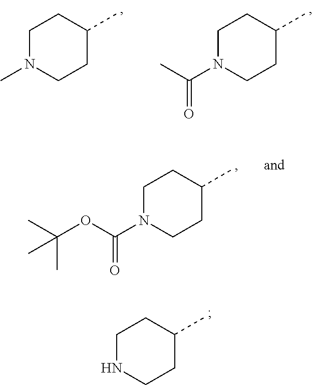

or

(iii) R₁₃ is a C₄-C₁₀ heteroaryl selected from

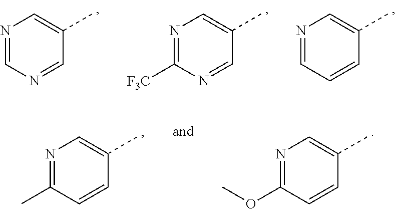

Embodiment 5 provides the compound of Embodiment 1, wherein the compound of formula (I) is a compound of formula (II):

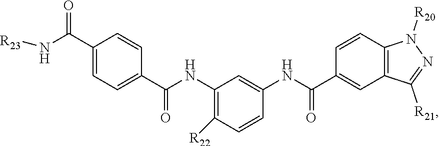

wherein

R₂₀ is C₁-C₁₂ alkyl;

R₂₁ is selected from C₆-C₁₂ aryl and C₄-C₁₀ heteroaryl, wherein each of which is optionally substituted with one or more substituents selected from C₁-C₁₂ alkyl, C₁-C₁₂ alkoxy, C₆-C₁₂ benzyloxy, C₄-C₁₀ heterocyclyl, halogen, and —NH₂;

R₂₂ is selected from C₁-C₁₂ alkyl and C₁-C₁₂ alkoxy; and

R₂₃ is C₁-C₁₂ alkyl.

Embodiment 6 provides the compound of Embodiment 5, wherein R₂₁ is selected from phenyl, indolyl, pyridyl, and pyrimidinyl, wherein each of which is independently optionally substituted with one or more substituents selected from C₁-C₁₂ alkyl, C₁-C₁₂ alkoxy, C₆-C₁₂ benzyloxy, C₄-C₁₀ heterocyclyl, halogen, and —NH₂.

Embodiment 7 provides the compound of Embodiment 5 or 6, wherein R₂₁ is selected from

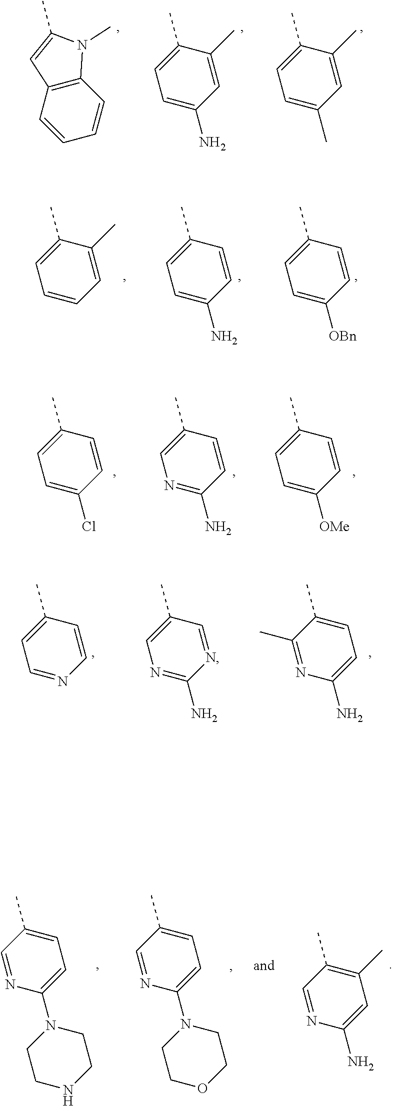

Embodiment 8 provides the compound of any one of Embodiments 5-7, wherein the compound is selected from:

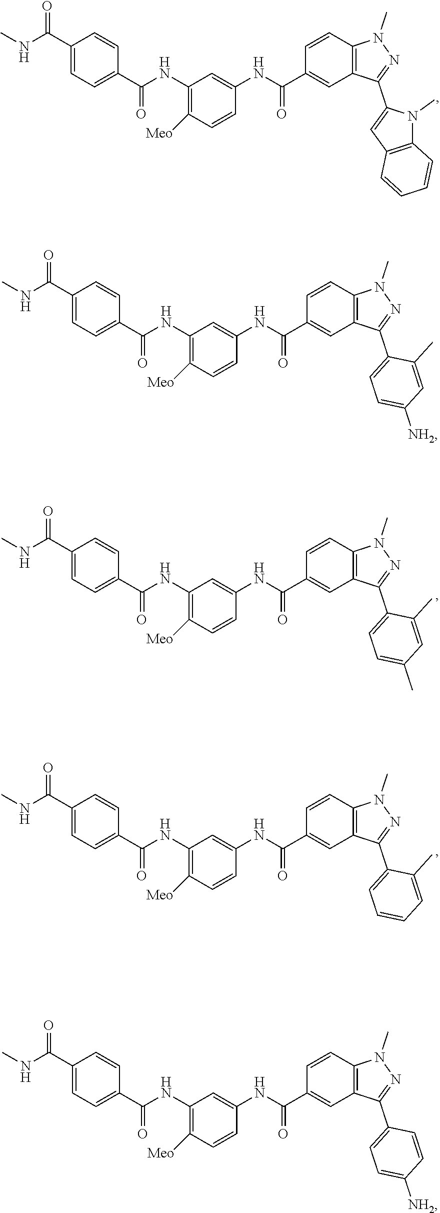

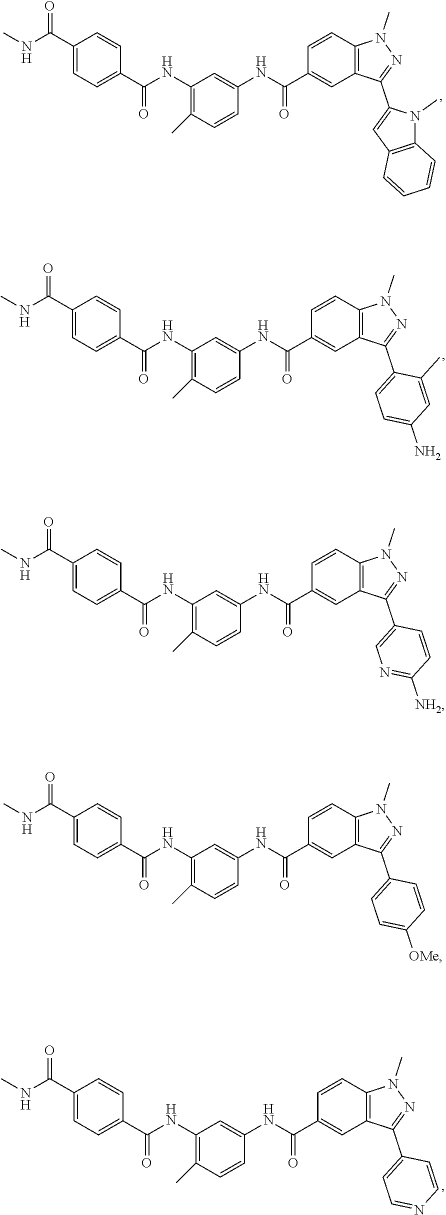

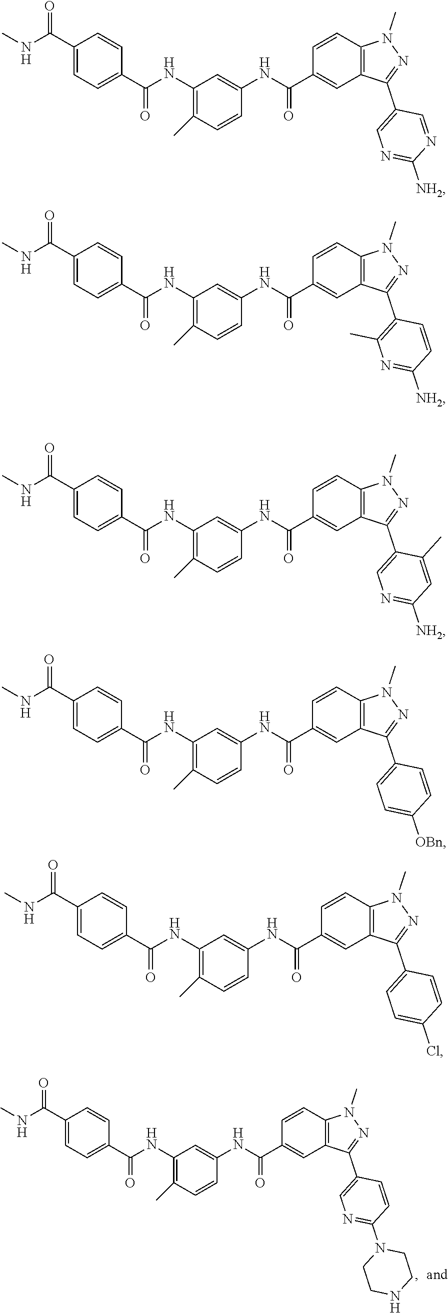

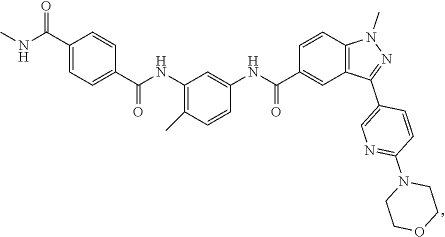 [SMILES: CNC(=O)c1ccc(C(=O)Nc2cc(NC(=O)c3ccc4c(c3)c(-c3ccc(N5CCOCC5)nc3)nn4C)ccc2C)cc1]

wherein OMe is methoxy and OBn is benzyloxy.

Embodiment 9 provides the compound of Embodiment 1, wherein the compound of formula (I) is a compound of formula (III):

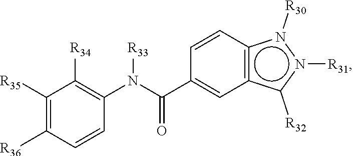

wherein
R₃₀ is present and R₃₁ is absent, or R₃₀ is absent and R₃₁ is present,
   and when present R₃₀ and R₃₁ are each independently C₁-C₁₂ alkyl;
R₃₂ is selected from C₆-C₁₂ aryl and C₄-C₁₀ heteroaryl, wherein each of which is independently optionally substituted with one or more substituents selected from C₁-C₁₂ alkyl, C₁-C₁₂ alkoxy, halogen, amide, and —NH₂;
R₃₃ is selected from hydrogen, deuterium, and C₁-C₁₂ alkyl;
R₃₄, R₃₅, and R₃₆ are each independently selected from hydrogen, deuterium, C₁-C₁₂ alkyl, C₁-C₁₂ alkoxy, C₆-C₁₂ aryl, —CN, —CO₂H, —NHS(═O)₂CH₃, —S(═O)₂NHCH₃, —S(═O)₂NH₂, —O—(CH₂)ₘ—C₃-C₈ cycloalkyl, —C(═O)—NH—R₃₇, —NHC(═O)—R₃₈, —C(R₃₉)₃, and halogen;
R₃₇ and R₃₈ are each independently selected from hydrogen, deuterium, optionally substituted C₁-C₁₂ alkyl, optionally substituted C₆-C₁₂ aryl, optionally substituted C₃-C₈ cycloalkyl, optionally substituted C₄-C₁₀ heterocyclyl,
each R₃₉ is independently selected from fluorine, chlorine, bromine, and iodine; and
m is 1, 2, 3, 4, or 5.

Embodiment 10 provides the compound of Embodiment 9, wherein R₃₂ is selected from phenyl, indolyl, and pyridyl, wherein each of which is independently optionally substituted with one or more substituents selected from C₁-C₁₂ alkyl, C₁-C₁₂ alkoxy, halogen, amide, and —NH₂.

Embodiment 11 provides the compound of Embodiment 9 or 10, wherein R₃₂ is selected from

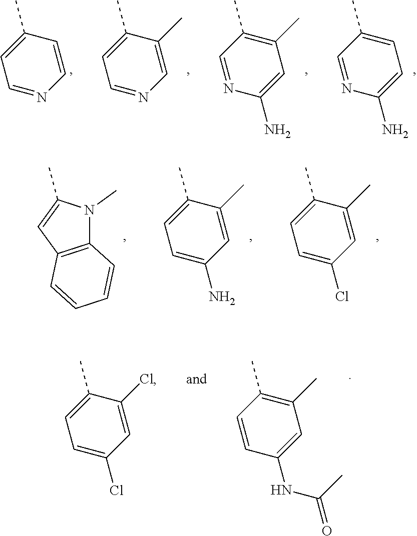

Embodiment 12 provides the compound of any one of Embodiments 9-11, wherein at least one applies:
(i) R₃₄ is selected from methyl, methoxy, —C(═O)NH₂, fluorine, and chlorine;
(ii) R₃₅ is selected from methyl, methoxy, phenyl, fluorine, chlorine, bromine, iodine, —CO₂H, —C(═O)NH₂, —NHS(═O)₂CH₃, —S(═O)₂NHCH₃, —S(═O)₂NH₂, —CF₃, —CN,

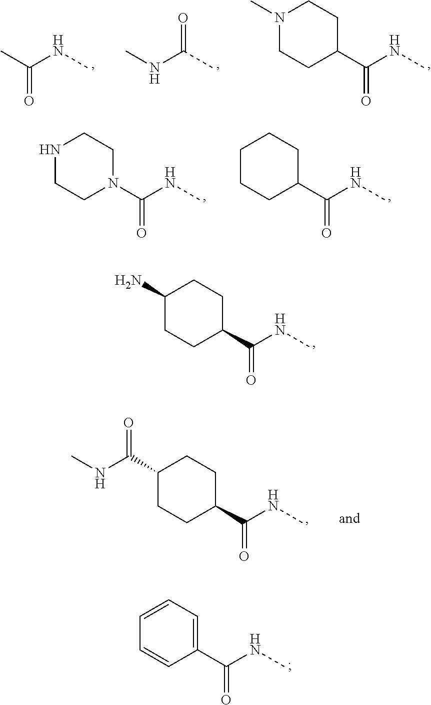

or
(iii) R₃₆ is selected from methyl, methoxy, ethoxy, —CF₃, fluorine, chlorine, and

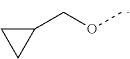

Embodiment 13 provides the compound of any one of Embodiments 9-12, wherein the compound is selected from:

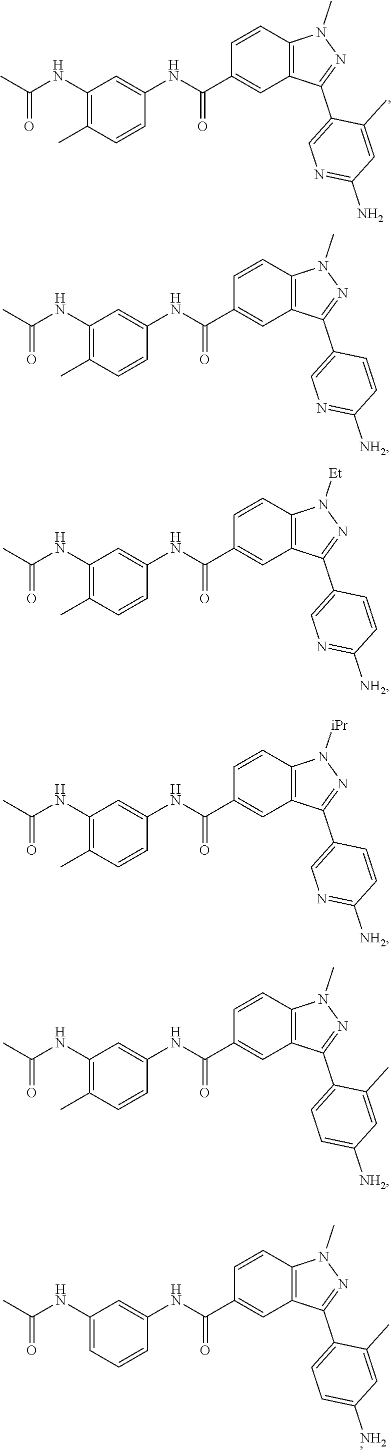

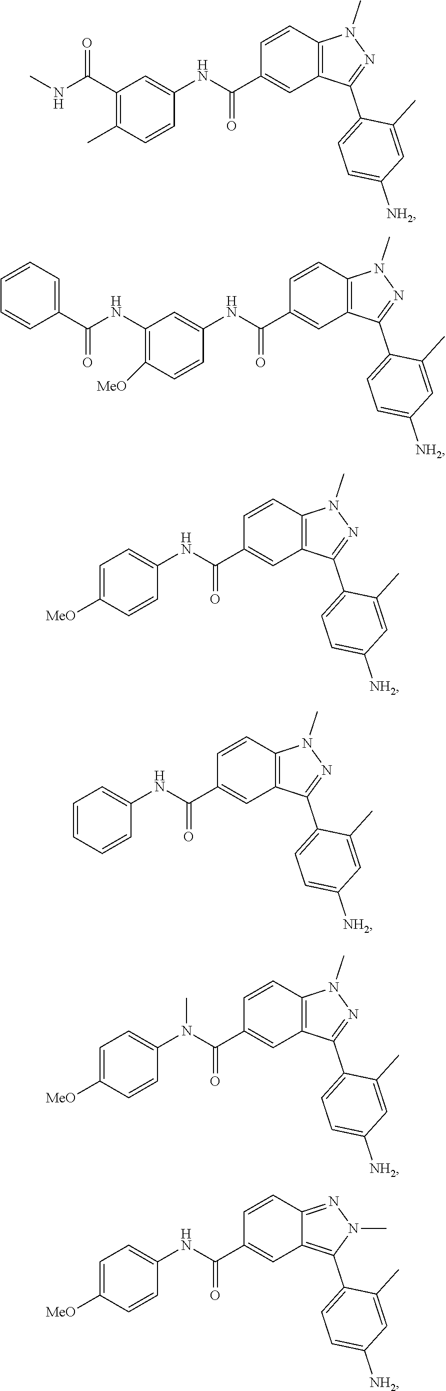

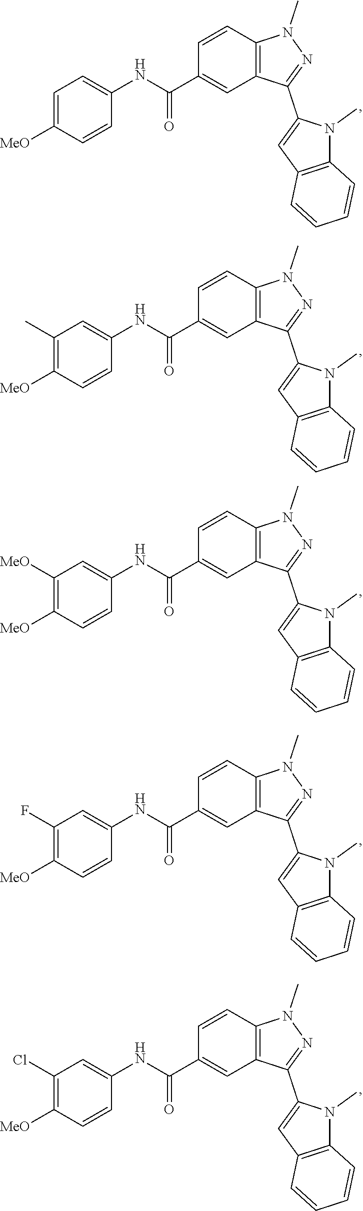

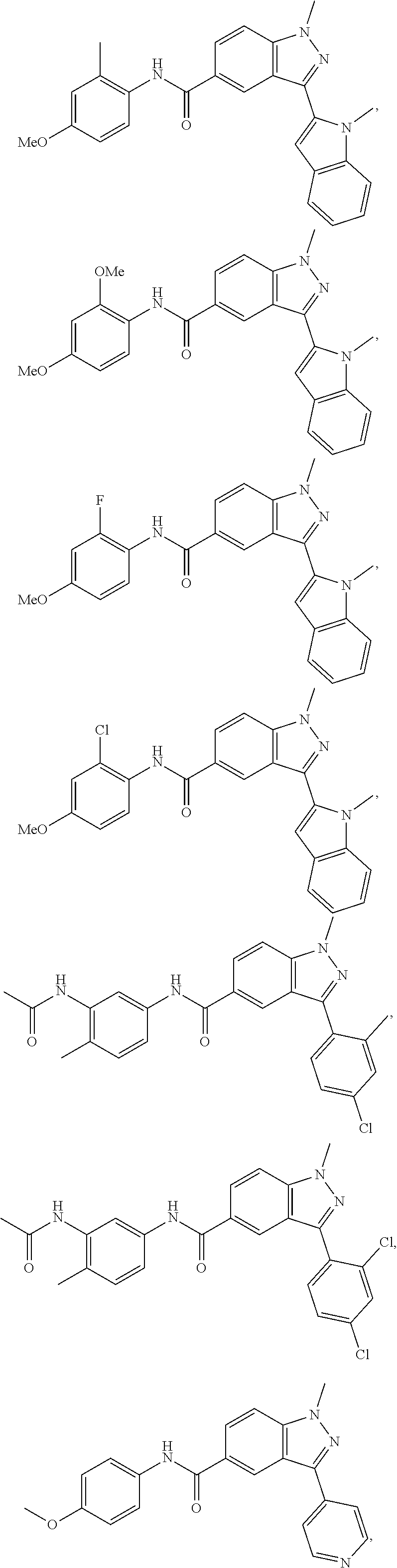

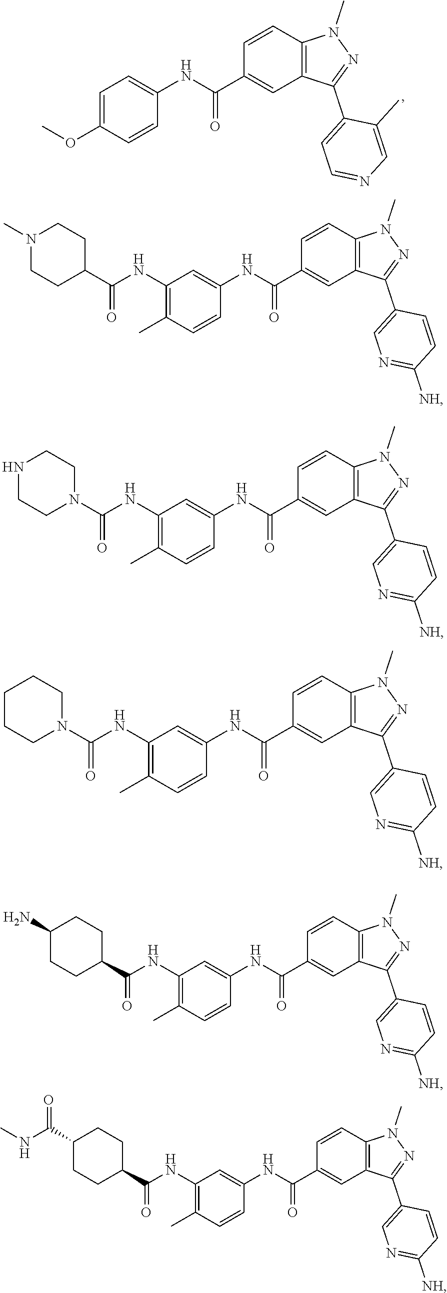

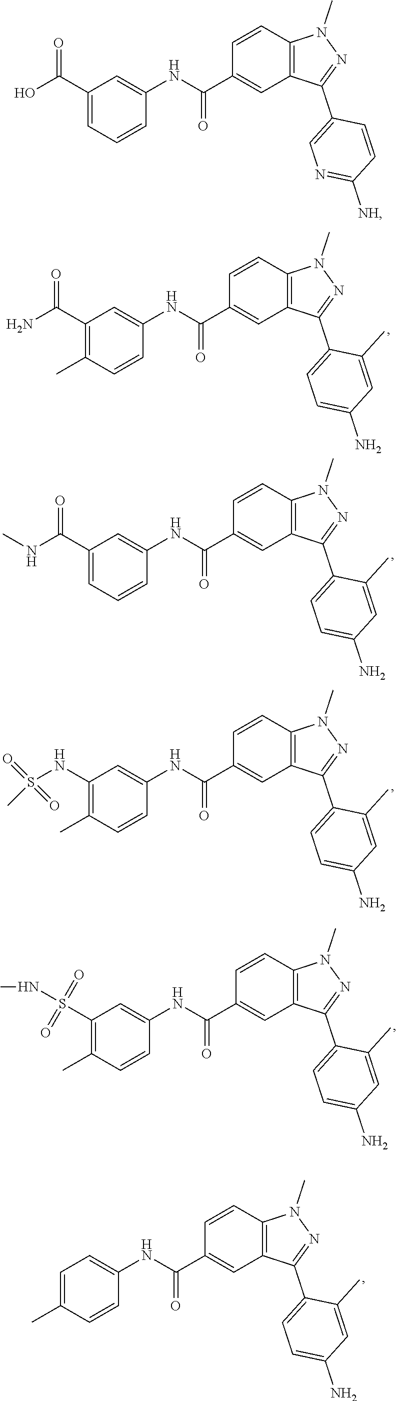

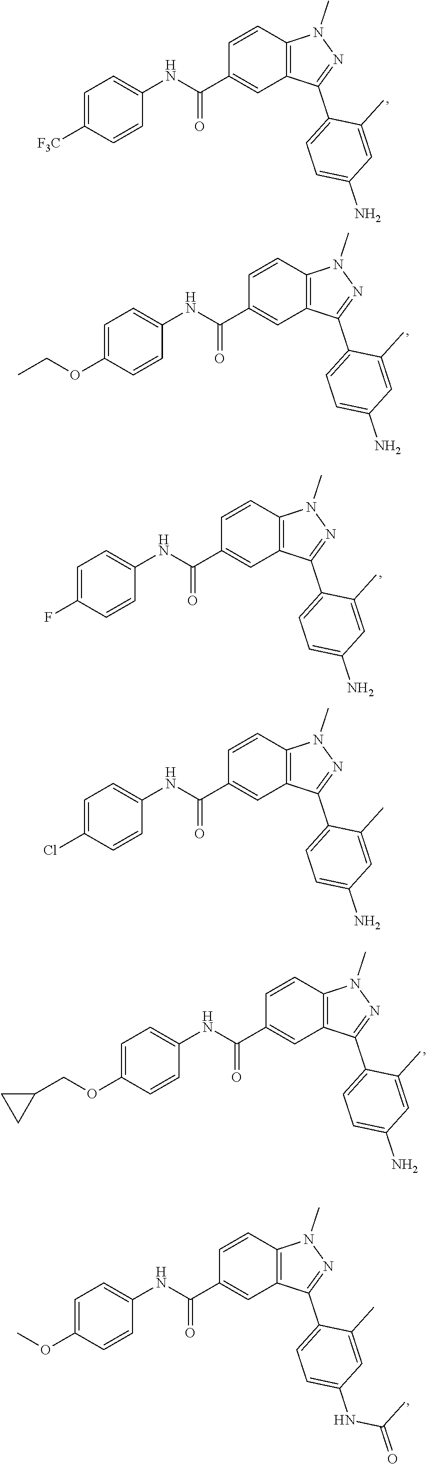

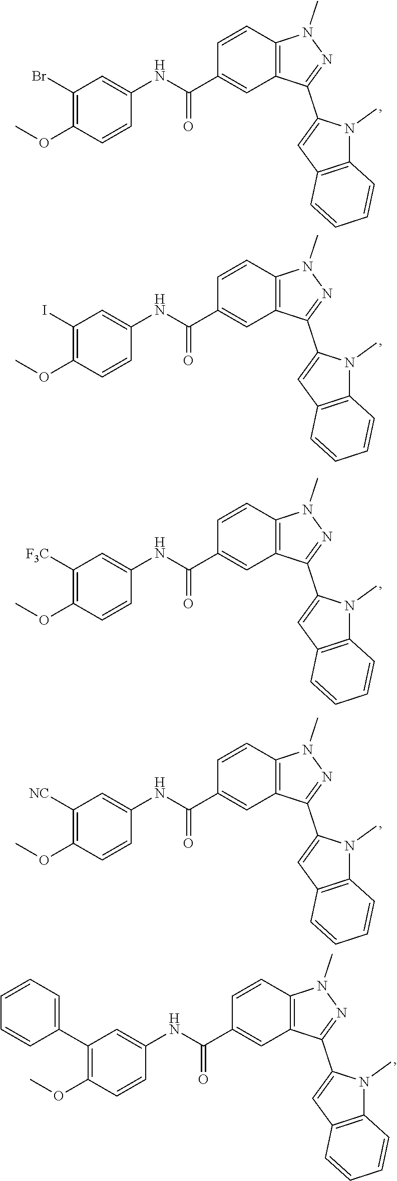

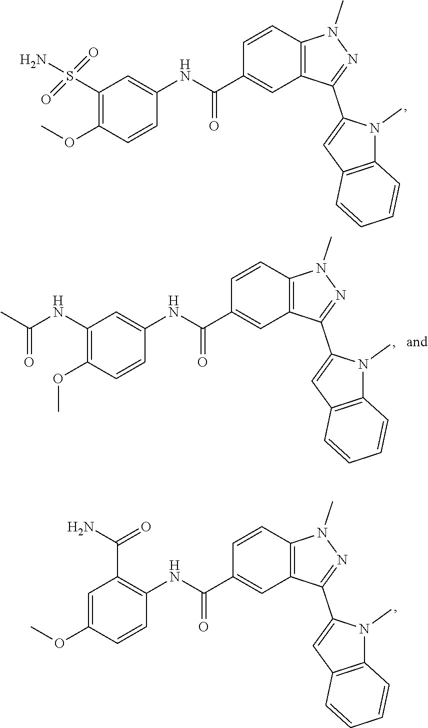

wherein Et is ethyl, iPr is isopropyl, and OMe is methoxy.

Embodiment 14 provides the compound of Embodiment 1, wherein the compound of formula (I) is a compound of formula (IV):

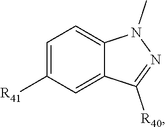

wherein
R₄₀ is selected from C₆-C₁₂ aryl and C₄-C₁₀ heteroaryl, each of which is independently optionally substituted with one or more substituents selected from C₁-C₁₂ alkyl and —NH₂;
R₄₁ is selected from —C(═O)NHR₄₂ and C₄-C₁₀ heteroaryl optionally substituted with one or more substituents selected from C₁-C₁₂ alkyl, C₁-C₁₂ alkoxy, and —C(R₄₃)₃;
R₄₂ is selected from C₁-C₁₂ alkyl and C₄-C₁₀ heterocyclyl optionally substituted with one or more substituents selected from C₁-C₁₂ alkyl and —C(═O)R₄₄;
each R₄₃ is independently selected from fluorine, chlorine, bromine, and iodine; and R₄₄ is selected from C₁-C₁₂ alkyl and C₁-C₁₂ alkoxy.

Embodiment 15 provides the compound of Embodiment 14, wherein R₄₀ is selected from phenyl, indolyl, and pyridyl, wherein each of which is independently optionally substituted with one or more substituents selected from C₁-C₁₂ alkyl and —NH₂.

Embodiment 16 provides the compound of Embodiment 14 or 15, wherein R₄₀ is selected from

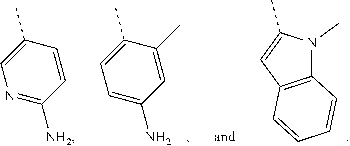

Embodiment 17 provides the compound of any one of Embodiments 14-16, wherein one of the following applies:
(i) R₄₁ is —C(═O)NHR₄₂, wherein R₄₂ is selected from methyl,

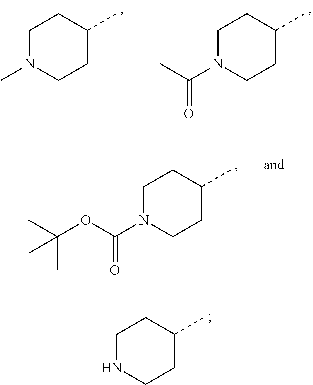

or
(ii) R₄₁ is selected from

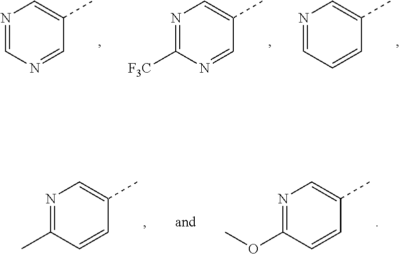

Embodiment 18 provides the compound of any one of Embodiments 14-17, wherein the compound is selected from:

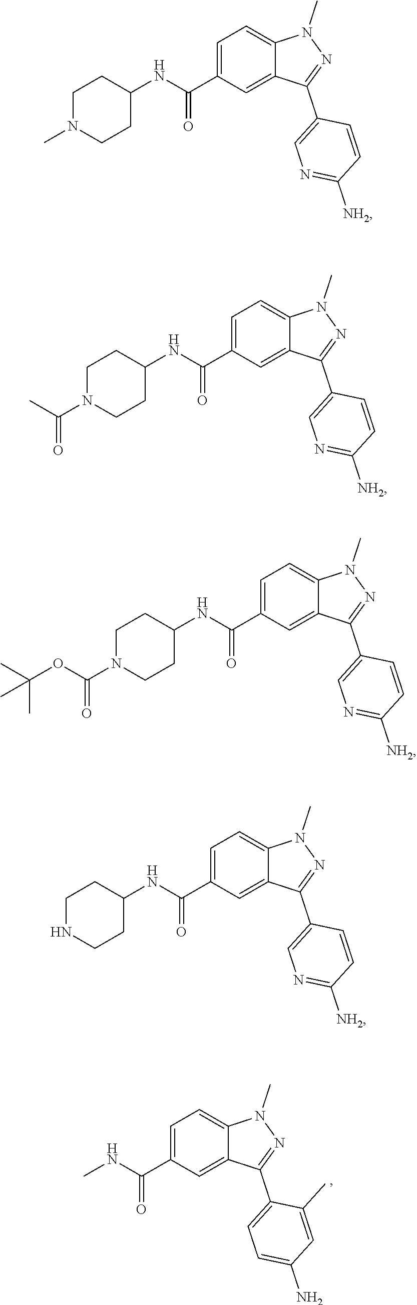

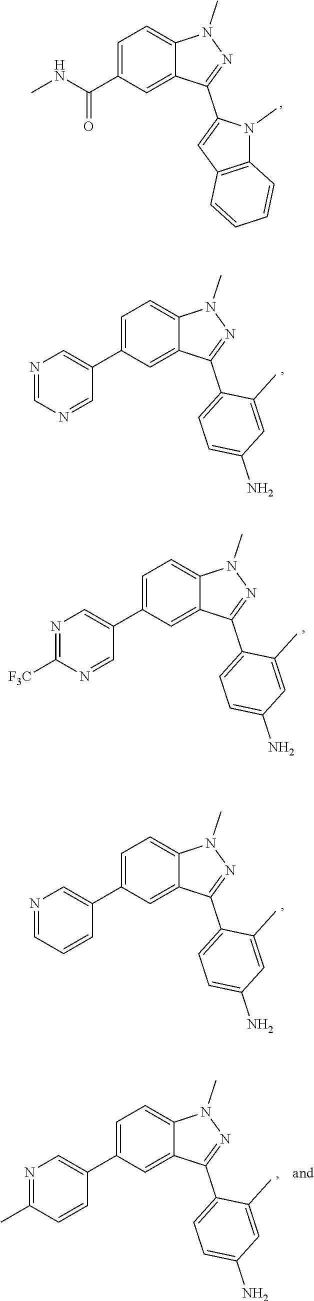

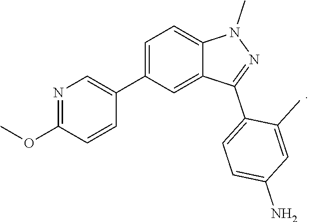

Embodiment 19 provides a pharmaceutical composition comprising the compound of any one of Embodiments 1-18 and a pharmaceutically acceptable carrier.

Embodiment 20 provides a method of inhibiting bromodomain testis (BRDT) in a male subject, the method comprising administering to the male subject a therapeutically effective amount of at least one compound of any one of Embodiments 1-18, and/or the pharmaceutical composition of Embodiment 19.

Embodiment 21 provides a method of promoting male contraception and/or infertility in a male subject, the method comprising administering to the male subject a therapeutically effective amount of at least one compound of any one of Embodiments 1-18, and/or the pharmaceutical composition of Embodiment 19.

Embodiment 22 provides a method of minimizing and/or reducing spermatozoa number and/or motility in a male subject, the method comprising administering to the male subject a therapeutically effective amount of at least one compound of any one of Embodiments 1-18, and/or the pharmaceutical composition of Embodiment 19.

Embodiment 23 provides the method of any one of Embodiments 20-22, wherein the compound inhibits BRDT bromodomain-2 (BRDT-BD2).

Embodiment 24 provides the method of any one of Embodiments 20-23, wherein the compound selectively inhibits BRDT-BD2 over BRDT bromodomain-1 (BRDT-BD1).

Embodiment 25 provides the method of any one of Embodiments 20-24, wherein the compound provides a contraceptive effect in the male subject.

Embodiment 26 provides the method of any one of Embodiments 20-25, wherein the compound is administered orally to the male subject.

The disclosures of each and every patent, patent application, and publication cited herein are hereby incorporated herein by reference in their entirety. While this disclosure has been disclosed with reference to specific embodiments, it is apparent that other embodiments and variations of this disclosure may be devised by others skilled in the art without departing from the true spirit and scope of the disclosure. The appended claims are intended to be construed to include all such embodiments and equivalent variations.

1. A compound of formula (I): 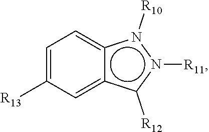
wherein
R₁₀ is present and R₁₁ is absent, or R₁₀ is absent and R₁₁ is present,
and when present R₁₀ and R₁₁ are each independently selected from hydrogen, deuterium, and C₁-C₁₂ alkyl;
R₁₂ is selected from optionally substituted C₆-C₁₂ aryl and optionally substituted C₄-C₁₀ heteroaryl;
R₁₃ is selected from 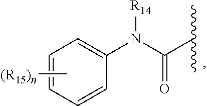
—C(═O)NHR₁₆, and optionally substituted C₄-C₁₀ heteroaryl;
R₁₄ is selected from hydrogen, deuterium, and C₁-C₁₂ alkyl;
each R₁₅ is independently selected from hydrogen, deuterium, halogen, C₁-C₁₂ alkyl, C₁-C₁₂ alkoxy, C₆-C₁₂ aryl, —CN, —CO₂H, —NHS(═O)₂CH₃, —S(═O)₂NHCH₃, —S(═O)₂NH₂, —O—(CH₂)ₘ—C₃-C₈ cycloalkyl, —NHC(═O)—R₁₇, —C(═O)NH—R₁₈, and —C(R₁₉)₃;
R₁₆ is selected from C₁-C₁₂ alkyl and optionally substituted C₄-C₁₀ heterocyclyl;
R₁₇ and R₁₅ are each independently selected from hydrogen, deuterium, C₁-C₁₂ alkyl, optionally substituted C₆-C₁₂ aryl, optionally substituted C₃-C₈ cycloalkyl, and optionally substituted C₄-C₁₀ heterocyclyl,
each R₁₉ is independently selected from fluorine, chlorine, bromine, and iodine;
m is 1, 2, 3, 4, or 5; and
n is 0, 1, 2, 3, 4, or 5;
or a salt, solvate, stereoisomer, tautomer, or geometric isomer thereof.

2. The compound of claim 1, wherein R₁₂ is selected from phenyl, indolyl, pyridyl, and pyrimidinyl, wherein each of which is independently optionally substituted with one or more substituents selected from morpholine, piperazine, halogen, C₁-C₁₂ alkyl, amide, C₁-C₁₂ alkoxy, C₆-C₁₂ benzyloxy, and —NH₂.

3. The compound of claim 1, wherein R₁₂ is selected from

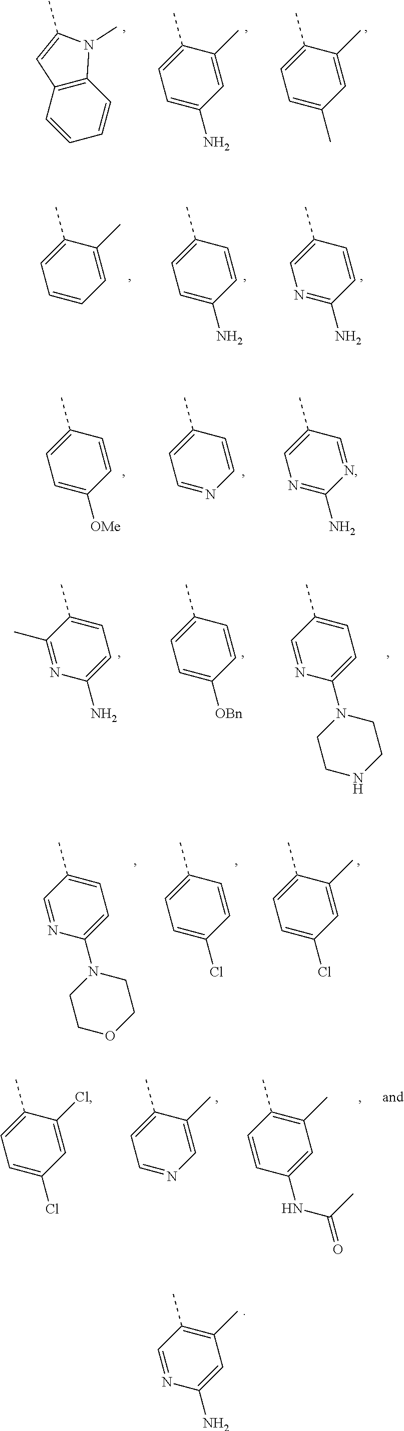

4. The compound of claim 1, wherein one of the following applies:
(i) R₁₃ is 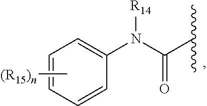
wherein at least one R₁₅ is selected from methyl, methoxy, ethoxy, phenyl, fluorine, chlorine, bromine, iodine, —CO₂H, —C(═O)NH₂, —NHS(═O)₂CH₃, —S(═O)₂NHCH₃, —S(═O)₂NH₂, —CF 3, —CN, 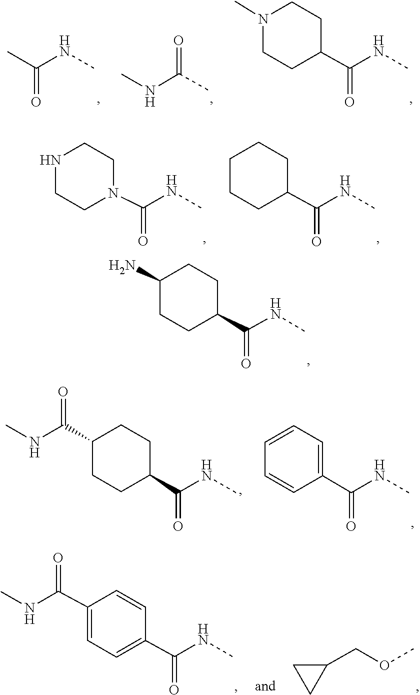
(ii) R₁₃ is —C(═O)NHR₁₆, wherein R₁₆ is selected from methyl, 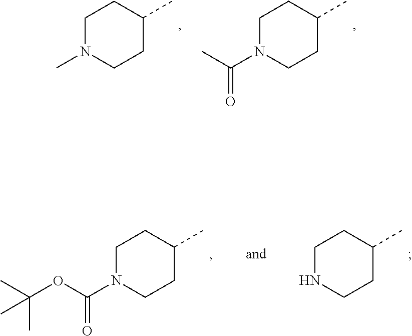
or
(iii) R₁₃ is a C₄-C₁₀ heteroaryl selected from

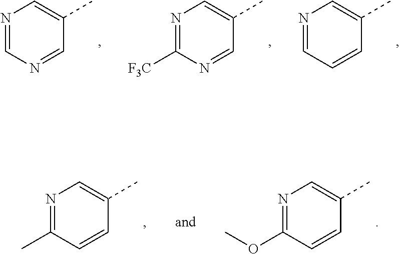

5. The compound of claim 1, wherein the compound of formula (I) is a compound of formula (II): 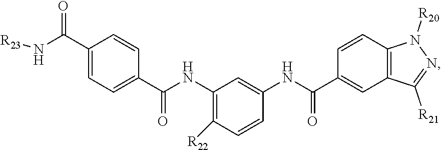
wherein
R₂₀ is C₁-C₁₂ alkyl;
R₂₁ is selected from C₆-C₁₂ aryl and C₄-C₁₀ heteroaryl, wherein each of which is optionally substituted with one or more substituents selected from C₁-C₁₂ alkyl, C₁-C₁₂ alkoxy, C₆-C₁₂ benzyloxy, C₄-C₁₀ heterocyclyl, halogen, and —NH₂;
R₂₂ is selected from C₁-C₁₂ alkyl and C₁-C₁₂ alkoxy; and
R₂₃ is C₁-C₁₂ alkyl.

6. The compound of claim 5, wherein R₂₁ is selected from phenyl, indolyl, pyridyl, and pyrimidinyl, wherein each of which is independently optionally substituted with one or more substituents selected from C₁-C₁₂ alkyl, C₁-C₁₂ alkoxy, C₆-C₁₂ benzyloxy, C₄-C₁₀ heterocyclyl, halogen, and —NH₂.

7. The compound of claim 5, wherein R₂₁ is selected from

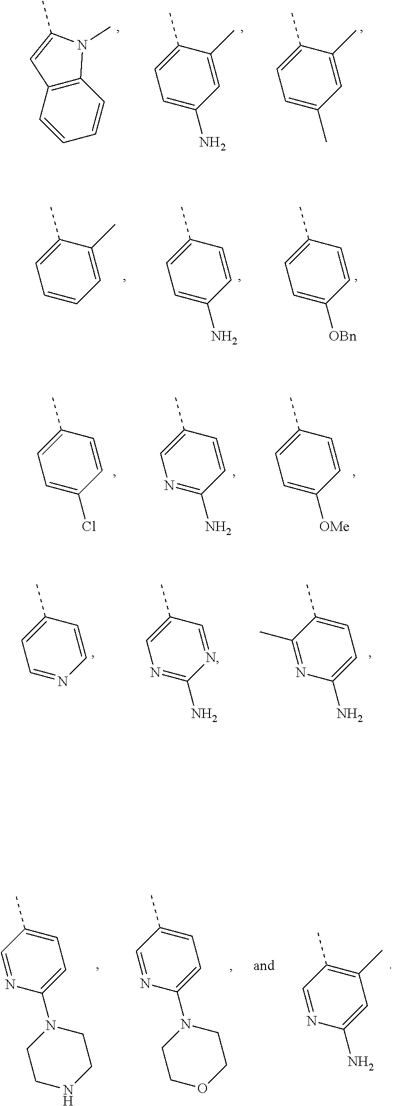

8. The compound of claim 5, wherein the compound is selected from: 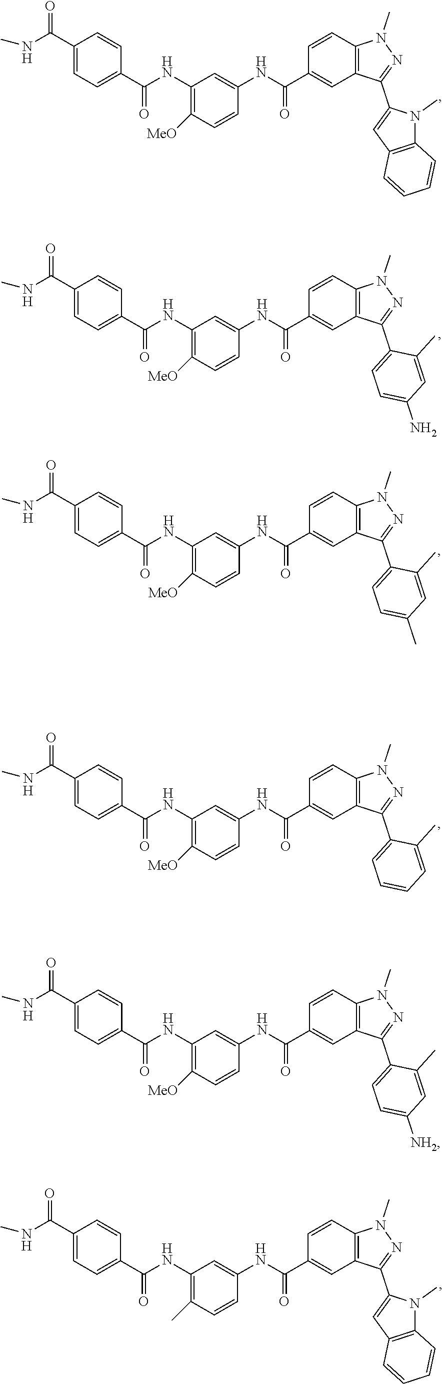 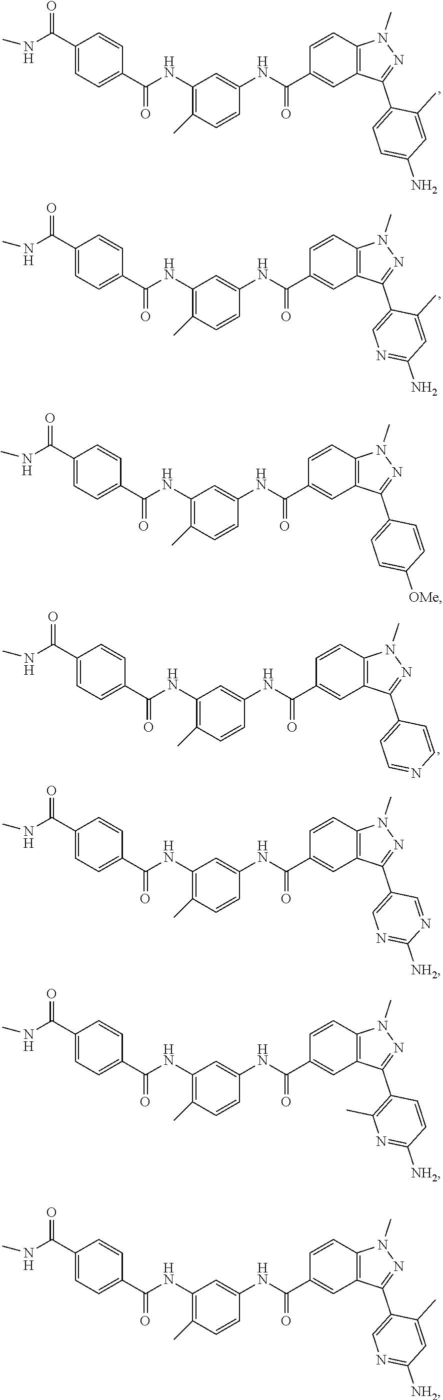 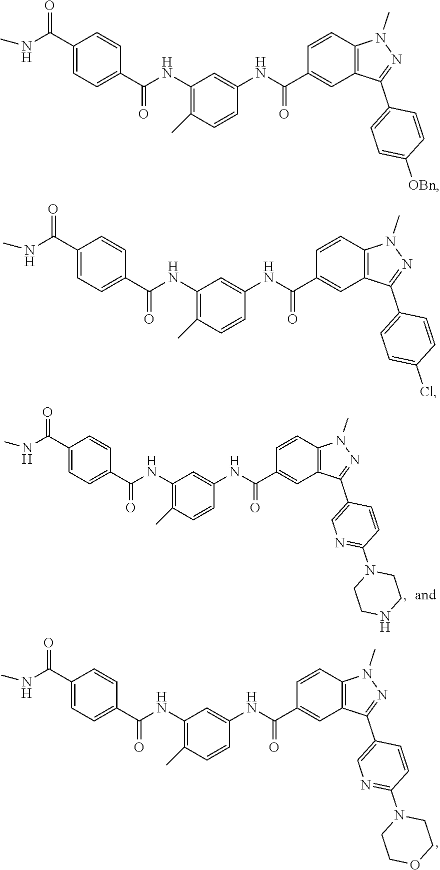
wherein OMe is methoxy and OBn is benzyloxy.

9. The compound of claim 1, wherein the compound of formula (I) is a compound of formula (III): 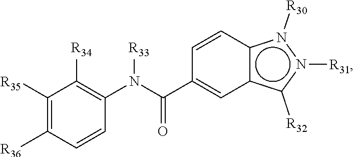
wherein
R₃₀ is present and R₃₁ is absent, or R₃₀ is absent and R₃₁ is present,
and when present R₃₀ and R₃₁ are each independently C₁-C₁₂ alkyl;
R₃₂ is selected from C₆-C₁₂ aryl and C₄-C₁₀ heteroaryl, wherein each of which is independently optionally substituted with one or more substituents selected from C₁-C₁₂ alkyl, C₁-C₁₂ alkoxy, halogen, amide, and —NH₂;
R₃₃ is selected from hydrogen, deuterium, and C₁-C₁₂ alkyl;
R₃₄, R₃₅, and R₃₆ are each independently selected from hydrogen, deuterium, C₁-C₁₂ alkyl, C₁-C₁₂ alkoxy, C₆-C₁₂ aryl, —CN, —CO₂H, —NHS(═O)₂CH₃, —S(═O)₂NHCH₃, —S(═O)₂NH₂, —O—(CH₂)ₘ—C₃-C₈ cycloalkyl, —C(═O)—NH—R₃₇, —NHC(═O)—R₃₈, —C(R₃₉)₃, and halogen;
R₃₇ and R₃₈ are each independently selected from hydrogen, deuterium, optionally substituted C₁-C₁₂ alkyl, optionally substituted C₆-C₁₂ aryl, optionally substituted C₃-C₈ cycloalkyl, optionally substituted C₄-C₁₀ heterocyclyl,
each R₃₉ is independently selected from fluorine, chlorine, bromine, and iodine; and
m is 1, 2, 3, 4, or 5.

10. The compound of claim 9, wherein R₃₂ is selected from phenyl, indolyl, and pyridyl, wherein each of which is independently optionally substituted with one or more substituents selected from C₁-C₁₂ alkyl, C₁-C₁₂ alkoxy, halogen, amide, and —NH₂.

11. (canceled)

12. The compound of claim 9, wherein at least one applies:
(i) R₃₂ is selected from 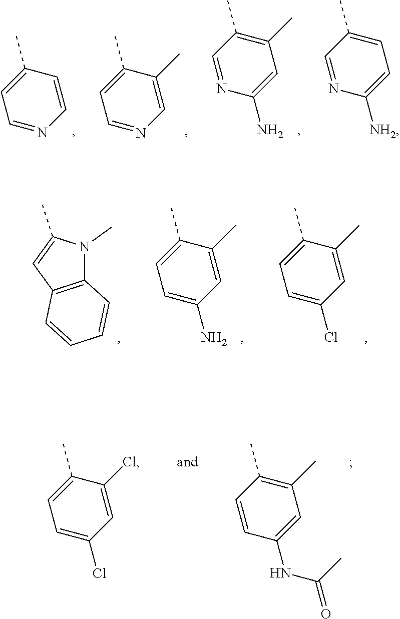
(ii) R₃₄ is selected from methyl, methoxy, —C(═O)NH₂, fluorine, and chlorine;
(iii) R₃₅ is selected from methyl, methoxy, phenyl, fluorine, chlorine, bromine, iodine, —CO₂H, —C(═O)NH₂, —NHS(═O)₂CH₃, —S(═O)₂NHCH₃, —S(═O)₂NH₂, —CF₃, —CN, 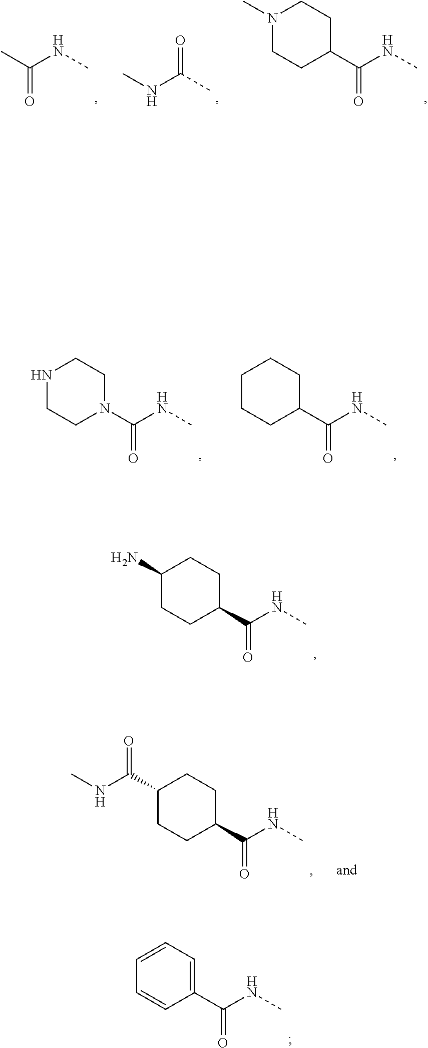
and
(iv) R₃₆ is selected from methyl, methoxy, ethoxy, —CF₃, fluorine, chlorine, and

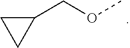

13. The compound of claim 9, wherein the compound is selected from: 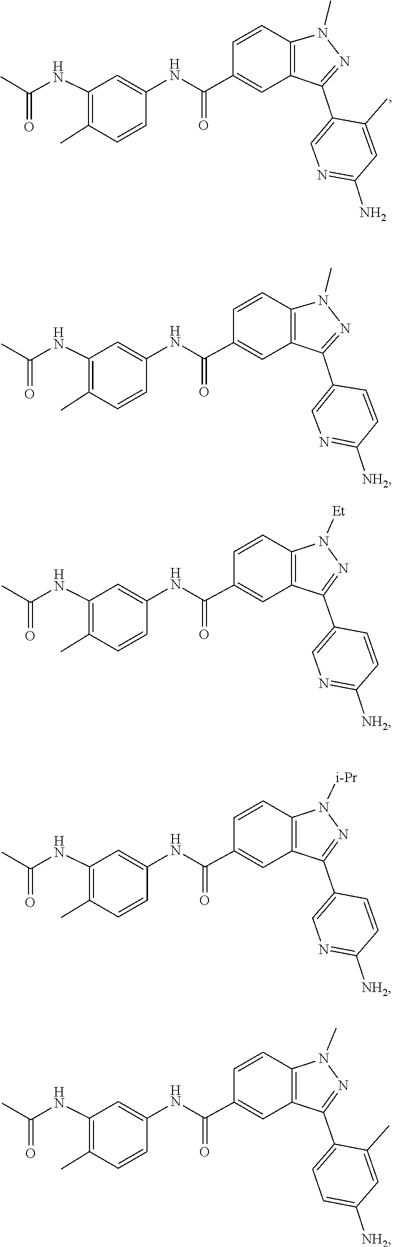 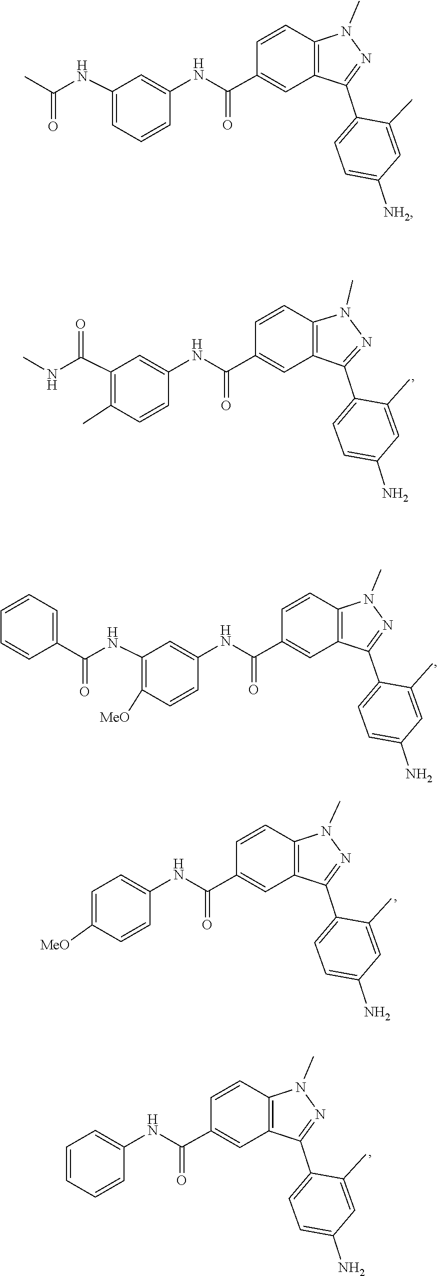 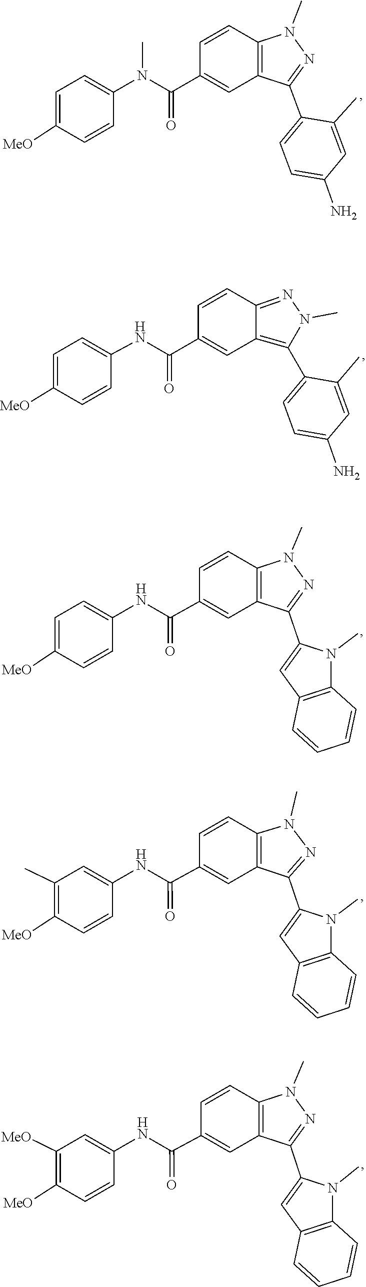 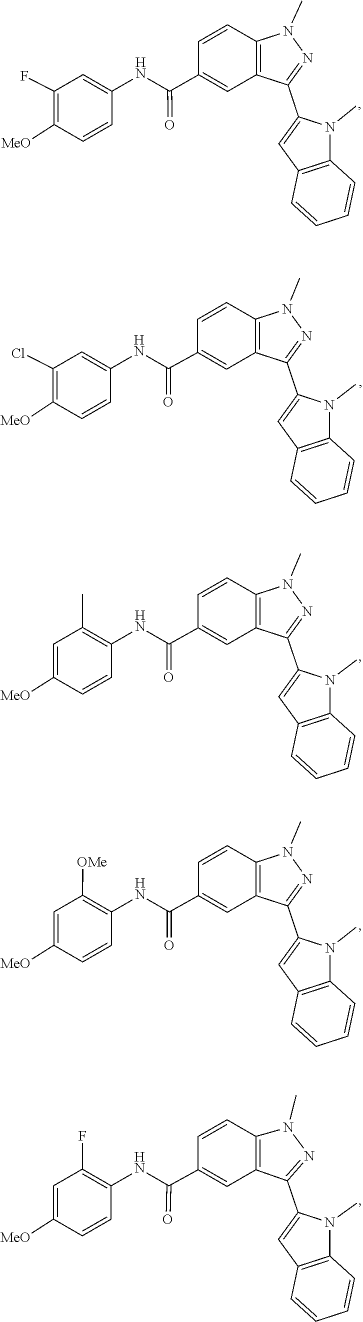 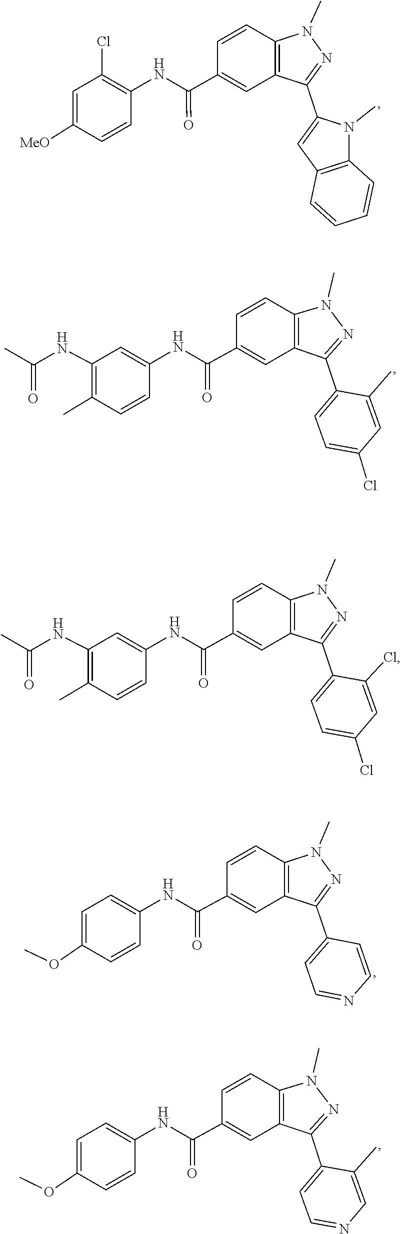 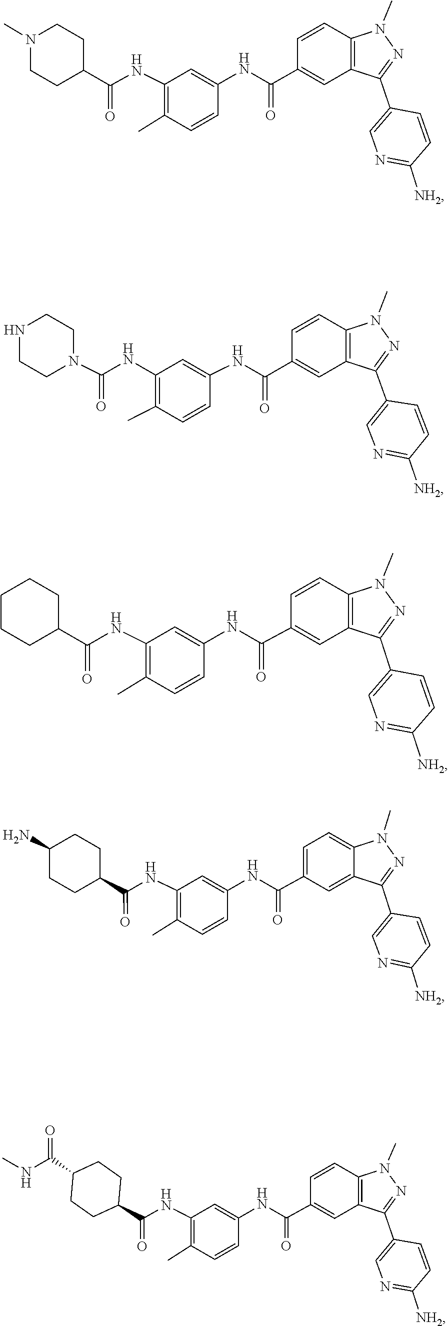 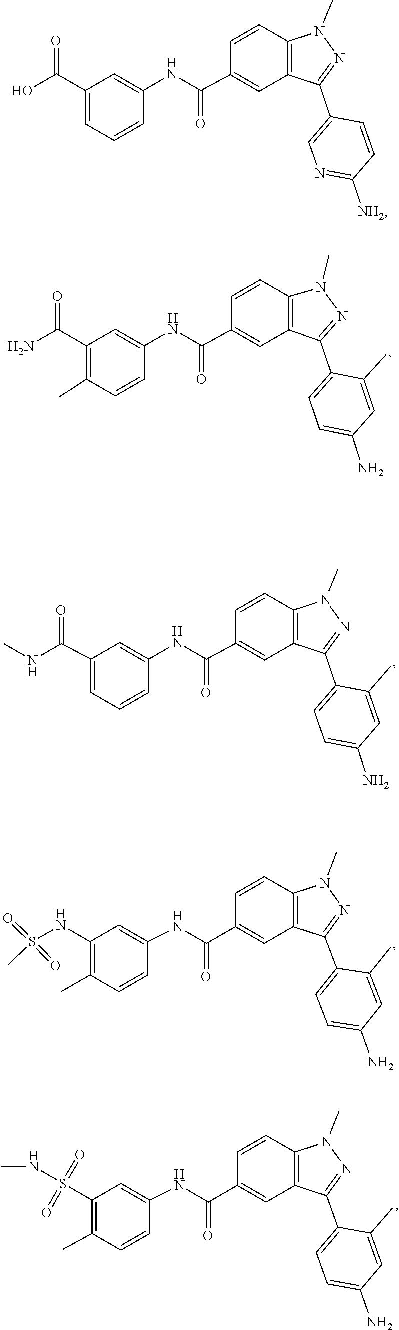 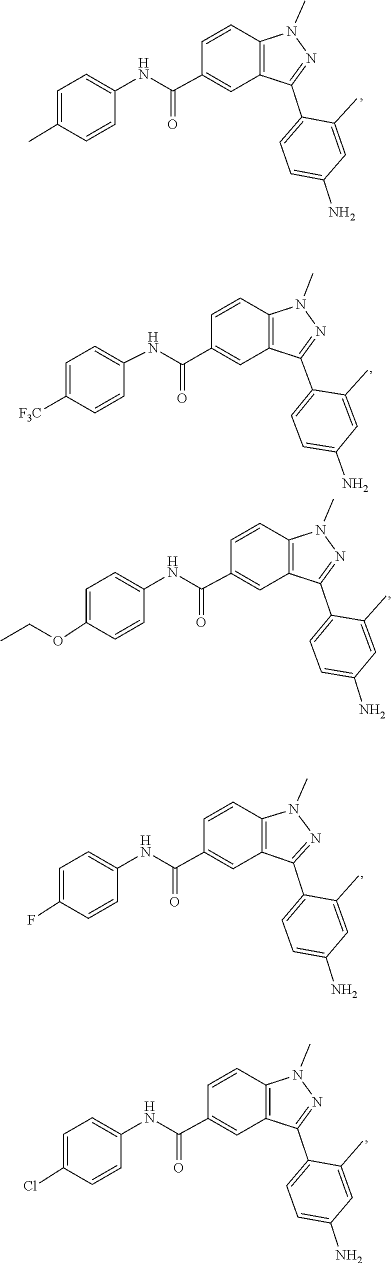 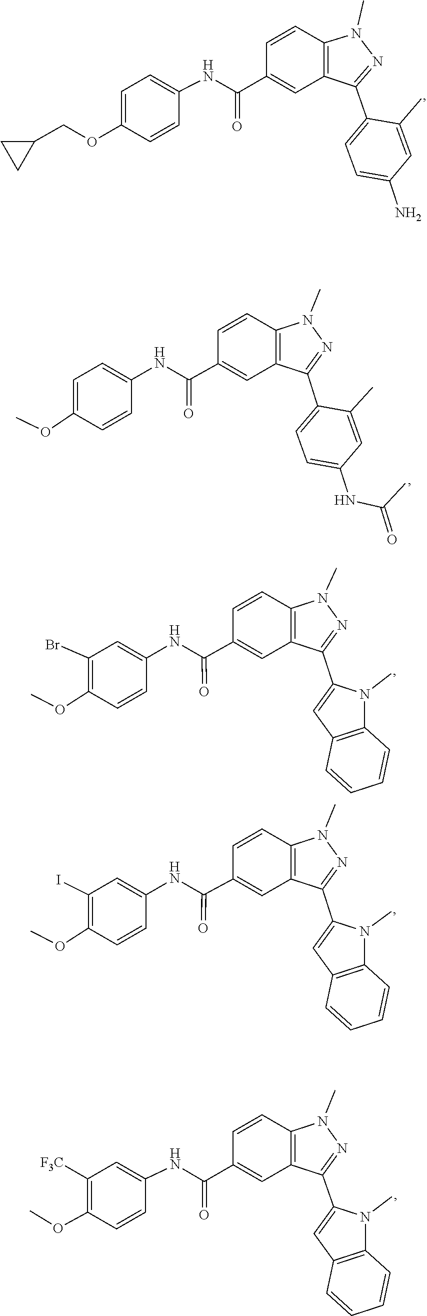 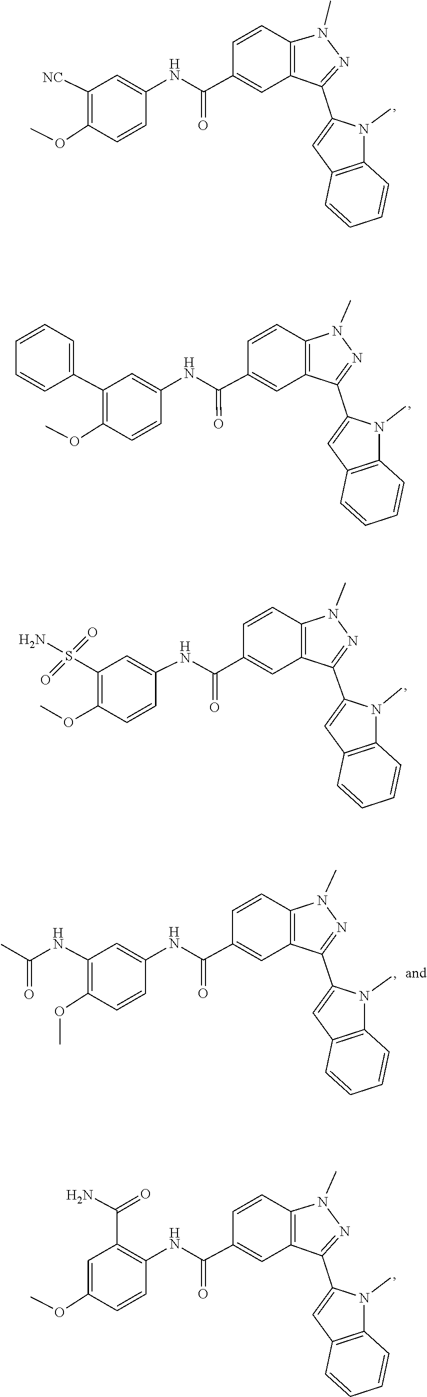
wherein Et is ethyl, iPr is isopropyl, and OMe is methoxy.

14. The compound of claim 1, wherein the compound of formula (I) is a compound of formula (IV): 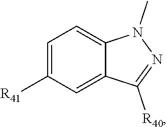
wherein
R₄₀ is selected from C₆-C₁₂ aryl and C₄-C₁₀ heteroaryl, each of which is independently optionally substituted with one or more substituents selected from C₁-C₁₂ alkyl and —NH₂;
R₄₁ is selected from —C(═O)NHR₄₂ and C₄-C₁₀ heteroaryl optionally substituted with one or more substituents selected from C₁-C₁₂ alkyl, C₁-C₁₂ alkoxy, and —C(R₄₃)₃;
R₄₂ is selected from C₁-C₁₂ alkyl and C₄-C₁₀ heterocyclyl optionally substituted with one or more substituents selected from C₁-C₁₂ alkyl and —C(═O)R₄₄;
each R₄₃ is independently selected from fluorine, chlorine, bromine, and iodine; and
R₄₄ is selected from C₁-C₁₂ alkyl and C₁-C₁₂ alkoxy.

15. The compound of claim 14, wherein R₄₀ is selected from phenyl, indolyl, and pyridyl, wherein each of which is independently optionally substituted with one or more substituents selected from C₁-C₁₂ alkyl and —NH₂.

16. The compound of claim 14, wherein R₄₀ is selected from

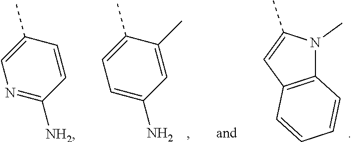

17. The compound of claim 14, wherein one of the following applies:
(i) R₄₁ is —C(═O)NHR₄₂, wherein R₄₂ is selected from methyl, 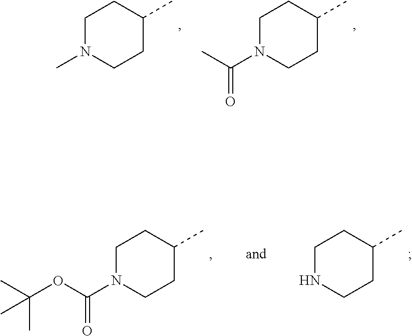
or
(ii) R₄₁ is selected from

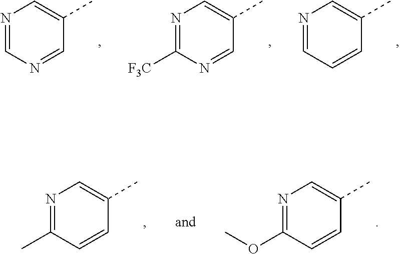

18. The compound of claim 14, wherein the compound is selected from:

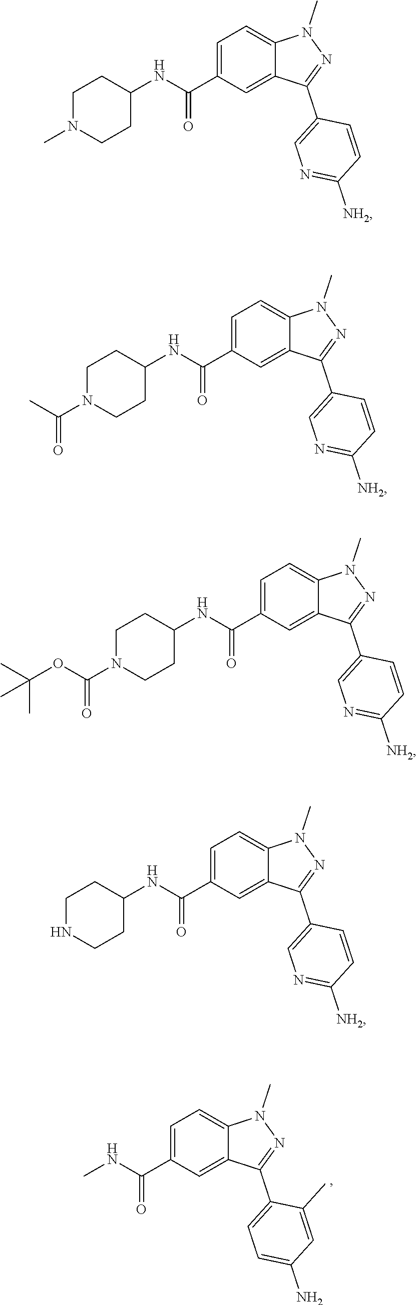

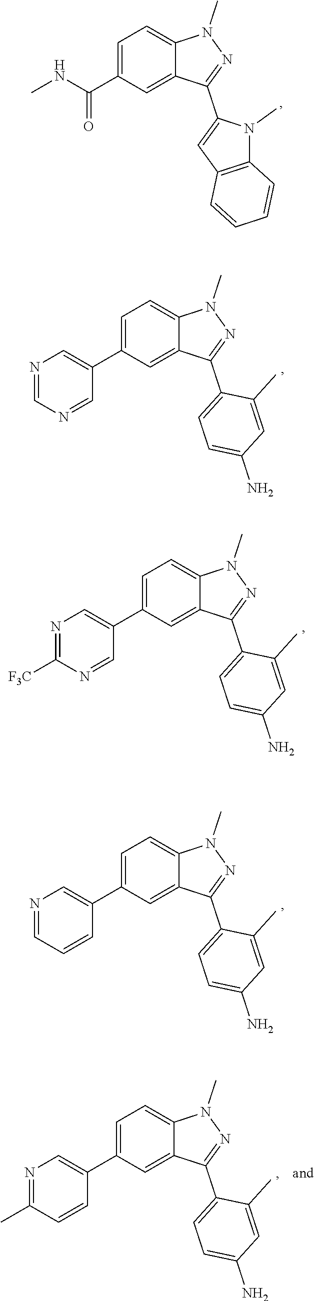

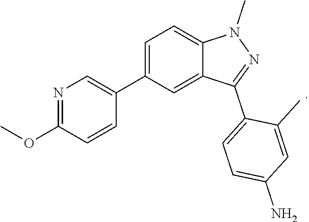

19. A pharmaceutical composition comprising the compound of claim 1 and a pharmaceutically acceptable carrier.

20. A method of inhibiting bromodomain testis (BRDT), promoting male contraception or infertility, or minimizing or reducing spermatozoa number or motility in a male subject, the method comprising administering to the male subject a therapeutically effective amount of at least one compound of claim 1 or a pharmaceutical composition thereof comprising a pharmaceutically acceptable carrier.

21-22. (canceled)

23. The method of claim 20, wherein at least one of the following applies:
(a) the compound inhibits BRDT bromodomain-2, optionally wherein the compound selectively inhibits BRDT-BD2 over BRDT bromodomain-1 (BRDT-BD1); and
(b) the compound is administered orally to the male subject.

24-26. (canceled)
